(19) United States
(12) Patent Application Publication
(10) Pub. No.: US 2026/0234648 A1
(43) Pub. Date: Aug. 13, 2026

(54) METHODS AND COMPOSITIONS FOR ENHANCED EXPRESSION OF EXOGENOUS CASEINS IN TRANSGENIC PLANTS

(71) Applicant: Mozza Foods, Inc., Los Angeles, CA (US)

(72) Inventor: Cory Tobin, Los Angeles, CA (US)

(21) Appl. No.: 19/470,807

(22) PCT Filed: Mar. 27, 2024
(86) PCT No.: PCT/US2024/021715

§ 371 Date: Sep. 30, 2025

Related U.S. Application Data
(60) Provisional application No. 63/456,432, filed on Mar. 31, 2023.
(60) Provisional application No. 63/457,287, filed on Apr. 5, 2023.
(60) Provisional application No. 63/461,147, filed on Apr. 21, 2023.
(60) Provisional application No. 63/471,222, filed on Jun. 5, 2023.
(60) Provisional application No. 63/513,938, filed on Jul. 17, 2023.
(60) Provisional application No. 63/587,683, filed on Oct. 3, 2023.
(60) Provisional application No. 63/594,208, filed on Oct. 30, 2023.
(60) Provisional application No. 63/597,728, filed on Nov. 10, 2023.
(60) Provisional application No. 63/609,368, filed on Dec. 13, 2023.
(60) Provisional application No. 63/617,008, filed on Jan. 2, 2024.
(60) Provisional application No. 63/548,980, filed on Feb. 2, 2024.

(51) Int. Cl. C12N 15/82; C07K 14/47; C07K 14/81; C12N 9/12; C12N 9/22; C12N 15/11; C12N 15/113; C12N 15/67

(52) U.S. Cl. CPC C12N 15/8251; C07K 14/4732; C07K 14/811; C12N 9/12; C12N 9/222; C12N 15/111; C12N 15/1137; C12N 15/67; C12N 15/8205; C12N 15/8207; C12N 15/8218; C07K 2319/95; C12N 2310/20; C12Y 203/02; C12Y 207/11001

(57) ABSTRACT

The present disclosure provides methods and plants by genetically modifying plants to enhance their nutritional quality. In some aspects, the present disclosure provides methods for reducing activity or expression of one or more proteases in a plant (e.g., soybean), leading to increased levels of casein proteins and casein micelles, resulting in plants with enhanced levels of essential amino acids and improved digestibility.

### CROSS-REFERENCE INFORMATION

The instant application is the United States National Phase Entry of International Application Serial No. PCT/US2024/021715, filed on Mar. 27, 2024, which incorporated by reference U.S. Provisional Patent Application Ser. No. 63/456,432, filed Mar. 31, 2023; U.S. Provisional Patent Application Ser. No. 63/457,287, filed Apr. 5, 2023, U.S. Provisional Patent Application Ser. No. 63/461,147, filed Apr. 21, 2023; U.S. Provisional Patent Application Ser. No. 63/471,222, filed Jun. 5, 2023; U.S. Provisional Patent Application Ser. No. 63/513,938, filed Jul. 17, 2023; U.S. Provisional Patent Application Ser. No. 63/587,683, filed Oct. 3, 2023; U.S. Provisional Patent Application Ser. No. 63/594,208, filed Oct. 30, 2023; U.S. Provisional Patent Application Ser. No. 63/597,728, filed Nov. 10, 2023; U.S. Provisional Patent Application Ser. No. 63/609,368, filed Dec. 13, 2023; U.S. Provisional Patent Application Ser. No. 63/617,008, filed Jan. 2, 2024; and U.S. Provisional Patent Application Ser. No. 63/548,980, filed Feb. 2, 2024. In some instances, the methods, systems, techniques, sequences, and compositions disclosed in the present application are utilized in conjunction with those delineated and described in said incorporated provisional applications.

### SEQUENCE LISTING

The instant application contains a Sequence Listing, which has been submitted herewith electronically in XML formation. The contents of the XML file, submitted electronically herewith, are contained within a computer readable format copy of the Sequence Listing, with a filename 713001013USNPPA, which was created on Sep. 29, 2025 and has a file size of 328 kilobytes, are incorporated herein by reference in their entirety:

### FIELD OF THE INVENTION

The present application pertains to the field of agricultural biotechnology and, more particularly, to the enhancement of protein expression in transgenic plants. Specifically, it addresses the challenge of increasing the stability and yield of transgenic proteins, such as casein, in soy plants, which are subject to degradation by endogenous proteases.

### BACKGROUND

Casein micelles account for more than 80% of the protein in bovine milk and are a key component of all dairy cheeses. Casein micelles include individual casein proteins that are produced in the mammary glands of bovines and other animals. The industrial scale production of the milk that is processed to yield these casein micelles, primarily in the form of curds for cheese production, typically occurs on large-scale dairy farms and is often inefficient, damaging to the environment, and harmful to the animals.

Dairy cows contribute substantially to greenhouse gasses, consume significantly more water than the milk they produce, and commonly suffer from dehorning, disbudding, mastitis, routine forced insemination, and bobby calf slaughter. Accordingly, there is a need for an in vivo plant-based casein expression system which allows for expression of casein proteins that is cost effective at industrial scale. Methods of expressing casein and casein micelles in plants are known in the art, for example, U.S. Pat. No. 11,326,176 discloses methods for expressing casein proteins in transgenic plants. U.S. Pat. No. 11,718,856 discloses a plant cell co-expressing at least two ruminant casein proteins. Nonetheless, mere production of some casein and/or casein micelles in a plant is insufficient to meet the enormous societal demand for these proteins unless and until solutions are developed to increase the expression level of exogenous proteins in transgenic plants.

Casein and casein micelles, due to their unique structural and functional properties, find applications across a wide array of fields, serving both traditional and innovative uses. In the food industry, they are utilized as nutritional supplements, emulsifying agents, and texture enhancers in dairy products, infant formula, and various processed foods, leveraging their ability to improve texture, flavor, and nutritional value. In some instances, casein is also employed in the production of cheese, where it acts as a primary protein source facilitating coagulation. Beyond nutritional applications, casein finds utility in the pharmaceutical sector as a vehicle for drug delivery systems, exploiting its biocompatibility and ability to form gels for controlled release formulations. In some cases, casein micelles are explored for their role in nanocarrier systems for targeted therapy, given their natural origin and ability to encapsulate bioactive compounds. In the field of bioplastics, casein-based polymers are investigated for their biodegradability and potential to reduce reliance on fossil fuels. In some cases, casein is used in the cosmetics industry as a hydrating and binding agent in skincare and haircare products, capitalizing on its moisturizing properties. In some instances, casein and casein micelles are applied in adhesive formulations, particularly in wood adhesives, due to their strong binding capabilities and eco-friendly nature. The textile industry utilizes casein in the production of fibers and fabric finishes, offering a sustainable alternative for synthetic materials. In some cases, casein is also found in artistic mediums, such as casein-based paints, prized for their durability and matte finish.

The expression of transgenic proteins in plants is a cornerstone of modern agricultural biotechnology, enabling the production of a wide range of substances, from industrial enzymes to pharmaceuticals. But there are several significant hurdles facing large-scale production of plant-derived casein and casein micelles, such as the degradation of these valuable proteins by plant proteases, the insufficiency of available proline in plants such as soy, a key amino-acid in casein formation, and the tendency of exogenous casein to under-express relative to other proteins in transgenic plants (e.g., Example 2). In the case of soy plants, transgenic casein is particularly vulnerable to degradation throughout its synthesis and storage process. These challenges can result in significant economic losses and reduced effectiveness of the transgenic proteins.

In addition to the challenge posed by plant proteases degrading transgenic proteins, another significant hurdle in the expression of exogenous casein in plants is the inherent difficulty of achieving satisfactory levels of casein expression. While the introduction of transgenic casein genes into plant genomes is a theoretically sound approach for producing non-animal-derived casein, practical implementation often encounters substantial obstacles. Specifically, casein genes derived from mammals do not naturally possess regulatory elements compatible with plant expression systems. This incompatibility can lead to poor transcriptional activity, resulting in minimal production of the casein protein within the plant cells.

What is needed, therefore, are methods to overcome the dual challenges of protease-induced degradation and the intrinsic difficulties in achieving effective casein expression and micelle assembly in plants by providing effective protection against proteolytic degradation throughout the protein's lifecycle, from synthesis to storage, while overcoming the limitations posed by the current understanding of protease pathways and the localization mismatch of protease inhibitors.

### SUMMARY

The current disclosure provides compositions, methods and systems for enhancing the levels of casein proteins and casein micelle formation in plants. In some instances, enhancing the levels of caseins and casein micelles in plants is achieved by inhibiting the expression or activity of certain protease genes. In some cases, inhibiting the expression or activity of protease genes is achieved by knockdown or knockout of certain protease genes. Contemplated proteases include one or more of a cysteine protease, an aspartyl proteases, a metalloprotease, a serine proteases, or any combination thereof. In some instances, the targeted gene is one or more proteases or anti-casein sequences identified by sequences 1 to 16 or 217 through 241. In some instances, the targeted gene is a sequence having at least 80%, at least 85%, at least 90%, at least 95%, or at least 99% sequence identity to any one of the sequences 1-16 or 217-241. Methods of knockdown or knockout of protease genes can be accomplished by any suitable method, for example, by using one or more of RNA interference (RNAi), small interfering RNA (siRNA), short-, medium-, or long-hairpin RNA (shRNA, mhRNA, lhRNA), or a bacterial RNA-guided endonuclease directed towards the protease gene. In some cases, inhibiting the expression or activity of protease genes is achieved by expressing proteins that bind to the proteases, for example, an antibody of the protease. In some cases, the plant is soybean. In some instances, levels of casein proteins are increased as compared to a counterpart without the protease knockdown, including any one or more the following casein proteins: K casein, α_{S1} casein, α_{S2} casein, or β casein.

The current disclosure also provides transgenic plants with reduced protease activity and increased levels of casein proteins, with increased levels of casein proteins or increased levels of casein micelle formation. In some cases, the transgenic plants can be used for making plant protein products with enhanced nutritional quality and digestibility. In some cases, the plant is soybean.

In some aspects, the current disclosure further provides transgenic soybean seeds and soy protein products with enhanced nutritional quality, as well as methods for producing these products. Additionally, the current disclosure discloses methods for introducing nucleic acid sequences encoding Cas proteins, zinc finger nucleases (ZFNs), or transcription activator-like effector nucleases (TALENs) into soybean cells, resulting in targeted mutagenesis or gene knockout of specific protease genes. Furthermore, the current disclosure provides methods for introducing modified promoters into soybean cells to increase the levels of casein proteins in soybean seeds. In some instances, any one of these methods disclosed herein can be combined (i.e., used simultaneously) to reduce the activity or expression of protease genes and to increase the level of casein proteins in plant seeds, resulting in transgenic plants with enhanced nutritional quality.

In some aspects, the current disclosure further provides method of enhancing in vivo assembly of a recombinant micelle, comprising co-expressing at least two ruminant casein proteins in a plant cell, and reducing a protease activity or expression in the plant cell; wherein the at least two ruminant casein proteins comprise a κ-casein and at least on of an αS1-casein, an αS2-casein, or a β-casein; wherein the κ-casein and at least one of the αS1-casein, the αS2-casein, or the β-casein form the recombinant micelle in the plant cell, and wherein reducing the protease activity or expression enhances in vivo assembly of a recombinant micelle. In some instances, reducing the protease activity or expression increases the number of recombinant micelles assembled in the plant cell. IN some cases, reducing the protease activity or expression comprises introducing an RNA interference construct targeting the protease gene. In some cases reducing the protease activity or expression comprises introducing a Cas protein and a guide RNA into the plant and inducing targeted mutagenesis or gene knockout of the protease gene. In some cases, reducing the protease activity or expression comprises introducing a nucleic acid sequence encoding a zinc finger nuclease (ZFN) or transcription activator-like effector nuclease (TALEN) into soybean cells, and inducing targeted mutagenesis or gene knockout of specific protease genes. In some cases, the protease is at least one of the cysteine protease, an aspartyl proteases, a metalloprotease, serine proteases, or any combination thereof, in some cases, the protease is one or more of sequences 1-16 or 217-241. In some cases, the protease comprises a peptide sequence that is at least 80%, at least 85%, at least 90%, at least 95%, or at least 99% identical to any one of the sequences in SEQ ID No. 1-16 or 217-241. In some cases, the disclosed method further comprises introducing a nucleic acid sequence encoding a modified promoter into soybean cells, wherein the modified promoter increases the level of casein proteins in the soybean seeds.

In some aspects, the present disclosure provides methods, compositions, and processes for enhancing expression levels and stability of casein proteins and casein micelles in transgenic plants (e.g., soybean). The methods described herein involve the use, simultaneously or sequentially, of one or more of the following techniques: a combination of reduced proteasomal expression, increased deubiquitination, lipidation, pegylation, protease inhibitors, mutated casein proteins, casein fusion proteins, promoter modification, codon optimization, gene copy number increase, post-transcriptional regulation, chromosomal location editing, chaperone proteins, translational enhancers, Scaffold/Matrix Attachment Region (SAR or Mar) addition, introns addition, hormonal induction, antisense RNAs, 3′ Untranslated Region (UTR) modification, signal peptide optimization, protease knockdown, and nutrient optimization.

Some aspects of the disclosure provide a method for enhancing expression levels and stability of a casein protein in a transgenic plant, comprising A) expressing the casein protein in the transgenic plant; and B) decreasing proteasomal activity or expression in the transgenic plant to decrease degradation of the casein protein expressed in the transgenic plant.

Some aspects of the disclosure provide a soy plant, wherein the soy plant: expresses a casein protein comprising αs1-casein, αs2-casein, β-casein, or κ-casein; and has reduced proteasomal activity or expression level compared to a wildtype soy plant. In some cases, the soy plant has decreased expression or activity of E1 (ubiquitin-activating enzyme), E2 (ubiquitin-conjugating enzyme), or E3 (ubiquitin ligase), or any combination thereof. In some cases, the soy plant has decreased expression or activity of ubiquitin/26S proteasome. In some cases, the soy plant comprises κ-casein and only one or two of αs1-casein, αs2-casein, and β-casein.

Some aspects of the disclosure provides a transgenic soy plant comprising a casein gene, wherein the transgenic soy plant exhibits reduced expression of the ubiquitin/26S proteasome compared to a non-transgenic soy plant, and wherein the reduced expression comprises reduced or no expression levels of E1 (ubiquitin-activating enzyme), E2 (ubiquitin-conjugating enzyme), E3 (ubiquitin ligase), or any combination thereof.

Some aspects of the disclosure provides a method for increasing casein protein expression in a transgenic soy plant, the method comprising: A) expressing the casein protein in the transgenic plant; and B) reducing the activity or expression of the ubiquitin/26S proteasome pathway in the soy plant or part thereof to increase the expression of the casein protein in the transgenic plant; wherein the reducing the expression comprises reduced or no expression levels of E1 (ubiquitin-activating enzyme), E2 (ubiquitin-conjugating enzyme), E3 (ubiquitin ligase), or any combination thereof.

Some aspects of the disclosure provide a transgenic soy plant, wherein the transgenic soy plant expresses a casein protein, and wherein the soy plant has increased activity or expression level of a deubiquitinating enzyme compared to a wildtype soy plant.

Some aspects of the disclosure provide a method for expressing a casein protein in a transgenic soy plant, the method comprising A) expressing the casein protein in the transgenic plant; and B) increasing the expression level of a deubiquitinating enzyme in the soy plant, such that the soy plant has increased expression of the casein protein compared to without increasing the expression level of the deubiquitinating enzyme.

Some aspects of the disclosure provide a method for increasing a casein protein expression in a transgenic soy plant, the method comprising A) expressing the casein protein in the transgenic plant; and B) enhancing the stability and prolonging the half-life of the casein protein in the soy plant through the use of chemical post-translational modifications (PTMs) comprising nitrosylation, methylation, acetylation, lipidation, or pegylation.

Some aspects of the disclosure provide a method for increasing casein protein expression in a transgenic soy plant, the method comprising A) expressing the casein protein in the transgenic plant; and B) conjugating the casein protein to a cross-linker, wherein the cross-linker is selected from the group consisting of Genipin, Ethylenediamine (C2H8N2), Cystamine (C4H12N2S2), Bis(sulfosuccinimidyl) suberate (BS3), or 1-Ethyl-3-(3-dimethylaminopropyl) carbodiimide (EDC), Glutaraldehyde (C5H802), ethylene glycol diglycidyl ether (EGDE), polyethylene glycol diglycidyl ether (PEGDE), toluene diisocyanate (TDI, C9H6N202), hexamethylene diisocyanate (HDI, C8H12N202), 1-ethyl-3-(3-dimethylaminopropyl) carbodiimide (EDC), N-Hydroxysuccinimide (NHS, C4H5NO3), dithiobis[succinimidyl propionate] (DSP), disuccinimidyl suberate (DSS), 3,3′-dithiobis[sulfosuccinimidylpropionate] (DTSSP), bis[2-(succinimidooxycarbonyloxy)ethyl] sulfone (BSOCOES), 1,5-difluoro-2,4-dinitrobenzene (DFDNB), formaldehyde (CH2O), dimethyl adipimidate (DMA), 1,5-difluoro-2,4-dinitrobenzene (DFDNB), genipin, ethylenediamine, cystamine, bismaleimidohexane (BMH), disuccinimidyl tartrate (DST), ethylene glycol bis[succinimidylsuccinate] (EGS), N,N′-ethylenebis(iodoacetamide) (EBI), N,N′-(1,3-phenylene)dibutyric acid di[succinimidyl ester] (DSS), N,N′-(ethylene-di-1,2-phenylene)bismaleimide, N,N′-1,4-phenylenebismaleimide, divinyl sulfone, diisopropylcarbodiimide, dicyclohexylcarbodiimide, diethylcarbodiimide, difluorodinitrobenzene, N,N′-disuccinimidyl carbonate (DSC), dithiobispropionimidate, glyoxal (C2H2O2), 1,5-difluoro-2,4-dinitrobenzene, N-succinimidyl(4-iodoacetyl)aminobenzoate (SIAB), N-succinimidyl(4-iodoacetyl)aminobenzoate (SIA), succinimidyl 4-(p-maleimidophenyl)butyrate (SMPB), and succinimidyl 6-[(beta-maleimidopropionamido)hexanoate] (SMPH). In some cases, the cross-linker is non-toxic.

Some aspects of the disclosure provide a casein molecule comprising a casein protein and a cross-linker, wherein the wherein the cross-linker is selected from the group consisting of Genipin, Ethylenediamine, Cystamine, Bis(sulfosuccinimidyl) suberate (BS3), or 1-Ethyl-3-(3-dimethylaminopropyl) carbodiimide (EDC), Glutaraldehyde, ethylene glycol diglycidyl ether (EGDE), polyethylene glycol diglycidyl ether (PEGDE), toluene diisocyanate (TDI), hexamethylene diisocyanate (HDI), 1-ethyl-3-(3-dimethylaminopropyl) carbodiimide (EDC), N-Hydroxysuccinimide (NHS), dithiobis[succinimidyl propionate] (DSP), disuccinimidyl suberate (DSS), 3,3′-dithiobis[sulfosuccinimidylpropionate] (DTSSP), bis[sulfosuccinimidyl] suberate (BS3), bis[2-(succinimidooxycarbonyloxy)ethyl]sulfone (BSOCOES), 1,5-difluoro-2,4-dinitrobenzene (DFDNB), formaldehyde, dimethyl adipimidate (DMA), 1,5-difluoro-2,4-dinitrobenzene (DFDNB), genipin, ethylenediamine, cystamine, bismaleimidohexane (BMH), disuccinimidyl tartrate (DST), ethylene glycol bis[succinimidylsuccinate] (EGS), N,N′-ethylenebis(iodoacetamide) (EBI), N,N′-(1,3-phenylene)dibutyric acid di[succinimidyl ester] (DSS), N,N′-(ethylene-di-1,2-phenylene)bismaleimide, N,N′-1,4-phenylenebismaleimide, divinyl sulfone, diisopropylcarbodiimide, dicyclohexylcarbodiimide, diethylcarbodiimide, difluorodinitrobenzene, N,N′-disuccinimidyl carbonate (DSC), dithiobispropionimidate, glyoxal, 1,5-difluoro-2,4-dinitrobenzene, N-succinimidyl(4-iodoacetyl)aminobenzoate (SIAB), N-succinimidyl(4-iodoacetyl)aminobenzoate (SIA), succinimidyl 4-(p-maleimidophenyl)butyrate (SMPB), and succinimidyl 6-[(beta-maleimidopropionamido)hexanoate] (SMPH).

Some aspects of the disclosure provide a method for increasing casein protein expression in a transgenic soy plant, the method comprising mutating the casein protein to comprise fewer Proline (P), Glutamic acid (E), Serine(S), or Threonine (T) compared to a wildtype casein protein.

Some aspects of the disclosure provide a fusion protein, comprising a casein protein and a peptide sequence that represents a ubiquitin-associated (UBA) domain.

Some aspects of the disclosure provide a method for increasing casein protein expression in a transgenic soy plant; the method comprising expressing a casein protein in the transgenic soy plant, wherein the casein protein is fused with a peptide sequence that represents a ubiquitin-associated (UBA) domain.

Some aspects of the disclosure provide a method for enhancing expression levels and stability of a casein protein in a transgenic plant, comprising expressing a casein protein in the transgenic soy plant, wherein the transgenic plant comprises a promoter that regulates the expression of the casein gene in the transgenic plant, and wherein the promoter is non-naturally occurring.

Some aspects of the disclosure provide a soy plant, wherein the soy plant: A) expresses a casein protein comprising αs1-casein, αs2-casein, β-casein, or κ-casein; and B) has a modified promoter of the casein gene compared to a wildtype soy plant.

Some aspects of the disclosure provide a method for enhancing expression levels and stability of a casein protein in a transgenic plant, comprising optimizing the codons of the casein gene in the plant.

Some aspects of the disclosure provide a soy plant, wherein the soy plant: A) expresses a casein protein comprising αs1-casein, αs2-casein, β-casein, or κ-casein; and B) has optimized codons of the casein gene compared to a wildtype soy plant.

Some aspects of the disclosure provide a method for enhancing expression levels and stability of a casein protein in a transgenic plant cell, comprising expressing the casein protein in the transgenic plant cell; wherein the transgenic plant comprises at least two, at least three, at least four, at least five, at least six copies, at least seven copies, or at least eight copies of DNA sequences coding the casein gene in the transgenic plant cell.

Some aspects of the disclosure provide a soy plant cell, wherein the soy plant cell expresses a casein protein comprising αs1 casein, αs2 casein, β casein, or K casein, wherein the soy plant cell comprises at least two, at least three, at least four, at least five, at least six copies, at least seven copies, or at least eight copies of DNA sequences coding the casein gene in the soy plant cell.

Some aspects of the disclosure provide a method for enhancing translation of a casein protein in a transgenic plant, comprising one or more of expressing the casein protein in the transgenic plant, wherein the transgenic plant comprises a mRNA coding the casein protein; and wherein the mRNA is post-transcriptionally modified to increase the translation of the casein protein.

Some aspects of the disclosure provide a soy plant, wherein the soy plant expresses a casein protein comprising αₛ₁ casein, αₛ₂ casein, β casein, or κ casein; and wherein the soy plant comprises a mRNA coding the casein protein; and wherein the mRNA is post-transcriptionally modified to increase the translation of the casein protein.

Some aspects of the disclosure provide a method for enhancing expression levels and stability of a casein protein in a transgenic plant, comprising mutating the sequence of the casein protein in the plant to increase the expression levels and stability of a casein protein in a transgenic plant.

Some aspects of the disclosure provide a soy plant, wherein the soy plant: A) expresses a casein protein comprising αₛ₁-casein, αₛ₂-casein, β-casein, or κ-casein; and B) has a mutated sequence of the casein protein compared to a wildtype casein protein.

Some aspects of the disclosure provide a method for enhancing expression levels and stability of a casein protein in a transgenic plant, comprising editing the chromosomal location of the casein gene in the plant to increase the expression levels of the casein protein in the transgenic plant.

Some aspects of the disclosure provide a soy plant, wherein the soy plant: A) expresses a casein protein comprising αₛ₁ casein, αₛ₂ casein, β casein, or κ casein; and B) has a different chromosomal location of the casein gene.

Some aspects of the disclosure provide a method for enhancing expression levels and stability of a casein protein in a transgenic plant, comprising using a chaperone protein to assist in the folding and assembly of the casein protein in the plant, such that the transgenic plant has increased expression levels and stability of the casein protein.

Some aspects of the disclosure provide a soy plant, wherein the soy plant: A) expresses a casein protein comprising αₛ₁ casein, αₛ₂ casein, β casein, or κ casein; and B) comprises a chaperone protein to assist in the folding and assembly of the casein protein.

Some aspects of the disclosure provide a method for enhancing expression levels and stability of a casein protein in a transgenic plant, comprising incorporating a translational enhancer in the sequence of the casein gene in the plant to increase the expression levels and stability of a casein protein in a transgenic plant.

Some aspects of the disclosure provide a soy plant, wherein the soy plant: A) expresses a casein protein comprising αₛ₁ casein, αₛ₂ casein, β casein, or K casein; and B) comprises a translational enhancer incorporated in the sequence of the casein gene.

Some aspects of the disclosure provide a method for enhancing expression levels and stability of a casein protein in a transgenic plant, comprising adding a Scaffold/Matrix Attachment Region (SAR or Mar) to the casein gene in the plant to increase the expression levels and stability of the casein protein in the transgenic plant.

Some aspects of the disclosure provide a soy plant, wherein the soy plant: A) expresses a casein protein comprising αₛ₁ casein, αₛ₂ casein, β casein, or κ casein; and B) comprises a Scaffold/Matrix Attachment Region (SAR or Mar) added to the casein gene.

Some aspects of the disclosure provide a method for enhancing expression levels and stability of a casein protein in a transgenic plant, comprising incorporating introns into the coding sequence of the casein gene in the plant to increase the expression levels and stability of the casein protein in the transgenic plant.

Some aspects of the disclosure provide a soy plant, wherein the soy plant: A) expresses a casein protein comprising αs1-casein, αs2-casein, β-casein, or κ-casein; and B) comprises introns incorporated into the coding sequence of the casein gene to increase the expression levels and stability of the casein protein in the transgenic plant.

Some aspects of the disclosure provide a method for enhancing expression levels and stability of a casein protein in a transgenic plant, comprising inducing hormonal changes in the plant to enhance casein gene expression.

Some aspects of the disclosure provide a soy plant, wherein the soy plant: A) expresses a casein protein comprising αs1 casein, αs2 casein, β casein, or κ casein; and B) wherein the soy plant has undergone hormonal induction to enhance casein gene expression.

Some aspects of the disclosure provide a method for enhancing expression levels and stability of a casein protein in a transgenic plant, comprising using antisense RNAs to suppress negative regulators of casein expression in the plant, to increase the expression levels and stability of the casein protein in the transgenic plant.

Some aspects of the disclosure provide a soy plant, wherein the soy plant: A) expresses a casein protein comprising αs1 casein, αs2 casein, β casein, or κ casein; and B) comprises an antisense RNA to suppress negative regulators of casein expression.

Some aspects of the disclosure provide a method for enhancing expression levels and stability of a casein protein in a transgenic plant, comprising modifying the 3′ Untranslated Region (UTR) of the casein gene in the plant, to increase the expression levels and stability of the casein protein in the transgenic plant.

Some aspects of the disclosure provide a soy plant, wherein the soy plant: A) expresses a casein protein comprising αₛ₁ casein, αₛ₂ casein, β casein, or κ casein; and B) comprises a modified 3′ Untranslated Region (UTR) of the casein gene.

Some aspects of the disclosure provide a method for enhancing expression levels and stability of a casein protein in a transgenic plant, comprising optimizing the signal peptide of the casein protein in the plant.

Some aspects of the disclosure provide a soy plant, wherein the soy plant: A) expresses a casein protein comprising αₛ₁ casein, αₛ₂ casein, β casein, or κ casein; and B) comprises an optimized signal peptide of the casein protein compared to a wildtype soy plant.

Some aspects of the disclosure provide a method for enhancing expression levels and stability of a casein protein in a transgenic plant, comprising fusing the casein protein with another protein in the plant, to increase the expression levels and stability of the casein protein in the transgenic plant.

Some aspects of the disclosure provide a soy plant, wherein the soy plant expresses a fusion of the casein protein with another protein compared to a wildtype soy plant.

Some aspects of the disclosure provide a method for enhancing expression levels and stability of a casein protein in a transgenic plant, comprising knocking down protease activity or expression in the plant to increase the expression levels and stability of the casein protein in the transgenic plant.

Some aspects of the disclosure provide a soy plant, wherein the soy plant: A) expresses a casein protein comprising αs1 casein, αs2 casein, β casein, or κ casein; and B) has reduced protease activity or expression level compared to a wildtype soy plant.

Some aspects of the disclosure provide a method for enhancing expression levels and stability of a casein protein in a transgenic plant, comprising optimizing the nutrient and environmental conditions of the plant to enhance casein gene expression.

Some aspects of the disclosure provide a soy plant, wherein the soy plant: A) expresses a casein protein comprising αₛ₁ casein, αₛ₂ casein, β casein, or κ casein; and B) is grown under optimized nutrient and environmental conditions to enhance casein gene expression compared to a wildtype soy plant.

Some aspects of the disclosure provide a method of expressing casein proteins in a plant, comprising performing any one of the methods or plants described herein, wherein the method results in increased expression of the casein gene in the soy plant or part thereof compared to a soy plant or part thereof not subjected to the method.

Some aspects of the disclosure provide a method of extracting casein proteins from the soybean plant; comprising providing using a buffer comprising a protease inhibitor selected from the group consisting of a Serine Protease Inhibitor, a Cysteine Protease Inhibitor, an Aspartic Protease Inhibitor, a Metalloprotease Inhibitor, a Covalent Protease Inhibitor, a Reversible Protease Inhibitor, a Peptide-based Protease Inhibitor, a Synthetic Protease Inhibitor, or a Natural Protease Inhibitor.

Some aspects of the disclosure provide a casein protein modified by lipidation, wherein the lipidation is cysteine prenylation, N-terminal glycine myristoylation, cysteine palmitoylation, or serine and lysine fatty acylation.

Some aspects of the disclosure provide a modified casein protein, comprising a casein protein and a polyethylene glycol (PEG), wherein the polyethylene glycol (PEG) is covalently attached to casein protein.

Some aspects of the disclosure provide a mutated alpha casein protein, wherein the mutated alpha casein protein is one of sequences 199 to 204.

Some aspects of the disclosure provide a mutated alpha casein protein, wherein the mutated alpha casein protein comprises at least 80% of one of sequences 199 to 204.

Some aspects of the disclosure provide a mutated beta-casein protein, wherein the mutated beta-casein protein comprises one of sequences 207 through 213.

Some aspects of the disclosure provide a mutated beta-casein protein, wherein the mutated beta-casein protein comprises at least 80% of any of sequences 207 through 213.

Some aspects of the disclosure provide a soybean comprising casein proteins, wherein the casein proteins comprise at least 0.03% of the total weight of the seed, at least 0.041% of the total weight of the seed, or at least 0.055% of the total weight of the seed, or at least 0.075% of the total weight of the seed, or at least 0.101% of the total weight of the seed, or at least 0.136% of the total weight of the seed, or at least 0.184% of the total weight of the seed, or at least 0.248% of the total weight of the seed, or at least 0.335% of the total weight of the seed, or at least 0.452% of the total weight of the seed, or at least 0.610% of the total weight of the seed, or at least 0.824% of the total weight of the seed, or at least 1.113% of the total weight of the seed, or at least 1.503% of the total weight of the seed, or at least 2.031% of the total weight of the seed, or at least 2.744% of the total weight of the seed, or at least 3.707% of the total weight of the seed, or at least 5.006% of the total weight of the seed, or at least 6.761% of the total weight of the seed, or at least 13% of the total weight of the seed.

Some aspects of the disclosure provide a soybean comprising casein proteins, wherein the casein proteins comprise at least 0.075% of the total protein content of the seed, or at least 0.103% of the total protein content of the seed, or at least 0.138% of the total protein content of the seed, or at least 0.188% of the total protein content of the seed, or at least 0.253% of the total protein content of the seed, or at least 0.340% of the total protein content of the seed, or at least 0.460% of the total protein content of the seed, or at least 0.620% of the total protein content of the seed, or at least 0.838% of the total protein content of the seed, or at least 1.130% of the total protein content of the seed, or at least 1.525% of the total protein content of the seed, or at least 2.060% of the total protein content of the seed, or at least 2.783% of the total protein content of the seed, or at least 3.758% of the total protein content of the seed, or at least 5.078% of the total protein content of the seed, or at least 6.860% of the total protein content of the seed, or at least 9.267% of the total protein content of the seed, or at least 12.503% of the total protein content of the seed, or at least 16.903% of the total protein content of the seed, or at least 32.5% of the total protein content of the seed.

Some aspects of the disclosure provide a soybean comprising casein micelles, wherein the casein micelles comprise at least 0.03% of the total weight of the seed, or at least 0.041% of the total weight of the seed, or at least 0.055% of the total weight of the seed, or at least 0.075% of the total weight of the seed, or at least 0.101% of the total weight of the seed, or at least 0.136% of the total weight of the seed, or at least 0.184% of the total weight of the seed, or at least 0.248% of the total weight of the seed, or at least 0.335% of the total weight of the seed, or at least 0.452% of the total weight of the seed, or at least 0.610% of the total weight of the seed, or at least 0.824% of the total weight of the seed, or at least 1.113% of the total weight of the seed, or at least 1.503% of the total weight of the seed, or at least 2.031% of the total weight of the seed, or at least 2.744% of the total weight of the seed, or at least 3.707% of the total weight of the seed, or at least 5.006% of the total weight of the seed, or at least 6.761% of the total weight of the seed, or at least 13% of the total weight of the seed.

Some aspects of the disclosure provide a soybean comprising casein micelles, wherein the casein micelles comprise at least 0.075% of the total protein content of the seed, or at least 0.103% of the total protein content of the seed, or at least 0.138% of the total protein content of the seed, or at least 0.188% of the total protein content of the seed, or at least 0.253% of the total protein content of the seed, or at least 0.340% of the total protein content of the seed, or at least 0.460% of the total protein content of the seed, or at least 0.620% of the total protein content of the seed, or at least 0.838% of the total protein content of the seed, or at least 1.130% of the total protein content of the seed, or at least 1.525% of the total protein content of the seed, or at least 2.060% of the total protein content of the seed, or at least 2.783% of the total protein content of the seed, or at least 3.758% of the total protein content of the seed, or at least 5.078% of the total protein content of the seed, or at least 6.860% of the total protein content of the seed, or at least 9.267% of the total protein content of the seed, or at least 12.503% of the total protein content of the seed, or at least 16.903% of the total protein content of the seed, or at least 32.5% of the total protein content of the seed.

Some aspects of the present disclosure, provide methods for increasing the expression of Glycelles, as distinct from casein micelles. “Glycelles” are defined by U.S. patent application Ser. No. 18/478,477, which is incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawings will be provided by the Office upon request and payment of the necessary fee.

The figures showing embodiments of the system are semi-diagrammatic, and not to scale and, particularly, some of the dimensions are for the clarity of presentation and are shown exaggerated in the figures. Similarly, although the views in the figures for ease of description generally show similar orientations, this depiction in the figures is arbitrary for the most part. Generally, the invention can be operated in any orientation.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts western blot data showing protease digestion of kappa-casein by plant proteases. “−” means Negative control. Positive control (+) is kappa casein purchased from Sigma-Aldrich. Lane 1 shows a sample of transgenic soy that has been altered to express alpha-, beta-, and kappa-casein. Lane 2 depicts Tris Extraction Buffer mixed with Leprino Micellar Casein Concentrate (MCC) in a 1:2 ratio at 4 degrees Celsius. Lane 3 shows transgenic soy mixed with Leprino Micellar Casein Concentrate (MCC) in a 1:2 ratio at 4 degrees Celsius. Lane 4 displays Tris Extraction Buffer mixed with Leprino Casein Concentrate (MCC) in a 1:2 ratio at room temperature. Lane 5 shows transgenic soy mixed with Leprino Micellar Casein Concentrate (MCC) in a 1:2 ratio at room temperature. Lane 6 depicts Tris Extraction Buffer mixed with Leprino Micellar Casein Concentrate (MCC) in a 1:2 ratio at 37 degrees Celsius. Lane 7 displays transgenic soy mixed with Leprino Micellar Casein Concentrate (MCC) in a 1:2 ratio at 37 degrees Celsius.

FIG. 2 depicts western blot data showing effectiveness of protease inhibitor. The labels mean the following:
1. “WT” means wild type and is used as a negative control.
2. “+” means positive control and is comprised of a peptide with antibodies targeting flag tag.
3. “A” is a sample prepared with the plasmid depicted in FIG. 3, which has been transiently transformed into soy to express alpha S1, Beta, and Kappa casein and in which protease activity is sufficiently low that casein proteins are detectable by western blot using a TEB buffer (a soluble extraction method).
4. “B” is a sample prepared with the same plasmid (FIG. 3), but where protease activity is such that the casein proteins are detectable using Urea extraction (i.e., an insoluble protein extracting method), and where the protease activity is undetectable by soluble extraction methods.
5. “C1” is a sample prepared using the same plasmid, but where protease activity is higher, such that casein proteins are not detectable by Western Blots when using either TEB buffer or Urea extraction.
6. “C2” is a sample prepared using the same criteria as C1, but with a different transformation date.
7. A prime symbol ′ is used to indicate that the sample was treated with Phenylmethylsulphonyl fluoride (PMSF).

The following protocol was utilized:
1. Weigh crushed and frozen soy tissue in a 2 mL Eppendorf tube.
2. In a separate tube, prepare a solution of TEB (Tissue Extraction Buffer) with 1 mM PMSF added.
3. Add 5 μL of the TEB+PMSF solution per milligram of soy tissue to the Eppendorf tube containing the frozen tissue, and vortex the mixture for 30 seconds.
4. Incubate the extraction reaction by rotating the tube for 25 minutes at 4° C.
5. Centrifuge the tubes at 16,100 RCF for 5 minutes at 4° C. to separate the tissue extract.
6. To stop the extraction process, immediately transfer 5 μL of the tissue extract to a new tube containing 15 μL of Laemmli buffer with added beta-mercaptoethanol (BME). Proceed to blot the sample as required.

A vector transformation is confirmed by the successful expression of the green fluorescent protein in all samples. The western blots are targeting flag tag. With this technique, it was observed that the addition of PMSF reduced the degradation of casein in the soy tissue.

FIG. 3 depicts the plasmid utilized in the experiment described above and associated with the Western Blots in FIG. 2.

FIG. 4 is a Flowchart diagram illustrating the use of Barcodes to identify Casein operative protease.

FIG. 5 shows a plasmid diagram, 1606+beta-CS-gBlock0+RNAi-CS. “NPTII” refers to the neomycin phosphotransferase II gene, a selectable marker for kanamycin resistance in plant transformation. “6xHis” is a hexa-histidine tag used for metal affinity chromatography to purify tagged proteins. “AtuMAS Pro+5′U” represents the Agrobacterium tumefaciens mannopine synthase promoter with an upstream region, which drives gene expression. “35S Term” is the terminator sequence from the Cauliflower Mosaic Virus 35S promoter, stopping transcription. “GmGY1 SP” is a signal peptide from the Glycine max gene, directing the protein to the endoplasmic reticulum. “V5 tag” is an epitope tag for the detection and purification of recombinant proteins. “SIRbcS3C Term” stands for the Solanum lycopersicum Rubisco small subunit terminator, signaling the end of transcription. “OCS Term” is the octopine synthase terminator from Agrobacterium, which halts transcription. “TMV Ω 5′UTR” is the Tobacco Mosaic Virus omega leader sequence, a translational enhancer for the downstream gene. “Cow Fam20C” is a kinase from the bovine Fam20 family that phosphorylates caseins. “RK2 T-DNA” refers to part of the vector backbone.

FIG. 6 shows a flowchart illustrating the process of analysis of the presence of particular barcodes and identifying corresponding RNAi constructs that target specific proteases involved in beta casein degradation.

FIG. 7 shows a graphical representation of ribosome profiling data obtained from soy that has been genetically modified to express at least three foreign genes. The horizontal axis is labeled “Red” and represents the intensity of red fluorescent protein (RFP) expression, controlled by the omega Tobacco Mosaic Virus 5′ Untranslated Region (“TMV Ω 5′ UTR”) and serving as a control for translational efficiency. The vertical axis is labeled “Green” and denotes the intensity of green fluorescent protein (GFP) expression, controlled by test UTRs from the specialized library constructed from preliminary Ribo-seq data. The chart is populated with a scatter plot of data points. These points represent individual protoplasts, each modified with a unique UTR controlling GFP expression. The distribution of these data points illustrates the relative translational efficiencies of the test UTRs when compared against the TMV Ω 5′ UTR control. Three distinct regions, labeled P4, P5, and P6, are delineated on the plot. Each region represents a specific range of translational efficiencies as indicated by the ratio of GFP to RFP expression. The region bounded by the label “Highest expressing” contains data points that exhibit the most elevated translational efficiencies, as indicated by a high GFP/RFP ratio. Conversely, the region near the label “Lowest expressing” contains data points showcasing the lowest translational efficiencies, manifested as a reduced GFP/RFP ratio.

FIG. 8 depicts three distinct vectors used for assessing translational efficiency in soy protoplasts. The top vector, labeled “Test Vector,” consists of a series of genetic elements arranged sequentially to transcribe and translate two fluorescent proteins. Both genes are transcribed under a 35S constitutive promoter, denoted by an arrow, and terminated by an NOS 3′ UTR. For mScarlet-I, the TMV Ω 5′ UTR, a viral UTR known to have high translational efficiency in plants, is placed directly in front of our mScarlet-encoding gene, while GFP harbors unique 5′ UTRs from our synthesized library. mScarlet should have similar translation levels across samples and serves as our normalization factor, while GFP should vary in its translation, depending on the 5′ UTR being used. The middle vector, labeled “Positive control Vector,” is identical to the Test vector, except that the GFP 5′UTR is also the TMV Ω 5′ UTR. The bottom vector, labeled “Negative control Vector,” is essentially identical to the Test and Positive control Vectors, except it has no 5′UTR before the GFP. In some instances, vectors may be modified to include alternative genetic elements, regulatory sequences, or reporter genes.

FIG. 9 shows a plasmid diagram, pMOZ2592 (L2)GB-704 13G170900+04G009900 5′UTR in 1928 backbone.

FIG. 10 shows a plasmid diagram. “NPTII” refers to the neomycin phosphotransferase II gene, a selectable marker for kanamycin resistance in plant transformation. “RB” denotes the Right Border, essential for DNA transfer from Agrobacterium into the plant genome. “EL6” is an “end linker” sequence used to assemble the plasmid. “AtuMAS Term” stands for Agrobacterium tumefaciens mannopine synthase terminator, which terminates transcription. “HDEL” is a peptide tag that retains proteins in the endoplasmic reticulum. “6xHis” is a hexa-histidine tag used for metal affinity chromatography to purify tagged proteins. “AtuMAS Pro+5′U” represents the Agrobacterium tumefaciens mannopine synthase promoter with an upstream region, which drives gene expression. “35S Term” is the terminator sequence from the Cauliflower Mosaic Virus 35S promoter, stopping transcription. “GmGY1 SP” is a signal peptide from the Glycine max gene, directing the protein to the endoplasmic reticulum. “V5 tag” is an epitope tag for the detection and purification of recombinant proteins. “SIRbcS3C Term” stands for the Solanum lycopersicum Rubisco small subunit terminator, signaling the end of transcription. “OCS Term” is the octopine synthase terminator from Agrobacterium, which halts transcription. “FLAG” is another epitope tag for the detection and purification of recombinant proteins. “TMV Ω 5′UTR” is the Tobacco Mosaic Virus omega leader sequence, a translational enhancer for the downstream gene. “Cow Fam20C” is a kinase from the bovine Fam20 family that phosphorylates caseins. “RK2 T-DNA” refers to part of the vector backbone.

FIG. 11 shows a plasmid diagram. “NPTII” refers to the neomycin phosphotransferase II gene that confers kanamycin resistance and is used as a selection marker in plant transformation. “RB” denotes the Right Border sequence essential for Agrobacterium-mediated DNA transfer into the plant genome. “EL6” is an “end linker” sequence used to assemble the plasmid. “AtuMAS Term” stands for the Agrobacterium tumefaciens mannopine synthase terminator, signaling the end of a gene's transcription. “GY1 VSD” is a variable signal domain of the Glycine max GY1 gene, involved in protein targeting or regulation. “GY1 10AA VSD” indicates a 10 amino acid vacuolar sorting determinant from the GY1 gene. “Strep Tag” is a peptide sequence used for the affinity purification of recombinant proteins. “G/S” represents a glycine/serine-rich linker or flexible region in a protein construct. “Cow Fam20C” refers to the bovine FAM20C kinase gene, possibly included for its role in phosphorylating proteins post-translationally. “CaMV 35S Short Promoter” is a truncated version of the 35S promoter from the Cauliflower Mosaic Virus, initiating gene expression. “At2S2 SP” is the signal peptide from Arabidopsis thaliana 2S2 protein, directing protein transport. “SIRbcS3C Term” is a terminator sequence for transcriptional termination. “OCS Term” is the octopine synthase terminator from Agrobacterium, used to halt transcription. “GmGY1 SP” is the signal peptide from a Glycine max GY1 gene, for protein localization. “V5” is an epitope tag for protein detection and purification. “6xHis” refers to a hexa-histidine tag. “AtuMAS Pro+5′U” denotes the Agrobacterium tumefaciens mannopine synthase promoter with an upstream region, for driving gene expression. “FLAG” is an epitope tag for the detection and purification of proteins. “GFP” stands for Green Fluorescent Protein, used as a visual marker for transgenic protein expression. “RK2 T-DNA” refers to part of the vector backbone.

FIG. 12 shows a plasmid diagram. “NPTII” refers to the neomycin phosphotransferase II gene, providing kanamycin resistance as a selection marker in plant transformation. “RB” denotes the Right Border, essential for DNA transfer from Agrobacterium into the plant genome. “AtuMAS Term” stands for Agrobacterium tumefaciens mannopine synthase terminator, which signals the end of gene transcription. “HDEL” is a retention signal for keeping proteins in the endoplasmic reticulum. “6xHis” is a hexa-histidine tag for protein purification. “GmBB1” refers to Bowman-Birk inhibitor. “GmGY1 SP” indicates the signal peptide from the Glycine max GY1 gene for protein targeting. “AtuMAS Pro+5′U” represents the Agrobacterium tumefaciens mannopine synthase promoter with an upstream region for gene expression. “CaMV 35S Short Promoter” is a truncated promoter from the Cauliflower Mosaic Virus, initiating gene expression. “TMV Ω 5′UTR” refers to the Tobacco Mosaic Virus omega leader sequence, enhancing translation. “FLAG” is an epitope tag for protein detection and purification. “SIRbcS3C Term” refers to a specific terminator sequence. “OCS Term” denotes the octopine synthase terminator from Agrobacterium, used to halt transcription. “V5 tag” is an epitope tag for detection and purification of recombinant proteins. “GFP” stands for Green Fluorescent Protein, a marker for expression and visualization of transgenic proteins. “RK2 T-DNA” refers to part of the vector backbone. “oriV” indicates the origin of vegetative replication, essential for plasmid maintenance and replication in bacterial cells. “Cow Fam20C” refers to bovine a FAM20C kinase gene.

FIG. 13 shows a plasmid diagram. “NPTII” refers to the neomycin phosphotransferase II gene for kanamycin resistance, used in selecting transformed plants. “RB” denotes the Right Border, integral for Agrobacterium-mediated plant genome transformation. “oriV” indicates the origin of vegetative replication, essential for plasmid replication in bacteria. “PUC ori” refers to the origin of replication from the pUC series of plasmid vectors. “RK2 T-DNA” refers to part of the vector backbone. “GmGY1 SP” is the signal peptide from the Glycine max GY1 gene, directing the transgenic protein to the endoplasmic reticulum. “V5 tag” is an epitope tag for detection and purification of the expressed protein. “6xHis” is a hexa-histidine tag for protein purification. “GY1 VSD” refers to the vacuolar sorting determinant of the Glycine max GY1 gene, potentially involved in protein targeting. “SIRbcS3C Term” is a terminator sequence from a plant gene or synthetic construct. “OCS Term” signifies the octopine synthase terminator from Agrobacterium, terminating transcription. “GFP” is the Green Fluorescent Protein gene, serving as a reporter for gene expression. “TMV Q 5′UTR” is the Tobacco Mosaic Virus omega leader sequence, enhancing translation. “AtuMAS Pro+5′U” denotes the Agrobacterium tumefaciens mannopine synthase promoter plus upstream region, driving gene expression. “GmGY1 SP” repeated indicates the use of the same signal peptide from Glycine max GY1. “At2S2 SP” is the signal peptide from Arabidopsis thaliana 2S2 protein, also for protein targeting. “SIH4 term” represents a terminator sequence for transcriptional termination. “FLAG” is an epitope tag for protein detection and purification. “GY1 VSD” repeated, suggests another instance of the variable signal domain for protein targeting. “G/S” could represent a glycine/serine-rich linker in the protein construct. “Strep Tag” is for affinity purification of proteins. “GY1 10AA VSD” refers to a 10 amino acid variant of the GY1 variable signal domain. “35S Term” stands for the Cauliflower Mosaic Virus 35S terminator, ending transcription. “Cow Fam20C” refers to the bovine FAM20C kinase gene, included for its role in phosphorylating proteins post-translationally.

FIG. 14 shows a plasmid diagram, pMOZ 2274.

FIG. 15 shows a plasmid diagram, pMOZ 2284.

FIG. 16 is a Bar Graph showing Casein (35S) Expression versus GFP (35S) Expression in Soybean Protoplasts.

FIG. 17 shows a plasmid diagram, pMOZ2327. showing the incorporation of Rb7 downstream of a Beta Casein gene.

FIG. 18 shows a graph of flow cytometry analysis performed on protoplasts derived from soybean cells and which serve as a negative control, that is involving protoplasts that underwent no transformation process.

FIG. 19 shows a graph of flow cytometry analysis performed on protoplasts derived from soybean cells wherein protoplasts were transformed with a plasmid harboring the 35S promoter, which was responsible for driving the expression of both the mScarlet and Green Fluorescent Protein (GFP) genes on a single plasmid.

FIG. 20 shows a graph of flow cytometry analysis performed on protoplasts derived from soybean cells where protoplasts in which the expression of the mScarlet gene was driven by the 35S promoter, whereas the GFP gene's expression was controlled by the STAP21 promoter (i.e. Seq. 164). The STAP21 promoter, in this context, refers to a specific DNA sequence recognized by transcription factors to initiate the transcription of the GFP gene.

FIG. 21 shows a graph of flow cytometry analysis performed on protoplasts derived from soybean cells incorporating the 35S promoter to drive the mScarlet gene, the STAP21 promoter for GFP gene expression, and an additional 35S promoter to drive the expression of the dTALE-2 gene.

FIG. 22 shows a chart showing how synthetic promoters are used in combination with transgenic ruminant alpha s1 casein, transgenic ruminant beta casein transgenic ruminant kappa casein, transgenic FAM20C, or some combination of those genes and illustrates the process of the regulation of casein expression levels via the interaction of a seed specific promoter with various synthetic promoters.

FIG. 23 shows a Bar Graph showing casein protein levels of both αₛ₁ casein, β casein, K casein in the sample containing the mhRNA-enhanced plasmid compared to a control sample.

### DETAILED DESCRIPTION

Certain aspects of the disclosure have other steps or elements in addition to or in place of those mentioned above. The steps or elements will become apparent to those skilled in the art from a reading of the following detailed description when taken with reference to the accompanying drawings.

While some embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The following embodiments are described in sufficient detail to enable those skilled in the art to make and use the invention. It is to be understood that other embodiments would be evident based on the present disclosure, and that system, process, or mechanical changes can be made without departing from the scope of an embodiment of the present disclosure.

In the following description, numerous specific details are given to provide a thorough understanding of the invention. However, it will be apparent that the invention can be practiced without these specific details. In order to avoid obscuring an embodiment of the present disclosure, some well-known techniques, system configurations, and process steps are not disclosed in detail. Throughout this disclosure, various publications, patents and published patent specifications are referenced by an identifying citation. The disclosures of these publications, patents and published patent specifications are hereby incorporated by reference into the present disclosure.

### Genomic and Epigenetic Strategies for Optimizing Casein Gene Expression

### Gene Copy Number Increase

In certain embodiments of the present invention, the overall expression of the exogenous gene is enhanced by introducing multiple copies of the gene into a plant, such as soy. This can be achieved by inserting these copies into different genomic loci, which can lead to an increase in the total production of the exogenous protein. In some embodiments, the disclosed methods result in the introduction of multiple copies of the exogenous gene into the plant, wherein the number of casein gene copies is at least 2, at least 3, at least 4, at least 5, at least 6, at least 7, at least 8, at least 9, at least 10, or more.

For instance, an iterative process can be employed to create a stable line of soy plants with multiple copies of the exogenous gene, such as casein. In this process, a plasmid carrying the casein gene is first transformed into the soy plant. Once a stable line carrying this initial insertion has been established, the plant is transformed again (a second transformation) with the same or a different plasmid carrying the exogenous gene. In some cases, the second transformation is targeted to a different genomic location. In some cases, the second transformation results in a plant with two copies of the exogenous gene at distinct loci. In some cases, this process is repeated multiple times to introduce additional copies of the exogenous gene, each time creating a new stable line. Each additional copy of the exogenous gene is expected to contribute to the overall production of the protein derived from the exogenous gene, thereby enhancing the total yield. In some cases, this process can be used for multiple exogenous genes.

### Scaffold/Matrix Attachment Region (SAR or Mar) Addition

In certain embodiments, Scaffold/Matrix Attachment Regions (SARs or MARs) are incorporated into the sequence of the casein gene to enhance its transcription, thereby increasing the overall production of casein protein in the genetically modified soy plant. In some embodiments, the disclosed methods involve the incorporation of at least one, at least two, at least three, at least four, at least five, up to and including ten or more SARs or MARs into the sequence of the casein gene. In some cases, the SAR from the chicken lysozyme gene is added to the casein gene. This is achieved by inserting the chicken lysozyme SAR sequence upstream of the casein gene, downstream of the gene, or both. The SAR sequence can also be inserted within an intron of the casein gene. In some cases, SARs from other genes or species are used. For example, the β-globin SAR or the Drosophila SAR can increase the expression of the casein gene. In some embodiments, multiple SAR sequences are added to the casein gene to further enhance its expression. In some cases, this involves the same SAR repeated multiple times; in other cases different SARs are combined to take advantage of their unique properties. In some cases, the SAR sequences are added in a specific order to create a SAR “cassette” that maximizes casein gene expression. In some cases, the incorporation of these SARs or MARs results in an increase in casein protein production by at least 10%, at least 20%, at least 30%, at least 40%, at least 50%, at least 60%, at least 70%, at least 80%, at least 90%, up to and including 100%, or more, compared to a plant not subjected to the disclosed methods.

FIG. 17 depicts a plasmid diagram and Sequence 172 depicts the Rb7 Matrix Attachment Region (MAR) sequence. The plasmid diagram incorporates Rb7 downstream of a Beta Casein gene (e.g., seqs. 213, 214). The sequence also incorporates Green Fluorescent Protein, with a 35S 3′ UTR. In some cases, the use of Rb7 downstream of the exogenous protein serves to increase the likelihood and magnitude of exogenous protein expression.

### Chromosomal Location Editing

In some aspects, the site where the casein gene is inserted in the plant genome impacts its expression, thereby affecting the overall production of the casein protein. The genomic context, including the proximity to enhancers, silencers, insulators, and other regulatory elements, as well as the local chromatin structure, can impact the accessibility of the inserted gene to the transcriptional machinery, and thus its expression level. For instance, insertion into a genomic region that is transcriptionally active, or “open,” can lead to higher levels of casein gene expression compared to insertion into a “closed” or transcriptionally silent region. Similarly, insertion near endogenous plant enhancers can boost casein gene expression, while insertion near silencers can reduce it.

In certain embodiments of the present invention, Agrobacterium-mediated transformation is employed as a vector to introduce the modified casein gene into a specific site within the soy plant genome. The modified casein gene, along with any other desired genetic elements (such as a strong promoter or a marker gene), is inserted into the T-DNA region of a binary plasmid within the EHA105 strain. In some embodiments, the T-DNA also includes a sequence that is homologous to the desired insertion site in the soy genome. This can promote targeted integration of the T-DNA at this site through a process known as homologous recombination. In certain embodiments of the present invention, the Agrobacterium tumefaciens strain EHA105 is utilized as a vector to deliver the modified casein gene into a specific locus within the soy plant genome. This process, known as Agrobacterium-mediated transformation, involves the insertion of the modified casein gene, along with any other desired genetic elements (such as a robust promoter or a marker gene), into the T-DNA region of a binary plasmid harbored by the EHA105 strain.

In some embodiments, the T-DNA is designed to include a sequence that shares homology with the targeted insertion site in the soy genome. This homologous sequence can facilitate the precise integration of the T-DNA at the desired genomic location through a process known as homologous recombination. This strategy allows for the controlled insertion of the casein gene, enhancing its expression by positioning it in a genomic context that is conducive to high levels of transcription.

In some embodiments, targeted gene insertion methods, such as those based on homologous recombination or site-specific nucleases (e.g., CRISPR/Cas9, TALENs, or zinc finger nucleases), can be used to insert the casein gene at a specific, pre-determined locus in the soy genome. This allows for the selection of an optimal insertion site that maximizes casein gene expression.

In some embodiments, the disclosed methods result in the insertion of the casein gene within at least 1 kilobase (kb), at least 2 kb, at least 3 kb, at least 4 kb, at least 5 kb, or more, upstream or downstream of a genomic enhancer or other regulatory element that boosts gene expression.

### Epigenetic Modifications

In some cases, the present invention employs epigenetic modifications to enhance the expression of the casein gene in transgenic plants. In some cases, the epigenetic modification involves changes in DNA methylation patterns. DNA methylation, which typically occurs at cytosine residues in a CpG context, is a key regulator of gene expression. In some cases, the methylation status of the casein gene or its promoter region is altered to enhance casein expression. In some cases, this involves demethylation to activate the gene or targeted methylation of at least one repressor binding site to prevent or reduce repression of the gene.

In some cases, the epigenetic modification involves changes in histone modification. In some cases, histones associated with the casein gene are acetylated to enhance casein expression. In some cases, histones are deacetylated or methylated at specific residues to modulate casein expression.

In some cases, the epigenetic modification involves the use of small non-coding RNAs, such as microRNAs or small interfering RNAs, which can guide epigenetic machinery to specific sites in the genome. In some cases, these small RNAs are used to target the casein gene or its regulatory regions for epigenetic modification.

In some cases, the epigenetic modification involves the use of chromatin remodeling complexes. In some cases, these complexes are directed to the casein gene to enhance its expression.

In some cases, the epigenetic modification involves the use of epigenetic editing tools, such as zinc finger proteins or CRISPR-dCas9 fused to epigenetic modifiers. In some cases, these tools are used to modify the epigenetic status of the casein gene or its regulatory regions to enhance casein expression.

### Transcriptional Regulation

### Engineering Synthetic Transcription Factors and Promoters for Targeted Casein Expression in Transgenic Soy Plants

Gene transcription is a complex process regulated by various transcription factors (TFs) that bind to specific DNA sequences, known as promoters. These TFs interact with RNA polymerase, the enzyme responsible for converting DNA into RNA, and influence whether and where a gene is transcribed.

To manipulate gene expression, synthetic TFs can be engineered to bind to specific promoter sequences. A library of synthetic promoters with varying binding affinities for the TF can be created, providing a collection of tools to control gene expression levels. For instance, selecting different promoters from the library can drive gene expression at different intensities, resulting in varying levels of RNA and protein production.

In the context of casein expression in transgenic soy plants, a synthetic TF (DTALE) and a collection of synthetic promoters can be employed. To restrict casein expression to seeds, a seed-specific promoter can be used to drive the expression of DTALE. This ensures that DTALE is only present in seeds, where it can interact with the library of synthetic promoters to regulate casein expression levels. Sequences 167 (DNA) and 168 (AA) provide an example.

By selecting promoters with different binding affinities for DTALE, the expression of casein can be fine-tuned, resulting in varying levels of casein production in seeds. This approach provides a versatile tool for manipulating casein expression in transgenic soy plants, enabling the production of desired amounts of casein in specific tissues.

In some embodiments synthetic promoters are used in combination with transgenic ruminant alpha s1 casein (e.g., Seq. nos. 205-206), transgenic ruminant beta casein (e.g., Seq. nos. 213-214), transgenic ruminant kappa casein (e.g., Seq. no. 121), transgenic FAM20C (e.g., Seq. No. 166), or some combination of those genes. In some embodiments, a seed specific promoter along with a transcription factor is also used. FIG. 22 and Seq. Nos. 167-168 illustrate the process of the regulation of casein expression levels via the interaction of a seed specific promoter with various synthetic promoters. In some embodiments synthetic promoters are used in combination with mutant casein proteins such as Seq. nos. 199-204, 207-212, or 215-216.

### Synthetic Transcriptional Regulation and Hormonal Modulation for Enhanced Casein Gene Expression

### Use of Synthetic Transcription Factors to Drive Expression of Casein Genes in Seeds

In the context of genetic regulation, a transcription factor is identified as a protein with the specific capacity to bind to designated DNA sequences, thereby directing the transfer of genetic information from DNA to messenger RNA (mRNA). Through either enhancement or suppression of gene transcription, transcription factors are pivotal in the modulation of gene expression.

Seqs. nos. 167-168 provide a modified dTALE-2, a distinct variant within the transcription activator-like effector nucleases (TALENs) family. TALENs represent a class of synthetic proteins engineered to engage with specific DNA sequences, facilitating precise genetic alterations. These proteins are characterized by a TALE DNA-binding domain, originally derived from Xanthomonas bacteria, coupled with a FokI nuclease domain, responsible for executing DNA cleavage at the designated locus.

Transcription factors, notably those derived from microbial sources, have been identified for their roles in either the activation or repression of transcription. A common attribute among these factors is the presence of protein segments capable of recognizing and binding to specific DNA sequences. Despite the general unpredictability of protein-DNA interactions, a subset of transcription factors exhibits a modular binding mechanism, enabling the synthetic creation of transcription factors aimed at specific DNA sequences.

Seqs. nos. 167-168, a synthetic transcriptional activator, exemplifies such innovation, with a design to recognize and bind to a DNA sequence approximately 12 nucleotides in length, subsequently activating the transcription of a downstream gene.

In some embodiments, dTALE-2 sequence is utilized in combination with various genes, such as casein, FAM20C, or other genes of interest. A library of sequences with varying recognition affinities by dTALE-2 allows for the fine-tuning of gene expression targeting, thereby expanding the potential applications of this technology in gene regulation and synthetic biology.

Example 4 demonstrates the utilization of flow cytometry to analyze the expression and regulatory effects of genes in soybean cell protoplasts, specifically highlighting how the dTALE-2 gene activates the STAP21 promoter to enhance GFP gene expression in soy.

In some cases, the dTALE-2 gene (e.g., Seq. no. 164) can be utilized to increase the expression of an exogenous gene by incorporating it alongside a highly specific promoter that is responsive to environmental stimuli, thereby enabling the controlled activation of gene expression in response to external conditions. In some instances, co-expression of the dTALE-2 gene with a synthetic transcriptional activator could further amplify the expression of the target gene. In some cases, the dTALE-2 gene is engineered to target multiple promoters simultaneously, enhancing the expression of a suite of beneficial genes in a coordinated manner. In some cases, the modification of the dTALE-2 gene to include inducible degradation tags allows for temporal control over its activation function, providing a dynamic tool for gene expression studies.

### Hormonal Induction

In some aspects, the genetically modified soy plant is further engineered to overproduce certain hormones that enhance casein gene expression. In some embodiments, the disclosed methods involve the overproduction of certain hormones in the genetically modified soy plant by at least 10%, at least 20%, at least 30%, at least 40%, at least 50%, at least 60%, at least 70%, at least 80%, at least 90%, up to and including 100%, or more, compared to a plant not subjected to the disclosed methods.

In some aspects, these hormones are externally applied to the plant's environment. In some cases, the external application of these hormones to the plant's environment is in a concentration range of 1 to 1000 μM, including all values and ranges therebetween, such as 5, 10, 50, 100, 200, 500 μM, or any range therein. Certain hormones, such as auxins, enhance gene expression and increase the production of casein protein in the soy plant. In some cases, the soy plant is genetically modified to overproduce auxins, a class of phytohormones that regulate various aspects of plant growth and development. In some cases, this is achieved by overexpressing a gene involved in auxin biosynthesis, such as the YUCCA genes in Arabidopsis. In some cases, the degradation of auxins is reduced by downregulating or knocking out a gene involved in auxin degradation, such as the GH3 genes.

In some cases, auxins are added to the growing media of the soy plant. For example, in some cases, natural auxin is applied, such as indole-3-acetic acid (IAA). In some cases, a synthetic auxin, such as 2,4-dichlorophenoxyacetic acid (2,4-D), is added to the soil or culture media in which the soy plant is grown.

In some cases, gibberellic acid, another class of phytohormones, is used to enhance casein gene expression. In some cases, the soy plant is genetically modified to overproduce gibberellic acid. In some cases, gibberellic acid is added to the plant's environment. In some cases, both of these techniques are used. In some cases, the overproduction or external application of these hormones results in an increase in casein protein production by at least 10%, at least 20%, at least 30%, at least 40%, at least 50%, at least 60%, at least 70%, at least 80%, at least 90%, up to and including 100%, or more, compared to a plant not subjected to the disclosed methods.

### Promoter Modification, Selection, and Enhancement

In certain embodiments of the present invention, the promoter of the casein gene is replaced with a more potent promoter to enhance the expression of casein in the genetically modified soy plant. Several potential promoters may be employed for this purpose. In some cases, the Cauliflower Mosaic Virus 35S (CaMV 35S) promoter is utilized to drive high-level, continuous expression of the casein gene. In some cases, the maize ubiquitin-1 (Ubi-1) promoter or the rice actin1 (Act1) promoter are used. In some cases, the elongation factor 1 alpha (EF1α) promoter is used. In some cases, the RD29A promoter is used. In some cases, the Nopaline Synthase (NOS) promoter is used. In some cases, soybean-derived promoters, such as the GmGPDH promoter, are used to increase the efficiency of casein gene expression. In some cases, a combination of promoters is used. In some embodiments, the disclosed methods result in a replacement of the native promoter of the casein gene with a more potent promoter, wherein the degree of replacement is at least 10%, at least 20%, at least 30%, at least 40%, at least 50%, at least 60%, at least 70%, at least 80%, at least 90%, up to and including 100%.

### RNA-Based Upregulation

### Post-Transcriptional Regulation

In certain embodiments of the present invention, post-transcriptional gene regulation methods, such as RNA interference (RNAi), are employed to suppress the activity of negative regulators of casein expression, thereby enhancing the overall production of casein protein in the genetically modified soy plant. In some embodiments, the disclosed methods result in the suppression of negative regulators of casein expression by at least 10%, at least 20%, at least 30%, at least 40%, at least 50%, at least 60%, at least 70%, at least 80%, at least 90%, up to and including 100%. In some cases, the suppression of negative regulators leads to an increase in casein protein expression by at least 10%, at least 20%, at least 30%, at least 40%, at least 50%, at least 60%, at least 70%, at least 80%, at least 90%, up to and including 100%, or more, as compared to a soy plant where the negative regulators are not suppressed.

RNAi is a biological process in which small interfering RNAs (siRNAs) mediate the degradation of specific messenger RNAs (mRNAs), thereby reducing the expression of the corresponding proteins. In the context of this invention, siRNAs can be designed to target the mRNAs of proteins that negatively regulate casein expression. These can include, for example, repressors that bind to the casein gene promoter and inhibit its activity, or proteins that promote the degradation of casein mRNA or protein.

By introducing these siRNAs into the soy plant, the mRNAs of the negative regulators can be selectively degraded, leading to a decrease in the levels of the negative regulator proteins. This, in turn, can relieve the repression of casein expression, resulting in higher levels of casein protein production.

In addition to RNAi, other post-transcriptional gene regulation methods can be used to achieve a similar effect. For instance, antisense RNAs or CRISPR interference (CRISPRi) can be used to inhibit the expression of the negative regulators.

This strategy of suppressing negative regulators provides a novel and effective approach to enhancing the production of non-native proteins in genetically modified plants.

### Alternative Splicing

In some cases, the present invention employs alternative splicing to enhance the expression of the casein gene in transgenic plants. In some cases, the casein gene is modified to include additional exons, which are the coding regions of the gene. These additional exons are included or excluded during splicing to produce different versions of the casein protein. In some cases, the casein gene is modified to include alternative splice sites. In some cases, the splicing machinery itself is modified to favor the production of the casein mRNA. In some cases, this involves overexpressing certain splicing factors. In some cases this involves modifying the splicing factors to preferentially recognize the casein gene. In some cases, small molecules or other factors are used to influence the splicing process; these bind to the splicing machinery or the mRNA itself to influence the splicing process and enhance casein expression. In some cases, the introns, or non-coding regions, of the casein gene are modified. In some cases, the alternative splicing process is manipulated using genetic engineering tools, such as CRISPR-Cas9.

### Riboswitches

In some cases, the present invention employs riboswitches to enhance the expression of the casein gene in transgenic plants. In some cases, a riboswitch is inserted into the mRNA of the casein gene. In some cases, this riboswitch is designed to bind to a specific small molecule, such as a nutrient or a hormone, and increase the expression of the casein gene in response to the presence of this molecule. In some cases, the riboswitch is designed to bind to a product of the casein gene itself, creating a feedback loop, where the production of casein leads to increased expression of the casein gene. In some cases, the riboswitch is designed to bind to a small molecule that indicates the environmental or developmental state of the plant. For example, the riboswitch is designed to increase casein expression in response to drought conditions, or during a specific stage of plant development. In some cases, multiple riboswitches are used in combination to finely tune the expression of the casein gene. In some cases, each riboswitch responds to a different signal, allowing the plant to adjust casein production in response to a variety of conditions. In some cases, the riboswitch is combined with other regulatory elements, such as promoters or enhancers, to further enhance the expression of the casein gene. In some cases, the riboswitch modulates the activity of these other elements, providing an additional layer of control over casein expression. In some cases, the riboswitch is engineered using synthetic biology techniques to respond to novel signals or to have improved performance. In some cases, this involves modifying the sequence of the riboswitch, or combining elements from different natural riboswitches to create a synthetic riboswitch with desired properties.

In some cases, a riboswitch is inserted into the mRNA of the casein gene within a range of −100 to +100 nucleotides relative to the gene's start codon. In some cases, the riboswitch is inserted within a range of −50 to +50 nucleotides relative to the gene's start codon. In some cases, the riboswitch is inserted within a range of −25 to +25 nucleotides relative to the gene's start codon. In some cases, riboswitches designed to enhance the expression of the casein gene in transgenic plants exhibit increased activity within specific ranges of nutrient concentrations. In some cases, the riboswitch responds to nutrient concentrations ranging from 0.1 mM to 1 mM. In some cases, the riboswitch exhibits increased activity within a range of nutrient concentrations from 0.5 mM to 2 mM. In some cases, the riboswitch is designed to respond to nutrient concentrations spanning from 0.01 mM to 0.1 mM.

In some cases, riboswitches designed to enhance the expression of the casein gene in transgenic plants exhibit increased activity within specific ranges of hormone concentrations. In some cases, the riboswitch responds to hormone concentrations ranging from 10 nM to 100 nM. In some cases, the riboswitch exhibits enhanced activity within a range of hormone concentrations from 1 μM to 10 μM. In some cases, the riboswitch is designed to respond to hormone concentrations spanning from 100 pM to 1 nM.

### Introns Addition

In certain embodiments, introns are incorporated into the coding sequence of the casein gene to enhance its expression, thereby increasing the overall production of casein protein in the genetically modified soy plant. Introns are non-coding sequences that are transcribed into pre-mRNA and then removed during the process of splicing. The presence of introns in a gene can enhance its expression, potentially due to the promotion of mRNA export from the nucleus, enhancement of mRNA stability, or improvement of translation efficiency. In some embodiments, the disclosed methods involve the incorporation of at least one, at least two, at least three, at least four, at least five, up to and including ten or more introns into the sequence of the casein gene.

In some cases, introns from the maize Adh1 gene are incorporated into the casein gene. This is achieved by inserting the Adh1 introns at appropriate positions within the casein coding sequence, taking care to ensure that the reading frame of the gene is maintained. In some cases, introns from the castor bean catalase gene is used. In some cases, the first intron of the rice actin gene is used. In some cases, the intron from the potato ST-LS1 is incorporated into the casein gene. In some cases, the third intron from the Arabidopsis thaliana Ubiquitin 10 (UBQ10) gene (AT4G05320.8) is used. The UBQ10 gene is highly expressed in Arabidopsis, and its introns enhance the expression of the casein gene.

In some cases, multiple introns are added to the casein gene to further enhance its expression. In some cases, this is the same intron repeated multiple times. In some cases, different introns are combined to take advantage of their unique properties. In some cases, the introns are added in a specific order to create an intron “cassette” that maximizes casein gene expression.

### Translational Enhancers

In some aspects, translational enhancers are incorporated in the sequence to enhance the efficiency of the translation process, thus increasing casein production. In some cases, an internal ribosome entry site (IRES) is inserted into the sequence of the casein gene. In some cases, the omega sequence from the Tobacco Mosaic Virus (TMV) is used as a translational enhancer. The TMV omega sequence is a 5′ untranslated region (UTR) that enhances the translation of downstream genes. By inserting the TMV omega sequence upstream of the casein gene, the initiation of translation can be improved, leading to increased production of the casein protein. In some cases, the use of a Kozak sequence, a specific sequence of nucleotides surrounding the start codon of a eukaryotic mRNA, can enhance the initiation of translation.

By optimizing the Kozak sequence surrounding the start codon of the casein gene, the binding of the ribosome can be improved, leading to increased translation efficiency. Examples of optimization of the Kozak sequence are known in the art, for example, see Xu, L., Liu, P., Dai, Z. et al. Fine-Tuning The Expression Of Pathway Gene In Yeast Using A Regulatory Library Formed By Fusing A Synthetic Minimal Promoter With Different Kozak Variants. Microb Cell Fact 20, 148 (2021). https://doi.org/10.1186/s12934-021-01641-z.

In some cases, untranslated regions (UTRs) from genes known to be highly expressed in soy or other plants are used as translational enhancers. These UTRs often contain elements that enhance translation, such as binding sites for proteins or small RNAs that promote ribosome recruitment or assembly. In some cases, certain coding sequences can act as translational enhancers by improving the efficiency of the elongation or termination stages of translation. For example, optimizing the codon usage of the casein gene to match the codon bias of highly expressed soy genes can enhance the speed and accuracy of translation elongation.

In some embodiments, the disclosed methods result in an increase in the efficiency of translation of the casein gene by at least 10%, at least 20%, at least 30%, at least 40%, at least 50%, at least 60%, at least 70%, at least 80%, at least 90%, up to and including 100%, or more, compared to a plant not subjected to the disclosed methods. In some cases, the increase in translation efficiency leads to an increase in casein protein production by at least 10%, at least 20%, at least 30%, at least 40%, at least 50%, at least 60%, at least 70%, at least 80%, at least 90%, up to and including 100%, or more, compared to a plant not subjected to the disclosed methods.

### 3′ Untranslated Region (UTR) Modification

In some cases, the 3′ untranslated region (3′ UTR) of the casein mRNA is optimized to enhance its expression, thereby increasing the overall production of casein protein in the genetically modified soy plant. Some cases involve the addition of a polyadenylation signal to the 3′ UTR. This process is typically signaled by a specific sequence in the 3′ UTR, such as the canonical AAUAAA signal. The poly(A) tail protects the mRNA from degradation, enhances its export from the nucleus, and promotes its translation, all of which increase the production of the casein protein. In some embodiments, the length of the poly(A) sitail added to the 3′ UTR of the casein mRNA is between 10 and 250 nucleotides, including all values and ranges therebetween, such as 20, 30, 40, 50, 60, 70, 80, 90, 100, 110, 120, 130, 140, 150, 160, 170, 180, 190, 200, 210, 220, 230, 240 nucleotides, or any range therein. In a preferred embodiment, at least one of sequences 88 through 117 are utilized as 3′ UTRs.

In some cases, uridylation is employed, wherein uridine residues are added to the 3′ end of the mRNA. In some cases, the addition of uridine protects the mRNA from degradation, enhances its export from the nucleus, and promotes its translation, all of which increase the production of the casein protein. In some cases, polyuridylation is employed, wherein multiple uridine residues are added. In some cases guanylation (i.e., addition of guanine nucleotide to the 3′ end of the mRNA), polyguanylation (i.e., addition of multiple guanine nucleotide), cytidylation (i.e., addition of cytosine residues to the 3′ end of the mRNA), or polycytidylation (i.e., addition of multiple cytosine) is utilized as an alternative.

In some cases, binding sites for microRNAs (miRNAs) that are known to be lowly expressed in the soy plant are added, thereby reducing the chance of miRNA-mediated repression of the casein mRNA. In some cases, binding sites for microRNAs (miRNAs) that are known to be highly expressed in the soy plant are added. In some cases, binding sites for RNA-binding proteins that stabilize the mRNA or enhance its translation are included. In some cases, the 3′ UTR includes binding sites for at least one, at least two, at least three, at least four, at least five, or more different microRNAs that are known to be lowly expressed in the soy plant. The specific number of different miRNAs targeted can be adjusted based on the desired level of casein protein production.

In some embodiments, the length of the 3′ UTR of the casein mRNA is optimized to be between 50 and 2000 nucleotides, including all values and ranges therebetween, such as 100, 200, 300, 400, 500, 600, 700, 800, 900, 1000, 1100, 1200, 1300, 1400, 1500, 1600, 1700, 1800, 1900 nucleotides, or any range therein.

In some cases, the 3′ UTR includes at least one, at least two, at least three, at least four, at least five, or more polyadenylation signals. The specific number of polyadenylation signals can be adjusted based on the desired level of casein protein production.

In some cases, the 3′ UTR includes at least one, at least two, at least three, at least four, at least five, or more binding sites for lowly expressed microRNAs. The specific number of miRNA binding sites can be adjusted based on the desired level of casein protein production.

In some cases, the 3′ UTR includes at least one, at least two, at least three, at least four, at least five, or more binding sites for RNA-binding proteins that stabilize the mRNA or enhance its translation. The specific number of RNA-binding protein sites can be adjusted based on the desired level of casein protein production.

In some instances, ribosome profiling is conducted on soy that has been genetically modified to expression at least three foreign genes from among, αs1-casein, αs2-casein, β-casein, κ-casein, and FAM20C, to identify optimal 3′ UTRs. In some embodiments, these casein are among those required to produce a casein micelle. In some embodiments, the soy has also been genetically modified to express a kinase, such as FAM20C, to enable phosphorylation.

The following sequences (Seq. Nos. 88-117 and 249-250) are shown to be effective 3′ UTRs to enhance expression of exogenous proteins such as casein and exogenous kinase in soy. In some instances, the 3′ UTR region utilized is at least 70%, 74%, 78%, 82%, 86%, 90%, 93%, 96%, 98%, or 99% identical to a sequence from among sequences 88 through 117.

### 5′ Untranslated Region

Ribosome profiling, commonly known as Ribo-seq, is a next-generation sequencing technique that provides a snapshot of ribosome positions along transcripts at a given moment. The methodology enables researchers to generate high-resolution, genome-wide maps of ribosome-mRNA interactions, facilitating the quantitative and qualitative assessment of translation (protein synthesis).

Ribosome profiling is conducted on soy that has been genetically modified to expression at least three foreign genes from among, αs1-casein, αs2-casein, β-casein, κ-casein, and FAM20C. In some embodiments, these casein are among those required to produce a casein micelle. In some embodiments, the soy has also been genetically modified to express a kinase, such as FAM20C, to enable phosphorylation

From the Ribo-seq data, preliminary data listing UTRs with optimal performance in translation is obtained. From this list, a specialized library of approximately 2000 high-performing UTRs is constructed. In some instances, the Kozak consensus sequence is appended to UTRs. In some instances, the Upstream Open Reading Frames (uORFs) are removed. The translational efficiency of the specialized library is further probed using a “two reporter” system. Green fluorescent protein (GFP) and red fluorescent protein (RFP) are utilized as reporters. Both proteins are controlled by the same constitutively active promoter, specifically the 35S promoter, ensuring that differences in expression levels are attributable to the UTRs rather than the promoters.

Referring to FIG. 7, The omega Tobacco Mosaic Virus 5′ Untranslated Region (“TMV Ω 5′ UTR”), known for its high translational efficiency, are attached to the red fluorescent protein, serving as a control. The test UTRs from the specialized library are attached to the green fluorescent protein. PEG-mediated transformation is employed to introduce the test UTRs into the system. This involves the plasmids containing the approximately 2000 UTRs being fired into the target cells, facilitating high-throughput screening for translational efficiency.

The analysis process quantifies translational efficiency by evaluating the ratio of green to red fluorescent proteins. A higher ratio indicates elevated translation, while a lower ratio suggests reduced translation efficiency. This fluorescence-based metric serves as a robust quantitative measure for assessing translational performance.

Vectors are introduced into a sample of approximately 100,000 protoplasts, achieving approximately a 10% transformation success rate, and thus resulting in approximately 10,000 transformed protoplasts. Fluorescent-activated cell sorting (FACS) is employed to segregate these protoplasts. Untranslated protoplasts are removed, and the remaining population is categorized into bins based on translational efficiency, such as the top 5%, 25%, and 50%. These high-performing protoplasts are then selected for subsequent sequencing.

Sequences 156 through 163 are DNA sequences of 5′ UTR's that have been shown to be effective at increasing the expression of casein in transgenic soy. In some instances, at least one sequence selected from the group consisting of sequences 156 through 163 is positioned at the 5′ end of a plasmid. In some instances, at least one of sequences 39 through 87, which represent UTR sequences, are positioned at the 5′ end of the plasmid. Said plasmid comprises at least one, two, or three genes chosen from the group consisting of αₛ₁-casein, αₛ₂-casein, β-casein, κ-casein, and a kinase, such as FAM20C. The integration of at least one of sequences 156 through 163 at the 5′ end serves to regulate or facilitate specific functions such as transcription initiation or translation efficiency. In some instances, these sequences may act as promoter or enhancer elements, thereby modulating the expression levels of the genes present in the plasmid. In some instances, alternative regulatory elements can be positioned at the 5′ end to achieve similar or varied outcomes, such as elements designed for tissue-specific expression or inducible expression systems. In some instances, the 5′ UTR region utilized is at least 70%, 74%, 78%, 82%, 86%, 90%, 93%, 96%, 98%, or 99% identical to a sequence from among sequences 39 through 87, or 156 through 163 inclusive.

In some instances, bi-directional promoters are employed within the genetic construct. Bo-directional promoters are specialized DNA sequences located between the 5′ ends of two genes that are oriented in a head-to-head configuration, meaning they are positioned in opposing directions on anti-parallel DNA strands. Said bi-directional promoters possess the capability to initiate the transcription of both flanking genes, frequently in a simultaneous manner. Utilization of bi-directional promoters serves to economize on genomic space and can facilitate the coordinated regulation of the adjoining genes. In a preferred embodiment, such promoters might be employed to simultaneously regulate the expression of both a casein gene and a kinase such as FAM20C, or two different caseins, thereby achieving a synergistic effect in processes like protein synthesis, phosphorylation, and casein micelle formation.

In some instances, alternative plants to soy may be utilized in conjunction with the methods described herein, such as lentils, chickpeas, black beans, and peanuts; grains like quinoa, amaranth, rice, and barley; pseudocereals such as buckwheat and teff; nuts and seeds including almonds, sunflower seeds, chia seeds, flaxseeds, and walnuts; edible fungi like Agaricus bisporus, shiitake, and oyster mushrooms; root vegetables comprising potatoes, sweet potatoes, and yams; leafy greens such as spinach, kale, collard greens, and Swiss chard; seaweeds including Spirulina, Nori, and Kelp; fruits like avocado, guava, and blackberries; tubers including cassava and taro; and other plants like asparagus, artichokes, and okra. In some instances, cruciferous vegetables like broccoli, cauliflower, and Brussels sprouts may be utilized. In some instances, herbs such as basil, cilantro, and mint can also be used as alternatives.

In some instances, additional alternative plant sources may be utilized. Cereals extend to wheat, corn, and millet; more legumes include mung beans, kidney beans, and navy beans; additional nuts and seeds consist of cashews, hazelnuts, and pumpkin seeds. In a preferred embodiment, more fruits may incorporate bananas, apples, oranges, mangoes, and pomegranates. Berries such as blueberries, strawberries, and raspberries present another subset. Root vegetables extend to turnips, beets, and radishes; while tubers like sunchokes and jicama are also viable. Additional leafy greens include arugula, romaine lettuce, and endive. More herbs and spices can range from parsley and oregano to rosemary and thyme. In some instances, members of the gourd family like zucchini, squash, and cucumber may be integrated. Other vegetables like eggplant, bell peppers, and tomatoes—technically fruits in botanical terms—can serve as alternatives. Edible flowers such as calendula, dandelions, and violets; and grains such as rye and sorghum, can also be considered. Aquatic plants like watercress and duckweed, and even unconventional sources like cattails and daylilies. Finally, tropical fruits like durian, lychee, and dragon fruit may be utilized.

In some instances, both 5′ untranslated regions (UTRs) and 3′ UTRs are utilized within a single genetic construct to optimize gene expression. The 5′ UTR sequences, selected from among sequences 39 to 87, are integrated at the 5′ end of a plasmid, serving to modulate functions such as transcription initiation and translation efficiency. In particular, sequence 79 (named “ΩSBC” by Applicant) has been identified as a particularly effective and novel 5′ sequence for use in expression of exogenous casein in transgenic soy. Concurrently, the 3′ UTR sequences, chosen from among sequences 88 to 117, are incorporated at the 3′ end of said plasmid to enhance the stability and translation of the mRNA. In some instances, the 5′ UTR region utilized is at least 70%, 74%, 78%, 82%, 86%, 90%, 93%, 96%, 98%, or 99% identical to a sequence from among sequences 39 through 87. Similarly, the 3′ UTR region utilized is at least 70%, 74%, 78%, 82%, 86%, 90%, 93%, 96%, 98%, or 99% identical to a sequence from among sequences 88 through 117. The co-utilization of both 5′ and 3′ UTR sequences enables a comprehensive modulation of gene expression, thereby offering a more robust control over processes like transcription, translation, and mRNA stability

Some aspects of the disclosure provide a soybean comprising casein proteins, wherein the casein proteins comprise at least 0.03% of the total weight of the seed, at least 0.041% of the total weight of the seed, or at least 0.055% of the total weight of the seed, or at least 0.075% of the total weight of the seed, or at least 0.101% of the total weight of the seed, or at least 0.136% of the total weight of the seed, or at least 0.184% of the total weight of the seed, or at least 0.248% of the total weight of the seed, or at least 0.335% of the total weight of the seed, or at least 0.452% of the total weight of the seed, or at least 0.610% of the total weight of the seed, or at least 0.824% of the total weight of the seed, or at least 1.113% of the total weight of the seed, or at least 1.503% of the total weight of the seed, or at least 2.031% of the total weight of the seed, or at least 2.744% of the total weight of the seed, or at least 3.707% of the total weight of the seed, or at least 5.006% of the total weight of the seed, or at least 6.761% of the total weight of the seed, or at least 13% of the total weight of the seed.

Some aspects of the disclosure provide a soybean comprising casein proteins, wherein the casein proteins comprise at least 0.075% of the total protein content of the seed, or at least 0.103% of the total protein content of the seed, or at least 0.138% of the total protein content of the seed, or at least 0.188% of the total protein content of the seed, or at least 0.253% of the total protein content of the seed, or at least 0.340% of the total protein content of the seed, or at least 0.460% of the total protein content of the seed, or at least 0.620% of the total protein content of the seed, or at least 0.838% of the total protein content of the seed, or at least 1.130% of the total protein content of the seed, or at least 1.525% of the total protein content of the seed, or at least 2.060% of the total protein content of the seed, or at least 2.783% of the total protein content of the seed, or at least 3.758% of the total protein content of the seed, or at least 5.078% of the total protein content of the seed, or at least 6.860% of the total protein content of the seed, or at least 9.267% of the total protein content of the seed, or at least 12.503% of the total protein content of the seed, or at least 16.903% of the total protein content of the seed, or at least 32.5% of the total protein content of the seed.

Some aspects of the disclosure provide a soybean comprising casein micelles, wherein the casein micelles comprise at least 0.03% of the total weight of the seed, or at least 0.041% of the total weight of the seed, or at least 0.055% of the total weight of the seed, or at least 0.075% of the total weight of the seed, or at least 0.101% of the total weight of the seed, or at least 0.136% of the total weight of the seed, or at least 0.184% of the total weight of the seed, or at least 0.248% of the total weight of the seed, or at least 0.335% of the total weight of the seed, or at least 0.452% of the total weight of the seed, or at least 0.610% of the total weight of the seed, or at least 0.824% of the total weight of the seed, or at least 1.113% of the total weight of the seed, or at least 1.503% of the total weight of the seed, or at least 2.031% of the total weight of the seed, or at least 2.744% of the total weight of the seed, or at least 3.707% of the total weight of the seed, or at least 5.006% of the total weight of the seed, or at least 6.761% of the total weight of the seed, or at least 13% of the total weight of the seed.

Some aspects of the disclosure provide a soybean comprising casein micelles, wherein the casein micelles comprise at least 0.075% of the total protein content of the seed, or at least 0.103% of the total protein content of the seed, or at least 0.138% of the total protein content of the seed, or at least 0.188% of the total protein content of the seed, or at least 0.253% of the total protein content of the seed, or at least 0.340% of the total protein content of the seed, or at least 0.460% of the total protein content of the seed, or at least 0.620% of the total protein content of the seed, or at least 0.838% of the total protein content of the seed, or at least 1.130% of the total protein content of the seed, or at least 1.525% of the total protein content of the seed, or at least 2.060% of the total protein content of the seed, or at least 2.783% of the total protein content of the seed, or at least 3.758% of the total protein content of the seed, or at least 5.078% of the total protein content of the seed, or at least 6.860% of the total protein content of the seed, or at least 9.267% of the total protein content of the seed, or at least 12.503% of the total protein content of the seed, or at least 16.903% of the total protein content of the seed, or at least 32.5% of the total protein content of the seed.

Example 5, below, describes an experimental approach to determine the most effective 5′ untranslated regions (5′ UTRs) for enhancing gene expression, utilizing techniques from synthetic biology, fluorescence-activated cell sorting (FACS), and RNA sequencing. The process initiated with the development of an extensive 5′ UTR library, incorporating both soybean-derived sequences and synthetic constructs, which were then inserted into a dual reporter vector for comparative expression analysis. Following transformation into zygotic protoplasts and FACS-based sorting, RNA sequencing was employed to identify 5′ UTRs correlated with high levels of gene expression. This method highlights a strategic evaluation of 5′ UTRs' impact on gene expression, offering a framework for optimizing gene expression in soybean genetics.

### CRISPR/Cas9-Mediated Precision Editing of Protease Genes

In some embodiments, the invention employs the CRISPR/Cas9 system, which includes Cas9 endonuclease and guide RNA (gRNA), for precise gene editing in soy plants. The gRNA guides Cas9 to specific DNA sequences within the protease gene, allowing for targeted modifications. This process involves unwinding the DNA and making precise cuts at designated sites within the protease gene. Post-editing, the modified cells undergo Polymerase Chain Reaction (PCR) to amplify and scrutinize the edited DNA, with a selection process to pinpoint cells that have successfully integrated the edit. To address the typically low efficiency of Homology-Directed Repair (HDR) in such applications, the method uses a DNA repair template to introduce specific changes at the Cas9/gRNA cut site. Additionally, the invention integrates ‘Lox’ sites, recognized by the Cre recombinase enzyme, which flank the Cas9 and gRNA components. This setup allows for the removal of intervening sequences, enabling conditional knockout or modification strategies. A significant aspect of this invention is the introduction of a modified form of the protease, rather than merely knocking out the native protease. This modification is aimed at altering the protease's function to enhance the expression of foreign proteins, such as casein, in transgenic soy.

Subtilisin, a serine protease, is known for its significant impact on casein, primarily due to its proteolytic activity and specificity. This interaction between subtilisin and casein is crucial in various biochemical processes. Subtilisin cleaves casein at specific peptide bonds, leading to the degradation or modification of casein proteins. This proteolytic action is vital in the dairy industry, particularly in cheese-making, where subtilisin's ability to efficiently hydrolyze casein aids in the coagulation process, transforming milk into cheese with desired textures and flavors. Additionally, the interaction between subtilisin and casein can lead to the release of bioactive peptides from casein, which are known for their health-promoting properties, including antimicrobial, immunomodulatory, and antihypertensive effects.

Although subtilisin is not naturally expressed in soy, endogenous subtilisin-like proteases are expressed in soy. In some embodiments, these proteases are targeted for modification in this inventive process due to their unique characteristics and functional roles in the cellular machinery of plants. These proteases belong to a family of serine proteases, known for their robustness and specificity in protein degradation and processing. Subtilisin-like proteases play a critical role in the regulation of protein turnover and processing. By modifying these proteases, it is possible to manipulate their activity, leading to an increased accumulation of desired foreign proteins, such as casein, in transgenic soy plants. This modification can potentially prevent the degradation of these foreign proteins, enhancing their overall expression and stability within the plant cells. In some instances, Thiol proteinase, also known as cysteine proteinase, are targeted for modification.

### Codon Optimization

In certain embodiments of the present invention, the coding sequence of the casein gene is optimized to align with the codon usage bias of the soy plant, thereby enhancing the efficiency of translation and subsequently, the production of the casein protein. Codon usage bias refers to the phenomenon where certain codons are used more frequently than others in the coding sequences of an organism. This bias can significantly influence the speed and accuracy of protein synthesis, as tRNAs corresponding to frequently used codons are typically more abundant in the cell.

In some cases, the codon optimization process involves substituting rare codons in the casein gene with codons that are more commonly used in the soy plant. For instance, if a particular amino acid in the casein protein is encoded by a rare codon in the casein gene, this codon can be replaced with a synonymous codon (i.e., a codon that codes for the same amino acid) that is more frequently used in soy. This can improve the efficiency of translation, as the ribosome is less likely to stall due to a lack of the corresponding tRNA.

This codon optimization strategy can be applied to the entire casein gene or to specific regions that are particularly rich in rare codons. The optimized casein gene is then introduced into the soy plant using standard genetic engineering techniques. The resulting genetically modified soy plant produces higher levels of the casein protein, due to the improved efficiency of translation. In some embodiments, the disclosed methods result in a codon optimization of the casein gene, wherein the degree of codon optimization is at least 10%, at least 20%, at least 30%, at least 40%, at least 50%, at least 60%, at least 70%, at least 80%, at least 90%, up to and including 100%.

It is contemplated by this invention that the DNA sequences that encode proteins in the accompanying XML file apply also to codon-optimized versions of the DNA sequences.

### Protein-Level Modifications and Strategies

### Signal Peptide Optimization

In some aspects, the signal peptide guides the protein to its correct location in the cell. The signal peptide is optimized to enhance protein production. For example, the signal peptide is replaced with a highly efficient one from another protein to guide the casein protein more efficiently to the endoplasmic reticulum for secretion.

In some cases, the signal peptide is optimized by introducing specific mutations that enhance its recognition by the signal recognition particle (SRP), a ribonucleoprotein complex that recognizes and targets proteins to the ER. In some cases, the signal peptide is replaced with a signal peptide from a protein that is known to be efficiently secreted in a specific tissue or at a specific stage of development. This allows for the targeted production of casein protein in specific tissues or at specific times, which is beneficial for the overall yield or quality of the protein. In some cases, the signal peptide is replaced with a dual-targeting signal peptide that targets the protein to multiple locations within or outside the cell. This increases the overall production of the protein by utilizing multiple protein synthesis and secretion pathways.

In some embodiments, the signal peptide is optimized by introducing at least one, at least two, at least three, at least four, at least five, or more mutations. In some embodiments, the mutations introduced are conservative (i.e., the new amino acid has similar properties to the original one). In other embodiments, the mutations are non-conservative (i.e., the new amino acid has different properties). In some embodiments, the signal peptide is optimized to target the casein protein to seed tissues, such as the cotyledon, endosperm, or embryo.

In some embodiments, the signal peptide is replaced with a dual-targeting signal peptide that targets the protein to at least two, at least three, at least four, or more locations within or outside the cell.

### Post-Translational Modification Engineering

In certain embodiments of the present invention, the sequence or structure of the casein protein is modified to enhance its stability, prevent its degradation, or improve its folding and assembly, thereby increasing the production of functional casein protein in the genetically modified soy plant.

One such modification involves the addition of a glycosylation site to the casein protein. This modification has several beneficial effects on the protein, including enhanced stability, improved folding, increased solubility, and protection from proteolytic degradation. A glycosylation site can be added to the casein protein by introducing a specific sequence of amino acids that is recognized by the glycosylation machinery in the soy plant. This sequence typically consists of the amino acid motif Asn-X-Ser/Thr, where X can be any amino acid except proline. Once the modified casein protein is expressed in the soy plant, it will be glycosylated at this site, which can increase its stability and enhance its production. In some embodiments, the disclosed methods result in the addition of at least one, at least two, at least three, at least four, at least five, or more glycosylation sites to the casein protein. In some cases, the addition of glycosylation sites leads to an increase in casein protein stability or production by at least 10%, at least 20%, at least 30%, at least 40%, at least 50%, at least 60%, at least 70%, at least 80%, at least 90%, up to and including 100%, or more, as compared to a casein protein without the added glycosylation sites.

In addition to glycosylation, other modifications can be made to the casein protein to enhance its stability or prevent its degradation. In some cases, disulfide bonds are introduced to stabilize the protein's three-dimensional structure. In some cases, protease cleavage sites are removed or modified to prevent proteolytic degradation.

### Fusion with Native Storage Protein Precursor Subunits

In some embodiments, challenges associated with the expression of exogenous proteins in plant seeds-such as proteolytic degradation, misrouting, and diminished expression levels are addressed through a novel strategy that leverages the endogenous processing pathways of plant seed storage proteins. Specifically, these embodiments contemplate the attachment of precursor subunits from key soybean storage proteins, such as glycinin and beta conglycinin, to either the N-terminal or C-terminal of foreign proteins, such as casein. This aims to improve the accurate routing of the foreign protein to the seed storage vacuoles, enhance its resistance to proteolytic degradation, and augment its expression levels by employing the plant's intrinsic protein processing mechanisms.

Seed storage proteins in soybeans, primarily glycinin and beta conglycinin, are synthesized as precursor polypeptides that undergo post-translational modifications, including cleavage by specific enzymes within the seed storage vacuole. These proteins are essential for the nutritional quality of seeds and have been extensively studied for their roles in seed development and germination. The precursor subunits of glycinin and beta conglycinin include various alpha (a), alpha-prime (a′), and beta (B) subunits for beta conglycinin, and acidic (A) and basic (B) subunits forming A-B pairs for glycinin. By attaching these precursor subunits to foreign proteins, such as casein, the plant's native mechanisms are harnessed to improve the expression and stability of foreign proteins in seeds.

In some cases, the degree of target protein localization to the intended compartment, such as the seed storage vacuole, achieves levels of at least 10%, 20%, 30%, 50%, 70%, or 99% of the total foreign protein expression. The reduction of mislocalized protein outside the target compartment is targeted at reductions of at least 10%, 20%, 30%, 50%, 70%, or 99% relative to controls. In some cases, enhancements in the foreign protein's half-life, indicative of improved proteolytic stability, are at levels of at least 10%, 20%, 30%, 50%, 70%, or 99%. In some cases, the expression levels of the foreign protein are increased at least 10%, 20%, 30%, 50%, 70%, or 99% compared to baseline expression levels. In some cases, the functional activity of the target protein, as a measure of successful routing, reaches at least 10%, 20%, 30%, 50%, 70%, or 99% of its expected activity based on in vitro or native context assessments.

In some embodiments, alternatives to the precursor subunits from glycinin and beta conglycinin are used, instead using the precursor subunits from any native proteins across different plant species. For example, prolamin and glutelin precursor subunits (common in cereal grains such as rice, maize, and wheat); legumin precursor subunits (found in leguminous plants like peas and lentils); albumin precursor subunits (e.g. soybeans and peanuts); zein precursor subunits (found in maize); or cruzipain precursor subunits (found in plants like papaya). This method can be tailored to optimize the expression levels, routing, and proteolytic stability of an array of foreign proteins, including but not limited to enzymes, antibodies, and other biologically active proteins that have applications in agriculture, medicine, and industry.

### Casein Fusion Proteins

In certain embodiments of the present invention, the casein proteins are fused with a peptide sequence that represents the ubiquitin-associated (UBA) domain. In some cases, the casein proteins are fused with peptide having a sequence that is at least 80% identical to any one of the sequences:

It is contemplated that applying one or more of the above disclosed methods will significantly extend the longevity of casein proteins made in a plant, which will increase the amount of usable caseins at harvest, thereby maximizing the utility and potential applications of the casein proteins derived from plants.

In some aspects, the casein gene is fused with a gene encoding a protein known to be highly expressed in soy, with the aim of enhancing casein expression. This strategy leverages the robust transcriptional and translational machinery already in place for these highly expressed genes, boosting the production of the casein protein in the genetically modified soy plant.

In some cases, the casein gene can be fused with the beta-conglycinin gene, a gene encoding a major storage protein in soy that is known to be highly expressed. This fusion involves placing the casein gene downstream of the beta-conglycinin gene, with a linker sequence in between to allow for the separate translation of the two proteins. In some cases, the casein gene is placed upstream of the beta-conglycinin gene. In some cases, the two genes are placed in a bicistronic arrangement, with an IRES or other translational enhancer in between.

In some cases, the casein gene is fused with the gene for ubiquitin. The casein-ubiquitin fusion is designed such that the casein protein is cleaved from ubiquitin after translation, resulting in the separate production of the two proteins.

### Lipidation

In some aspects, the present disclosure provides a method for enhancing the stability and prolonging the half-life of casein proteins in soybeans through the use of chemical post-translational modifications (PTMs), for example, nitrosylation, methylation, acetylation, lipidation, and pegylation.

In some cases, lipid molecules are added to casein proteins, for example, by cysteine prenylation, N-terminal glycine myristoylation, cysteine palmitoylation, and serine and lysine fatty acylation. In some cases, either a farnesyl or a geranylgeranyl group is added to a cysteine residue at or near the C-terminus of the casein protein. In some cases, a prenyl group is attached by a prenyltransferase enzyme. In some cases, a palmitate, a 16-carbon saturated fatty acid, is added to a cysteine residue. In some cases, the palmitate is attached by a palmitoyltransferase enzyme. In some cases, a myristate is added to an N-terminal glycine residue. In some cases, the myristate is attached by the enzyme N-myristoyltransferase. In some cases, a GPI anchor is added to the C-terminus of the protein. In some cases, the GPI anchor is attached in the endoplasmic reticulum by a complex of enzymes (the GPI transamidase complex).

In some cases, the necessary enzymes to carry out the lipidation are naturally present in the cell. In some cases the enzymes necessary to carry out lipidation are introduced into the plant by genetic engineering. In some embodiments, the disclosed methods result in a degree of post-translational modifications (PTMs) or lipidation of the casein proteins of at least 10%, at least 20%, at least 30%, at least 40%, at least 50%, at least 60%, at least 70%, at least 80%, at least 90%, up to and including 100%.

### Protein Folding Enhancers and Chaperone Proteins

In some cases, the present invention employs protein folding enhancers to increase the stability and functionality of the casein proteins in transgenic plants. In some cases, the protein folding enhancers are molecular chaperones, which are proteins that assist in the folding of other proteins. In some cases, these are overexpressed in the transgenic plant to increase the proportion of casein proteins that fold correctly. In some cases, the protein folding enhancers are small molecules that bind to the casein proteins and stabilize their structure. In some cases, these are applied to the plant or included in the plant's growth medium, allowing them to enter the plant's cells and interact with the casein proteins. The concentration of the small molecules in the growth medium can vary, such as from 1 to 10 μM, from 10 to 50 μM, or from 50 to 100 μM. The application frequency can also vary, such as daily, every 2-3 days, or weekly.

In some cases, the protein folding enhancers are co-expressed with the casein proteins. In some cases, these involve introducing a gene that encodes the protein folding enhancer into the same genetic construct as the casein gene, ensuring that the two proteins are produced in the same cells and at the same time. The expression level of the chaperone genes can vary, such as at least 1.5 times higher expression compared to endogenous levels or at least 10 times higher expression. The gene copy number for the folding enhancer can also vary, such as at least 1-2 copies, at least 2-3 copies, or at least 3-5 copies per genome. In some cases, the protein folding enhancers are engineered to have increased specificity or efficacy. In some cases, this involves modifying the sequence of the protein folding enhancer to improve its binding affinity for the casein proteins or to increase its stability and prevent its degradation. In some cases, the binding affinity enhancement is at least 10-20 times, at least 20-30 times, or at least 30-50 times higher affinity. In some cases, the half-life of the enhancer is increased through engineering. In some cases, the enhanced half-life is at least 2-2.5 times, at least 2.5-3 times, or at least 3-5 times longer half-life compared to the original enhancer. In some cases, the protein folding enhancers are used to prevent the aggregation of the casein proteins. In some cases, the concentration of the folding enhancer is at least 1-10 μM, at least 10-50 μM, or at least 50-100 μM.

In some aspects, chaperone proteins are co-expressed to assist in the proper folding of casein, increasing its stability and overall expression. In some aspects, co-expression of BiP (Binding Immunoglobulin Protein) is used. In some aspects, co-expression of Hsp70 (Heat Shock Protein 70) is used. In some aspects, co-expression of Hsp90 (Heat Shock Protein 90) is used. In some aspects, co-expression of PDI (Protein Disulfide Isomerase) is used. In some aspects, co-expression of FKBP (FK506-binding protein) is used. In some aspects, co-expression of GroEL/GroES (Chaperonin complex) is used. In some aspects, co-expression of Hsp40 (Heat Shock Protein 40) is used. In some aspects, co-expression of Hsp60 (Heat Shock Protein 60) is used. In some aspects, co-expression of Calnexin is used. In some aspects, co-expression of Calreticulin is used. In some aspects, co-expression of Prefoldin is used. In some aspects, co-expression of Cyclophilin is used. In some aspects, co-expression of PPIase (Peptidyl-prolyl cis-trans isomerase) is used. In some aspects, co-expression of Grp94 (Glucose-regulated protein 94) is used. In some aspects, co-expression of Hsc70 (Heat Shock Cognate Protein 70) is used. In some aspects, co-expression of TRIC (TCP-1 Ring Complex) is used. In some aspects, co-expression of CHIP (Carboxyl-Terminus of Hsp70-Interacting Protein) is used. In some aspects, co-expression of DnaK/DnaJ/GrpE (Hsp70 chaperone system) is used. In some aspects, co-expression of ClpB (Caseinolytic peptidase B) is used. In some aspects, co-expression of Bag-1 (Bcl-2-associated athanogene 1) is used.

In various embodiments, it is contemplated that a combination of the aforementioned chaperone proteins may be co-expressed to further enhance the proper folding, assembly, and stability of casein proteins in genetically modified soy plants. The specific combination of chaperones can be optimized based on their synergistic effects, compatibility with soy plants, and their ability to maximize the expression and quality of casein proteins.

In some cases, the strength of the promoter driving the expression of chaperone genes is modulated to fine-tune the levels of chaperone proteins produced and optimize their impact on casein protein folding and stability. In some cases, regulatory elements are incorporated to control the timing of chaperone protein expression. In some cases, tissue-specific promoters are implemented to restrict chaperone protein expression to specific plant tissues or organs, such as developing seeds or storage tissues. In some cases chaperone proteins are engineered to enhance their efficiency, substrate specificity, or tolerance to environmental conditions. Site-directed mutagenesis or protein engineering techniques are employed to generate chaperone variants with improved properties for assisting casein protein folding. In some cases, the expression levels of endogenous chaperones in soy are manipulated to complement the exogenous chaperones. Upregulating the expression of specific endogenous chaperones known to be involved in protein folding synergistically enhances the chaperone network and improves casein protein stability.

In some cases, the present invention employs heat shock proteins to enhance the expression of the casein gene in transgenic plants. In some cases, the transgenic plant is genetically modified to overexpress heat shock proteins. In some cases, this involves introducing an additional copy of a heat shock protein gene. In some cases, this involves modifying the promoter of a heat shock protein gene to increase its expression. In some cases, the heat shock proteins are targeted specifically to the casein protein. In some cases, this involves fusing the heat shock protein to a sequence that binds specifically to the casein protein. In some cases, this involves modifying the heat shock protein to recognize the casein protein. In some cases, the heat shock proteins are modified to enhance their chaperone activity. In some cases, this involves mutating the heat shock protein to increase its binding affinity for the casein protein. In some cases, this involves increasing its ability to prevent protein aggregation. In some cases, the heat shock response of the plant is manipulated to increase the production of heat shock proteins. In some cases, this involves exposing the plant to stress conditions that induce the heat shock response. In some cases, this involves genetically modifying the plant to have a heightened heat shock response. In some cases, small molecules or other factors are used to induce the heat shock response and increase the production of heat shock proteins. In some cases, these bind to specific receptors in the plant and trigger the heat shock response.

In some embodiments, the disclosed methods result in an increase in the expression level of one or more chaperone proteins by at least 10%, at least 20%, at least 30%, at least 40%, at least 50%, at least 60%, at least 70%, at least 80%, at least 90%, up to and including 100%, or more, compared to a plant not subjected to the disclosed methods. In some cases, the increase in chaperone protein expression leads to an increase in casein protein stability and expression by at least 10%, at least 20%, at least 30%, at least 40%, at least 50%, at least 60%, at least 70%, at least 80%, at least 90%, up to and including 100%, or more, compared to a plant not subjected to the disclosed methods.

In some embodiments, one or more of sequences 123 through 141, which represent chaperone amino acid sequences, are co-expressed with one or more casein sequences. In some embodiments, the disclosed methods express a sequences that is at least 50%, 60%, 70%, 80%, 90%, or 95% identical with sequences 123 through 141, co-expressed with at least one exogenous protein, such as casein or FAM20C, in a plant.

### Targeting Anti-Casein

Proteases are enzymes that break down proteins into smaller peptides and amino acids. Proteases play important roles in seed development and maturation, as well as in the post-harvest processing of soybeans. The current disclosure provides compositions, methods and systems for enhancing the levels of casein proteins and casein micelle formation in plants. In some instances, enhancing the levels of caseins and casein micelles in plants is achieved by inhibiting the expression or activity of certain protease genes. In some cases, inhibiting the expression or activity of protease genes is achieved by knockdown or knockout of certain protease genes. Contemplated proteases include one or more of a cysteine protease, an aspartyl proteases, a metalloprotease, a serine proteases, or any combination thereof. In some instances, the targeted protease gene is one or more proteases or anti-casein identified by sequences 1-16, 217-241. In some instances, the targeted protease gene is a protease having at least 80%, at least 85%, at least 90%, at least 95%, or at least 99% sequence identity to any one of the sequences in SEQ Nos. 1-16, 217-241. Methods of knockdown or knockout of protease genes can be accomplished by any suitable method, for example, by using one or more of RNA interference (RNAi), small interfering RNA (siRNA), short-, medium-, or long-hairpin RNA (shRNA, mhRNA, lhRNA), or a bacterial RNA-guided endonuclease directed towards the protease gene. In some cases, inhibiting the expression or activity of protease genes is achieved by expressing proteins that bind to the proteases, for example, an antibody of the protease. In some cases, the plant is soybean. In some instances, levels of casein proteins are increased as compared to a counterpart without the protease knockdown, including any one or more the following casein proteins: K casein, αₛ₁ casein, αₛ₂ casein, or β casein.

In an ideal embodiment, the proteases or anti-casein targeted for knockdown or knock-out are those that break down exogenous casein proteins. Example 1 provides an experiment for determining the operative proteases involved in the breakdown of exogenous casein in soy. In some cases, the proteases targeted for knockdown or knock-out are late-stage proteases. In accordance with the embodiments disclosed herein, it is observed that specific proteases exhibit either negligible or no expression during the initial, or green, stage of plant development, yet demonstrate significantly increased expression levels during subsequent developmental stages, notably the yellow and brown stages. Such proteases, enumerated in sequences 1 through 16, are of particular interest for targeted gene silencing interventions, including both knockdown and knockout strategies. This interest is predicated on the recognition that these proteases are implicated in the degradation processes occurring during the latter stages of plant development. Consequently, a strategic focus on these proteases, categorized based on the developmental stage at which they manifest elevated expression levels within the plant, presents a novel approach for mitigating undesirable degradation phenomena. This methodology facilitates the preservation of plant integrity and quality throughout the entire developmental spectrum, thereby offering significant advancements in agricultural and biotechnological applications.

In some cases, inhibiting expression of a protease comprises using a short hairpin RNA targeting at least a portion of a polynucleotide sequence encoding the protease. In some cases, inhibiting expression of a protease comprises using a nuclease (e.g., Cas9 endonuclease) coupled to a guide RNA targeting at least a portion of a polynucleotide sequence encoding the protease. In some cases, inhibiting expression of a protease comprises inserting a silencer sequence near a polynucleotide sequence encoding the protease. In some cases, inhibiting function of a protease comprises expressing a protease neutralizing antibody. In some cases, inhibiting function of a protease comprises using a small molecule inhibitor of the protease.

In some instances, RNA interference (RNAi) constructs for protease knockdown are designed to target specific protease genes in soybean, leading to their knockdown or knock out of protease genes. In some cases, the RNAi constructs can be introduced into soybean plants using Agrobacterium-mediated transformation or biolistic bombardment. The resulting transgenic soybean plants will have reduced protease activity and increased levels of casein proteins and casein micelles.

In some instances, ubiquitin ligases are targeted for knockdown. These ligases are integral in marking proteins, including caseins, for degradation by proteases. By suppressing the activity or expression of one or more ubiquitin ligases, degradation of casein by the 26S proteasome is reduced, thereby enhancing casein yield. This strategy is particularly advantageous as it allows the 26S proteasome, a component of the ubiquitin-proteasome system (UPS), to maintain its essential role in preserving cellular homeostasis, without compromising the stability and availability of the 26S proteasome. This approach provides a balanced and targeted (i.e., less disruptive) solution to enhance the nutritional quality of our transgenic plants by enhancing casein content in the plants. For example, DOD-type homing endonuclease domain-containing protein (uniprot is AOAOROISD2) or HECT-type E3 ubiquitin transferase (uniprot is K715L6) are targeted for knockdown or knockout.

In some instances, gene editing for protease knockdown can use editing techniques such as CRISPR/Cas9 or TALENs to knock down specific protease genes in soybean. The resulting transgenic soybean plants will have reduced protease activity and increased levels of casein and casein micelles.

In some cases, Ethyl Methanesulfonate (EMS) mutagenesis is utilized as an alternative to CRISPR/Cas9 or TALENs gene editing technology to generate transgenic soybean plants with desired traits. In some instances, soybean seeds are treated with EMS, a chemical mutagen that induces random point mutations in the genome. The treated seeds are then screened for mutations that result in the desired trait, for example, decreased protease activity.

Another aspect of the present disclosure provides the insertion of a gene encoding a protease that specifically targets and degrades the protease that targets casein within transgenic soy plants. The introduced protease gene is operably linked to a promoter that enables expression in the desired plant tissue. Upon expression, the introduced protease targets and cleaves the endogenous protease responsible for the degradation of casein in the soybean seeds.

Another aspect of the present disclosure provides the modification of the caseins in transgenic soybean plants to remove or reduce their susceptibility to protease degradation. In some instances, the genes encoding the major casein proteins (alpha-S1, alpha-S2, beta-, and delta-caseins) are modified to remove the specific sites that are susceptible to protease degradation. The modified genes are then introduced into soybean plants through genetic engineering techniques, resulting in the expression of modified casein proteins that are resistant to protease degradation. The modified caseins have an increased resistance to degradation by proteases, resulting in higher levels of casein in the soybeans and soy products.

In some cases, the modification of soybean plants to enable the expression of protease in a specific cellular compartment other than the organelle where the casein is synthesized and stored. In some instances, the soybean plant genome is modified to introduce a protease gene that is fused with a signal peptide sequence that directs the protease to a specific subcellular compartment, such as the endoplasmic reticulum or the vacuole. The modified protease gene is driven by a promoter that is active in the desired cellular compartment, allowing for the selective expression of protease in that location. By expressing the protease in a specific cellular compartment, the protease can be sequestered away from the organelles where casein is synthesized and stored, reducing the risk of unwanted degradation of other proteins or cellular components.

Another aspect of the present disclosure provides the modification of soybean plants to alter the environmental conditions within the organelles where casein is synthesized and stored, such that the protease that targets casein is no longer active. In some instances, the soybean plant genome is modified to introduce a gene that encodes a protein or enzyme that alters the pH or other environmental conditions within the organelles, such as the endoplasmic reticulum or the Golgi apparatus. The modified gene is expressed under the control of a promoter that is active in the specific organelle, allowing for the selective expression of the protein or enzyme in that location. The altered environmental conditions within the organelle can affect the activity of the protease that targets casein, rendering it inactive or less effective. This can lead to an increase in the amount of casein in the soybeans and soy products, as the casein is no longer degraded by the protease. This embodiment provides a method for enhancing the nutritional value of soybeans and soy products by increasing the level of casein expression, while minimizing the activity of the protease that targets casein. By altering the environmental conditions within the organelle, unwanted protease activity can be reduced or eliminated, resulting in a higher yield of casein and improved nutritional properties of soy products.

Another aspect of the present disclosure provides promoter engineering for casein gene upregulation. In some instances, the promoters of casein genes are modified to increase their activity in soybean seeds. This can be achieved by replacing the native promoter with a strong constitutive promoter or by fusing it with an inducible promoter that can be activated specifically in seeds. The resulting transgenic soybean plants will have increased levels of casein proteins, leading to enhanced nutritional quality of soy protein.

Another aspect of the present disclosure provides the combination of protease knockdown and promoter engineering. In some instances, both protease knockdown and promoter engineering strategies are used to enhance the level of casein proteins in soybean seeds. The resulting transgenic soybean plants will have reduced protease activity and increased levels of casein proteins, leading to further enhanced nutritional quality of soy protein.

Another aspect of the present disclosure provides the modification of soybean plants to express a protease inhibitor within the seed, which can prevent the degradation of casein by the protease that targets it. In some instances, the soybean plant genome is modified to introduce a gene that encodes a protease inhibitor that is specific to the protease that targets casein. The modified gene is expressed under the control of a promoter that is active in the seed, allowing for the selective expression of the protease inhibitor within the seed. Upon expression, the protease inhibitor can specifically inhibit the activity of the protease that targets casein, preventing its degradation and allowing for the accumulation of higher levels of casein within the seed. This can result in an increase in the amount of casein in the soybeans and soy products.

Protease inhibitor treatment during soybean seed processing: In some instances, soybean seeds from transgenic soybean plants with reduced protease activity and increased levels of casein proteins are treated with a protease inhibitor during the seed processing stage. The protease inhibitor is added to the soybean meal produced during seed processing, and it is designed to inhibit proteases that can degrade casein and other proteins with nutritional value. The treated soybean meal can then be used to produce soy protein products with enhanced nutritional quality. The specific protease inhibitor used, as well as the concentration and duration of the treatment, can be optimized based on the proteases targeted and the desired level of inhibition. The use of a protease inhibitor during soybean seed processing can improve the nutritional quality of soy protein products, as it can prevent the degradation of important proteins and preserve their nutritional value.

Another aspect of the present disclosure provides a protease inhibitor during the extraction process. In some instances, at least one protease inhibitor is added to the soybean slurry before or during the extraction process to prevent the degradation of soybean proteins with nutritional value, such as caseins. In one embodiment, a mixture of protease inhibitors, such as PMSF (phenylmethylsulfonyl fluoride), EDTA (ethylenediaminetetraacetic acid), and pepstatin A, can be used to inhibit the activity of serine proteases, metalloproteases, and aspartic proteases, respectively. The protease inhibitor cocktail can be optimized to specifically target the proteases known to degrade the targeted proteins, while minimizing the impact on the overall protein yield and quality. The protease inhibitor cocktail can be added to the soybean slurry in a concentration range of 0.1 to 5 mM, depending on the specific proteases targeted and the desired level of inhibition. The soybean slurry can then be subjected to conventional extraction processes, such as precipitation or ultrafiltration, to obtain soy protein products with enhanced nutritional quality.

Another aspect of the present disclosure provides stacking with other traits. In some instances, the transgenic soybean plants with reduced protease activity and increased levels of casein proteins are stacked with other traits such as herbicide tolerance or insect resistance to create plants with multiple desirable traits. These plants can be used for the production of soy protein products with enhanced nutritional quality and improved agronomic traits.

The strategy of knocking down native proteins plays a critical role, particularly in the study and potential treatment of various biological conditions. While a significant focus has been on proteases—enzymes that catalyze the breakdown of proteins by hydrolyzing peptide bonds—this approach is not exclusive to them. Among the diverse targets for knockdown are ubiquitin ligases, crucial components in the proteostasis network that initiate the degradation of proteins through the ubiquitin-proteasome system. These enzymes tag proteins with ubiquitin, signaling them for degradation and thus regulating protein levels within the cell, impacting numerous cellular functions and pathways.

Moreover, the knockdown strategy extends to specific transcription factors, such as FLC (FLOWERING LOCUS C) from Arabidopsis thaliana. FLC is a well-characterized transcription factor that plays a pivotal role in the regulation of flowering time by repressing the floral transition under certain conditions. Targeting such a transcription factor through knockdown approaches allows for the investigation of its role in plant development and the underlying genetic and epigenetic mechanisms controlling gene expression related to flowering.

In some instances, enhancing the levels of caseins and casein micelles in plants is achieved by inhibiting the expression or activity of certain anti-casein genes. In some cases, inhibiting the expression or activity of anti-casein genes is achieved by knockdown or knockout of certain genes. In some cases, the gene targeted for knockdown or knockout is at least one of the Sequences 1 through 16 or 217 through 241. In a preferred embodiment, knockdown or knock-out of sequence 229, known as “Glyma.09G206500,” has been found to be effective at improving the expression levels of exogenous proteins such as casein in a plant, such as soy. In some cases, inhibiting the expression or activity of protease genes is achieved by knockdown or knockout of certain ubiquitin ligases, which reduces the ubiquitination of casein, reducing the extent to which the 26S proteasome degrades the protein into small peptides. In some cases, the transgenic casein proteins are fused with a SUMO tag to mask potential ubiquitin attachment sites on the casein protein. Contemplated proteases include one or more of a cysteine protease, an aspartyl proteases, a metalloprotease, a serine proteases, or any combination thereof. Methods of knockdown or knockout of protease genes can be accomplished by any suitable method, for example, by using one or more of RNA interference (RNAi), small interfering RNA (siRNA), short-, medium-, or long-hairpin RNA (shRNA, mhRNA, lhRNA), or a bacterial RNA-guided endonuclease directed towards the protease gene. In some cases, inhibiting the expression or activity of protease genes is achieved by expressing proteins that bind to the proteases, for example, an antibody of the protease. In some instances, levels of casein proteins are increased as compared to a counterpart without the protease knockdown, including any one or more the following casein proteins: K casein, α_{S1} casein, α_{S2} casein, or β casein.

### Bowman Birk Inhibitor with a PSV Localization Tag

In some embodiments of the present invention, methods for enhancing the expression of foreign casein genes in soybeans involve using Bowman Birk Protease Inhibitors (BBPIs).

In some instances, the invention involves a method for boosting the expression of foreign casein genes in soybeans. This method involves the co-expression of casein genes and BBPIs in a soybean plant. The BBPIs, being serine protease inhibitors, bind to the active sites of serine proteases which could potentially degrade the casein. By co-localizing these inhibitors with the casein proteins, degradation of the casein is reduced, thereby enhancing its expression in the soybean plant.

In some instances, the invention includes a specific plasmid configuration for achieving the enhanced expression of casein in soybeans. As shown in FIG. 14, In one embodiment, a plasmid, referred to as Pmoz 2274, is designed to express all caseins and a kinase in soybean. Referring to FIG. 15, another embodiment involves a plasmid, Pmoz 2284, which includes the same components as Pmoz 2274 with the addition of BBPIs. These plasmids are used to transform soybean tissue, and the transformation efficiency is assessed using GFP as a marker. The presence of a peptide tag, V5, on the end of the casein, allows for the quantification of alpha casein expression.

Experimental results indicated that the expression of alpha casein was approximately four times greater in the presence of BBPIs than in their absence. This enhancement is attributed to the BBPIs' ability to inhibit proteases that might degrade the casein proteins.

Moreover, the invention includes the addition of a GY1-VSD sequence to the casein genes. This sequence, derived from the GY1 gene, acts as a vesicle sorting determinant, targeting the expressed proteins to the vacuole for better processing and stability

It has been observed that the initial 19 amino acids of the Bowman Birk are cleaved off in the endoplasmic reticulum (E.R.), suggesting modifications post-translation that may affect the functionality of the expressed proteins

In some embodiments, the invention extends to employing various peptide tags, such as His-tag, FLAG-tag, or HA-tag, alongside or in lieu of the V5 tag for enhanced quantification and monitoring of casein expression. The selection of a specific tag is tailored to the experimental requirements and the detection methodologies at hand, facilitating precise tracking and purification of the expressed proteins.

Moreover, the invention encompasses the utilization of diverse promoters within the plasmid constructs to regulate the expression of casein and kinase genes. This includes the use of the 35S CaMV promoter for constitutive expression or the deployment of tissue-specific promoters to achieve targeted expression in desired soybean tissues, optimizing protein production efficiency and quality.

In some embodiments, the invention incorporates the co-expression of chaperone proteins within the soybean, aimed at assisting the correct folding and assembly of the expressed casein proteins. This strategy is pivotal in overcoming challenges related to protein misfolding or aggregation, thereby augmenting the stability and functionality of the recombinant proteins.

Furthermore, the invention explores various transformation techniques such as Agrobacterium-mediated transformation, biolistic particle delivery, or electroporation for the introduction of plasmids into soybean tissue. Each method offers unique benefits in terms of transformation efficiency, suitability for different soybean varieties, and adaptability to diverse tissue types

Additionally, the invention provides a comprehensive analysis of the post-translational modifications experienced by the expressed casein proteins, with a particular focus on those modifications occurring within the endoplasmic reticulum and Golgi apparatus. Such analysis is crucial for enhancing the understanding of protein stability, solubility, and bioactivity, which in turn could lead to soybeans with improved nutritional values.

In some embodiments, the invention addresses the environmental and regulatory considerations associated with the introduction of BBPIs and caseins into soybeans. This includes thorough assessments of potential allergenicity, environmental impacts, and adherence to regulatory guidelines, ensuring that the biotechnological advancements are applied responsibly and safely within the agricultural sector.

In some embodiments, the methods for enhancing the expression of foreign casein genes in soybeans will involve utilizing Bowman Birk DNA sequences represented by sequences identified in SEQ ID 242 through 248.

### Selection of Broad-Spectrum Protease Inhibitors

The protease inhibitors suitable for this method are selected based on their spectrum of activity against the classes of proteases present in soy plants. These inhibitors must exhibit inhibitory effects against a wide range of endogenous proteases, including but not limited to serine proteases, cysteine proteases, aspartic proteases, and metalloproteases. The selection process involves screening for inhibitors that are effective in the specific pH and ionic environments of the soy plant cellular compartments where casein is synthesized, processed, and stored.

The selected protease inhibitors are genetically engineered to include signal peptides that mirror those of the transgenic casein. This engineering ensures that the inhibitors emulate the intracellular trafficking pathway of casein proteins. By co-localizing with casein, the inhibitors offer direct and immediate protection against degradation. In an ideal embodiment, genetic constructs for the chosen protease inhibitors include a promoter active in soy plant cells, preferably a strong constitutive promoter for ubiquitous expression, or a seed-specific promoter for targeted expression in tissues where casein accumulation is desired. These constructs are introduced into the soy genome using established transformation techniques, and stable transgenic lines are selected based on inhibitor expression levels and casein stability.

In some embodiments, protease inhibitors are trafficked from the ER to the Golgi apparatus, then to the pre-vacuolar compartment (PVC), and finally to the PSV. In some embodiments, protease inhibitors are trafficked from the ER to the Golgi apparatus, then to dense vesicles (DV), then to the PVC, and finally to the PSV. In some embodiments, protease inhibitors are trafficked from the ER to the Golgi apparatus, then to multivesicular bodies (MVB), then to the PVC, and finally to the PSV. In some embodiments, protease inhibitors are trafficked from the ER directly to the PVC and finally to the PSV. In some embodiments, protease inhibitors are trafficked from the ER to vacuolar sorting receptor (VSR)-positive vesicles and finally to the PSV. In some embodiments, protease inhibitors are trafficked from the ER to autophagosomes and finally to the PSV. In some embodiments, protease inhibitors are trafficked from the ER to the Golgi apparatus, then to the plasma membrane, then endocytosed, then to the PVC, and finally to the PSV. In some embodiments, protease inhibitors are trafficked from the ER to the Golgi apparatus, then to DV, then to the plasma membrane, then endocytosed, then to the PVC, and finally to the PSV. In some embodiments, protease inhibitors are trafficked from the ER to the Golgi apparatus, then to MVB, then to the plasma membrane, then endocytosed, then to the PVC, and finally to the PSV. In some embodiments, protease inhibitors are trafficked from the ER to the Golgi apparatus, then to exosomes, then to the extracellular space, then endocytosed, then to the PVC, and finally to the PSV. In some embodiments, protease inhibitors are trafficked from the ER to the Golgi apparatus, then to lysosomes, then to autophagosomes, and finally to the PSV.

In some embodiments, casein proteins, such as κ-casein, α_{S1}-casein, α_{S2}-casein, or β-casein are trafficked from the ER to the Golgi apparatus, then to the pre-vacuolar compartment (PVC), and finally to the PSV. In some embodiments, casein proteins are trafficked from the ER to the Golgi apparatus, then to dense vesicles (DV), then to the PVC, and finally to the PSV. In some embodiments, casein proteins are trafficked from the ER to the Golgi apparatus, then to multivesicular bodies (MVB), then to the PVC, and finally to the PSV. In some embodiments, casein proteins are trafficked from the ER directly to the PVC and finally to the PSV. In some embodiments, casein proteins are trafficked from the ER to vacuolar sorting receptor (VSR)-positive vesicles and finally to the PSV. In some embodiments, casein proteins are trafficked from the ER to autophagosomes and finally to the PSV. In some embodiments, casein proteins are trafficked from the ER to the Golgi apparatus, then to the plasma membrane, then endocytosed, then to the PVC, and finally to the PSV. In some embodiments, casein proteins are trafficked from the ER to the Golgi apparatus, then to DV, then to the plasma membrane, then endocytosed, then to the PVC, and finally to the PSV. In some embodiments, casein proteins are trafficked from the ER to the Golgi apparatus, then to MVB, then to the plasma membrane, then endocytosed, then to the PVC, and finally to the PSV. In some embodiments, casein proteins are trafficked from the ER to the Golgi apparatus, then to exosomes, then to the extracellular space, then endocytosed, then to the PVC, and finally to the PSV. In some embodiments, casein proteins are trafficked from the ER to the Golgi apparatus, then to lysosomes, then to autophagosomes, and finally to the PSV.

In some embodiments, casein proteins and protease inhibitors are concomitantly trafficked along the same pathway from the endoplasmic reticulum (ER) to the protein storage vacuoles (PSVs), but the shared route is maintained for only a segment of the journey and a divergence occurs wherein casein proteins continue to the PSVs, while the protease inhibitors do not complete the entire path to these vacuoles.

Quantitative analysis, including but not limited to immunoblotting techniques, is conducted to measure the transgenic casein levels in the presence of the expressed protease inhibitors. Further assessments, such as in-vitro protease inhibition assays and in vivo degradation assays, are performed to determine the effectiveness of these inhibitors in protecting casein proteins from breakdown. By providing ubiquitous or targeted protease inhibition that parallels the life cycle of casein within the soy plant, the present invention aims to create a transgenic plant system with significantly enhanced yields of stable, functional casein protein. These improvements in casein stability offer substantial benefits for the production of soy-based products with high nutritional and functional value.

As shown in FIGS. 12 and 13, in some embodiments of the present invention, a myriad of protease inhibitors may be evaluated and potentially utilized for safeguarding the expression and stability of transgenic casein in soy plants. In some instances, natural plant-derived inhibitors such as the Bowman-Birk Inhibitor (BBI) with dual inhibitory functions against trypsin and chymotrypsin-like proteases might be chosen. In some instances, Kunitz-type inhibitors, renowned for their potent inhibitory properties, could be engineered for an enhanced effect against specific proteases threatening casein stability. In some instances, variants of the Soybean Trypsin Inhibitor (STI), tailored for heightened specificity and efficiency, may be used. In some instances, Potato Inhibitor II (PI-II) derivatives might be modified to navigate and concentrate in cellular compartments alongside casein. In some instances, the extensive and adaptable inhibiting spectrum of Alpha-2-Macroglobulin (α2M) could be harnessed for its unique mechanism of protease trapping and inactivation. In some instances, synthetic inhibitors designed from scratch using computational tools and peptide modeling could provide novel and bespoke protection mechanisms. In some instances, modified serpins with altered reaction centers or cofactor binding sites may be utilized for their ability to change conformation upon protease interaction, irreversibly inhibiting the enzyme. In some instances, cystatin family members with natural cysteine protease inhibiting functions might be employed. In some instances, plant defensins, small cysteine-rich proteins with inhibitory effects on a diverse range of enzymes, could be explored. In other instances, specific inhibitors of aspartic proteases such as pepstatin-insensitive plant aspartic proteases (PIPs) variants may be considered for their targeted action. In some instances, inhibitors targeting the subtilase family or other plant-specific proteolytic pathways could be advantageous. Each of these inhibitors presents, in some instances, a strategic approach to effectively insulating casein within the plant system from detrimental proteolytic activity; thereby, such instances extend the frontier of the invention's scope in this field.

### Use of Broad-Spectrum Protease Inhibitors

In some embodiments of the present invention, the invention provides for enhancing the expression of exogenous proteins in transgenic plants, wherein said plants are genetically modified to express one or more exogenous proteins of interest, such as casein, alongside one or more broad-spectrum protease inhibitors. The exogenous proteins of interest, for example, casein proteins, are introduced into said transgenic plants, including but not limited to soy plants, to produce desired proteins that are not native to the host plant species.

In some cases, the present invention incorporates a genetic construct comprising a sequence encoding the exogenous protein of interest operably linked to a promoter active in the plant cells. Concurrently, the genetic construct includes one or more sequences, identified herein as sequences 17 to 38, each encoding a broad-spectrum protease inhibitor. In some cases, these sequences are similarly operably linked to promoters active in the plant cells, ensuring the co-expression of the broad-spectrum protease inhibitors with the exogenous protein of interest.

As shown in FIGS. 10 and 11, in some embodiments of the present invention, protease inhibitors such as Oryzacystatin-I, GmaxBBI, or sPI-II may be optionally included in the co-expression system at various concentration ranges. For Oryzacystatin-I, originally derived from rice, concentrations may range from 1 to 10 μM, 10 to 50 μM, 50 to 100 μM, 100 to 500 μM, or 500 to 1000 μM. GmaxBBI, sourced from soybean, may be employed at concentrations ranging from 0.5 to 5 μM, 5 to 25 μM, 25 to 50 μM, 50 to 250 μM, or 250 to 500 μM. Similarly, sPI-II, from the Solanum genus, can be used at concentrations of 0.1 to 1 μM, 1 to 10 μM, 10 to 100 μM, 100 to 500 μM, or 500 to 1000 μM. These protease inhibitors offer alternative options for inhibiting a range of serine and cysteine proteases and can be particularly effective in enhancing the stability of target proteins like casein by reducing their degradation.

In some aspects, specialized signal peptides or tags, such as HDEL or VSD, may be used to direct the co-expressed proteins to specific cellular locations. HDEL serves as a well-known endoplasmic reticulum (ER) retention signal but other ER-targeting sequences may also be used. VSD, on the other hand, is one of several domains used for targeting proteins to the plasma membrane. These tags are optional but can be crucial for effective co-localization of casein proteins and protease inhibitors within cellular compartments.

In some aspects, the strategy of co-localization is utilized with varying efficiencies of interaction between the target proteins and protease inhibitors. Co-localization efficiencies may range from 50% to 70%, 70% to 85%, 85% to 95%, or even up to 99%. These efficiency percentages are calculated based on the proportion of casein proteins that are found in the same cellular compartment as the protease inhibitors. Specifically, these percentages may be determined using advanced microscopy techniques, such as confocal microscopy, followed by image analysis that quantifies the degree of overlap between the fluorescent signals emitted by the casein proteins and the protease inhibitors. The aim of these efficiency ranges is to facilitate optimal interaction between casein proteins and protease inhibitors when they are co-localized in the same cellular compartments. This approach can be particularly useful in biotechnological applications and can be achieved through the optional use of specific signal peptides or tags like HDEL and VSD.

In some embodiments, transient transformation is employed as an experimental technique, allowing for the temporary introduction of casein proteins and protease inhibitors into bean plants. This approach is optional but offers the advantage of quick data collection and evaluation of the co-expression system. It is an alternative to more labor-intensive stable transformations.

Quantification methods, such as Immunoblotting and qPCR, are optionally incorporated into the experimental design. Immunoblotting serves as one among various techniques for protein quantification, while qPCR provides an optional method for assessing RNA levels of the expressed genes. For Immunoblotting, sensitivity ranges can vary, with options including 0.1 to 1 ng/mL, 1 to 5 ng/mL, 5 to 10 ng/mL, or even 10 to 50 ng/mL, thereby accommodating different requirements for the specific detection of casein proteins. Similarly, qPCR offers multiple accuracy ranges quantified as cycle threshold (Ct) values, which could be within a range of +0.5 to +1 cycles, +1 to +1.5 cycles, or +1.5 to +2 cycles for the target genes.

Additional techniques, namely In-gel Degradation Assays and Fluorometric Assays, may optionally be used to assess protease activity. These assays serve as valuable adjuncts to primary quantification methods and can be particularly useful in certain embodiments for corroborating the mechanism of protease inhibition.

In some cases, the broad-spectrum protease inhibitors are selected based on their capability to inhibit a wide range of plant proteases, thereby reducing the degradation of the exogenous proteins and enhancing their accumulation within the plant tissues. The protease inhibitors encoded by sequences 17 to 38 are chosen for their broad-spectrum activity, compatibility with the plant host, and minimal adverse effects on the plant's growth and development.

The method further involves the transformation of target plant cells with the aforementioned genetic construct, followed by the regeneration of these cells into mature plants that stably express both the exogenous protein of interest and the broad-spectrum protease inhibitors. This co-expression strategy significantly improves the yield of the exogenous protein, making the transgenic plants a viable system for the production of valuable proteins such as casein.

In some cases, alternative strategies for introducing the genetic construct into the plant cells may be employed, including but not limited to Agrobacterium-mediated transformation, biolistic (particle bombardment) methods, or direct DNA transfer techniques. Furthermore, alternative embodiments of the invention may utilize different promoters, sequences encoding other broad-spectrum protease inhibitors, or target additional plant species beyond soy, provided that such modifications contribute to the efficient expression and accumulation of the exogenous proteins.

### Selection of Promoter

Selecting an appropriate promoter is crucial for enhancing the expression of transgenic proteins, including casein, in soybean plants through genetic engineering. In some instances, the use of strong constitutive promoters may be preferred, and in such instances, the CaMV 35S promoter derived from Cauliflower Mosaic Virus or the FMV 34S promoter from Figwort Mosaic Virus could be utilized. In some instances, promoters from ubiquitin genes, such as the ubiquitin-1 (Ubi-1) promoter from maize, might be selected for their robust activity across different plant tissues. In some instances, actin promoters are chosen due to their high activity levels; these include the rice Act1 and maize Act1 promoters.

In some instances, light-inducible promoters such as those derived from the Rubisco Small Subunit, including the spinach or pea rbcS promoters, are favorable, whereas in other instances, moderate promoter strength is sufficient, which can be provided by the Nopaline Synthase (NOS) promoter from Agrobacterium tumefaciens. In some instances, the promoters are required to be stress-inducible, like the RD29A promoter from Arabidopsis thaliana which responds to drought or salinity stress. In a preferred embodiment, where seed-specific expression is of interest, soybean promoters such as those from Glycinin or β-conglycinin genes may be applied.

In some instances, controlled expression is necessary, which can be achieved with inducible promoters such as the maize Alcohol Dehydrogenase (Adh1) promoter for hypoxia response or tissue-specific promoters to target specific plant parts. In some instances, heat-inducible expression is sought, leading to the selection of promoters such as soybean hsp17.5-E or maize hsp70. In some instances, chemical inducibility is preferred, and the patent contemplates the use of promoters like the XVE system, which is estrogen-inducible, or the GR system inducible by glucocorticoids. Finally, in some instances, the expression needs are unique and may necessitate the construction of synthetic promoters, engineered with multiple regulatory elements to produce a desired and tailored expression profile.

### Antisense RNAs

In certain embodiments of the present invention, methods are employed to silence the expression of negative regulators of casein expression, thereby indirectly enhancing the overall production of casein protein in the genetically modified soy plant. Negative regulators include proteins that repress the transcription of the casein gene, proteins that promote the degradation of casein mRNA or protein, or proteins that inhibit the translation of casein mRNA.

One such method involves the use of antisense RNAs-RNA molecules that are complementary to the messenger RNAs (mRNAs) of the negative regulators. When an antisense RNA binds to its target mRNA, it can prevent the mRNA from being translated into protein, effectively knocking down the expression of the negative regulator. For instance, if a particular protein acts as a repressor by binding to the casein gene promoter and inhibiting its activity, an antisense RNA can be designed to target the mRNA of this repressor protein. By reducing the levels of the repressor protein, the repression of the casein gene can be relieved, leading to increased casein expression.

In some cases, small interfering RNAs (siRNAs) or short hairpin RNAs (shRNAs) are used to induce RNA interference (RNAi), a process that leads to the degradation of the target mRNAs. In some cases, CRISPR interference (CRISPRi) is used to inhibit the transcription of the negative regulators. In some embodiments, the disclosed methods involve the silencing of negative regulators of casein expression by at least 10%, at least 20%, at least 30%, at least 40%, at least 50%, at least 60%, at least 70%, at least 80%, at least 90%, up to and including 100%, or more, compared to a plant not subjected to the disclosed methods.

In some cases, the degree of silencing is achieved by adjusting the concentration of antisense RNAs, siRNAs, shRNAs, or CRISPRi components used. The concentration can range from 1 nM to 100 μM, including all values and ranges therebetween, such as 10 nM, 100 nM, 1 μM, 10 μM, 50 μM, or any range therein.

In some cases, the silencing of negative regulators results in an increase in casein protein production by at least 10%, at least 20%, at least 30%, at least 40%, at least 50%, at least 60%, at least 70%, at least 80%, at least 90%, up to and including 100%, or more, compared to a plant not subjected to the disclosed methods.

### MicroRNAs (miRNAs)

In some cases, the present invention employs microRNAs (miRNAs) to enhance the expression of the casein gene in transgenic plants. In some cases, miRNAs are used to downregulate the expression of negative regulators of the casein gene. In some cases, this involves introducing a miRNA that targets a repressor protein that binds to the casein promoter, or a protein that degrades the casein mRNA or protein. In some cases, miRNAs are used to upregulate the expression of positive regulators of the casein gene. This involves introducing a miRNA that targets a protein that inhibits a transcription factor or other positive regulator of the casein gene.

In some cases, the miRNAs are engineered to have increased specificity or efficacy. This involves modifying the sequence of the miRNA to improve its binding affinity for its target mRNA, or to increase its stability and prevent its degradation. In some cases, the miRNAs are delivered to the plant using a viral vector. This could involve inserting the miRNA into a plant virus, which is then used to infect the plant and deliver the miRNA. In some cases, the miRNAs are expressed from a transgene introduced into the plant. This involves inserting a gene that encodes the miRNA into the plant genome, under the control of a strong promoter or a promoter that is induced under specific conditions.

### shRNA, mhRNA, and lhRNA with Multiple Targets

As disclosed herein, the term “shRNA” refers to Short hairpin RNA, a sequence of RNA characterized by its ability to form a tight hairpin turn, facilitating gene expression silencing through the RNA interference (RNAi) pathway. Said shRNA is synthesized from a DNA template, resulting in a single-stranded RNA molecule that self-anneals to create a double-stranded structure via complementary base pairing. This configuration is identified by cellular mechanisms, culminating in the assembly of an RNA-induced silencing complex (RISC). The RISC employs the guide strand of the shRNA to bind and subsequently cleave complementary messenger RNA (mRNA) molecules, inhibiting their use in protein synthesis.

In some instances, shRNA finds application in both research and therapeutic contexts to diminish the expression of specific genes, thereby facilitating the examination of gene functionality and the potential amelioration of diseases through the inactivation of deleterious genes. In a preferred embodiment, shRNA vectors are engineered to integrate stably into the host genome, promoting the prolonged suppression of the target gene. Alternatively, transient expression of shRNA may be achieved utilizing plasmid vectors or viral vectors, enabling temporary gene silencing.

Typically, a 100 base pair (BP) sequence, homologous to a target such as GY1, is transcribed, folding back upon itself to generate a complementary structure, which is then segmented into fragments for the purpose of RNA-mediated interference. The innovation presented herein lies in the utilization of this mechanism to concurrently target two distinct genes within a single hairpin structure. Specifically, the double-target shRNA design facilitates the expression of casein in soy. Alternatively, said shRNA may target proteases or seed proteins.

It is noteworthy that the capability of shRNA to affect multiple targets due to homology among them is recognized; however, the novelty of the current invention is anchored in the targeting of two non-homologous genes using a singular shRNA construct. This approach may be employed for proteome rebalancing or the knockdown of proteases.

In some instances, mhRNA (medium hairpin RNA) serves as an alternative embodiment to shRNA for gene expression silencing via the RNA interference (RNAi) pathway. Unlike shRNA, mhRNA comprises a medium-length RNA molecule, characterized by greater than 25 nucleotides but fewer than 200 nucleotides, allowing for a very tight hairpin turn. This increased length can provide enhanced specificity and stability, potentially reducing off-target effects associated with shorter RNA molecules. The synthesis of mhRNA from a DNA template also results in a single-stranded RNA molecule that self-anneals to form a double-stranded structure through complementary base pairing. Similar to shRNA, mhRNA is recognized by cellular mechanisms, leading to the assembly of the RNA-induced silencing complex (RISC). The RISC utilizes the guide strand of mhRNA to target and cleave complementary mRNA molecules, thereby inhibiting their translation into proteins. The application of mhRNA, like shRNA, is vast, ranging from research endeavors to therapeutic interventions aimed at silencing specific genes to elucidate gene function or to mitigate disease by inactivating harmful genes.

Furthermore, lhRNA (long hairpin RNA) represents another alternative embodiment, distinguished by its large size, exceeding 200 nucleotides. The extensive length of lhRNA facilitates the formation of a tight hairpin turn and potentially allows for the inclusion of multiple silencing targets within a single RNA molecule, offering a versatile tool for complex gene silencing strategies. The synthesis and mechanism of action of lhRNA parallel those of shRNA and mhRNA, with the formation of a double-stranded RNA structure that prompts the assembly of the RISC. The lhRNA's guide strand directs the RISC to specific mRNA targets, leading to their cleavage and subsequent suppression of gene expression. Given its size, lhRNA can be engineered to target multiple, non-homologous genes simultaneously, providing a potent strategy for comprehensive gene silencing applications. This capability makes lhRNA particularly suitable for applications requiring the silencing of several genes concurrently, such as in the study of gene networks or the treatment of diseases with complex genetic bases.

In some embodiments, the present disclosure employs longer hairpins than are known or typical in the art, dubbed mhRNA and lhRNA. In a preferred embodiment, these longer (relative to shRNA) hairpins allow for multiple non-overlapping siRNA to be generated for a single target. The extended length of lhRNA, in particular, offers flexibility in design, especially for complex genetic targets. This flexibility allows for the inclusion of multiple siRNA sequences within a single lhRNA molecule, potentially enhancing the silencing of genes that are resistant to knockdown by conventional shRNA or single-target approaches. Generating multiple non-overlapping siRNA increases the chances of successfully knocking down or knocking out the target gene of interest. The capability of mhRNA and lhRNA to house multiple siRNA sequences targeting different parts of a gene or multiple genes within the same pathway, in some cases, leads to synergistic effects, increasing the potency of gene silencing.

The structural properties of mhRNA and lhRNA lend themselves to the development of modular and customizable gene silencing platforms. Researchers can theoretically design these molecules with interchangeable sequences targeting different genes, facilitating rapid adaptation to diverse research and therapeutic needs.

### Increased Deubiquitinating Enzymes (DUBs) Activity

In some cases, the disclosed transgenic plants have increased expression level of Deubiquitinating Enzymes (DUBs) through genetic editing, epigenetic modification, transcriptional regulation, post-transcriptional regulation, protein stabilization, nutrient deprivation, or any combination thereof. For example, overexpression using plasmids or viral vectors can be used to increase the amount of DUBs in a cell. In some cases, CRISPR/Cas9 system can be used to enhance the expression of genes associated with DUBs. In some cases, methylation and/or histone modification can increase expression of DUBs; for example, demethylating agents or histone deacetylase inhibitors can be used to increase gene expression. In some cases, the stability of the DUBs themselves can be increased to enhance their levels by inhibiting the pathways that degrade these enzymes or by using small molecules that stabilize these proteins. In some cases, ubiquitin-specific proteases (UBPs) are targeted. In some cases, ubiquitin c-terminal hydrolases (UCHs) are targeted.

In some aspects, the transgenic plant has at least a 1% increase in expression level of deubiquitinating enzymes. In some aspects, the transgenic plant has at least a 3% increase in expression level of deubiquitinating enzymes. In some aspects, the transgenic plant has at least a 7% increase in expression level of deubiquitinating enzymes. In some aspects, the transgenic plant has at least a 15% increase in expression level of deubiquitinating enzymes. In some aspects, the transgenic plant has at least a 30% increase in expression level of deubiquitinating enzymes. In some aspects, the transgenic plant has at least a 60% increase in expression level of deubiquitinating enzymes. In some aspects, the transgenic plant has at least a 120% increase in expression level of deubiquitinating enzymes. In some aspects, the transgenic plant has at least a 170% increase in expression level of deubiquitinating enzymes. In some aspects, the transgenic plant has at least a 230% increase in expression level of deubiquitinating enzymes. In some aspects, the transgenic plant has at least a 300% increase in expression level of deubiquitinating enzymes.

### Reduced Proteasomal Expression

In some cases, the disclosed transgenic plants have reduced expression level of ubiquitin/26S proteasome through genetic editing, RNA interference, proteasome inhibitors, nutrient deprivation, or some combination of these. In some cases, the disclosed transgenic plants have reduced or no expression levels of E1 (ubiquitin-activating enzyme), E2 (ubiquitin-conjugating enzyme), E3 (ubiquitin ligase), or any combination thereof. As used herein, proteasomal activity is inversely correlated with the residual casein protein content expressed in a plant (e.g., soybean).

In some aspects, the transgenic plant has at least a 10% reduction in proteasomal activity or expression. In some aspects, the transgenic plant has at least a 12.59% reduction in proteasomal activity or expression. In some aspects, the transgenic plant has at least a 15.85% reduction in proteasomal activity or expression. In some aspects, the transgenic plant has at least a 19.95% reduction in proteasomal activity or expression. In some aspects, the transgenic plant has at least a 25.12% reduction in proteasomal activity or expression. In some aspects, the transgenic plant has at least a 31.62% reduction in proteasomal activity or expression. In some aspects, the transgenic plant has at least a 39.81% reduction in proteasomal activity or expression. In some aspects, the transgenic plant has at least a 50.12% reduction in proteasomal activity or expression. In some aspects, the transgenic plant has at least a 63.10% reduction in proteasomal activity or expression. In some aspects, the transgenic plant has at least a 79.43% reduction in proteasomal activity or expression. In some aspects, the transgenic plant has at least a 100% reduction in proteasomal activity or expression.

In some aspects, the transgenic plant has at least a 10% reduction in ubiquitin-activating enzyme activity or expression. In some aspects, the transgenic plant has at least a 12.59% reduction in ubiquitin-activating enzyme activity or expression. In some aspects, the transgenic plant has at least a 15.85% reduction in ubiquitin-activating enzyme activity or expression. In some aspects, the transgenic plant has at least a 19.95% reduction in ubiquitin-activating enzyme activity or expression. In some aspects, the transgenic plant has at least a 25.12% reduction in ubiquitin-activating enzyme activity or expression. In some aspects, the transgenic plant has at least a 31.62% reduction in ubiquitin-activating enzyme activity or expression. In some aspects, the transgenic plant has at least a 39.81% reduction in ubiquitin-activating enzyme activity or expression. In some aspects, the transgenic plant has at least a 50.12% reduction in ubiquitin-activating enzyme activity or expression. In some aspects, the transgenic plant has at least a 63.10% reduction in ubiquitin-activating enzyme activity or expression. In some aspects, the transgenic plant has at least a 79.43% reduction in ubiquitin-activating enzyme activity or expression. In some aspects, the transgenic plant has at least a 100% reduction in ubiquitin-activating enzyme activity or expression.

In some aspects, the transgenic plant has at least a 10% reduction in ubiquitin-conjugating enzyme activity or expression. In some aspects, the transgenic plant has at least a 12.59% reduction in ubiquitin-conjugating enzyme activity or expression. In some aspects, the transgenic plant has at least a 15.85% reduction in ubiquitin-conjugating enzyme activity or expression. In some aspects, the transgenic plant has at least a 19.95% reduction in ubiquitin-conjugating enzyme activity or expression. In some aspects, the transgenic plant has at least a 25.12% reduction in ubiquitin-conjugating enzyme activity or expression. In some aspects, the transgenic plant has at least a 31.62% reduction in ubiquitin-conjugating enzyme activity or expression. In some aspects, the transgenic plant has at least a 39.81% reduction in ubiquitin-conjugating enzyme activity or expression. In some aspects, the transgenic plant has at least a 50.12% reduction in ubiquitin-conjugating enzyme activity or expression. In some aspects, the transgenic plant has at least a 63.10% reduction in ubiquitin-conjugating enzyme activity or expression. In some aspects, the transgenic plant has at least a 79.43% reduction in ubiquitin-conjugating enzyme activity or expression. In some aspects, the transgenic plant has at least a 100% reduction in ubiquitin-conjugating enzyme activity or expression.

In some aspects, the transgenic plant has at least a 10% reduction in ubiquitin ligase activity or expression. In some aspects, the transgenic plant has at least a 12.59% reduction in ubiquitin ligase activity or expression. In some aspects, the transgenic plant has at least a 15.85% reduction in ubiquitin ligase activity or expression. In some aspects, the transgenic plant has at least a 19.95% reduction in ubiquitin ligase activity or expression. In some aspects, the transgenic plant has at least a 25.12% reduction in ubiquitin ligase activity or expression. In some aspects, the transgenic plant has at least a 31.62% reduction in ubiquitin ligase activity or expression. In some aspects, the transgenic plant has at least a 39.81% reduction in ubiquitin ligase activity or expression. In some aspects, the transgenic plant has at least a 50.12% reduction in ubiquitin ligase activity or expression. In some aspects, the transgenic plant has at least a 63.10% reduction in ubiquitin ligase activity or expression. In some aspects, the transgenic plant has at least a 79.43% reduction in ubiquitin ligase activity or expression. In some aspects, the transgenic plant has at least a 100% reduction in ubiquitin ligase activity or expression.

In some aspects, the transgenic plant has at least a 10% increase in proteasomal activity or expression. In some aspects, the transgenic plant has at least a 12.59% reduction in proteasomal activity or expression. In some aspects, the transgenic plant has at least a 15.85% reduction in proteasomal activity or expression. In some aspects, the transgenic plant has at least a 19.95% reduction in proteasomal activity or expression. In some aspects, the transgenic plant has at least a 25.12% reduction in proteasomal activity or expression. In some aspects, the transgenic plant has at least a 31.62% reduction in proteasomal activity or expression. In some aspects, the transgenic plant has at least a 39.81% reduction in proteasomal activity or expression. In some aspects, the transgenic plant has at least a 50.12% reduction in proteasomal activity or expression. In some aspects, the transgenic plant has at least a 63.10% reduction in proteasomal activity or expression. In some aspects, the transgenic plant has at least a 79.43% reduction in proteasomal activity or expression. In some aspects, the transgenic plant has at least a 100% reduction in proteasomal activity or expression.

In some aspects, the disclosure provides a soybean having reduced proteasome activity or expression and increased casein content as a result of reduced proteasome activity or expression.

### Ubiquitous Protein Inhibition

This application relates to increasing the stability and levels of transgenic casein produced in soy plants by preventing their breakdown by proteases. These endogenous soy proteases can degrade the casein at any stage, from its initial synthesis in the endoplasmic reticulum (ER) to its storage in the protein storage vacuoles (PSVs). The pathway of casein from the ER to the PSVs is not fully understood, which complicates the identification and targeting of specific proteases for inhibition.

In some instances, native soy protease inhibitors, which are predominantly localized within vacuoles and storage compartments, could prevent the degradation of caseins. However, these inhibitors may not be co-localized with the transgenic caseins, particularly in compartments downstream of protein synthesis, thereby limiting their protective efficacy.

A proposed strategy to circumvent the requirement for precise localization of endogenous inhibitors involves the constitutive expression of broad-spectrum protease inhibitors, engineered with identical localization signals as the transgenic caseins. This approach is designed to provide ubiquitous protection against proteases throughout the entire lifespan of the casein proteins, from synthesis to storage.

For the purpose of this strategy, serine protease inhibitors and cysteine protease inhibitors are identified as potential candidates. These inhibitors exhibit a broad specificity towards proteases and, when co-expressed with casein, may offer a generalized defensive mechanism against proteolytic activity.

To empirically assess the effectiveness of this methodology, a prophetic experiment is proposed. This experiment would entail the quantitative analysis of transgenic casein levels using immunoblot techniques, comparing the abundance of caseins in the presence and absence of the broad-spectrum protease inhibitors. Additionally, a combinatorial approach involving the simultaneous expression of multiple protease inhibitors and the use of RNA interference (RNAi) to downregulate specific proteases or seed storage proteins may be explored to amplify the protective effect.

### Quantification and Optimization of Casein Expression in Transgenic Seeds

### Expression Levels

A seed comprising casein micelles or casein protein can be produced using any of the methods disclosed herein. In some cases, casein protein comprises at least 0.03% of the total weight of the seed. In some cases, casein protein comprises at least 0.041% of the total weight of the seed. In some cases, casein protein comprises at least 0.055% of the total weight of the seed. In some cases, casein protein comprises at least 0.075% of the total weight of the seed. In some cases, casein protein comprises at least 0.101% of the total weight of the seed. In some cases, casein protein comprises at least 0.136% of the total weight of the seed. In some cases, casein protein comprises at least 0.184% of the total weight of the seed. In some cases, casein protein comprises at least 0.248% of the total weight of the seed. In some cases, casein protein comprises at least 0.335% of the total weight of the seed. In some cases, casein protein comprises at least 0.452% of the total weight of the seed. In some cases, casein protein comprises at least 0.610% of the total weight of the seed. In some cases, casein protein comprises at least 0.824% of the total weight of the seed. In some cases, casein protein comprises at least 1.113% of the total weight of the seed. In some cases, casein protein comprises at least 1.503% of the total weight of the seed. In some cases, casein protein comprises at least 2.031% of the total weight of the seed. In some cases, casein protein comprises at least 2.744% of the total weight of the seed. In some cases, casein protein comprises at least 3.707% of the total weight of the seed. In some cases, casein protein comprises at least 5.006% of the total weight of the seed. In some cases, casein protein comprises at least 6.761% of the total weight of the seed. In some cases, casein protein comprises at least 13% of the total weight of the seed.

In some cases, casein protein comprises at most 0.1% of the total weight of the seed. In some cases, casein protein comprises at most 0.1337% of the total weight of the seed. In some cases, casein protein comprises at most 0.1789% of the total weight of the seed. In some cases, casein protein comprises at most 0.2396% of the total weight of the seed. In some cases, casein protein comprises at most 0.3205% of the total weight of the seed. In some cases, casein protein comprises at most 0.4293% of the total weight of the seed. In some cases, casein protein comprises at most 0.5745% of the total weight of the seed. In some cases, casein protein comprises at most 0.7697% of the total weight of the seed. In some cases, casein protein comprises at most 1.0306% of the total weight of the seed. In some cases, casein protein comprises at most 1.3801% of the total weight of the seed. In some cases, casein protein comprises at most 1.8475% of the total weight of the seed. In some cases, casein protein comprises at most 2.4743% of the total weight of the seed. In some cases, casein protein comprises at most 3.3136% of the total weight of the seed. In some cases, casein protein comprises at most 4.4333% of the total weight of the seed. In some cases, casein protein comprises at most 5.9305% of the total weight of the seed. In some cases, casein protein comprises at most 7.9365% of the total weight of the seed. In some cases, casein protein comprises at most 10.6249% of the total weight of the seed. In some cases, casein protein comprises at most 14.2361% of the total weight of the seed. In some cases, casein protein comprises at most 19.0505% of the total weight of the seed. In some cases, casein protein comprises at most 40% of the total weight of the seed.

In some cases, casein protein comprises at least 0.075% of the total protein content of the seed. In some cases, casein protein comprises at least 0.103% of the total protein content of the seed. In some cases, casein protein comprises at least 0.138% of the total protein content of the seed. In some cases, casein protein comprises at least 0.188% of the total protein content of the seed. In some cases, casein protein comprises at least 0.253% of the total protein content of the seed. In some cases, casein protein comprises at least 0.340% of the total protein content of the seed. In some cases, casein protein comprises at least 0.460% of the total protein content of the seed. In some cases, casein protein comprises at least 0.620% of the total protein content of the seed. In some cases, casein protein comprises at least 0.838% of the total protein content of the seed. In some cases, casein protein comprises at least 1.130% of the total protein content of the seed. In some cases, casein protein comprises at least 1.525% of the total protein content of the seed. In some cases, casein protein comprises at least 2.060% of the total protein content of the seed. In some cases, casein protein comprises at least 2.783% of the total protein content of the seed. In some cases, casein protein comprises at least 3.758% of the total protein content of the seed. In some cases, casein protein comprises at least 5.078% of the total protein content of the seed. In some cases, casein protein comprises at least 6.860% of the total protein content of the seed. In some cases, casein protein comprises at least 9.267% of the total protein content of the seed. In some cases, casein protein comprises at least 12.503% of the total protein content of the seed. In some cases, casein protein comprises at least 16.903% of the total protein content of the seed. In some cases, casein protein comprises at least 32.5% of the total protein content of the seed.

In some cases, casein protein comprises at most 0.25% of the total protein content of the seed. In some cases, casein protein comprises at most 0.3343% of the total protein content of the seed. In some cases, casein protein comprises at most 0.4473% of the total protein content of the seed. In some cases, casein protein comprises at most 0.599% of the total protein content of the seed. In some cases, casein protein comprises at most 0.8013% of the total protein content of the seed. In some cases, casein protein comprises at most 1.0733% of the total protein content of the seed. In some cases, casein protein comprises at most 1.4363% of the total protein content of the seed. In some cases, casein protein comprises at most 1.9243% of the total protein content of the seed. In some cases, casein protein comprises at most 2.5765% of the total protein content of the seed. In some cases, casein protein comprises at most 3.4503% of the total protein content of the seed. In some cases, casein protein comprises at most 4.6188% of the total protein content of the seed. In some cases, casein protein comprises at most 6.1858% of the total protein content of the seed. In some cases, casein protein comprises at most 8.284% of the total protein content of the seed. In some cases, casein protein comprises at most 11.0833% of the total protein content of the seed. In some cases, casein protein comprises at most 14.8263% of the total protein content of the seed. In some cases, casein protein comprises at most 19.8413% of the total protein content of the seed. In some cases, casein protein comprises at most 26.5623% of the total protein content of the seed. In some cases, casein protein comprises at most 35.5903% of the total protein content of the seed. In some cases, casein protein comprises at most 47.6263% of the total protein content of the seed. In some cases, casein protein comprises at most 100% of the total protein content of the seed.

In some cases, casein micelles comprise at least 0.03% of the total weight of the seed. In some cases, casein micelles comprise at least 0.041% of the total weight of the seed. In some cases, casein micelles comprise at least 0.055% of the total weight of the seed. In some cases, casein micelles comprise at least 0.075% of the total weight of the seed. In some cases, casein micelles comprise at least 0.101% of the total weight of the seed. In some cases, casein micelles comprise at least 0.136% of the total weight of the seed. In some cases, casein micelles comprise at least 0.184% of the total weight of the seed. In some cases, casein micelles comprise at least 0.248% of the total weight of the seed. In some cases, casein micelles comprise at least 0.335% of the total weight of the seed. In some cases, casein micelles comprise at least 0.452% of the total weight of the seed. In some cases, casein micelles comprise at least 0.610% of the total weight of the seed. In some cases, casein micelles comprise at least 0.824% of the total weight of the seed. In some cases, casein micelles comprise at least 1.113% of the total weight of the seed. In some cases, casein micelles comprise at least 1.503% of the total weight of the seed. In some cases, casein micelles comprise at least 2.031% of the total weight of the seed. In some cases, casein micelles comprise at least 2.744% of the total weight of the seed. In some cases, casein micelles comprise at least 3.707% of the total weight of the seed. In some cases, casein micelles comprise at least 5.006% of the total weight of the seed. In some cases, casein micelles comprise at least 6.761% of the total weight of the seed. In some cases, casein micelles comprise at least 13% of the total weight of the seed.

In some cases, casein micelles comprise at most 0.1% of the total weight of the seed. In some cases, casein micelles comprise at most 0.1337% of the total weight of the seed. In some cases, casein micelles comprise at most 0.1789% of the total weight of the seed. In some cases, casein micelles comprise at most 0.2396% of the total weight of the seed. In some cases, casein micelles comprise at most 0.3205% of the total weight of the seed. In some cases, casein micelles comprise at most 0.4293% of the total weight of the seed. In some cases, casein micelles comprise at most 0.5745% of the total weight of the seed. In some cases, casein micelles comprise at most 0.7697% of the total weight of the seed. In some cases, casein micelles comprise at most 1.0306% of the total weight of the seed. In some cases, casein micelles comprise at most 1.3801% of the total weight of the seed. In some cases, casein micelles comprise at most 1.8475% of the total weight of the seed. In some cases, casein micelles comprise at most 2.4743% of the total weight of the seed. In some cases, casein micelles comprise at most 3.3136% of the total weight of the seed. In some cases, casein micelles comprise at most 4.4333% of the total weight of the seed. In some cases, casein micelles comprise at most 5.9305% of the total weight of the seed. In some cases, casein micelles comprise at most 7.9365% of the total weight of the seed. In some cases, casein micelles comprise at most 10.6249% of the total weight of the seed. In some cases, casein micelles comprise at most 14.2361% of the total weight of the seed. In some cases, casein micelles comprise at most 19.0505% of the total weight of the seed. In some cases, casein micelles comprise at most 40% of the total weight of the seed.

In some cases, casein micelles comprise at least 0.075% of the total protein content of the seed. In some cases, casein micelles comprise at least 0.103% of the total protein content of the seed. In some cases, casein micelles comprise at least 0.138% of the total protein content of the seed. In some cases, casein micelles comprise at least 0.188% of the total protein content of the seed. In some cases, casein micelles comprise at least 0.253% of the total protein content of the seed. In some cases, casein micelles comprise at least 0.340% of the total protein content of the seed. In some cases, casein micelles comprise at least 0.460% of the total protein content of the seed. In some cases, casein micelles comprise at least 0.620% of the total protein content of the seed. In some cases, casein micelles comprise at least 0.838% of the total protein content of the seed. In some cases, casein micelles comprise at least 1.130% of the total protein content of the seed. In some cases, casein micelles comprise at least 1.525% of the total protein content of the seed. In some cases, casein micelles comprise at least 2.060% of the total protein content of the seed. In some cases, casein micelles comprise at least 2.783% of the total protein content of the seed. In some cases, casein micelles comprise at least 3.758% of the total protein content of the seed. In some cases, casein micelles comprise at least 5.078% of the total protein content of the seed. In some cases, casein micelles comprise at least 6.860% of the total protein content of the seed. In some cases, casein micelles comprise at least 9.267% of the total protein content of the seed. In some cases, casein micelles comprise at least 12.503% of the total protein content of the seed. In some cases, casein micelles comprise at least 16.903% of the total protein content of the seed. In some cases, casein micelles comprise at least 32.5% of the total protein content of the seed.

In some cases, casein micelles comprise at most 0.25% of the total protein content of the seed. In some cases, casein micelles comprise at most 0.3343% of the total protein content of the seed. In some cases, casein micelles comprise at most 0.4473% of the total protein content of the seed. In some cases, casein micelles comprise at most 0.599% of the total protein content of the seed. In some cases, casein micelles comprise at most 0.8013% of the total protein content of the seed. In some cases, casein micelles comprise at most 1.0733% of the total protein content of the seed. In some cases, casein micelles comprise at most 1.4363% of the total protein content of the seed. In some cases, casein micelles comprise at most 1.9243% of the total protein content of the seed. In some cases, casein micelles comprise at most 2.5765% of the total protein content of the seed. In some cases, casein micelles comprise at most 3.4503% of the total protein content of the seed. In some cases, casein micelles comprise at most 4.6188% of the total protein content of the seed. In some cases, casein micelles comprise at most 6.1858% of the total protein content of the seed. In some cases, casein micelles comprise at most 8.284% of the total protein content of the seed. In some cases, casein micelles comprise at most 11.0833% of the total protein content of the seed. In some cases, casein micelles comprise at most 14.8263% of the total protein content of the seed. In some cases, casein micelles comprise at most 19.8413% of the total protein content of the seed. In some cases, casein micelles comprise at most 26.5623% of the total protein content of the seed. In some cases, casein micelles comprise at most 35.5903% of the total protein content of the seed. In some cases, casein micelles comprise at most 47.6263% of the total protein content of the seed. In some cases, casein micelles comprise at most 90% of the total protein content of the seed.

In some cases, proteins in casein micelles comprise at least 0.075% of the total protein content of the seed. In some cases, proteins in casein micelles comprise at least 0.103% of the total protein content of the seed. In some cases, proteins in casein micelles comprise at least 0.138% of the total protein content of the seed. In some cases, proteins in casein micelles comprise at least 0.188% of the total protein content of the seed. In some cases, proteins in casein micelles comprise at least 0.253% of the total protein content of the seed. In some cases, proteins in casein micelles comprise at least 0.340% of the total protein content of the seed. In some cases, proteins in casein micelles comprise at least 0.460% of the total protein content of the seed. In some cases, proteins in casein micelles comprise at least 0.620% of the total protein content of the seed. In some cases, proteins in casein micelles comprise at least 0.838% of the total protein content of the seed. In some cases, proteins in casein micelles comprise at least 1.130% of the total protein content of the seed. In some cases, proteins in casein micelles comprise at least 1.525% of the total protein content of the seed. In some cases, proteins in casein micelles comprise at least 2.060% of the total protein content of the seed. In some cases, proteins in casein micelles comprise at least 2.783% of the total protein content of the seed. In some cases, proteins in casein micelles comprise at least 3.758% of the total protein content of the seed. In some cases, proteins in casein micelles comprise at least 5.078% of the total protein content of the seed. In some cases, proteins in casein micelles comprise at least 6.860% of the total protein content of the seed. In some cases, proteins in casein micelles comprise at least 9.267% of the total protein content of the seed. In some cases, proteins in casein micelles comprise at least 12.503% of the total protein content of the seed. In some cases, proteins in casein micelles comprise at least 16.903% of the total protein content of the seed. In some cases, proteins in casein micelles comprise at least 32.5% of the total protein content of the seed.

In some cases, proteins in casein micelles comprise at most 0.25% of the total protein content of the seed. In some cases, proteins in casein micelles comprise at most 0.3343% of the total protein content of the seed. In some cases, proteins in casein micelles comprise at most 0.4473% of the total protein content of the seed. In some cases, proteins in casein micelles comprise at most 0.599% of the total protein content of the seed. In some cases, proteins in casein micelles comprise at most 0.8013% of the total protein content of the seed. In some cases, proteins in casein micelles comprise at most 1.0733% of the total protein content of the seed. In some cases, proteins in casein micelles comprise at most 1.4363% of the total protein content of the seed. In some cases, proteins in casein micelles comprise at most 1.9243% of the total protein content of the seed. In some cases, proteins in casein micelles comprise at most 2.5765% of the total protein content of the seed. In some cases, proteins in casein micelles comprise at most 3.4503% of the total protein content of the seed. In some cases, proteins in casein micelles comprise at most 4.6188% of the total protein content of the seed. In some cases, proteins in casein micelles comprise at most 6.1858% of the total protein content of the seed. In some cases, proteins in casein micelles comprise at most 8.284% of the total protein content of the seed. In some cases, proteins in casein micelles comprise at most 11.0833% of the total protein content of the seed. In some cases, proteins in casein micelles comprise at most 14.8263% of the total protein content of the seed. In some cases, proteins in casein micelles comprise at most 19.8413% of the total protein content of the seed. In some cases, proteins in casein micelles comprise at most 26.5623% of the total protein content of the seed. In some cases, proteins in casein micelles comprise at most 35.5903% of the total protein content of the seed. In some cases, proteins in casein micelles comprise at most 47.6263% of the total protein content of the seed. In some cases, proteins in casein micelles comprise at most 90% of the total protein content of the seed.

### Combination of Strategies

In certain embodiments of the present invention, a combination of the strategies described herein is employed to maximize the production of casein protein in the genetically modified soy plant. By combining these strategies, the present invention provides a comprehensive and effective approach to enhancing the production of non-native proteins in genetically modified plants. This approach involves the simultaneous or sequential application of multiple strategies described herein.

In some cases, it is understood that the principles and techniques described herein can be applied to other plant species besides soy as well. The choice of plant species can be guided by various factors, including but not limited to, the plant's natural protein content, its amenability to genetic manipulation, its growth characteristics, and its suitability for large-scale cultivation. For instance, other leguminous plants like peas, lentils, or chickpeas, which have high protein content, could be suitable candidates. Similarly, cereal grains such as rice, wheat, or maize, which are widely cultivated and have well-established methods for genetic modification, could also be considered. The specific strategies for enhancing casein expression, such as promoter modification, codon optimization, use of chaperone proteins, and others, can be adapted as needed based on the specific characteristics of the chosen plant species. Therefore, the scope of the present invention extends beyond soy plants to include any plant species in which enhanced expression of casein proteins can be achieved through genetic manipulation.

In some cases, the plant species used for the expression of transgenic casein could include, but are not limited to, soybean (Glycine max), maize (Zea mays), rice (Oryza sativa), wheat (Triticum aestivum), barley (Hordeum vulgare), oats (Avena sativa), rye (Secale cereale), sorghum (Sorghum bicolor), millet (Panicum miliaceum), canola (Brassica napus), flax (Linum usitatissimum), sunflower (Helianthus annuus), cotton (Gossypium hirsutum), alfalfa (Medicago sativa), clover (Trifolium spp.), peas (Pisum sativum), lentils (Lens culinaris), chickpeas (Cicer arietinum), beans (Phaseolus vulgaris), peanuts (Arachis hypogaea), potatoes (Solanum tuberosum), tomatoes (Solanum lycopersicum), peppers (Capsicum annuum), eggplant (Solanum melongena), cucumbers (Cucumis sativus), squash (Cucurbita spp.), melons (Cucumis melo), watermelons (Citrullus lanatus), pumpkins (Cucurbita pepo), apples (Malus domestica), oranges (Citrus sinensis), lemons (Citrus limon), grapefruit (Citrus paradisi), limes (Citrus aurantifolia), strawberries (Fragaria×ananassa), raspberries (Rubus idaeus), blueberries (Vaccinium corymbosum), cranberries (Vaccinium macrocarpon), grapes (Vitis vinifera), peaches (Prunus persica), plums (Prunus domestica), cherries (Prunus avium), pears (Pyrus communis), pineapples (Ananas comosus), bananas (Musa spp.), kiwis (Actinidia deliciosa), mangoes (Mangifera indica), papayas (Carica papaya), and avocados (Persea americana).

In some cases, the specific casein targeted for enhanced expression in the transgenic plant is alpha-S1-casein. In some cases, the specific casein targeted is alpha-S2-casein. In some cases, the specific casein targeted is Beta-casein. In some cases, the specific casein targeted is Kappa-casein. In some cases, a combination of these caseins is targeted for enhanced expression. In some cases, the methods contemplated by this invention are applied to other dairy components beside caseins, including but not limited to, beta-lactoglobulin protein, alpha-lactalbumin protein, immunoglobulins, lactoferrin, serum albumin, osteopontin, mucins, growth factors, and other dairy enzymes such as lysozyme and lactoperoxidase. In some instances, the methods may also be applied to enhance the expression of minor proteins found in milk, such as lactadherin, butyrophilin, xanthine oxidase, and fatty acid-binding protein.

In some cases, the dairy proteins contemplated herein are derived from bovine (cows), caprine (goats), ovine (sheep), bubaline (buffaloes), camelid (camels), equid (horses or donkeys), cervidae (reindeer), bovidae (yaks), or hominidae (humans). In some instances, the dairy proteins are at least 80% homologous to those of bovine, caprine, ovine, bubaline, camelid, equid, cervidae, bovidae, or human origin. In other cases, the dairy proteins contemplated herein are at least 60% homologous to those derived from bovine, caprine, ovine, bubaline, camelid, equid, cervidae, bovidae, or human sources.

The properties of milk derived from casein that are produced by one or more of the methods described herein include, but are not limited to, texture (ranging from thin and watery to thick and creamy), flavor profile (from mild to rich, sweet to savory), aroma (from fresh to fermented), color (from nearly transparent to creamy white), nutritional profile (including protein, calcium, vitamin, and mineral content), digestibility, allergenicity, fat content (from skim to full-fat), lactose content (from high to low or lactose-free), shelf-life (from fresh to long-life or UHT), susceptibility to spoilage microorganisms, pH level (acidity), heat stability (important for pasteurization and cooking), foaming properties (important for frothed milk drinks), emulsifying properties (important for the stability of milk fat globules), coagulation properties (important for cheese making), solubility (important for powdered milk), hydration properties, whipping properties (important for cream), freezing properties (important for ice cream), and the presence of any additional flavors or ingredients such as vitamins, minerals, probiotics, or flavorings. Furthermore, the milk's properties can influence the characteristics of derivative products such as cheese, yogurt, butter, cream, ice cream, and milk powders, affecting their texture, flavor, color, shelf-life, and other attributes.

The properties of cheese derived from casein that are produced by one or more of the methods described herein include, but are not limited to, texture (ranging from soft and creamy to hard and crumbly), moisture content (from very dry to very moist), flavor profile (from mild to sharp, tangy to sweet, buttery to nutty, simple to complex), aroma (from mild to pungent, fresh to fermented), color (from white to yellow or orange, with potential for blue or green mold), ripening time (from fresh to aged over several years), melting characteristics (from non-melting to smooth melting, with variations in browning and blistering behavior), stretchability (important for cheeses used in cooking), fat content (from low-fat to full-fat), protein content, shelf-life (from days to years), susceptibility to mold and bacterial growth, nutritional profile (including calcium and protein content, vitamin content, mineral content), digestibility, allergenicity, salt content, pH level (acidity), firmness, elasticity, cohesiveness, adhesiveness, springiness, gumminess, chewiness, resilience, fracture properties (how the cheese breaks or splits), free oil content (oil that seeps out of the cheese), browning potential, blistering behavior, flowability when melted, and formation of crystals (such as calcium lactate or tyrosine crystals).

In some cases, the methods described herein for increased expression of casein protein are employed in the context of microbial expression systems, such as bacteria, yeast, or fungi. In some cases, the methods described herein for increased expression of casein protein are employed in the context of cell culture systems.

### Nutrient and Environmental Optimization

In certain embodiments of the present invention, the growth conditions of the genetically modified soy plant are optimized to enhance the expression of the casein gene, thereby increasing the overall production of casein protein. The growth conditions, including but not limited to temperature, nutrients, and water, significantly influence gene expression and protein production in plants.

In some cases, the nutrient conditions are optimized. Providing a nutrient-rich environment, with optimal levels of macronutrients (such as nitrogen, phosphorus, and potassium) and micronutrients (such as iron, zinc, and copper), promotes plant growth and protein production. In some cases, specific nutrients are added that are known to enhance the expression or stability of the casein protein.

In some cases, temperature and water conditions are adjusted. In some cases, the temperature is set to the optimal range for soy growth. In some cases, the temperature is varied in a controlled manner to induce stress responses that enhance casein expression. In some cases, the water conditions, including the soil moisture level and the frequency and amount of watering, are optimized to prevent water stress and promote healthy growth.

### Cross-Linkers During Extraction

In some aspects, the disclosure provides methods for enhancing the quantity of recoverable casein in transgenic soy by using cross-linkers during extraction of the casein proteins. In some cases, a non-toxic or low-toxicity cross-linker is used. In some cases, a toxic cross-linker can be chemically modified to reduce its toxicity or encapsulated or conjugated to another molecule to reduce its toxicity. In some cases, a non-toxic derivative of the cross-linker is used, which is then converted into the active, cross-linking form.

In some instances, the cross-linker is at least one of Genipin, Ethylenediamine, Cystamine, Bis(sulfosuccinimidyl) suberate (BS3), or 1-Ethyl-3-(3-dimethylaminopropyl) carbodiimide (EDC). In some instances, the cross-linker is used in conjunction with a carbodiimide cross-linker, such as EDC, to create “zero-length” crosslinks between carboxylic acid and amine groups on proteins. In some instances, the cross-linker is used in combination with N-Hydroxysuccinimide (NHS).

In some instances the cross-linker is at least one of the following: Glutaraldehyde, ethylene glycol diglycidyl ether (EGDE), polyethylene glycol diglycidyl ether (PEGDE), toluene diisocyanate (TDI), hexamethylene diisocyanate (HDI), 1-ethyl-3-(3-dimethylaminopropyl) carbodiimide (EDC), N-Hydroxysuccinimide (NHS), dithiobis[succinimidyl propionate] (DSP), disuccinimidyl suberate (DSS), 3,3′-dithiobis[sulfosuccinimidylpropionate] (DTSSP), bis[sulfosuccinimidyl] suberate (BS3), bis[2-(succinimidooxycarbonyloxy)ethyl]sulfone (BSOCOES), 1,5-difluoro-2,4-dinitrobenzene (DFDNB), formaldehyde, dimethyl adipimidate (DMA), 1,5-difluoro-2,4-dinitrobenzene (DFDNB), genipin, ethylenediamine, cystamine, bismaleimidohexane (BMH), disuccinimidyl tartrate (DST), ethylene glycol bis[succinimidylsuccinate] (EGS), N,N′-ethylenebis(iodoacetamide) (EBI), N,N′-(1,3-phenylene)dibutyric acid di[succinimidyl ester] (DSS), N,N′-(ethylene-di-1,2-phenylene)bismaleimide, N,N′-1,4-phenylenebismaleimide, divinyl sulfone, diisopropylcarbodiimide, dicyclohexylcarbodiimide, diethylcarbodiimide, difluorodinitrobenzene, N,N′-disuccinimidyl carbonate (DSC), dithiobispropionimidate, glyoxal, 1,5-difluoro-2,4-dinitrobenzene, N-succinimidyl(4-iodoacetyl)aminobenzoate (SIAB), N-succinimidyl(4-iodoacetyl)aminobenzoate (SIA), succinimidyl 4-(p-maleimidophenyl)butyrate (SMPB), succinimidyl 6-[(beta-maleimidopropionamido)hexanoate] (SMPH).

### Protease Inhibitor During Extraction

In some cases, a protease inhibitor is used during the extraction process of casein proteins from soy. The presence of a protease inhibitor can protect the caseins from degradation by endogenous soy proteases, further preserving the casein yield.

In some cases, the protease inhibitor is a Serine Protease Inhibitor, which specifically targets serine proteases and modulates their activity. In some cases, the protease inhibitor is a Cysteine Protease Inhibitor, designed to inhibit cysteine proteases and prevent protein degradation. In some cases, the protease inhibitor is an Aspartic Protease Inhibitor, which selectively inhibits aspartic proteases involved in various biological processes. In some cases, the protease inhibitor is a Metalloprotease Inhibitor, targeting metalloproteases that require metal ions for their enzymatic activity. In some cases, the protease inhibitor is a Covalent Protease Inhibitor, forming irreversible covalent bonds with the target proteases. In some cases, the protease inhibitor is a Reversible Protease Inhibitor, forming non-covalent interactions with the protease and allowing for reversible inhibition. In some cases, the protease inhibitor is a Peptide-based Protease Inhibitor, designed as short peptides that mimic the substrate of the protease and block its activity. In some cases, the protease inhibitor is a Synthetic Protease Inhibitor, chemically synthesized to specifically inhibit target proteases. In some cases, the protease inhibitor is a Natural Protease Inhibitor, derived from natural sources, such as plants, animals, or microorganisms, and inhibits protease activity.

Examples of protease inhibitors include: Aprotinin, Leupeptin, Pepstatin A, E-64, PMSF (Phenylmethanesulfonyl fluoride), EDTA (Ethylenediaminetetraacetic acid), Bestatin, AEBSF (4-(2-Aminoethyl)benzenesulfonyl fluoride hydrochloride), TLCK (Nα-p-Tosyl-L-lysine chloromethyl ketone), TPCK (Nα-p-Tosyl-L-phenylalanine chloromethyl ketone), Benzamidine, Antipain, Chymostatin, Elastatinal, Antithrombin III, Bowman-Birk inhibitor, Soybean trypsin inhibitor, Pefabloc (4-(2-Aminoethyl)benzenesulfonyl fluoride hydrochloride), SBTI (Streptomyces griseus trypsin inhibitor), Castanospermine, MG132, Calpain Inhibitor I (ALLN), E64d, Lactacystin, Iodoacetamide, Antipain hydrochloride, Erythromycin, Chymotrypsin Inhibitor 2, Nafamostat Mesylate, E-64c, Ro 31-8220, Z-FA-FMK (Caspase-1 inhibitor), Pepstatin B, Chymostatin A, Marimastat (BB-2516), Boc-D-FMK (Caspase-3 inhibitor), Soybean Bowman-Birk inhibitor, PPACK (D-phenylalanyl-L-prolyl-L-arginine chloromethylketone), Z-VAD-FMK (pan-caspase inhibitor), AEBSF·HCl (4-(2-Aminoethyl)benzenesulfonyl fluoride hydrochloride).

### Increasing Proline Synthesis for Enhanced Casein Expression in Soy

In some instances, the present invention is directed towards a novel method of increasing the synthesis of proline in plants, such as soybeans (Glycine max), to facilitate the enhanced expression of exogenous proteins, notably casein. Given the composition of casein, which contains a higher percentage of proline than average soy proteins, the availability of proline is hypothesized to be a limiting factor in casein synthesis within soybeans. For example, alpha casein contains 8.5% proline, beta casein contains 16.5%, and kappa casein contains 11.8%, compared to the soy proteins GY1 and CG1, which contain 5.9% and 5.6% proline, respectively.

To address this limitation, the invention proposes the overexpression of genes involved in the proline synthesis pathway to a level that is significantly elevated compared to their natural expression in soybeans. The glutamate→proline synthesis pathway, well-characterized in both humans and Arabidopsis thaliana, involves key proteins such as ALDH18A1 and PYCR (which has two slightly different but functionally redundant forms). In soybeans, homologs for these proteins have been identified, suggesting a similar pathway is present and can be manipulated for enhanced proline production.

In some embodiments, the invention involves the overexpression of one or more soy homologs of the ALDH18A1 and PYCR genes. Alternative strategies include the expression of the corresponding Arabidopsis thaliana or even human genes within the soybean seeds, with the rationale that such cross-species gene expression could potentially offer improved proline synthesis efficiency or other beneficial traits not observed with the native soy genes.

Additionally, recognizing that the mere synthesis of proline may not suffice for its incorporation into the newly synthesized casein proteins, the invention further contemplates the overexpression of proline-tRNA-ligase (also known as proline-tRNA-synthetase). These enzymes play a critical role in attaching proline to its corresponding transfer RNA (tRNA), a prerequisite for the ribosome-mediated incorporation of proline into proteins during translation. In some embodiments, homologs of Arabidopsis proline-tRNA-ligase genes, such as AT3G62120, AT5G10880, and AT5G52520, are targets for overexpression alongside the proline synthesis pathway genes.

In some instances, the method may also necessitate the increased expression of tRNA molecules specific for proline, thereby ensuring the availability of the necessary components for the translation machinery to efficiently produce casein proteins with high proline content.

This method presents a comprehensive approach to manipulating the metabolic and translational pathways in soybeans or similar plants for the purpose of producing exogenous proteins such as casein with higher efficiency and yield. Through the strategic overexpression of key genes in the proline synthesis pathway and the proline tRNA charging system, this invention offers a novel solution to the challenges associated with producing high-proline content proteins in plant systems.

### Genetically Engineered Casein Proteins: Modifications, Alternatives, and Functional Analogs

### Mutated Casein Proteins

Synthetic Alpha and Beta Casein Alternatives. The present disclosure provides for mutated caseins. In some cases, the mutated caseins demonstrate reduced degradation when expressed as exogenous protein in a plant such as soy, as compared to exogenous unmutated ruminant casein expressed in a plant.

In some cases, the mutated casein protein is a mutated α_{S1} or α_{S2} casein protein. Sequences 199 to 204, inclusive, are novel mutated α_{S1} casein. Sequences 199, 201, and 203 are DNA sequences; and sequences 200, 202, 204 are the corresponding amino-acid sequences. Mutated casein sequences 199 to 204 were derived by genetically modifying naturally-occurring bovine α_{S1} casein, i.e., sequences 205 (DNA) and 206 (Amino Acid).

In some cases, the mutated casein protein is a mutated β casein protein. Sequences 207 to 212, inclusive, are novel mutated β casein. Sequences 207, 209, and 211 are DNA sequences; and sequences 208, 210, 212 are the corresponding amino-acid sequences. Mutated casein sequences 207 to 212 were derived by modifying naturally-occurring bovine β casein, i.e., sequences 213 (DNA) and 214 (Amino Acid).

In some cases, the mutated casein (seqs. 119-204, 207-212, inclusive) confers greater resistance to proteasomal activity as compared to the corresponding wildtype casein protein (seqs. 205, 206, 213, 214). In some cases, the disclosed methods result in a degree of modification or mutation in the casein protein that confers resistance to proteasomal activity of at least 10%, at least 20%, at least 30%, at least 40%, at least 50%, at least 60%, at least 70%, at least 80%, at least 90%, up to and including 100%, as compared to a wildtype casein protein.

In some cases, mutated casein protein comprises fewer Proline (P), Glutamic acid (E), Serine(S), or Threonine (T) compared to a wildtype casein protein. The reduction in these residues can be at least 10%, at least 20%, at least 30%, at least 40%, at least 50%, at least 60%, at least 70%, at least 80%, at least 90%, up to and including 100%.

Modern genetic engineering techniques, such as CRISPR-Cas9 or other appropriate gene-editing tools, can be used to introduce targeted mutations into the PEST region of the casein genes in the soy plant. The mutated casein genes are then introduced into soy plants using established methods of plant transformation, such as Agrobacterium-mediated transformation or biolistic methods. Transgenic soy plants expressing the mutated casein genes are selected and propagated under appropriate conditions.

In certain embodiments of the present invention, the amino acid sequence of at least one casein protein is modified to prevent it or reduce the likelihood of it being targeted for ubiquitination, thereby enhancing its stability and overall production in the genetically modified soy plant. Ubiquitination is a post-translational modification process that often serves as a signal for protein degradation. Proteins that are tagged with ubiquitin are recognized and degraded by the proteasome, a large protein complex that breaks down unneeded or damaged proteins. The degree of modification can be at least 10%, at least 20%, at least 30%, at least 40%, at least 50%, at least 60%, at least 70%, at least 80%, at least 90%, up to and including 100%, as compared to a wildtype casein protein.

In the context of this invention, specific amino acids in the casein protein that are targets for ubiquitination (typically lysines) are identified and replaced with other amino acids that cannot be ubiquitinated. For instance, a lysine can be replaced with an arginine, which is structurally similar but cannot be ubiquitinated. By preventing ubiquitination of the casein protein, its degradation can be reduced, leading to an increase in the steady-state level of the protein in the soy plant.

In some cases, the mutated caseins demonstrate one or more functional properties of their corresponding wildtype caseins, such as, but not limited to, the ability to form casein micelles, binding and stabilization of calcium and phosphate ions, contributing to the buffering capacity of milk, participating in the emulsification of fats, facilitating the transport of minerals and small molecules, providing structural elements crucial for the formation of cheese and yogurt through coagulation processes, serving as a source of bioactive peptides upon hydrolysis, which may have immunomodulatory, antihypertensive, antimicrobial, or opioid-like activities, and acting as carriers for fat-soluble vitamins and bioactive compounds.

Synthetic Kappa Casein Alternatives. In some instances, the modification of casein proteins to express more casein protein in soy involves the application of mutagenesis primers within a precise genetic engineering framework. This process begins with the identification and isolation of the genes responsible for casein protein production in a donor organism, such as a dairy-producing animal. Following the isolation, specific mutagenesis primers are designed to introduce targeted modifications into these casein genes to enhance their expression levels or functionality when introduced into the soy genome.

The mutagenesis primers are synthesized to carry nucleotide sequences that either increase the efficiency of casein protein production or ensure the stable integration and expression of the casein genes within the soy plant's genome. These primers might include sequences that enhance promoter activity, increase mRNA stability, or facilitate the integration of the casein genes into regions of the soy genome that are highly active in protein synthesis.

Subsequently, the modified casein genes are inserted into the soy genome using a vector—a DNA molecule used as a vehicle to artificially carry foreign genetic material into another cell, where it can be replicated and expressed. Techniques such as Agrobacterium-mediated transformation or particle bombardment are commonly employed for this purpose. The mutagenesis primers play a critical role in ensuring that the casein genes are accurately and efficiently integrated into the soy genome.

In Example 6 below, differential immunoblotting signals for kappa-casein, using rabbit or goat antibodies, led to the hypothesis of selective proteolytic cleavage affecting antibody recognition sites. Through site-directed mutagenesis, 11 mutant kappa-casein constructs were created and four were expressed in E. coli. One mutant sequence in particular showed resistance to proteolysis in degradation assays, pinpointing critical sites within kappa-casein for antibody specificity: sequences 215 (DNA) and 216 (AA) represent the DNA and Amino Acid sequence for a novel mutated kappa casein sequence that demonstrates improved resistance as compared with bovine kappa casein (e.g., Seq. 121).

In some instances, an alternative strategy to increase casein protein expression in soy could involve the use of site-directed mutagenesis to modify existing soy proteins to mimic the nutritional and functional properties of casein proteins. This approach would leverage mutagenesis primers to induce specific amino acid changes in soy proteins, aiming to replicate the behavior of casein proteins in terms of solubility, binding, and nutritional content.

After the introduction of the modified casein genes or the modified soy proteins, the transgenic soy plants undergo a series of selection and screening processes to identify those that successfully express the desired casein protein characteristics. These plants are then subjected to further analysis to confirm the stability of the trait across generations, as well as to assess the safety and nutritional value of the modified casein protein.

### Phosphomimetic Alternatives to Casein

In some embodiments, phosphomimetic alternatives to α_{S1} casein, α_{S2} casein, β casein, or K casein are utilized. Sequences 142, 143, 144, 145, 146, 147, provide examples of phosphomimetic amino acid and DNA sequences for α_{S1} Casein, β Casein, and κ Casein. In some embodiments, at least one phosphomimetic alternative sequence for αₛ₁ Casein is used, for example, a sequence with at least 70%, 80%, 90%, 95%, or 99% identity to sequence 142 (amino acid sequence) or sequence 145 (DNA sequence). In some embodiments, at least one phosphomimetic alternative sequence for β-Casein is used, for example, a sequence with at least 70%, 80%, 90%, 95%, or 99% to sequence 143 (amino acid) or sequence 146 (DNA).

In some embodiments, at least one phosphomimetic alternative sequence for κ-Casein is used, for example, a sequence with at least 70%, 80%, 90%, 95%, or 99% identity to sequence 144 (amino acid) or sequence 147 (DNA). In some embodiments multiple phosphomimetic alternatives are utilized simultaneously. In some embodiments, a phosphomimetic alternative to κ Casein, such as a sequence with at least 70%, 80%, 90%, 95%, or 99% identity to sequence 144 or sequence 147, is used with at least one of a phosphomimetic alternative to α_{S1} casein, α₅₂ casein, or β-casein. The use of phosphomimetic alternatives to casein sequences allows in some cases for casein micelles to form without the need for the addition of a kinase, such as FAM20C. In some embodiments, phosphomimetic alternatives are utilized without kinase. In some embodiments, phosphomimetic alternatives to casein are utilized with non-phosphomimetic caseins, for example, phosphomimetic κ-Casein with at least one of non-phosphomimetic α_{S1} Casein, αs2 Casein, or β Casein. Some embodiments comprise a non-phosphorylated casein micelle comprising phosphomimetic α_{S1} Casein, αs2 Casein, β Casein, or κ Casein, such as a sequence with at least 70%, 80%, 90%, 95%, or 99% identity to sequences 142, 143, 144, 145, 146, 147.

In some embodiments phospho-ablated alternatives to casein sequences are utilized. In some embodiments, at least one phospho-ablated alternative sequence for α_{S1} Casein is used, for example, sequence 148 (amino acid sequence) or sequence 151 (DNA). In some embodiments, at least one phospho-ablated alternative sequence for β Casein is used, for example, sequence 149 (amino acid) or sequence 152 (DNA). In some embodiments, at least one phospho-ablated alternative sequence for κ-Casein is used, for example, sequence 150 (amino acid) or sequence 153 (DNA). In some embodiments multiple phosphomimetic alternatives are utilized simultaneously.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Barcodes to Identify Casein Operative Protease

shRNA Library. As referenced in the flowchart contained in FIG. 4, a library is created of short hairpin RNAs (shRNAs) complementary to five variations of approximately 1800 target protease mRNA sequences (≈9000 total). Each sequence is 65 base pair, followed by a “temp loop,” (Seq. 154) followed by its reverse-compliment 65 base pair. The inclusion of the temp loop is needed due to technological constraints that limit the synthesis to 200 base pair oligonucleotides. The temp loop serves as a strategic solution to ultimately accommodate a larger sequence without exhausting the synthesis nucleotides. It is shorter, consumes fewer nucleotides, and includes restriction sites.

Restriction cloning. The shRNAs are inserted into the plasmid vector (FIG. 5) at recognition site 1 via restriction cloning, producing a library of such plasmids.

Replace Temp Loop with Real Loop. Through the process of Golden Gate Restriction Cloning, the temporary loops (Seq. 154) are systematically replaced with the IV2 intron sequence (Seq. 155).

Clone Barcode Library. The barcode library is cloned via Golden Gate Restriction Cloning into the shRNA Library at recognition site 2. Importantly, the barcodes are fused to the Beta Casein. The library is now fully prepared, with plasmids containing β-Casein, κ-Casein, αS1-casein, FAM20C kinase, unique barcodes, and RNAi complementary to 1800 proteases.

Amplify plasmids via Escherichia coli (E. coli). Introduce the plasmids into competent E. coli cells using electroporation. Incubate and scale-up culture using known procedure.

Purify the DNA using a plasmid extraction procedure (i.e., collect the cultured E. coli cells by centrifugation, resuspend in a lysis solution to disrupt the cell membrane and denature proteins, add a neutralizing solution leaving the plasmid DNA in solution, centrifuge to separate the precipitated proteins and genomic DNA from the plasmid DNA in the supernatant, etc.

High-Throughput Sequencing Platform. Employ a high-throughput sequencing platform to concurrently sequence both the barcode regions and the corresponding RNAi sequences from each plasmid within the library. This generates a comprehensive database “codex” that maps each RNAi sequence to one or more associated barcodes. Importantly, each single sequencing read captures both the barcode and the short hairpin RNA (shRNA) on the same molecular entity.

Polyethylene glycol (PEG) transform soybean protoplasts. Isolate protoplasts from soybean tissue using enzymatic digestion, creating an enzyme mixture containing cellulase and pectinase to remove the cell wall, leaving the protoplasts. Then, perform the PEG transformation (pre-treatment, PEG/DNA mixture, protoplast addition, incubation, and washing), and recover and culture.

Harvest and extract the RNA and protein. The RNA is measured to normalize the data. The RNA is measured to normalize the data. When dealing with two identical plasmids introduced into different cells (cell 1 and cell 2), both receiving the same cell sequence, variations in beta casein production may occur. For instance, cell 2 might produce more beta casein due to higher transcription rates of RNA. A simple comparison of the barcodes could lead to incorrect conclusions regarding quantity, a difference that is not related to degradation but rather to differential beta casein production between the cells. To address this issue, it is vital to extract the RNA and quantify the number of RNA molecules bearing each barcode. This quantification allows for normalization, accounting for variations in transcription, such as a barcode that was transcribed 10 times more frequently. In the absence of degradation, this would be expected to appear in 10-fold greater quantities. This normalization is carried out using RNA sequencing, ensuring an accurate understanding of the underlying biological processes.

Cleave the barcode. The barcodes are cleaved through enzymatic digestion, carefully designed to specifically target the barcode region without affecting other parts of the molecule.

Mass spectrometry. Conduct mass spectrometry on the resulting barcodes.

Analysis. Casein degradation is believed to be caused by a specific subset of the roughly 3000 proteases, though the specific identities are unknown. By analyzing the presence of particular barcodes, we identify corresponding RNAi constructs that target specific proteases involved in beta casein degradation. Note: This works because we believe that proteases that degrade Beta casein will also degrade barcode fused to Beta casein. The abundance of certain barcodes indicates the targeting of proteases responsible for degrading casein. We then map the identified barcodes to their corresponding RNAi using the codex database, allowing us to pinpoint the specific proteases responsible for casein degradation. See FIG. 6.

### Example 2: Comparing Expression Levels of GFP with Casein

An experiment was designed to compare the expression behavior of Green Fluorescent Protein (GFP) with that of Casein protein. FIG. 16 shows the comparison of expression levels of GFP with Casein.

A pre-enzyme solution of 400 mL was prepared by combining 46.7 g of mannitol, 1.7 g of MES, and 600 mg of KCl. Then, 350 mL of DI water was added, mixed thoroughly, and the pH was adjusted to 5.2. The volume was made up to 400 mL and the solution was sterilized by autoclaving.

A solution of CuSO4 5H2O was prepared by dissolving 2.49 mg of copper sulfate pentahydrate in 10 mL of DI water, subsequently labeled as “CuSO4 stock,” and stored in a 15 mL falcon tube.

For the preparation of 1000 mL of CPW9M, 27.2 mg of KH2PO4, 101 mg of KNO3, 1.47 g of CaCl2 2H2O, 246 mg of MgSO4 7H2O, and 100 μL of a 1 mM stock of CuSO4 5H2O were dissolved along with 93.2 g of mannitol and 980 mg of MES in 900 mL of water. After dissolving the solids and adjusting the pH to 5.7 with KOH, the volume was brought up to 1000 mL with DI water and autoclaved. Once cooled, the solution was divided into 50 mL aliquots.

A total of 100 mL of MMG solution was prepared by combining 9.05 g of mannitol, 142.8 mg of MgCl2, and 78 mg of MES in a beaker with 90 mL of water and a stir bar. The mixture was stirred until dissolved, the pH adjusted to 5.7 with KOH, and the volume was brought up to 100 mL with DI water. After autoclaving for sterilization, the solution was divided into 10 mL aliquots upon cooling.

A PEG solution of 5 mL was freshly prepared on the day of transformation, with the pH allowed to stabilize for a few hours on a rotisserie. A mixture of 74 mg of CaCl2 2H2O, 133 mg of mannitol, and 2 g of PEG 4000 was weighed and transferred to a 15 mL falcon tube, where DI water was added until the solids were just covered. The tube was rotated until a viscous solution formed, after which the volume was adjusted to 5 mL.

An enzyme solution was created by mixing 75 mg of cellulase R-10 and 50 mg of macerozyme R-10 with 10 mg of BSA and 14 mg of CaCl2, followed by vortexing. This mixture was then added to 10 mL of the pre-enzyme solution in a 15 mL conical tube and vortexed again for thorough mixing.

Additionally, a solution containing 0.65M mannitol and 100 mg/L of propidium iodide was prepared for experimental use.

The process of protoplast extraction from soybean cotyledons was initiated by heating a water bath to 55° C. For each preparation, 10 mL of enzyme solution was prepared and incubated in the water bath for approximately 10 minutes before filter sterilization and aliquoting into a 15 mL falcon tube.

Seed pods from Williams 82 soybean plants, containing beans in the 7-8.5 mm range, were collected. The size of the beans was estimated by illuminating the pods and comparing the shadow of the beans to a ruler.

The pods were sterilized with a 10% bleach solution for about 5 minutes under sterile conditions and rinsed three times with autoclaved DI water.

Beans were extracted from the pods using sterile scalpel and forceps, placed in a clean petri dish, and the seed coats removed from approximately 8 beans, discarding the coats afterward.

Explants were sliced into thin strips using a scalpel or razor blade and transferred to a 15 mL falcon tube containing the enzyme solution. This procedure was replicated for the remaining explants and for each protoplast preparation.

The samples underwent vacuum infiltration for 15 minutes and were then incubated overnight at 60 RPM in the dark at room temperature, with the tubes laid on their sides to evenly distribute the tissue.

The next day, protoplasts were filtered through a 100 μm cell strainer (for non-FACS purposes) or a 40 μm cell strainer (for FACS) into a 50 mL falcon tube, centrifuged at 100×g for 5 minutes to form a pellet, and the supernatant was removed.

### Example 3: Protease Digestion of Kappa Casein by Endogenous Proteases

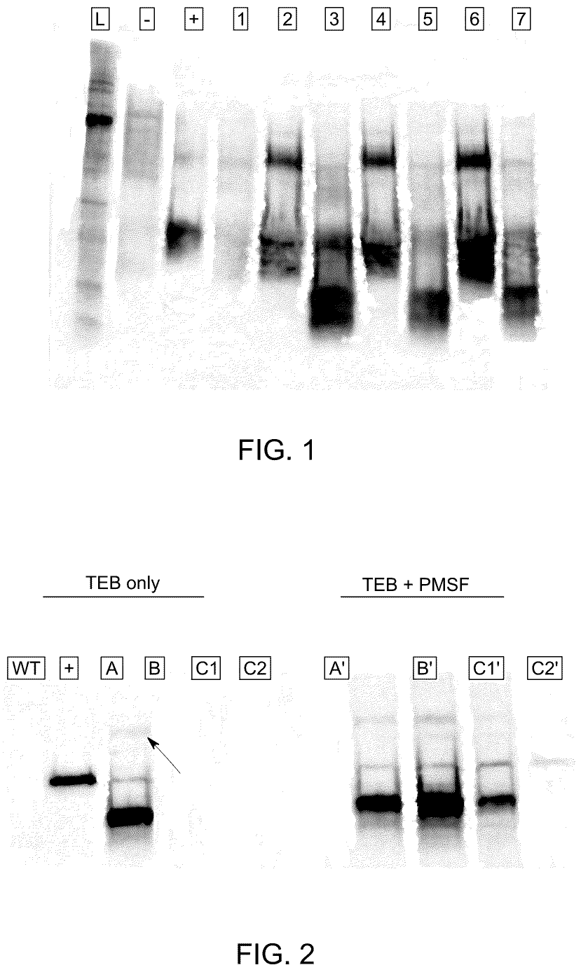

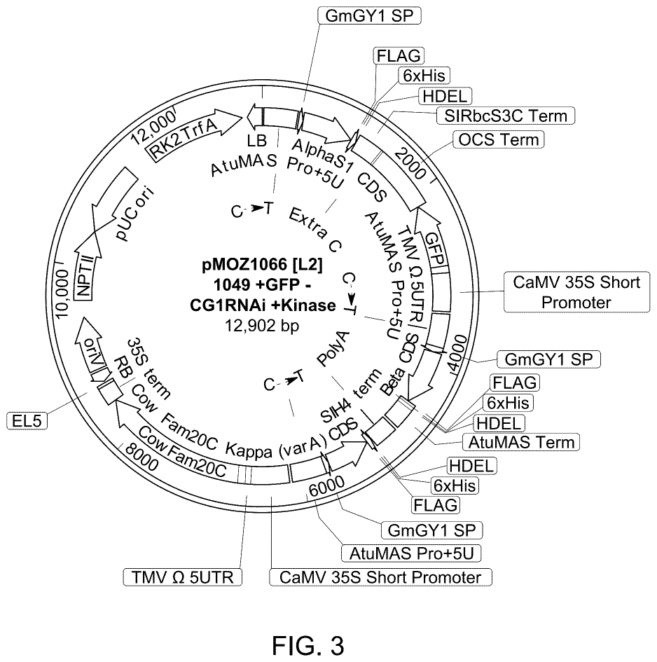

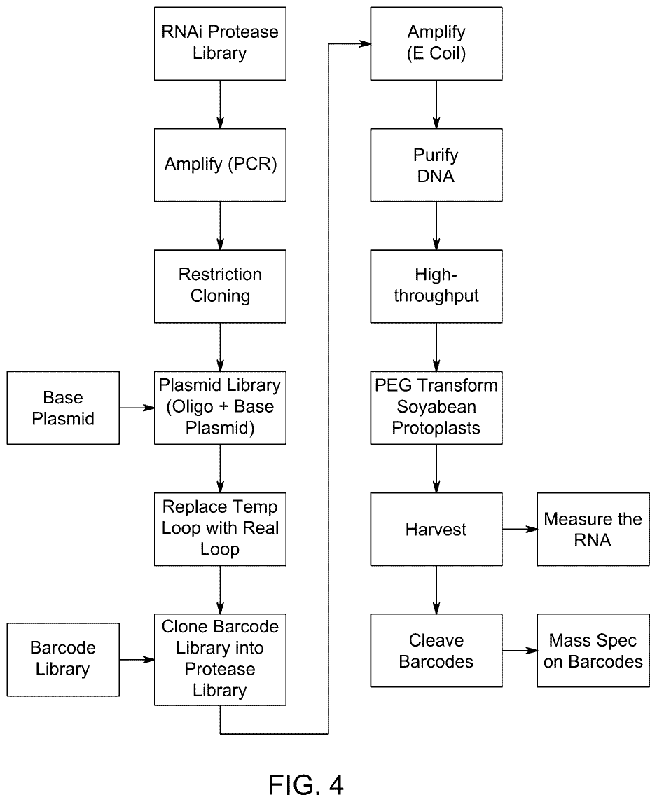

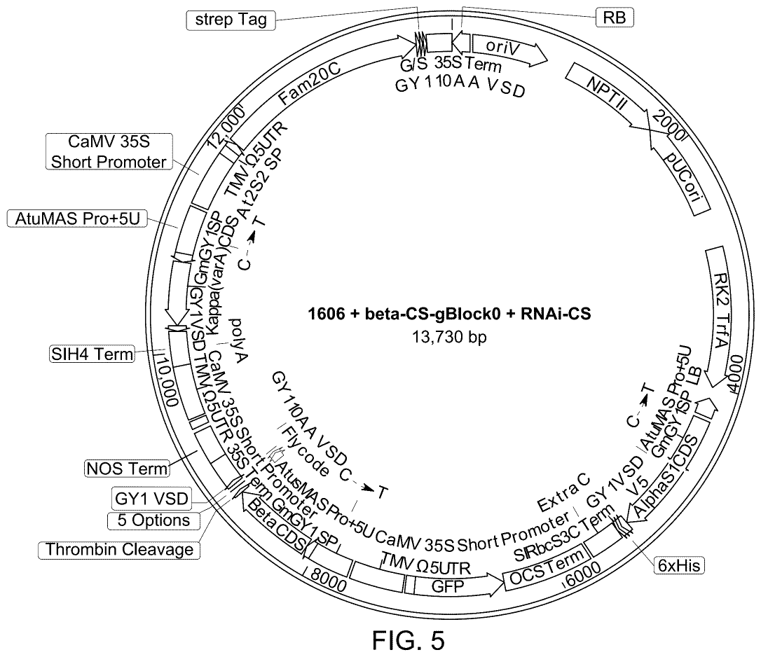

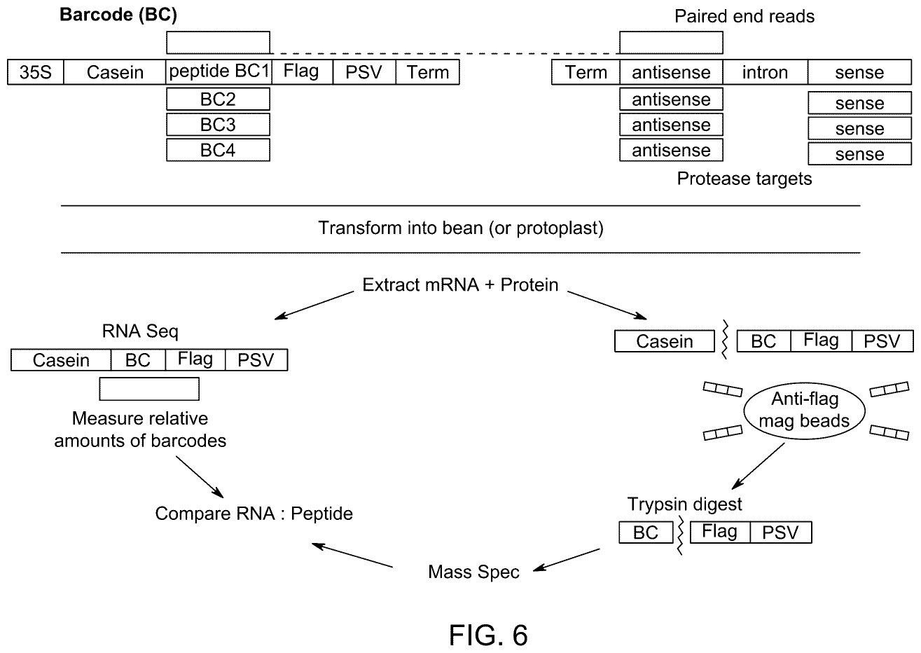

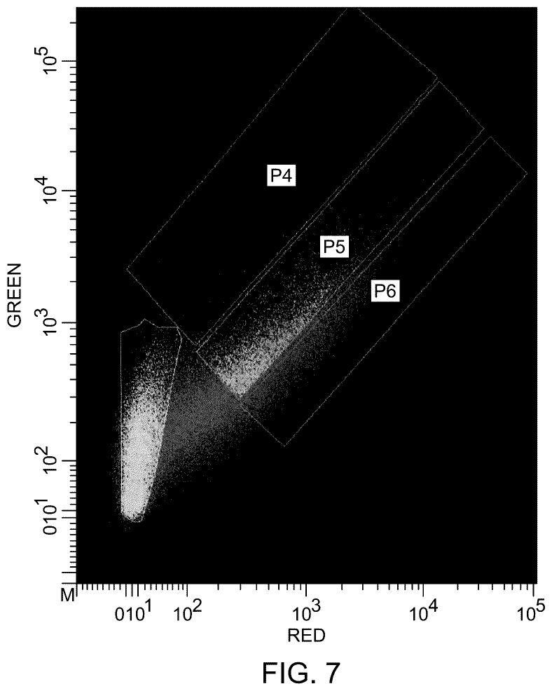

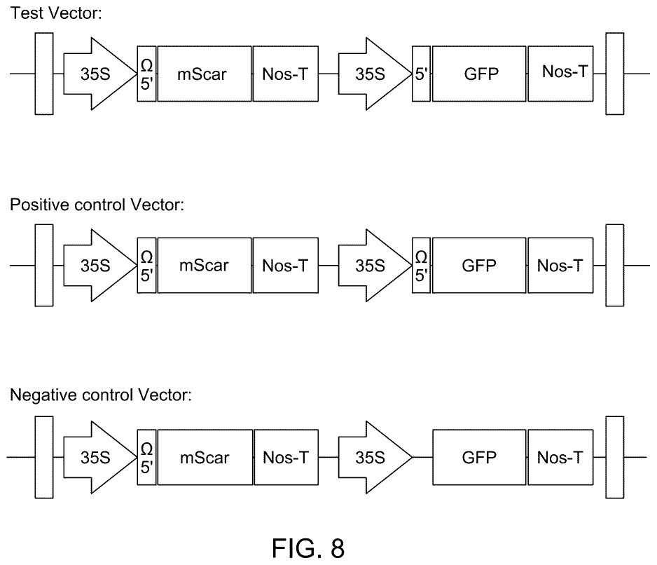

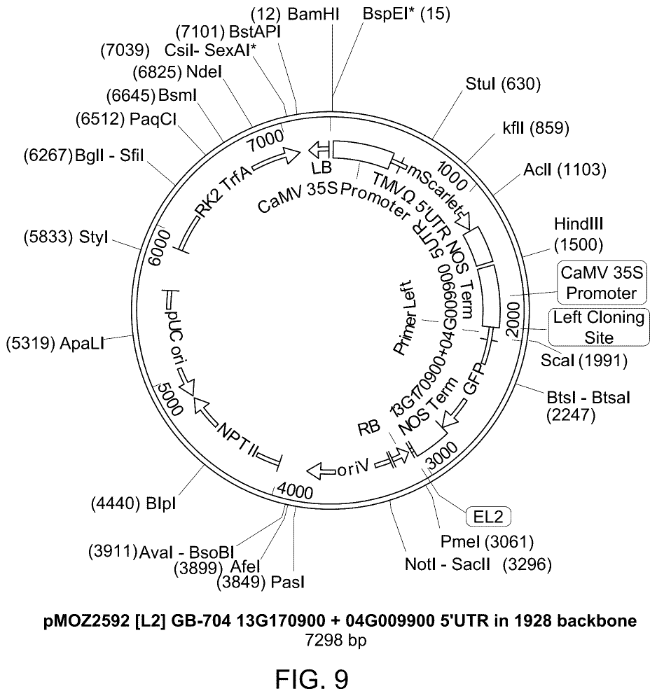

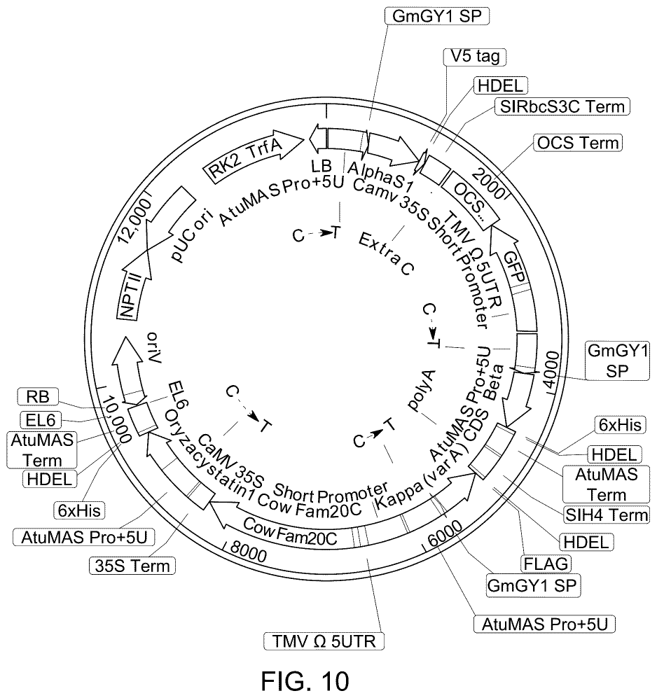

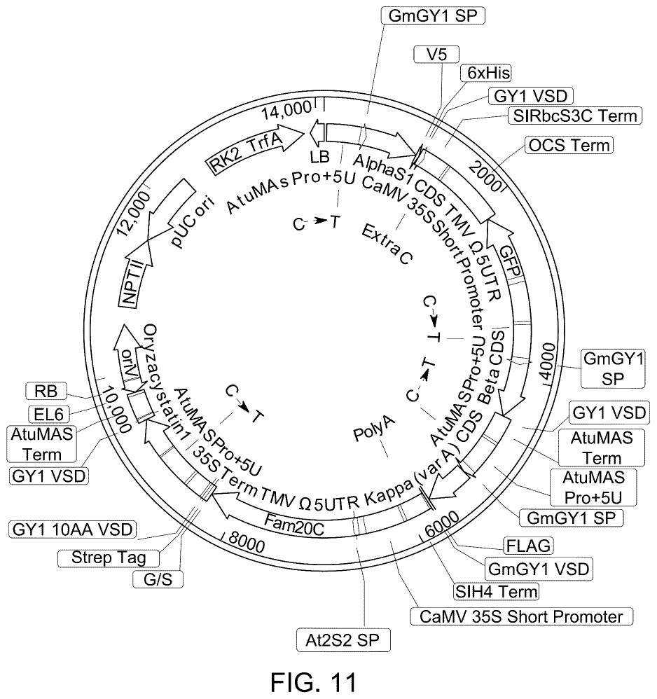

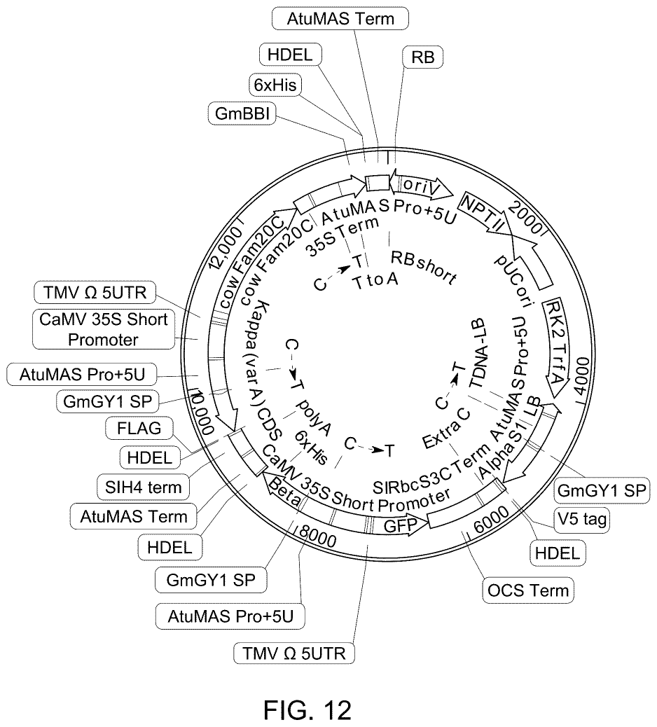

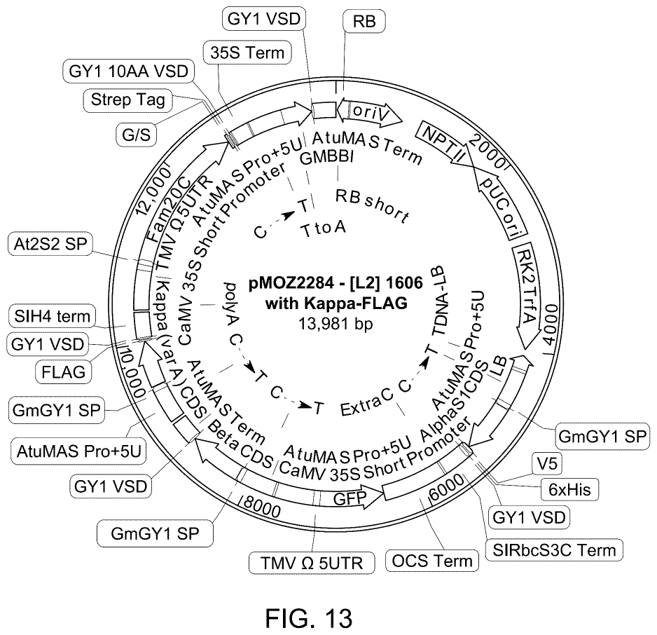

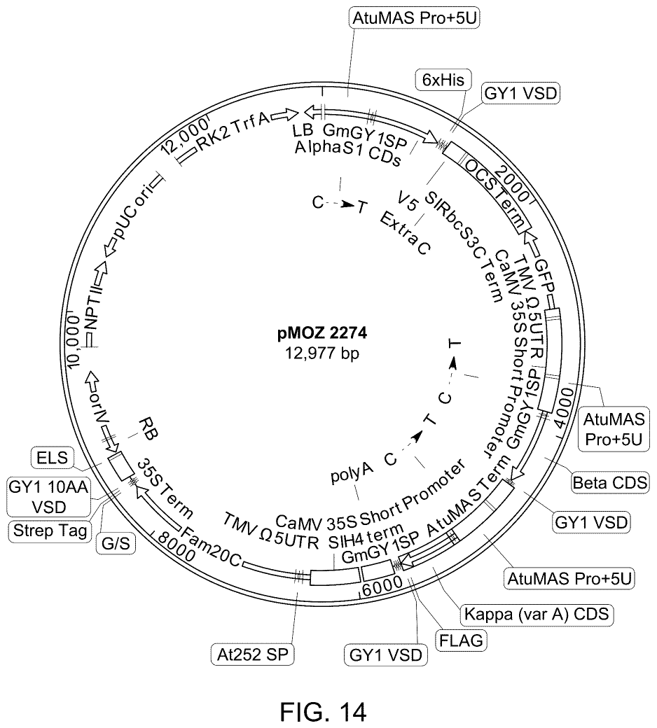

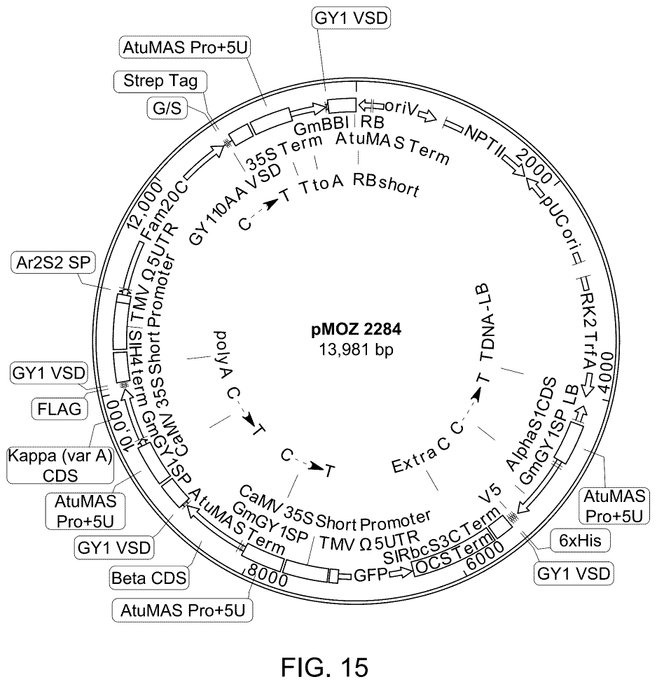

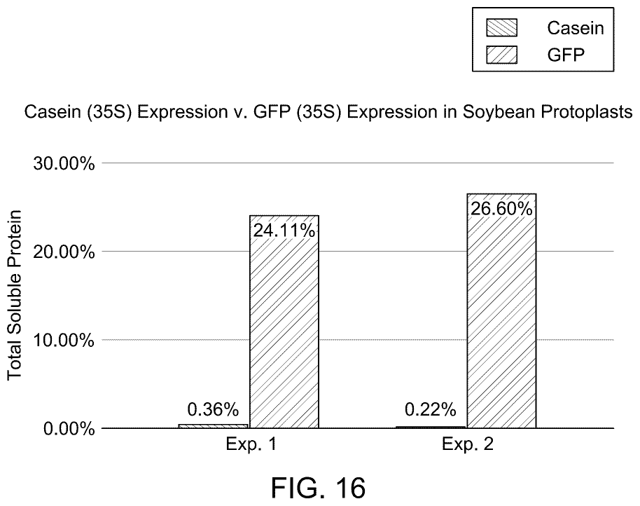

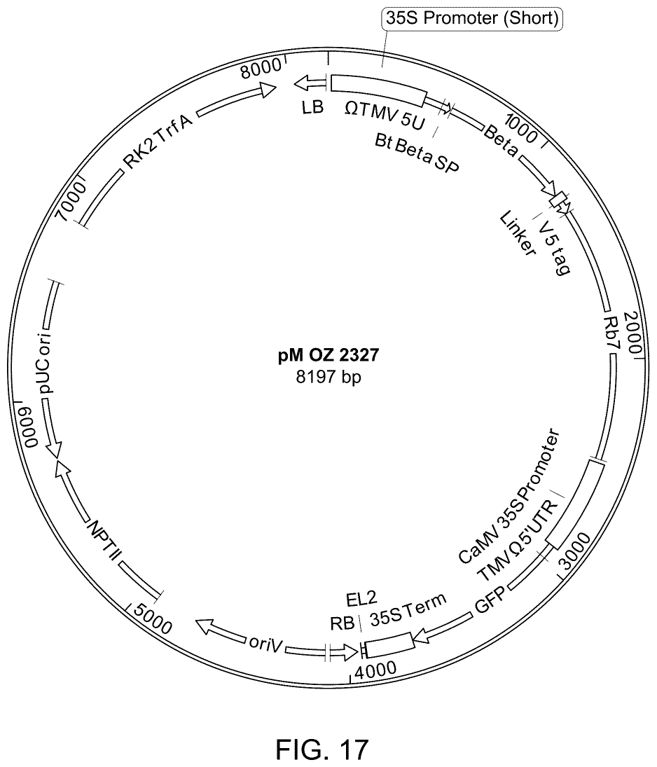

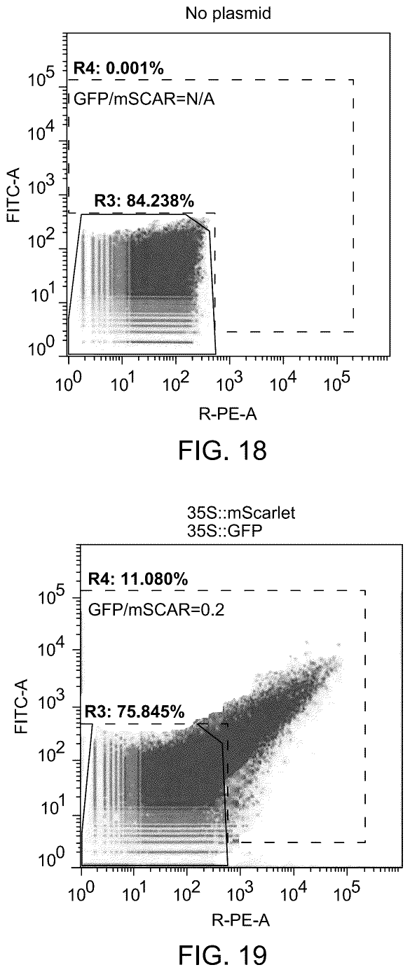

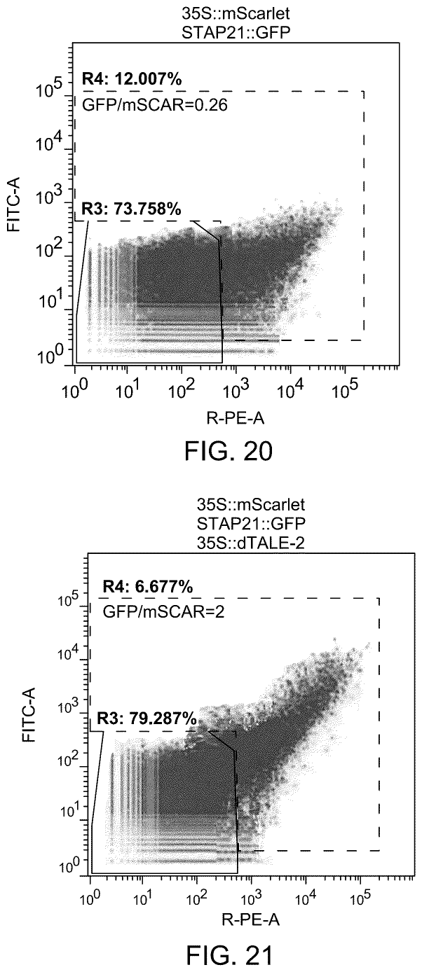

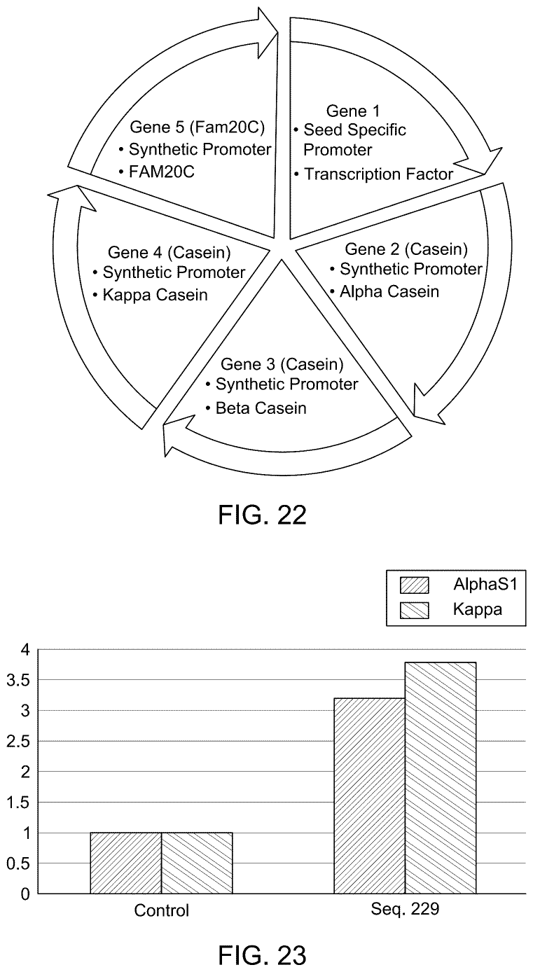

FIG. 1 depicts western blot data showing protease digestion of kappa-casein by plant proteases. “−” means Negative control. Positive control (+) is kappa casein purchased from Sigma-Aldrich. Lane 1 shows a sample of transgenic soy that has been altered to express alpha-, beta-, and kappa-casein. Lane 2 depicts Tris Extraction Buffer mixed with Leprino Micellar Casein Concentrate (MCC) in a 1:2 ratio at 4 degrees Celsius. Lane 3 shows transgenic soy mixed with Leprino Micellar Casein Concentrate (MCC) in a 1:2 ratio at 4 degrees Celsius. Lane 4 displays Tris Extraction Buffer mixed with Leprino Casein Concentrate (MCC) in a 1:2 ratio at room temperature. Lane 5 shows transgenic soy mixed with Leprino Micellar Casein Concentrate (MCC) in a 1:2 ratio at room temperature. Lane 6 depicts Tris Extraction Buffer mixed with Leprino Micellar Casein Concentrate (MCC) in a 1:2 ratio at 37 degrees Celsius. Lane 7 displays transgenic soy mixed with Leprino Micellar Casein Concentrate (MCC) in a 1:2 ratio at 37 degrees Celsius.

FIG. 2 depicts western blot data showing effectiveness of protease inhibitor. The labels mean the following: 1. “WT” means wild type and is used as a negative control. 2. “+” means positive control and is comprised of a peptide with antibodies targeting flag tag. 3. “A” is a sample prepared with the plasmid depicted in FIG. 3, which has been transiently transformed into soy to express alpha S1, Beta, and Kappa casein and in which protease activity is sufficiently low that casein proteins are detectable by western blot using a TEB buffer (a soluble extraction method).4. “B” is a sample prepared with the same plasmid (FIG. 3), but where protease activity is such that the casein proteins are detectable using Urea extraction (i.e., an insoluble protein extracting method), and where the protease activity is undetectable by soluble extraction methods. 5. “C1” is a sample prepared using the same plasmid, but where protease activity is higher, such that casein proteins are not detectable by Western Blots when using either TEB buffer or Urea extraction. 6. “C2” is a sample prepared using the same criteria as C1, but with a different transformation date. 7. A prime symbol ′ is used to indicate that the sample was treated with Phenylmethylsulphonyl fluoride (PMSF).

The following protocol was utilized:
1. Weigh crushed and frozen soy tissue in a 2 mL Eppendorf tube.
2. In a separate tube, prepare a solution of TEB (Tissue Extraction Buffer) with 1 mM PMSF added.
3. Add 5 μL of the TEB+PMSF solution per milligram of soy tissue to the Eppendorf tube containing the frozen tissue, and vortex the mixture for 30 seconds.
4. Incubate the extraction reaction by rotating the tube for 25 minutes at 4° C.
5. Centrifuge the tubes at 16,100 RCF for 5 minutes at 4° C. to separate the tissue extract.
6. To stop the extraction process, immediately transfer 5 μL of the tissue extract to a new tube containing 15 μL of Laemmli buffer with added beta-mercaptoethanol (BME). Proceed to blot the sample as required.

A vector transformation is confirmed by the successful expression of the green fluorescent protein in all samples. The western blots are targeting flag tag. With this technique, it was observed that the addition of PMSF reduced the degradation of casein in the soy tissue.

FIG. 3 depicts the plasmid utilized in the experiment described above and associated with the Western Blots in FIG. 2.

### Example 4: dTALE-2 in Soy

In the conducted experiment, flow cytometry analysis was performed on protoplasts derived from soybean cells, as depicted in FIGS. 18 to 21, which illustrate four distinct panels. Initially, FIG. 18 served as a negative control, involving protoplasts that underwent no transformation process. FIG. 19 displayed protoplasts transformed with a plasmid harboring the 35S promoter, which was responsible for driving the expression of both the mScarlet and Green Fluorescent Protein (GFP) genes on a single plasmid. In some instances, the 35S promoter is utilized due to its ability to strongly initiate transcription in plant cells.

Subsequently, FIG. 20 illustrated protoplasts in which the expression of the mScarlet gene was driven by the 35S promoter, whereas the GFP gene's expression was controlled by the STAP21 promoter (i.e. Seq. 164). The STAP21 promoter, in this context, refers to a specific DNA sequence recognized by transcription factors to initiate the transcription of the GFP gene. Lastly, FIG. 21 demonstrated a more complex transformation, incorporating the 35S promoter to drive the mScarlet gene, the STAP21 promoter for GFP gene expression, and an additional 35S promoter to drive the expression of the dTALE-2 gene. The dTALE-2 gene, in this experiment, is defined as a genetic construct designed to specifically activate the STAP21 promoter.

The flow cytometry analysis provided critical insights into the regulatory effects of the dTALE-2 gene on the STAP21 promoter's activity. The results unequivocally indicated that the presence of the dTALE-2 gene significantly activates the STAP21 promoter, as evidenced by the enhanced expression of the GFP gene in the protoplasts corresponding to FIG. 21. This experimental outcome underscores the potential of using the dTALE-2 gene as a molecular tool for controlling gene expression in soybean cells, offering avenues for further research into gene regulation mechanisms within plant systems.

### Example 5: Synthetic 5′ Untranslated Regions on Gene Expression

This subsection details an experimental procedure conducted to identify optimal 5′ untranslated regions (5′ UTRs) for enhanced gene expression. The experiment leverages a combination of synthetic biology, fluorescence-activated cell sorting (FACS), and RNA sequencing to assess the efficiency of various 5′ UTRs derived from the soybean genome and synthetic constructs.

Generation of 5′ UTR Library. The experiment commenced with the creation of a comprehensive 5′ UTR library. This library included 5′ UTRs sourced from existing gBlocks in the laboratory and those selected from high-efficiency genes identified in a soybean Ribo-Seq dataset. Due to size constraints, any 5′ UTR exceeding 161 base pairs was truncated to this length, starting from upstream of the start codon. Additionally, synthetic 5′ UTRs were constructed by fusing two different 5′ UTR sequences, especially when individual 5′ UTRs were longer than 80 base pairs. These combinations were made randomly, without specific criteria guiding the selection process.

Dual Reporter Vector Construction. The diverse library of 5′ UTRs was cloned into a specially designed dual reporter vector. This vector consisted of a standardized mScarlet cassette and a GFP cassette. The GFP cassette was engineered for the insertion of the 5′ UTRs under investigation. This setup aimed to facilitate the comparative analysis of GFP expression levels influenced by different 5′ UTRs, with mScarlet serving as a normalization control. See FIGS. 8 and 9.

Protoplast Transformation and FACS Sorting. The dual reporter vectors, each harboring a unique 5′ UTR, were introduced into zygotic protoplasts using polyethylene glycol (PEG)-mediated transformation. Subsequently, these transformed protoplasts underwent FACS based on the ratiometric measurement of GFP to mScarlet fluorescence. The protoplasts were sorted into three distinct categories reflecting high, medium, and low GFP expression levels.

RNA Sequencing and Analysis. Post-sorting, RNA was extracted from each of the categorized protoplast groups and subjected to RNA sequencing. This analysis aimed to correlate specific 5′ UTRs with their respective expression categories, particularly focusing on identifying 5′ UTRs that consistently resulted in high GFP expression.

Further Validation Using Individual 5′ UTRs. 5′ UTRs that were predominantly associated with high GFP expression levels in the initial screening were then individually cloned back into the dual reporter vector for more detailed analysis. These constructs were again transformed into protoplasts and analyzed using either FACS or flow cytometry to

The conducted experiment represents a meticulous approach to deciphering the influence of 5′ UTRs on gene expression efficiency. The combination of synthetic 5′ UTR construction, advanced sorting techniques, and in-depth sequencing analysis provides a robust framework for identifying optimal 5′ UTRs, particularly in the context of soybean genetics.

### Example 6: Mutagenesis Primers for Kappa-Casein

Variations were observed in the immunoblotting signals for kappa-casein when blending soy protein extracts with micellar casein concentrate depending on the use of either kappa-casein-specific rabbit or goat antibodies. These antibodies are known to bind to disparate epitopes on the kappa-casein molecule. The differential binding led to the hypothesis that proteolytic cleavage occurring within the kappa-casein molecule selectively impacts the epitope recognized by the goat-derived antibody, while sparing the epitope recognized by the rabbit-derived antibody. An approximate region of 40 amino acids was identified as the potential site of this differential cleavage.

To further clarify the mechanism underlying this observation, a series of site-directed mutagenesis experiments were undertaken. The strategy involved the sequential modification of kappa-casein sequences, targeting sections of five amino acids at a time. This was achieved utilizing the NEB Q5 Site-Directed Mutagenesis Kit, in conjunction with a set of custom-designed primers, referred to herein as sequences 173 to 198. These primers facilitated the generation of 11 mutant kappa-casein constructs.

Subsequent to the mutagenesis procedure, four of the mutant constructs were selected for transformation into Escherichia coli (E. coli) systems. This transformation yielded four mutant kappa-casein-flag fusion proteins, which were successfully purified for further analysis.

The purified mutant proteins were then subjected to degradation assays in the presence of type C extract, employing cell-free synthesized kappa-casein-flag protein as a reference standard. Through these assays, certain primers (sequences 175 and 176), produced mutant kappa casein (Sequences 215, 216) that demonstrated resistance to proteolytic degradation across experimental replications.

### Example 7: Protease Knockdown

The experiment detailed in FIG. 23 investigated the effect of medium-hairpin RNA (mhRNA) on casein protein production within green zygotic soybean seeds. Researchers compared two distinct plasmids: the first, a control plasmid, comprised αₛ₁ casein, β casein, κ-casein, and FAM20C, without the inclusion of any mhRNA sequences; the second plasmid was similarly composed of αₛ₁ casein, β casein, κ casein, and FAM20C but was uniquely augmented with mhRNA sequences targeting Anti-Casein Seq. No. 229 (Glyma.09G206500), aimed at enhancing casein protein synthesis.

Both plasmids were engineered to include unique peptide tags for subsequent immunodetection of the expressed proteins. The transfection process was carried out on green zygotic soybean seeds, which were then incubated for a duration of seven days to allow for the expression and accumulation of casein proteins. Following this incubation period, casein proteins were extracted from the seeds for quantitative analysis.

The quantification of casein proteins was conducted through western blotting techniques. The amount of casein protein present in each sample was normalized to the control to ensure accurate comparative analysis.

The results depicted in FIG. 23 demonstrate an increase in casein protein levels of both αₛ₁ casein, β casein, κ casein in the sample containing the mhRNA-enhanced plasmid, with over threefold the amount of casein produced compared to the control sample.

### Definitions

These and other valuable aspects of the embodiments of the present disclosure consequently further the state of the technology to at least the next level. While the disclosure has been described in conjunction with a specific best mode, it is to be understood that many alternatives, modifications, and variations will be apparent to those skilled in the art in light of the descriptions herein. Accordingly, it is intended to embrace all such alternatives, modifications, and variations that fall within the scope of the included claims. All matters set forth herein or shown in the accompanying drawings are to be interpreted in an illustrative and non-limiting sense.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular and not limited to sequential steps. Accordingly, terms such as “first” and “second” do not necessarily imply priority, order of importance, or order of acts.

As used herein, the term “about” or the symbol “~” when referring to a number or a numerical range means that the number or numerical range referred to is an approximation within experimental variability (or within statistical experimental error), and the number or numerical range may vary from, for example, from 1% to 10% of the stated number or numerical range. Unless otherwise indicated by context, the term “about” refers to +10% of a stated number or value.

As used herein, the term “approximately” means within an acceptable error range for the particular value as determined by one of ordinary skill in the art, which will depend in part on how the value is measured or determined, e.g., the limitations of the measurement system. For example, “approximately” can mean within 1 or more than 1 standard deviation, per the practice in the given value. Where particular values are described in the application and claims, unless otherwise stated the term “approximately” should be assumed to mean an acceptable error range for the particular value.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. All language such as “up to,” “at least,” “greater than,” “less than,” and the like include the number recited and refer to ranges which can be subsequently broken down into sub-ranges as discussed above. Finally, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Whenever the term “at least,” “greater than,” “greater than or equal to”, or a similar phrase precedes the first numerical value in a series of two or more numerical values, the term “at least,” “greater than,” “greater than or equal to” or similar phrase applies to each of the numerical values in that series of numerical values. For example, “at least 1, 2, or 3” is equivalent to “at least 1, at least 2, and/or at least 3.”

Whenever the term “no more than,” “less than,” “less than or equal to,” “no greater than,” “at most” or a similar phrase, precedes the first numerical value in a series of two or more numerical values, the term “no more than,” “less than,” “less than or equal to,” “no greater than,” “at most,” or similar phrase applies to each of the numerical values in that series of numerical values. For example, “less than 3, 2, or 1” is equivalent to “less than 3, less than 2, and/or less than 1.”

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

Definition of standard chemistry terms may be found in reference works, including but not limited to, Carey and Sundberg “Advanced Organic Chemistry 4th Ed.” Vols. A (2000) and B (2001), Plenum Press, New York.

Where a definition or use of a term in an incorporated reference is inconsistent or contrary to the definition of that term provided herein, the definition of that term provided herein applies and the definition of that term in the reference does not apply.

When referencing nucleic acid or protein sequences, “identity” indicates the degree of similarity between two sequences. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et at (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. BLASTP protein searches can be performed using default parameters. See, blast.ncbi.nlm.nih.gov/Blast.cgi.

As used herein, the phrase “essentially free of” is used to indicate the indicated component, if present, is present in an amount that does not contribute, or contributes only in a de minimus fashion, to the properties of the composition. In various embodiments, where a composition is essentially free of a particular component, the component is present in less than a functional amount. In various embodiments, the component may be present in trace amounts. Particular limits will vary depending on the nature of the component, but may be, for example, selected from less than 10% by weight, less than 9% by weight, less than 8% by weight, less than 7% by weight, less than 6% by weight, less than 5% by weight, less than 4% by weight, less than 3% by weight, less than 2% by weight, less than 1% by weight, less than 0.5% by weight, less than 0.1% by weight, or less than 0.05% by weight, or less than 0.01% by weight.

As used herein, the term “a detectable amount” refers to an amount of a composition (e.g., a molecule) that can be detected using the most sensitive analytical techniques up to date, including for example, liquid chromatography methods (e.g., reverse phase HPLC, size exclusion, normal phase chromatography), mass spectrometry (e.g., electrospray tandem mass spectrometry, and electrospray FT-ICR mass spectrometry), or a combination of analytical techniques (e.g., liquid chromatography-tandem mass spectrometry (LC-MS/MS)). In some cases, a detectable amount is at a concentration above 10⁻² mol/L, 10⁻³ mol/L, 10⁻⁴ mol/L, 10⁻⁵ mol/L, 10⁻⁶ mol/L, 10⁻⁷ mol/L, 10⁻⁸ mol/L, 10⁻⁹ mol/L, or 10⁻¹⁰ mol/L.

As used herein, the term “Anti-Casein” refers to any substance, compound, or agent that degrades casein or casein micelles or inhibits the expression of casein or inhibits the formation of casein micelles. In some instances, a substance may be a biological enzyme that specifically targets the peptide bonds in casein proteins, leading to their degradation. Alternatively, the compound might be a chemical inhibitor that interferes with the transcriptional or translational machinery necessary for the expression of casein genes, thereby reducing the overall production of casein in the organism.

As used herein, the term “Barcode” refers to a unique sequence of nucleotides used to identify and track specific molecules within a complex mixture.

As used herein, the term “Base Pair” refers to a pair of complementary nucleotides in a double-stranded nucleic acid molecule.

As used herein, the term “Casein” refers to a family of phosphoproteins commonly found in mammalian milk, comprising α_{S1} casein, α_{S2} casein, β Casein, and K Casein. The terms “alpha s1 casein” and “α_{S1} casein” are used interchangeably. The terms “alpha s2 casein” and “αs2 casein” are used interchangeably. The terms “β Casein” and “Beta Casein” are used interchangeably. The terms “K Casein” and “Kappa casein” are used interchangeably.

As used herein, the term “casein micelle” refers to a micelle formed by casein proteins. Examples of casein micelles are described in U.S. Pat. No. 11,326,176, filed on Jan. 13, 2020, titled “Recombinant micelle and method of in vivo assembly,” U.S. Pat. No. 11,718,856, and U.S. patent application Ser. No. 17/826,021 filed on May 26, 2022, which are incorporated herein by reference in its entirety. Recombinant casein micelles can be made in vivo or in vitro using the methods described therein.

As used herein, the term “Centrifugation” refers to a process of separating components within a liquid by spinning them at high speed.

As used herein, the terms “Escherichia coli” and “E. coli” are used interchangeably and refer to a common bacterium used in the laboratory for cloning and amplification of DNA.

As used herein, the term “Electroporation” refers to a method used to introduce DNA or other molecules into cells by applying an electrical field.

As used herein, the term “Enzymatic Digestion” refers to the breakdown of molecules using enzymes.

As used herein, the term “Expression” refers to the process by which genetic information is used to synthesize a protein.

As used herein, the term “FAM20C” refers to a kinase enzyme known as Family with sequence similarity 20, member C. It plays a critical role in the phosphorylation of proteins in the secretory pathway, including the phosphorylation of casein and other substrates. It has been associated with various biological processes and is relevant in the context of certain medical conditions and biochemical research.

As used herein, the term “Foreign gene” refers to a gene that is not naturally found in an organism.

As used herein, the term “Fusion Protein” refers to a protein comprising at least two constituent complete proteins that are encoded by separate genes, and that have been joined so that they are transcribed and translated as a single polypeptide. Fusion of a complete protein, such as casein, to a fragment of a protein is known in the art. See, e.g., U.S. Pat. No. 11,326,176.

As used herein, the term “Gateway Restriction Cloning” refers to a specific cloning technique that allows the insertion of DNA fragments into a plasmid vector.

As used herein, the term “Gene knockout” refers to a genetic technique for inactivating a specific gene.

As used herein, the term “7S” refers to β-conglycinin (or one or more subunits of β-conglycinin), and/or any other member within the 7S globulin family of proteins, whether derived from soybean or other plant species. As used herein, the term “11S” denotes glycinin (or one or more subunits of glycinin), and/or any other member within the 11S globulin family of proteins, whether sourced from soybean or other plant species. In some cases, “11S” consists of glycinin. In some cases, “7S” consists of β-conglycinin, small amounts of γ-conglycinin and basic 7S globulin (Bg7S). β-Conglycinin has three unique peptides, α, α′ and β, that associate as trimers. In some cases, “7S” refers to one or more subunits of β-conglycinin, for example a subunit, a′ subunit, β subunit, or any combination thereof, or in combination with β-conglycinin. Glycinin is a hexameric protein composed of six similar subunits. In some cases, “11S” denotes one or more subunits of glycinin, or in combination of glycinin.

As used herein, the term “High-Throughput Sequencing Platform” refers to a collection of sequencing technologies that enable the simultaneous sequencing of large numbers of nucleic acid fragments. Non-limiting examples of High-Throughput Sequencing Platforms include Illumina's HiSeq and MiSeq systems, Thermo Fisher's Ion Torrent, Pacific Biosciences' SMRT (Single Molecule, Real-Time) sequencing, Oxford Nanopore Technologies' MinION.

As used herein, the term “homogenous” means of uniform structure or composition throughout, such that individual components (e.g., probiotics, particles) cannot be separately observed with naked eye.

As used herein, the term “In vivo assembly” refers to the process of assembling a recombinant protein or protein complex within a living cell or organism.

As used herein, the term “in-vitro” means outside a living organism.

As used herein, the term “Knockdown” refers to a genetic technique for reducing the expression or activity of a specific gene.

As used herein, the term “Lipidation” refers to a post-translational modification process in which a lipid molecule is covalently attached to a protein. This modification serves to increase the hydrophobicity of the protein, potentially promoting its association with cell membranes. Lipidation can occur in several forms, including prenylation, myristoylation, palmitoylation, and the addition of a glycosylphosphatidylinositol (GPI) anchor. Each form of lipidation involves the attachment of a specific type of lipid to a specific residue of the protein, and this can significantly influence the protein's function, stability, localization, and interactions with other proteins.

As used herein, the terms “Mass Spectrometry” and “mass spec” are used interchangeably and refer to an analytical technique used to measure the mass-to-charge ratio of ions, often used to identify and quantify molecules.

As used herein, the term “milk” means a liquid composition that contains soluble casein micelles and where the weight of soluble casein micelles is equal to or greater than 1% of the total protein weight in the composition. As used herein, the term “cheese curd” is a solid or semi-solid mass made by gelating, coagulating, or curdling milk. As used herein, the term “cheese” is a food made from cheese curds. As used herein, the term “milk solids” refers to the powder that would be left after milk is dried out and the water is removed. “Milk particles” comprise casein proteins, for example, α_{S1} casein, α_{S2} casein, β casein, and κ-casein, whey proteins, for example, Beta-lactoglobulin, Alpha-lactalbumin, Serum albumin, as well as lactose, colloidal calcium phosphate (CCP), water, magnesium, citrate, and alkaline phosphatase. Except where otherwise noted, reference to “alpha casein” refer to alpha S1 casein.

As used herein, the terms “mRNA” and “messenger RNA” are used interchangeably and refer to a molecule that carries the genetic information from DNA and acts as a template for protein synthesis.

As used herein, “mScarlet” refers to the monomeric red fluorescent protein.

As used herein, the terms “mutated casein” and “synthetic casein” are used interchangeably and encompass proteins derived from any casein subtype (α_{S1}, α_{S2}, β, κ) that have been modified or synthesized to exhibit a genetic identity that is at least 75% but less than 100% compared to the corresponding naturally-occurring casein sequence. The modifications to achieve said genetic identity may include, but are not limited to, point mutations, insertions, or deletions. The resulting mutated or synthetic caseins retain one or more functional properties of the corresponding natural casein, such as nutritional value, solubility, or structural roles in food matrices. In some instances, the production of mutated or synthetic caseins may involve genetic engineering techniques, chemical synthesis, or other biotechnological approaches to achieve the desired properties and genetic similarity.

As used herein, the term “naturally occurring” means without genetic modification. For example, a naturally occurring ratio of two plant proteins means a ratio of the two plant proteins found in plant (e.g., plant seed), where the plant is not genetically modified to manipulate the expression levels of the two proteins.

As used herein, the term “Nucleic acid sequence” refers to a sequence of nucleotides (A, T, C, and G) that encode genetic information.

As used herein, the term “Oligonucleotide” refers to a short sequence of nucleotides, typically with a defined sequence and length.

As used herein, the term “PCR” and “Polymerase Chain Reaction” are used interchangeably and refer to a method to rapidly replicate a specific DNA sequence in vitro. As used herein, the terms “PEG Transformation” and “Polyethylene glycol Transformation” are used interchangeably and refer to a method for introducing DNA into cells using polyethylene glycol, which promotes the cell membrane's permeability.

As used herein, the term “Pegylation” refers to the covalent attachment of polyethylene glycol (PEG), a non-toxic and non-immunogenic polymer, to a molecule, typically a therapeutic protein or peptide. This modification process can significantly enhance the pharmacokinetic properties of the molecule, extending its circulating half-life in the body, reducing immunogenicity, and improving solubility and stability.

As used herein, the term “phosphomimetic” means a molecular modification wherein a non-phosphorylated amino acid residue is substituted with a residue or compound that mimics the electrostatic and/or structural properties of a phosphorylated amino acid. This substitution aims to simulate the biological activity or functional state normally conferred by phosphorylation, and may be achieved through various methods including, but not limited to, site-directed mutagenesis, chemical modification, or peptide synthesis. As used herein, the term “phospho-ablation” means the targeted removal or inhibition of phosphate groups from specific amino acid residues within a protein or enzyme, thereby modulating its biological activity or function. Phospho-ablation may be achieved through various methods including, but not limited to, chemical treatment, enzymatic dephosphorylation, or genetic modification.

As used herein, the term “Plasmid Vector” refers to a small, circular DNA molecule used as a vehicle to transfer genetic material into a cell.

As used herein, the term “Promoter” refers to a DNA sequence that controls the initiation of transcription of a gene.

As used herein, the term “Protease” refers to an enzyme that catalyzes the breakdown of proteins into smaller peptides or amino acids.

As used herein, the term “protease inhibitor” refers to a molecule or compound that inhibits the activity of proteases, which are enzymes that cleave peptide bonds in proteins. Protease inhibitors can bind to and block the active site of a protease, preventing it from carrying out its enzymatic function. Protease inhibitors can be classified based on their mechanism of inhibition, which can include irreversible binding, reversible binding, or allosteric regulation. Protease inhibitors are known in the art and find applications in research and medical settings, where they can be used to investigate protease function, as well as to treat diseases such as HIV and hepatitis C, which depend on the activity of specific proteases for replication. Protease inhibitors can also be used in industrial settings to protect proteins from degradation, or to enhance the nutritional quality of protein products.

As used herein, the term “Protoplast” refers to a plant or bacterial cell with its cell wall removed.

As used herein, the term “recombinant” refers to nucleic acids or proteins formed by laboratory methods of genetic recombination (e.g., molecular cloning) to bring together genetic material from multiple sources, creating sequences that would otherwise not be found in the genome. Recombinant proteins may be expressed in vivo in various types of host cells, including plant cells, bacterial cells, fungal cells, avian cells, and mammalian cells. Recombinant proteins may also be generated in vitro.

As used herein, the terms “RNAi” and “RNA interference” are used interchangeably and refer to a biological process that inhibits gene expression by destroying specific mRNA molecules.

As used herein, the terms “shRNA” and “short hairpin RNA” are used interchangeably and refer to a small (i.e. fewer than 25 nucleotides) RNA molecule with a tight hairpin turn that can be used to silence gene expression via RNA interference. As used herein, the terms “mhRNA” and “medium hairpin RNA” are used interchangeably and refer to a medium-length RNA molecule (i.e., greater than or equal to 25 nucleotides, but fewer than 200) with a very tight hairpin turn that can be used to silence gene expression via RNA interference. As used herein, the terms “lhRNA” and “long hairpin RNA” are used interchangeably and refer to a large RNA molecule (i.e., greater than or equal to 200 nucleotides), featuring a tight hairpin turn that can be used to silence gene expression via RNA interference.

As used herein, the term “Soy protein product” refers to a product derived from soybeans that contains one or more soy protein fractions.

As used herein, the term “stably expressed” refers to expression and accumulation of a protein in a plant cell over time. As an example, a recombinant protein may accumulate because it is not degraded by endogenous plant proteases. As a further example, a recombinant protein is considered to be stably expressed in a plant if it is present in the plant in an amount of 1% or higher per total protein weight of soluble protein extractable from the plant.

As used herein, the term “SUMOylation” refers to a post-translational modification process where a Small Ubiquitin-like Modifier (SUMO) protein is covalently attached to a target protein. This process is critical for the modulation of various cellular functions, including but not limited to, protein stability, cellular localization, transcriptional regulation, and DNA repair. The enzymatic cascade involved in SUMOylation comprises of an E1 activating enzyme, an E2 conjugating enzyme, and typically an E3 ligase enzyme that facilitates the transfer of the SUMO moiety to the target protein.

As used herein, the terms “TALEN” and “Transcription activator-like effector nuclease” are used interchangeably and refer to a DNA-cutting enzyme that uses a transcription activator-like effector protein to bind to a specific DNA sequence.

As used herein, the term “Targeted gene modification” refers to a genetic technique for introducing specific modifications into a gene.

As used herein, the term “Targeted mutagenesis” refers to a genetic technique for introducing specific mutations into a gene.

As used herein, the term “transgenic plant” means a plant that has been transformed with one or more exogenous nucleic acids. “Transformation” refers to a process by which a nucleic acid is stably integrated into the genome of a plant cell. “Stably transformed” refers to the permanent, or non-transient, retention, expression, or a combination thereof of a polynucleotide in and by a cell genome. A stably integrated polynucleotide is one that is a fixture within a transformed cell genome and can be replicated and propagated through successive progeny of the cell or resultant transformed plant. Transformation can occur under natural or artificial conditions using various methods. Transformation can rely on any method for the insertion of nucleic acid sequences into a prokaryotic or eukaryotic host cell, including Agrobacterium-mediated transformation as illustrated in U.S. Pat. Nos. 5,159,135; 5,824,877; 5,591,616 and 6,384,301, all of which are incorporated herein by reference in its entirety. Methods for plant transformation also include microprojectile bombardment as illustrated in U.S. Pat. Nos. 5,015,580; 5,550,318; 5,538,880; 6,153,812; 6,160,208; 6,288,312 and 6,399,861, all of which are incorporated herein by reference in its entirety. Recipient cells for the plant transformation include meristem cells, callus, immature embryos, hypocotyls explants, cotyledon explants, leaf explants, and gametic cells such as microspores, pollen, sperm and egg cells, and any cell from which a fertile plant can be regenerated, as described in U.S. Pat. Nos. 6,194,636; 6,232,526; 6,541,682, 6,603,061, and 7,151,204, all of which are incorporated herein by reference in its entirety.

As used herein, the term “Transgenic soybean” refers to a genetically modified soybean plant that has been transformed with one or more foreign genes.

As used herein, the term “ubiquitin ligase” refers to a class of enzymes that play a critical role in the ubiquitin-proteasome system (UPS), a cellular pathway responsible for protein degradation. Ubiquitin ligases, also known as E3 enzymes, are responsible for the recognition of specific protein substrates and the facilitation of the transfer of ubiquitin, a small regulatory protein, to these substrates. This process, known as ubiquitination, marks proteins for degradation by the 26S proteasome.

As used herein, the term “vector” means a plasmid comprising operably linked polynucleotide sequences that facilitate expression of a coding sequence in a particular host organism (e.g., a bacterial expression vector or a plant expression vector). Polynucleotide sequences that facilitate expression in prokaryotes can include, e.g., a promoter, an enhancer, an operator, and a ribosome binding site, often along with other sequences. Eukaryotic cells can use promoters, enhancers, termination and polyadenylation signals and other sequences that are generally different from those used by prokaryotes.

As used herein, the terms “ZFN” and “Zinc finger nuclease” are used interchangeably and refer to a DNA-cutting enzyme that uses a zinc finger protein to bind to a specific DNA sequence.

What is claimed is:

1. A mutated alpha casein protein represented by any of the sequences identified in SEQ I.D. Nos. 199-204.

2. A mutated alpha casein protein, wherein the mutated alpha casein protein comprises at least 80% of any of the sequences identified in SEQ ID Nos. 199-204.

3. A mutated beta-casein protein represented by any of the sequences identified in SEQ ID Nos. 207-212.

4. A mutated beta-casein protein, wherein the mutated beta-casein protein comprises at least 80% of any of the sequences identified in SEQ. ID Nos. 207-212.

5. A mutated kappa casein represented by DNA sequence identified in SEQ ID No. 215 and Amino Acid Sequence identified in SEQ ID No. 216.

6. A method for promoting the expression of a casein protein in a plant, comprising inhibiting an expression or activity of a protease in the plant, wherein a reduced activity or expression of the protease increases the expression of the casein protein in the plant.

7. The method in claim 6, wherein reducing the expression or activity of a protease gene in the plant comprises introducing an RNA interference construct targeting the protease gene.

8. The method of claim 7, wherein the RNA interference construct comprises a promoter, a hairpin RNA sequence, and a terminator.

9. The method of claim 7, wherein the RNA interference construct is introduced into the plant by Agrobacterium-mediated transformation or biolistic bombardment.

10. The method of any one of claims 6-9, wherein reducing the expression or activity of a protease gene in the plant comprises introducing a nucleic acid sequence encoding a Cas protein and a guide RNA into the plant and inducing targeted mutagenesis or gene knockout of the protease gene.

11. The method of any one of claims 6-10, comprising introducing a nucleic acid sequence encoding a zinc finger nuclease (ZFN) or transcription activator-like effector nuclease (TALEN) into soybean cells, and inducing targeted mutagenesis or gene knockout of specific protease genes.

12. The method of any one of claims 6-11, comprising introducing a nucleic acid sequence encoding a modified promoter into soybean cells, wherein the modified promoter drives the level of casein proteins in soybean seeds.

13. The method of claim 12, wherein the modified promoter comprises a constitutive promoter or an inducible promoter.

14. A transgenic soybean plant produced by the method in any one of claims 6-13.

15. A method for enhancing expression levels and stability of a casein protein in a transgenic plant, comprising A) expressing the casein protein in the transgenic plant; and B) decreasing proteasomal activity or expression in the transgenic plant to decrease degradation of the casein protein expressed in the transgenic plant.

16. A soy plant, wherein the soy plant: 1) expresses a casein protein comprising alpha-S1-casein, alpha-S2-casein, beta-casein or kappa-casein; and 2) has reduced proteasomal activity or expression level compared to a wildtype soy plant.

17. The soy plant in claim 16, wherein the soy plant has decreased expression or activity of E1 (ubiquitin-activating enzyme), E2 (ubiquitin-conjugating enzyme), or E3 (ubiquitin ligase), or any combination thereof.

18. The soy plant in claim 16, wherein the soy plant has decreased expression or activity of ubiquitin/26S proteasome.

19. A transgenic soy plant comprising a casein gene, wherein the transgenic soy plant exhibits reduced expression of the ubiquitin/26S proteasome compared to a non-transgenic soy plant, and wherein the reduced expression comprises reduced or no expression levels of E1 (ubiquitin-activating enzyme), E2 (ubiquitin-conjugating enzyme), E3 (ubiquitin ligase), or any combination thereof.

20. A method for increasing casein protein expression in a transgenic soy plant, the method comprising: A) expressing the casein protein in the transgenic plant; and B) reducing the activity or expression of the ubiquitin/26S proteasome pathway in the soy plant or part thereof to increase the expression of the casein protein in the transgenic plant; wherein the reducing the expression comprises reduced or no expression levels of E1 (ubiquitin-activating enzyme), E2 (ubiquitin-conjugating enzyme), E3 (ubiquitin ligase), or any combination thereof.

21. A transgenic soy plant, wherein the transgenic soy plant expresses a casein protein, and wherein the soy plant has increased activity or expression level of a deubiquitinating enzyme compared to a wildtype soy plant.

22. A method for expressing a casein protein in a transgenic soy plant, the method comprising A) expressing the casein protein in the transgenic plant; and B) increasing the expression level of a deubiquitinating enzyme in the soy plant, such that the soy plant has increased expression of the casein protein compared to without increasing the expression level of the deubiquitinating enzyme.

23. A method for increasing a casein protein expression in a transgenic soy plant, the method comprising A) expressing the casein protein in the transgenic plant; and B) enhancing the stability and prolonging the half-life of the casein protein in the soy plant through the use of chemical post-translational modifications (PTMs) comprising nitrosylation, methylation, acetylation, lipidation, or pegylation.

24. A method for increasing casein protein expression in a transgenic soy plant, the method comprising A) expressing the casein protein in the transgenic plant; and B) conjugating the casein protein to a cross-linker, wherein the cross-linker is selected from the group consisting of Genipin, Ethylenediamine, Cystamine, Bis(sulfosuccinimidyl) suberate (BS3), or 1-Ethyl-3-(3-dimethylaminopropyl) carbodiimide (EDC), Glutaraldehyde, ethylene glycol diglycidyl ether (EGDE), polyethylene glycol diglycidyl ether (PEGDE), toluene diisocyanate (TDI), hexamethylene diisocyanate (HDI), 1-ethyl-3-(3-dimethylaminopropyl) carbodiimide (EDC), N-Hydroxysuccinimide (NHS), dithiobis[succinimidyl propionate] (DSP), disuccinimidyl suberate (DSS), 3,3′-dithiobis[sulfosuccinimidylpropionate] (DTSSP), bis[sulfosuccinimidyl] suberate (BS3), bis[2-(succinimidooxycarbonyloxy)ethyl]sulfone (BSOCOES), 1,5-difluoro-2,4-dinitrobenzene (DFDNB), formaldehyde, dimethyl adipimidate (DMA), 1,5-difluoro-2,4-dinitrobenzene (DFDNB), genipin, ethylenediamine, cystamine, bismaleimidohexane (BMH), disuccinimidyl tartrate (DST), ethylene glycol bis[succinimidylsuccinate] (EGS), N,N′-ethylenebis(iodoacetamide) (EBI), N,N′-(1,3-phenylene)dibutyric acid di[succinimidyl ester] (DSS), N,N′-(ethylene-di-1,2-phenylene)bismaleimide, N,N′-1,4-phenylenebismaleimide, divinyl sulfone, diisopropylcarbodiimide, dicyclohexylcarbodiimide, diethylcarbodiimide, difluorodinitrobenzene, N,N′-disuccinimidyl carbonate (DSC), dithiobispropionimidate, glyoxal, 1,5-difluoro-2,4-dinitrobenzene, N-succinimidyl(4-iodoacetyl)aminobenzoate (SIAB), N-succinimidyl(4-iodoacetyl)aminobenzoate (SIA), succinimidyl 4-(p-maleimidophenyl)butyrate (SMPB), and succinimidyl 6-[(beta-maleimidopropionamido)hexanoate] (SMPH).

25. The method of claim 24, wherein the cross-linker is non-toxic.

26. A casein molecule comprising a casein protein and a cross-linker, wherein the wherein the cross-linker is selected from the group consisting of Genipin, Ethylenediamine, Cystamine, Bis(sulfosuccinimidyl) suberate (BS3), or 1-Ethyl-3-(3-dimethylaminopropyl) carbodiimide (EDC), Glutaraldehyde, ethylene glycol diglycidyl ether (EGDE), polyethylene glycol diglycidyl ether (PEGDE), toluene diisocyanate (TDI), hexamethylene diisocyanate (HDI), 1-ethyl-3-(3-dimethylaminopropyl) carbodiimide (EDC), N-Hydroxysuccinimide (NHS), dithiobis[succinimidyl propionate] (DSP), disuccinimidyl suberate (DSS), 3,3′-dithiobis[sulfosuccinimidylpropionate] (DTSSP), bis[sulfosuccinimidyl] suberate (BS3), bis[2-(succinimidooxycarbonyloxy)ethyl]sulfone (BSOCOES), 1,5-difluoro-2,4-dinitrobenzene (DFDNB), formaldehyde, dimethyl adipimidate (DMA), 1,5-difluoro-2,4-dinitrobenzene (DFDNB), genipin, ethylenediamine, cystamine, bismaleimidohexane (BMH), disuccinimidyl tartrate (DST), ethylene glycol bis[succinimidylsuccinate] (EGS), N,N′-ethylenebis(iodoacetamide) (EBI), N,N′-(1,3-phenylene)dibutyric acid di[succinimidyl ester] (DSS), N,N′-(ethylene-di-1,2-phenylene)bismaleimide, N,N′-1,4-phenylenebismaleimide, divinyl sulfone, diisopropylcarbodiimide, dicyclohexylcarbodiimide, diethylcarbodiimide, difluorodinitrobenzene, N,N′-disuccinimidyl carbonate (DSC), dithiobispropionimidate, glyoxal, 1,5-difluoro-2,4-dinitrobenzene, N-succinimidyl(4-iodoacetyl)aminobenzoate (SIAB), N-succinimidyl(4-iodoacetyl)aminobenzoate (SIA), succinimidyl 4-(p-maleimidophenyl)butyrate (SMPB), and succinimidyl 6-[(beta-maleimidopropionamido)hexanoate] (SMPH).

27. A method for increasing casein protein expression in a transgenic soy plant, the method comprising mutating the casein protein to comprise fewer Proline (P), Glutamic acid (E), Serine(S), or Threonine (T) compared to a wildtype casein protein.

28. A fusion protein, comprising a casein protein and a peptide sequence that represents a ubiquitin-associated (UBA) domain.

29. A method for increasing casein protein expression in a transgenic soy plant; the method comprising expressing a casein protein in the transgenic soy plant, wherein the casein protein is fused with a peptide sequence that represents a ubiquitin-associated (UBA) domain.

30. A method for enhancing expression levels and stability of a casein protein in a transgenic plant, comprising expressing a casein protein in the transgenic soy plant, wherein the transgenic plant comprises a promoter that regulates the expression of the casein gene in the transgenic plant, and wherein the promoter is non-naturally occurring.

31. A soy plant, wherein the soy plant: A) expresses a casein protein comprising alpha-S1-casein, alpha-S2-casein, beta-casein or kappa-casein; and B) has a modified promoter of the casein gene compared to a wildtype soy plant.

32. A method for enhancing expression levels and stability of a casein protein in a transgenic plant, comprising optimizing the codons of the casein gene in the plant.

33. A soy plant, wherein the soy plant: A) expresses a casein protein comprising alpha-S1-casein, alpha-S2-casein, beta-casein or kappa-casein; and B) has optimized codons of the casein gene compared to a wildtype soy plant.

34. A method for enhancing expression levels and stability of a casein protein in a transgenic plant cell, comprising expressing the casein protein in the transgenic plant cell; wherein the transgenic plant comprises at least two, at least three, at least four, at least five, at least six copies, at least seven copies, or at least eight copies of DNA sequences coding the casein gene in the transgenic plant cell.

35. A soy plant cell, wherein the soy plant cell expresses a casein protein comprising alpha-S1-casein, alpha-S2-casein, beta-casein or kappa-casein, wherein the soy plant cell comprises at least two, at least three, at least four, at least five, at least six copies, at least seven copies, or at least eight copies of DNA sequences coding the casein gene in the soy plant cell.

36. A method for enhancing translation of a casein protein in a transgenic plant, comprising one or more of expressing the casein protein in the transgenic plant, wherein the transgenic plant comprises a mRNA coding the casein protein; and wherein the mRNA is post-transcriptionally modified to increase the translation of the casein protein.

37. A soy plant, wherein the soy plant expresses a casein protein comprising alpha-S1-casein, alpha-S2-casein, beta-casein or kappa-casein; and wherein the soy plant comprises a mRNA coding the casein protein; and wherein the mRNA is post-transcriptionally modified to increase the translation of the casein protein.

38. A method for enhancing expression levels and stability of a casein protein in a transgenic plant, comprising mutating the sequence of the casein protein in the plant to increase the expression levels and stability of a casein protein in a transgenic plant.

39. A soy plant, wherein the soy plant: A) expresses a casein protein comprising alpha-S1-casein, alpha-S2-casein, beta-casein or kappa-casein; and B) has a mutated sequence of the casein protein compared to a wildtype casein protein.

40. A method for enhancing expression levels and stability of a casein protein in a transgenic plant, comprising editing the chromosomal location of the casein gene in the plant to increase the expression levels of the casein protein in the transgenic plant.

41. A soy plant, wherein the soy plant: A) expresses a casein protein comprising alpha-S1-casein, alpha-S2-casein, beta-casein or kappa-casein; and B) has a different chromosomal location of the casein gene.

42. A method for enhancing expression levels and stability of a casein protein in a transgenic plant, comprising using a chaperone protein to assist in the folding and assembly of the casein protein in the plant, such that the transgenic plant has increased expression levels and stability of the casein protein.

43. A soy plant, wherein the soy plant: A) expresses a casein protein comprising alpha-S1-casein, alpha-S2-casein, beta-casein or kappa-casein; and B) comprises a chaperone protein to assist in the folding and assembly of the casein protein.

44. A method for enhancing expression levels and stability of a casein protein in a transgenic plant, comprising incorporating a translational enhancer in the sequence of the casein gene in the plant to increase the expression levels and stability of a casein protein in a transgenic plant.

45. A soy plant, wherein the soy plant: A) expresses a casein protein comprising alpha-S1-casein, alpha-S2-casein, beta-casein or kappa-casein; and B) comprises a translational enhancer incorporated in the sequence of the casein gene.

46. A method for enhancing expression levels and stability of a casein protein in a transgenic plant, comprising adding a Scaffold/Matrix Attachment Region (SAR or Mar) to the casein gene in the plant to increase the expression levels and stability of the casein protein in the transgenic plant.

47. A soy plant, wherein the soy plant: A) expresses a casein protein comprising alpha-S1-casein, alpha-S2-casein, beta-casein or kappa-casein; and B) comprises a Scaffold/Matrix Attachment Region (SAR or Mar) added to the casein gene.

48. A method for enhancing expression levels and stability of a casein protein in a transgenic plant, comprising incorporating introns into the coding sequence of the casein gene in the plant to increase the expression levels and stability of the casein protein in the transgenic plant.

49. A soy plant, wherein the soy plant: A) expresses a casein protein comprising alpha-S1-casein, alpha-S2-casein, beta-casein or kappa-casein; and B) comprises introns incorporated into the coding sequence of the casein gene to increase the expression levels and stability of the casein protein in the transgenic plant.

50. A method for enhancing expression levels and stability of a casein protein in a transgenic plant, comprising inducing hormonal changes in the plant to enhance casein gene expression.

51. A soy plant, wherein the soy plant: A) expresses a casein protein comprising alpha-S1-casein, alpha-S2-casein, beta-casein or kappa-casein; and B) wherein the soy plant has undergone hormonal induction to enhance casein gene expression.

52. A method for enhancing expression levels and stability of a casein protein in a transgenic plant, comprising using antisense RNAs to suppress negative regulators of casein expression in the plant, to increase the expression levels and stability of the casein protein in the transgenic plant.

53. A soy plant, wherein the soy plant: A) expresses a casein protein comprising alpha-S1-casein, alpha-S2-casein, beta-casein or kappa-casein; and B) comprises an antisense RNA to suppress negative regulators of casein expression.

54. A method for enhancing expression levels and stability of a casein protein in a transgenic plant, comprising modifying the 3′ Untranslated Region (UTR) of the casein gene in the plant, to increase the expression levels and stability of the casein protein in the transgenic plant.

55. A soy plant, wherein the soy plant: A) expresses a casein protein comprising alpha-S1-casein, alpha-S2-casein, beta-casein or kappa-casein; and B) comprises a modified 3′ Untranslated Region (UTR) of the casein gene.

56. A method for enhancing expression levels and stability of a casein protein in a transgenic plant, comprising optimizing the signal peptide of the casein protein in the plant.

57. A soy plant, wherein the soy plant: A) expresses a casein protein comprising alpha-S1-casein, alpha-S2-casein, beta-casein or kappa-casein; and B) comprises an optimized signal peptide of the casein protein compared to a wildtype soy plant.

58. A method for enhancing expression levels and stability of a casein protein in a transgenic plant, comprising fusing the casein protein with another protein in the plant, to increase the expression levels and stability of the casein protein in the transgenic plant.

59. A soy plant, wherein the soy plant expresses a fusion of the casein protein with another protein compared to a wildtype soy plant.

60. A method for enhancing expression levels and stability of a casein protein in a transgenic plant, comprising knocking down protease activity or expression in the plant to increase the expression levels and stability of the casein protein in the transgenic plant.

61. The method of claim 60, wherein the enhancing of expression levels and stability of a casein protein in a transgenic plant is achieved by the knocking down of sequence identified in SEQ. I.D. 229.

62. A soy plant, wherein the soy plant: A) expresses a casein protein comprising alpha-S1-casein, alpha-S2-casein, beta-casein or kappa-casein; and B) has reduced protease activity or expression level compared to a wildtype soy plant.

63. A method for enhancing expression levels and stability of a casein protein in a transgenic plant, comprising optimizing the nutrient and environmental conditions of the plant to enhance casein gene expression.

64. A soy plant, wherein the soy plant: A) expresses a casein protein comprising alpha-S1-casein, alpha-S2-casein, beta-casein or kappa-casein; and B) is grown under optimized nutrient and environmental conditions to enhance casein gene expression compared to a wildtype soy plant.

65. A method of expressing casein proteins in a plant, wherein the method results in increased expression of the casein gene in the soy plant or part thereof compared to a soy plant or part thereof not subjected to the method.

66. A casein protein modified by lipidation, wherein the lipidation is cysteine prenylation, N-terminal glycine myristoylation, cysteine palmitoylation, or serine and lysine fatty acylation.

67. A modified casein protein, comprising a casein protein and a polyethylene glycol (PEG), wherein the polyethylene glycol (PEG) is covalently attached to casein protein.

68. A method of extracting casein proteins from the soybean plant; comprising providing using a buffer comprising a protease inhibitor selected from the group consisting of a Serine Protease Inhibitor, a Cysteine Protease Inhibitor, an Aspartic Protease Inhibitor, a Metalloprotease Inhibitor, a Covalent Protease Inhibitor, a Reversible Protease Inhibitor, a Peptide-based Protease Inhibitor, a Synthetic Protease Inhibitor, or a Natural Protease Inhibitor.

69. A soybean seed comprising casein proteins, wherein the casein proteins comprise at least 0.03% of the total weight of the seed, at least 0.041% of the total weight of the seed, or at least 0.055% of the total weight of the seed, or at least 0.075% of the total weight of the seed, or at least 0.101% of the total weight of the seed, or at least 0.136% of the total weight of the seed, or at least 0.184% of the total weight of the seed, or at least 0.248% of the total weight of the seed, or at least 0.335% of the total weight of the seed, or at least 0.452% of the total weight of the seed, or at least 0.610% of the total weight of the seed, or at least 0.824% of the total weight of the seed, or at least 1.113% of the total weight of the seed, or at least 1.503% of the total weight of the seed, or at least 2.031% of the total weight of the seed, or at least 2.744% of the total weight of the seed, or at least 3.707% of the total weight of the seed, or at least 5.006% of the total weight of the seed, or at least 6.761% of the total weight of the seed, or at least 13% of the total weight of the seed.

70. A soybean seed comprising casein proteins, wherein the casein proteins comprise at least 0.075% of the total protein content of the seed, or at least 0.103% of the total protein content of the seed, or at least 0.138% of the total protein content of the seed, or at least 0.188% of the total protein content of the seed, or at least 0.253% of the total protein content of the seed, or at least 0.340% of the total protein content of the seed, or at least 0.460% of the total protein content of the seed, or at least 0.620% of the total protein content of the seed, or at least 0.838% of the total protein content of the seed, or at least 1.130% of the total protein content of the seed, or at least 1.525% of the total protein content of the seed, or at least 2.060% of the total protein content of the seed, or at least 2.783% of the total protein content of the seed, or at least 3.758% of the total protein content of the seed, or at least 5.078% of the total protein content of the seed, or at least 6.860% of the total protein content of the seed, or at least 9.267% of the total protein content of the seed, or at least 12.503% of the total protein content of the seed, or at least 16.903% of the total protein content of the seed, or at least 32.5% of the total protein content of the seed.

71. A soybean seed comprising casein micelles, wherein the casein micelles comprise at least 0.03% of the total weight of the seed, or at least 0.041% of the total weight of the seed, or at least 0.055% of the total weight of the seed, or at least 0.075% of the total weight of the seed, or at least 0.101% of the total weight of the seed, or at least 0.136% of the total weight of the seed, or at least 0.184% of the total weight of the seed, or at least 0.248% of the total weight of the seed, or at least 0.335% of the total weight of the seed, or at least 0.452% of the total weight of the seed, or at least 0.610% of the total weight of the seed, or at least 0.824% of the total weight of the seed, or at least 1.113% of the total weight of the seed, or at least 1.503% of the total weight of the seed, or at least 2.031% of the total weight of the seed, or at least 2.744% of the total weight of the seed, or at least 3.707% of the total weight of the seed, or at least 5.006% of the total weight of the seed, or at least 6.761% of the total weight of the seed, or at least 13% of the total weight of the seed.

72. A soybean seed comprising casein micelles, wherein the casein micelles comprise at least 0.075% of the total protein content of the seed, or at least 0.103% of the total protein content of the seed, or at least 0.138% of the total protein content of the seed, or at least 0.188% of the total protein content of the seed, or at least 0.253% of the total protein content of the seed, or at least 0.340% of the total protein content of the seed, or at least 0.460% of the total protein content of the seed, or at least 0.620% of the total protein content of the seed, or at least 0.838% of the total protein content of the seed, or at least 1.130% of the total protein content of the seed, or at least 1.525% of the total protein content of the seed, or at least 2.060% of the total protein content of the seed, or at least 2.783% of the total protein content of the seed, or at least 3.758% of the total protein content of the seed, or at least 5.078% of the total protein content of the seed, or at least 6.860% of the total protein content of the seed, or at least 9.267% of the total protein content of the seed, or at least 12.503% of the total protein content of the seed, or at least 16.903% of the total protein content of the seed, or at least 32.5% of the total protein content of the seed.

73. A method for enhancing translational efficiency in genetically modified organism, comprising the use of both 5′ and 3′ untranslated regions (UTRs) within a single genetic construct.

74. The method of claim 73, wherein the 5′ UTR is selected from sequences 1 through 16.

75. The method of claim 73, wherein the 3′ UTR is selected from sequences 17 through 70.

76. The method of claim 74 wherein the 5′ UTR is at least 70%, 74%, 78%, 82%, 86%, 90%, 93%, 96%, 98%, or 99% identical to a sequence from among sequences 1 through 16.

77. The method of claim 75 wherein the 3′ UTR is at least 70%, 74%, 78%, 82%, 86%, 90%, 93%, 96%, 98%, or 99% identical to a sequence from among sequences 17 through 70.

78. The method of claim 73, wherein the genetic construct comprises genes selected from the group consisting of an αs1-casein, αs2-casein, β-casein, κ-casein, and a kinase, such as FAM20C.

79. The method of claim 78, wherein the kinase is FAM20C.

80. The method of claim 73, wherein a fluorescence-based metric is used for assessing translational efficiency.

81. The method of claim 73, wherein ribosome profiling is employed to assess translational efficiency.

82. The method of claim 80, wherein green fluorescent protein (GFP) and red fluorescent protein (RFP) are utilized as fluorescent markers.

83. A genetic construct designed for enhanced translational efficiency, comprising both a 5′ UTR and a 3′ UTR.

84. The genetic construct of claim 83, wherein the 5′ UTR is selected from sequences 1 to 16.

85. The genetic construct of claim 83, wherein the 3′ UTR is selected from sequences 17 to 70.

86. The genetic construct of claim 83, wherein it comprises genes selected from the group consisting of an αs1-casein, αs2-casein, β-casein, κ-casein, and a kinase, such as FAM20C.

87. The genetic construct of claim 86, wherein the kinase is FAM20C.

88. The genetic construct of claim 83, wherein PEG-mediated transformation is employed to introduce the genetic construct into target cells.

89. The genetic construct of claim 83, wherein bi-directional promoters are utilized for the regulation of gene expression.

90. The genetic construct of claim 83, wherein the 5′ and 3′ UTRs are configured to regulate a casein gene and a kinase gene simultaneously.

91. The genetic construct of claim 83, wherein fluorescent-activated cell sorting (FACS) is employed to segregate transformed cells based on translational efficiency.

92. A method for enhancing the stability of transgenic casein in soy plants, comprising expressing a broad-spectrum protease inhibitor within said soy plants.

93. The method of claim 92, wherein the broad-spectrum protease inhibitor is engineered with a localization signal identical to that of the transgenic casein.

94. The method of claim 92, further comprising the step of quantitatively analyzing the level of transgenic casein in the soy plants using immunoblot techniques.

95. A soy plant genetically modified to express transgenic casein and a broad-spectrum protease inhibitor, wherein the protease inhibitor provides protection against proteolytic degradation.

96. The soy plant of claim 95, wherein the broad-spectrum protease inhibitor is selected from the group consisting of serine protease inhibitors and cysteine protease inhibitors.

97. A plasmid construct for use in transforming soy plants to express transgenic casein, the construct comprising a broad-spectrum protease inhibitor gene and a casein gene, each under the control of a constitutive promoter.

98. The plasmid construct of claim 97, wherein the constitutive promoter is selected from the group consisting of the Cauliflower Mosaic Virus 35S promoter and the mannopine synthase promoter.

99. A method for producing soy-based products with enhanced casein content, comprising cultivating the soy plant of claim 106 and harvesting the soy plant material containing the stabilized transgenic casein.

100. A method for inhibiting protease activity in transgenic soy plants expressing casein, comprising the step of downregulating endogenous proteases using RNA interference technology.

101. The method of claim 100, wherein the RNA interference targets one or more proteases selected from the group consisting of serine proteases, cysteine proteases, aspartic proteases, and metalloproteases.

102. A transgenic soy plant with increased casein stability, wherein the plant has reduced activity or expression of proteases through the introduction of a broad-spectrum protease inhibitor gene.

103. The transgenic soy plant of claim 102, wherein the broad-spectrum protease inhibitor is co-localized with the transgenic casein in the endoplasmic reticulum and protein storage vacuoles.

104. A method for selecting a broad-spectrum protease inhibitor for use in the method of claim 92, comprising screening potential inhibitors for efficacy in the pH and ionic environments of soy plant cellular compartments.

105. The method of claim 104, wherein the potential inhibitors are screened for inhibitory effects against endogenous soy plant proteases that degrade casein proteins.

106. A kit for transforming soy plants to produce stable transgenic casein, comprising a vector with a broad-spectrum protease inhibitor gene, a casein gene, and instructions for use in a plant transformation protocol.

107. The kit of claim 106, wherein the vector further comprises a selectable marker gene for kanamycin resistance.

108. A transgenic soy plant produced by the method of claim 92, wherein the plant exhibits a statistically significant increase in casein protein levels compared to a control plant without the broad-spectrum protease inhibitor.

109. A method for producing transgenic casein in soy plants for use in cheese production, the method comprising expressing a bovine casein gene alongside a broad-spectrum protease inhibitor in the soy plant.

110. The method of claim 109, further comprising the step of purifying the transgenic casein from the soy plant material for use in dairy substitute products.

111. A soy plant with reduced expression of endogenous proteases, produced by a method comprising the steps of transforming the soy plant with a plasmid containing a broad-spectrum protease inhibitor gene and cultivating the transformed plant under conditions that promote the expression of the inhibitor and casein proteins.

112. A method for modifying endogenous protease in a plant to enhance the expression of transgenic casein.

113. The method of claim 112, wherein the modification includes the alteration of the active site cysteine residue.

114. A plant comprising a modified protease and a transgenic casein.

115. The genetically engineered organism of claim 114, wherein the plant is soy.

116. The method of claim 112, wherein the endogenous protease is a subtilisin-like protease.

117. The method of claim 112, wherein the endogenous protease is a modified thiol proteinase.

118. The plant of claim 114, wherein the modified protease is a subtilisin-like protease.

119. The method of claim 112 or 113, wherein the modifications to endogenous protease and transgenic casein are related to casein proteins for applications in the food industry.

120. A method for producing a modified thiol proteinase with reduced ability to cleave intracellular proteins.

121. A method for enhancing the expression of foreign casein genes in soybeans, comprising the steps of co-expressing said casein genes with Bowman Birk Protease Inhibitors (BBPIs) in a soybean plant, wherein said BBPIs are serine protease inhibitors that reduce the degradation of said casein proteins, thereby increasing their expression within the plant.

122. The method of claim 121, wherein a plasmid configuration, including a specific plasmid referred to as Pmoz 2274, is utilized for the expression of said casein genes and a kinase in the soybean plant.

123. The method of claim 122, further involving the use of an alternative plasmid, Pmoz 2284, which comprises the components of Pmoz 2274 with the addition of BBPIs. to transform soybean tissue and enhance the expression of said casein genes.

124. The method of claim 121, wherein the transformational efficiency is assessed using a GFP marker, and the presence of a peptide tag, V5, at the end of the casein allows for quantification of alpha casein expression, with the expression of alpha casein being significantly increased in the presence of BBPIs.

125. The method of claim 121, further comprising the addition of a GY1-VSD sequence to the casein genes, said sequence acting as a vesicle sorting determinant for targeting the expressed proteins to the vacuole for improved processing and stability.

126. The method of claim 121, wherein various peptide tags, including but not limited to His-tag, FLAG-tag, or HA-tag, are employed alongside or in lieu of the V5 tag to facilitate enhanced quantification and monitoring of casein expression.

127. The method of claim 121, further comprising the use of diverse promoters within the plasmid constructs to regulate the expression of casein and kinase genes, including the use of the 35S CaMV promoter for constitutive expression or tissue-specific promoters for targeted expression in desired soybean tissues.

128. The method of claim 121, wherein the co-expression of chaperone proteins within the soybean is employed to assist in the correct folding and assembly of the expressed casein proteins, thereby enhancing the stability and functionality of the recombinant proteins.

129. The method of claim 121, further comprising the use of various transformation techniques, including Agrobacterium-mediated transformation, biolistic particle delivery, or electroporation, for the introduction of plasmids into soybean tissue, tailored to different soybean varieties and tissue types.

130. The method of claim 121, further involving a comprehensive analysis of the post-translational modifications experienced by the expressed casein proteins, particularly those occurring within the endoplasmic reticulum and Golgi apparatus, to enhance understanding of protein stability, solubility, and bioactivity, and to ensure responsible and safe application within the agricultural sector.

131. A transgenic plant comprising 1) a reduced level of protease activity or expression, as compared to a wild-type counterpart; and 2) an increased levels of casein, as compared to a counterpart without the reduced level of protease activity or expression.

132. The transgenic plant in claim 131, wherein the plant is soybean.

133. The transgenic plant in claim 131 or 132, wherein the casein is at least one of κ-casein, αS1-casein, αS2-casein, β-casein, or any combination thereof.

134. The transgenic plant in any one of the claims 131-133, wherein the transgenic plant has enhanced levels of essential amino acids as compared to a wild-type counterpart.

135. The transgenic plant in any one of claims 131-134, wherein the transgenic plant has improved digestibility as compared to a wild-type counterpart.

136. The transgenic plant in any one of claims 131-135, wherein the transgenic plant has enhanced levels of casein micelle formation as compared to a counterpart without the reduced level of protease activity or expression.

137. The transgenic plant in any one of claims 131-136, wherein the protease is at least one of cysteine protease, an aspartyl proteases, a metalloprotease, serine proteases, or any combination thereof.

138. The transgenic plant in any one of claims 131-137, wherein the protease is one or more the proteases identified in the Sequences in SEQ ID No. 1-16 or 217-241.

139. The transgenic plant in any one of claims 131-138, wherein the protease comprises a peptide sequence that is at least at least 80%, at least 85%, at least 90%, at least 95%, or at least 99% sequence identity to any one of the sequences in SEQ ID. No. 1-16 or 217-241.

140. A composition comprising an ingredient derived from any one of claims 131-139.

141. The composition in claim 140, further comprising at least one of carbohydrates, fats, vitamins, minerals, or flavorings.


SEQUENCE LISTING (250 sequences)

SEQ ID NO: 1 - protein, 507 aa, Glycine max
MGNMSNVVVFCFCLWTLLFSLVFCAPNDGLGRIGLKKVKLNTHDVEGLKEFRSSIRKHHLQNILGGAEETDVVALKNYLDAQYYGEIAIGTPPQKFTVIFDTGSSNLWVPSSKCYFSIACFMHARYRSSQSSTYRENGTSAAIQYGTGAISGFFSNDDVKVGDIVVKDQEFIEATREPGVTFVAAKFDGILGLGFQDISVGYAVPVWYSMVEQGLVKDPVFSFWLNRKPEEENGGELVFGGADPAHYKGKHTYVPVTRKGYWQFDMGDVLIAGKPTGYCADDCSAIADSGTSLLAGPTTVVTMINQAIGASGVVSKECRSVVNQYGQTILELLLAEAKPKKICSQIGLCTFDGTHGVSMGIESVVDKNERKSSGSIRDAGCSACEMAVIWMQNQLRQNQTEDRIIDYANELCDKLPNPMGQSSVDCEKLSSMPIVSFTIGGKVFDLSPQEYILKVGEGPEAQCISGFTALDVPPPRGPLWILGDVFMGRYHTIFDYGKLRVGFAEAA

SEQ ID NO: 2 - protein, 499 aa, Glycine max
MEHCNCYMFTLSLLLLLLLTPPSLSDDLGFDFPAKKLIRDLNLFPSEDVNIVPRHSNSHANKIVEKPLRFPNLVPSDSGISLDDLAHRAGYYLIPHSHAAKMFYFFFESRNSKKDPVVIWLTGGPGCSSELAVFYENGPFKIANNMSLVWNEYGWDKVSNLLYVDQPTGTGFSYSTDKRDIRHDEEGVSNDLYDFLQAFFAEHPEYVKNDFFITGESYAGHYIPAFAARVHRGNKAKEGIHINLKGFAIGNGLTDPGIQYKAYTDYALDMGIIQKADYERINKVMVPACEMAIKLCGTDGKIACTASYFVCNTIFNSIMSHAGDINYYDIRKKCEGSLCYDFSNLEKYLNQKSVRDALGVGDIDFVSCSSTVYQAMLVDWMRNLEVGIPALLEDGINMLVYAGEFDLICNWLGNSKWVHAMEWSGQQEFVVSSEVPFTVDDSEAGLLKKYGPLSFLKVHDAGHMVPMDQPKASLEMLKRWTQGTLSESAADAEKLVAEL

SEQ ID NO: 3 - protein, 472 aa, Glycine max
MKNLATWKTMAITVVLLQLSFSLEIFCLSYHADRIVRLPGQPNIGFQQFSGYVTVDDMKHKALFYYFVESETDPASKPLVLWLNGGPGCSSLGVGAFSENGPFRPNGEVLIKNEYSWNRETNMLYLETPVGVGFSYAKGGSSYDTVNDETTVLLISQARDNLVFLQRWFNKFPHYRHTDLFLAGESYAGHYVPQLAKLMIEINKKEKMFNLKGIALGNPVLEYATDFNSRAEFFWSHGLISDSTYKLFTTGCNYSRYVSEYYRDSISPLCSKVMKQVSRETSKFVDKYDVTLDVCISSVLSQSKAICPQSQQTNESIDVCVDDKVTNYLNRKDVQEALHAKLVGVQKWNVCSTILDYDMLNLEVPTLPIVGSLIKAGVRVLIYSGDQDSVIPLTGSRTLVQKLARQLRLNTTIHYRVWFEGQQVGGWTQVYGNILSFATVRGASHEAPFSQPERSLVLFKSFLEDRPLPEIF

SEQ ID NO: 4 - protein, 481 aa, Glycine max
MFPQPWIFLIATIIAISLFMSSLVESFPVADKVKSLPEQSPVSFQQFAGFVPVDDKNQRALFYYFVEAETNPASKPLVLWLNGGPGCTSVGVGAFTEHGPFVTNQGEAIEKNQYSWNKEANILYLESPAGVGFSYSLNLSFYKTLNDEITARDSLVFLRRWFAKFPEYKNRDFYITGESYGGHYVPQLAELIIKSKVNFNLKGIAIGNPLLDFDTDMNAVDEYYWSHGIISDYAYKIRTSLCNSSRVLREYFSGQISKDCLVAAQKVSEEYSFTNFIDPYYVVGEKCLSYNVSQAGFLRETLNSGMFQFRNSHYVLQTEEPDQQVDECNLKYSEMYLNRKDVQKALHARLEGTTKYRLCSKIVQTNYDPLNREIPTINVVGFLVKSGLRVIVYSGDQDSVIPFMGTRRLVDRLAKTLGLKTTLPYSAWFVDKQVGGWTKVYGNHLTYTTIRGASHGTPATQPKRSFVLFNAFLQGKPLPKA

SEQ ID NO: 5 - protein, 467 aa, Glycine max
MAQRQIFAHVVILLLMVLVGARFAKAEGGGEAADRILELPGQPKVSFQQFSGYVTVNKVAGRALFYWLTEAAQNPLTKPLVIWLNGGPGCSSVAYGASEEIGPFRINKTASGLYINKFSWNTVANLLFLEAPAGVGFSYANRSSDLLNTGDRRTAQDSLEFVIQWLERFPRYKNRELYITGESYAGHYVPQLAKEILTYNAKTKHPINLKGIMVGNAVTDNYYDNLGTVTYWWSHAMISDQTYRQLMSTCDFHRQKESDECESVYSYAMDQEFGNIDQYNIYAPPCNNSDGSSSSANRRTMRLPHRPHVDFSHWSGYDPCTEKYAEIYYNRPDVQKALHANKTGIPYRWTACSEVLNRNWNDTDVSVLPIYRELIAHGIRVWVFSGDVDSVVPVTATRYALAQLKLSTKIPWYPWYVKNQVGGWTEVYEGVTFATVRGAGHEVPLFKPRAALQLFKSFLEGKPLPKS

SEQ ID NO: 6 - protein, 362 aa, Glycine max
MMSLQRTKLFPFFIVLVSFTCSLSLAMSSNQLEQFASEEEVFQLFQAWQKEHKREYGNQEEKAKRFQIFQSNLRYINEMNAKRKSPTTQHRLGLNKFADMSPEEFMKTYLKEIEMPYSNLESRKKLQKGDDADCDNLPHSVDWRDKGAVTEVRDQGKCQSHWAFSVTGAIEGINKIVTGNLVSLSVQQVVDCDPASHGCAGGFYFNAFGYVIENGGIDTEAHYPYTAQNGTCKANANKVVSIDNLLVVVGPEEALLCRVSKQPVSVSIDATGLQFYAGGVYGGENCSKNSTKATLVCLIVGYGSVGGEDYWIVKNSWGKDWGEEGYLLIKRNVSDEWPYGVCAINAAPGFPIIKEVASSSAI

SEQ ID NO: 7 - protein, 217 aa, Glycine max
MKSTIFFALFLFCAFTTSYLPSAIADFVLDNEGNPLENGGTYYILSDITAFGGIRAAPTGNERCPLTVVQSRNELDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESLAKKNHGLSRSE

SEQ ID NO: 8 - protein, 763 aa, Glycine max
MLQAVLCFFCFWLCLLQKLAFAFPPRIWIGWQVYVVYMGSKSGEHPDDILKENHQILASVHSGSIEEAQASHIYTYKHGFRGFAAKLSDEQASQISKMPGVVSVFPNSKRKLHTTHSWDFMGLLDDQTMETLGYSIRNQENIIIGFIDTGIWPESPSFSDTDMPAVPPGWKGQCQSGEGFNASSCNRKVIGARYYRSGYEAAEGDSDAKKSFISARDSTGHGSHTASIAAGRFVANMNYKGLASGGARGGAPMARIAVYKTCWDSGCYDVDLLAAFDDAIRDGVHILSLSLGAESPQGDYFSDAISVGSFHAASRGVLVVASAGNEGSAGSATNLAPWMLTVAASSTDRDFTSDIILGNGAKIMGESLSLFEMNASTRIISASAANGGYFTPYQSSYCLESSLNKTKSKGKVLVCRHAESSTESKVLKSKIVKAAGGVGMILIDETDQDVAIPFVIPSAIVGNKIGEKILSYLRTTRKPVSRIFGAKTVLGAHPAPRVAAFSSKGPNALNPEILKPDVTAPGLNILAAWSPAAGNMFNILSGTSMACPHVTGIATLVKAVHPSWSPSAIKSAIMTTATVLDKHHRPITADPEQRRANAFDYGSGFVNPARVLDPGLIYDSKPADFVAFLCSLGYDQRSLHQVTRDNSTCDRAFSTASDLNYPSIAVPNLKDNFSVTRIVTNVGKARSVYKAVVSSPPGVRVSVIPNRLIFTRIGQKINFTVNFKLSAPSKGYAFGFLSWRNRISQVTSPLVVRVAPGKNGLVR

SEQ ID NO: 9 - protein, 770 aa, Glycine max
MGKQVLNSFPLIVFFFILFSTVSADEVSKTFIFRVDSQSKPTVFPTHYHWYTSEFAQETSILHLYDTVFCGFSAVLTSHQVASISQHPSVLAVFEDRRRQLHTTRSPQFLGLRNQRGLWSESDYGSDVIVGVFDTGVWPERRSFSDLNLGPIPRRWKGACETGASFSPKNCNRKLIGARFFSKGHEAGAGSGPLNPINETVEFRSPRDADGHGTHTASTAAGRYAFQASMSGYAAGIAKGVAPKARLAVYKVCWKNSGCFDSDILAAFDAAVNDGVDVISISIGGGDGIASPYYLDPIAIGSYGAVSRGVFVSSSAGNDGPSGMSVTNLAPWLTTVGAGTIDREFPSQVILGDGRRLSGVSLYAGAALKGKMYQLVYPGKSGILGDSLCMENSLDPSMVKGKIVICDRGSSPRVAKGLVVKKAGGVGMILANGISNGEGLVGDAHLLPACAVGANEGDLIKKYISSSKNPTATLDFKGTILGIKPAPVIASFSARGPNGLNPEILKPDLIAPGVNILAAWTEAVGPTGLDSDTRRTEFNILSGTSMACPHVSGAAALLKSAHPDWSPAAIRSAMMTTATVLDNRNKTMTDEATGNSSTPYDFGAGHLNLGRAMDPGLVYDITNNDYVNFLCGIGYGPKVIQVITRAPASCPVRRPAPENLNYPSFVALFPVSSKRVASKTFIRTVSNVGPANSVYRVSVEAPASGVTVKVKPSRLVFSEAVKKRSYAVTVAGDTRNLKMGQSGAVFGSLTWTDGKHVVRSPIVVSQIEPL

SEQ ID NO: 10 - protein, 782 aa, Glycine max
MFMSPITPMEKMVLILASYLLLSTLFSANAEFVKKTYIIQMDKSAKPDTFSNHLDWYSSKVKSILSKSVEAEMDKEERIIYTYQTAFHGVAAKLSQEEAEKLEAEEGVVAIFPDTKYQLHTTRSPTFLGLEPTQSTNNVWSEKLANHDVIVGVLDTGVWPESESFNDTGMRPVPSHWKGACETGRGFRKHHCNNKIVGARMFYHGYEAATGKIDEQAEYKSPRDQDGHGTHTAATVAGSPVHGANLLGYAYGTARGMAPGARIAAYKVCWTGGCFSSDILSAVDRAVDDGVDVLSISLGGGVSSYYRDSLSVASFGAMEKGVFVSCSAGNAGPDPVSLTNVSPWITTVGASTMDRDFPADVSLGNGRKITGTSLYKGRSMLSVKKQYPLVYMGDTNSSIPDPKSLCLEGTLDRRMVSGKIVICDRGISPRVQKGQVVKNAGGVGMILINTAANGEELVADCHLLPAVAIGEKEGKELKHYVLTSKKKATATLGFRATRLGVRPSPVVAAFSSRGPNFLTLEILKPDVVAPGVNILAAWSEAIGPSSLPTDHRRVKFNILSGTSMSCPHVSGIAALLKARHPDWSPAAIKSALMTTAYVHDNTIKPLRDASNAEASTPYDHGAGHINPRRALDPGLVYDIQPQDYIEFLCSLKLTTSELGVFAKYSNRTCRHSLSSPGDLNYPAISVVFPLKNSTSVLTVHRTATNVGLPVSKYHVVVSSFKGASVKVEPDTLSFTRKYQKLSYKVTFTTQSRQTEPEFGGLVWKDGVQKVRSAIVITYLPPI

SEQ ID NO: 11 - protein, 695 aa, Glycine max
MAASSACLVGNGLSTRGNRITLKKDFNGRYLYSPWRLSSLNNKASEAFSIKSSLEQRQQQEGRRGFLKLLLGNVGVGLPALLASGKAYADEQGVSSSRMSYSRFLEYLDKDRVKKVDLYDNGNTAVVEAVSPELGNRLQYVRVQLPGLNQELLQKFREKNIDFAAHSPQEESGSLLANLIGNLAFPLILIGGLFLLSRRSGGMGGPGGPGFPLAFGQSKAKFQMEPNTGVTFDDVAGVDEAKQDFMEVVEFLKKPERFTAVGARIPKGVLLVGPPGTGKTLLAKAIAGEAGVPFFSISGSEFVEMFVGVGASRVRDLFKKAKENAPCIVFVDEIDAVGRQRGTGIGGGNDEREQTLNQLLTEMDGFEGNTGIIVVAATNRADILDSALLRPGRFDRQVTVDVPDIRGRTEILKVHASNKKFDADVSLEVIAMRTPGFSGADLANLLNEAAILAGRRGKTGISSKEIDDSIDRIVAGMEGTVMTDGKSKSLVAYHEVGHAICGTLTPGHDAVQKVTLVPRGQARGLTWFIPNDDPTLISKQQLFARIVGGLGGRAAEEIIFGEPEVTTGAAGDLQQITGLAKQMVTTFGMSDIGPWSLMEASAQSGDVIMRMMARNSMSERLAEDIDAAIKRISDEAYEIALDHIRNNREAIDKIVEVLLEKETLTGDEFRAILSEFAEIPVENRVPPSTPVPATV

SEQ ID NO: 12 - protein, 471 aa, Glycine max
MKQRQIFARVVILLLMFLVGARFAKAKEGGEEAADRILKLPGQPKVSFKQFSGYVTVNKVAGRALFYWLAEAAQNPLTKPLVIWLNGGPGCSSVAYGASEEIGPFRINKTASGLYKNKFSWNSVANLLFLEAPAGVGFSYTNRSSDLLDTGDRRTAQDSLEFVIQWLERFPRYKTRELYITGESYAGHYVPQLAKEIMTYNAKTKHPINLKGIMVGNAVTDNYYDNLGTVTYWWSHAMISDQTFRQLMSRCDFHRQKESDECESVYSYAMDQEFGNIDQYNIYDPPCNNSDGSSSGSGSATRRTMRLPHRPHVAFRHWSGYDPCTEKYAEIYYNRPDVQKALHANKTGIPYRWTACSEVLNRNWNDTDVSVLPIYRELIAHGIRVWVFSGDVDSVVPVTATRYALAQLKLSTKIPWYPWYVKNQVGGWTEVYEGVTFATVRGAGHEVPLFKPRAALQLFTSFLTGKPLPKS

SEQ ID NO: 13 - protein, 736 aa, Glycine max
MISLRLSLLFTLICCLIINQTYSKNDRKTYIVYMGDHPKGMDSASLPSLHITMAQKVLGSDFEPEAILHSYKKSFNGFVIKLTEEEAQRMAEMDNVVSVFPNRKSRLQTTRSWDFIGVSQQIQRTSLERDIIVGVIDSGLWPESKSFSDEGFGPPPSKWKGSCHNFTCNKKIIGAKYFNIEGDYAKEDSISPRDVQGHGSHTASTIAGNLVKSSSLLGFASGTARGGVPSARIAIYKVCWIKIGCPQAETLAAFDEAIADGVDIISISTGLTSIVYIPYFQSAFDIGSFHAMKRGILTSKSADNSGPGLSSITTYSPWILSVAASTIGRKFLTKVQLGNGMVFEGVSINTFDLKNKMFPLVYAGDVPNTADGYNSSTSRFCYVNSVDKHLVKGKIVLCDGNASPKKVGDLSGAAGMLLGATDVKDAPFTYALPTAFISLRNFKLIHSYMVSLRNSTATIFRSDEDNDDSQTPFIVSFSSRGPNPLTPNTLKPDLAAPGVNILAAWSPVYTISEFKGDKRAVQYNIESGTSMACPHVSAAAAYVKSFHPNWSPAMIKSALMTTATPMSPTLNPDAEFAYGAGLINPLKAANPGLVYDISEADYVKFLCGEGYTDEMLRVLTKDHSRCSKHAKKEAVYDLNLPSLALYVNVSSFSRIFHRTVTNVGLATSSYKAKVVSPSLIDIQVKPNVLSFTSIGQKKSFSVIIEGNVNPDILSASLVWDDGTFQVRSPIVVYGDT

SEQ ID NO: 14 - protein, 508 aa, Glycine max
MACHSWLCNISLLLLLLSLASQSCHALNSFGFDIHHRFSDPVKEILGVHDLPDKGTRLYYVVMAHRDRIFRGRRLAAAVHHSPLTFVPANETYQIGAFGFLHFANVSVGTPPLSFLVALDTGSDLFWLPCNCTKCVRGVESNGEKIAFNIYDLKGSSTSQTVLCNSNLCELQRQCPSSDSICPYEVNYLSNGTSTTGFLVEDVLHLITDDDETKDADTRITFGCGQVQTGAFLDGAAPNGLFGLGMGNESVPSILAKEGLTSNSFSMCFGSDGLGRITFGDNSSLVQGKTPFNLRALHPTYNITVTQIIVGGNAADLEFHAIFDSGTSFTHLNDPAYKQITNSFNSAIKLQRYSSSSSDELPFEYCYDLSSNKTVELPINLTMKGGDNYLVTDPIVTISGEGVNLLCLGVLKSNNVNIIGQNFMTGYRIVFDRENMILGWRESNCYVDELSTLAINRSNSPAISPAIAVNPEETSNQSNDPELSPNLSFKIKPTSAFMMALLVLLTIL

SEQ ID NO: 15 - protein, 356 aa, Glycine max
MASTLLPLATFFLVLSASYLQIAGAKAQPLTSLKLNSPILQESIAKEINENPEAGWEAAINPHFSNYTVEQFKRLLGVKPTPKKELRSTPAISHPKSLKLPKNFDARTAWSQCSTIGRILDQGHCGSCWAFGAVESLSDRFCIHFDVNISLSVNDLLACCGFLCGSGCDGGYPLYAWQYLAHHGVVTEECDPYFDQIGCSHPGCEPAYRTPKCVKKCVSGNQVWKKSKHYSVNAYRVSSDPHDIMTEVYKNGPVEVAFTVYEDFAHYKSGVYKHITGYELGGHAVKLIGWGTTEDGEDYWLLANQWNREWGDDGYFKIRRGTNECGIEEDVTAGLPSTKNLVREVTDMDADAAVSF

SEQ ID NO: 16 - protein, 354 aa, Glycine max
MARLITVVWCAVAVLLCAAAGASWVEEAANPIRMVSGVEAEVVRVIGECRRALKFARFVSRFGKSYQSEEEMKERYEIFSQNLRFIRSHNKKRLPYTLSVNHFADWTWEEFKRHRLGAAQNCSATLNGNHKLTDAVLPPTKDWRKEGIVSSVKDQGSCGSCWTFSTTGALEAAYAQAFGKSISLSEQQLVDCAGPFNNFGCHGGLPSQAFEYIKYNGGLETEEAYPYTGKDGVCKFSAENVAVQVLDSVNITLGAEDELKHAVAFVRPVSVAFQVVNGFHFYENGVFTSDTCGSTSQDVNHAVLAVGYGVENGVPYWLIKNSWGESWGENGYFKMELGKNMCGVATCASYPIVA

SEQ ID NO: 17 - protein, 87 aa, unidentified
MAKLVFSLCFLLFSGCCFACPRNCDPRIAYGICPLAEEKKNDRICTNCCAGKKGCKYFSDDGTFVCEGESDPKNPKAPQESQKRAVA

SEQ ID NO: 18 - protein, 105 aa, unidentified
MAKLVFSLCFLLFSGCCFAIDFPLPMASERGQLDKTIVECLKNVNKCWFLSYIKPSEPICGSDQVTYSSDCHLCSKILFEGLNITKLYDGQCENSPQESQKRAVA

SEQ ID NO: 19 - protein, 208 aa, unidentified
MAKLVFSLCFLLFSGCCFAEPVLDTNKQEIRPGYTYYILPATTANGGGLTLAKGENGSCPLDVFQAKNVQSTGLPLKFLMVNSSAGLVIDENEDINIKFAAPRYVSICNKSTVWKIEDGFVSTGGIKGGSENGTATSLFTIQKYEDVYALQYCPRATGCSFICPRLLCGYIGISPAANGSRRLAVNRPVFKIVFKKVGPQESQKRAVA

SEQ ID NO: 20 - protein, 210 aa, unidentified
MAKLVFSLCFLLFSGCCFAQDVPEPVLDVSGNPVRTGVNYFILPAGRGNGGGLQVASIRNRTNPLVVSQHADESSIGGYLQFSPVNPNENIIRISTDLNVKFTSIHISDSSTVWRINTEIIPQRYLVTVGGVEGNPGRETLSNWFSIDRYEDAYKLVYCPGVCETCRPFCGDIGILVEGSKRVLFLRSDKPLKVTFHKIGPQESQKRAVA

SEQ ID NO: 21 - protein, 222 aa, unidentified
MAKLVFSLCFLLFSGCCFAVPNPSRLLAGSSLSPVLDINGDKVQVGLNYFVLPVIRGRGGGLYPSNVKQNNTCPRDIIQEADEVQQGLPVVFTPFDAKKGVVRLSTDLNVRFFTPTICARETIWKLGTYDDKLKQYFIVTGGVEGNPGPQTLSSWFKIEKLGTDYKFVFCPSVCKICKVICKDVGIYSGNDGVRFMALSDTPFRVMFKKTFGPQESQKRAVA

SEQ ID NO: 22 - protein, 63 aa, unidentified
MAKLVFSLCFLLFSGCCFASSPCTVQGKFLLIFHLVLLLILPLVRIAIVTVSRPQESQKRAVA

SEQ ID NO: 23 - protein, 244 aa, unidentified
MAKLVFSLCFLLFSGCCFASSFTSTNPIVLPTTTDDDKGLPIPRFPEVLDINGEPLHVGEEYHIISAIWGVGGGGVYLTFVGNTKCPNGVLQHQRDSLPGMPVKFFTTHPRPLNVVRENTDINIMFSVATTRLCVNETVWKVGDPEPIARLTRFVLTGGTLGNLRPENIDSWFKIERVTKQGPFYKLRYCPATIDSRPVVCSDVGVTHYFGVRRLALTNQPFVVVFRKFQKTDGPQESQKRAVA

SEQ ID NO: 24 - protein, 142 aa, unidentified
MAKLVFSLCFLLFSGCCFARKVGGRTPIKDVKANQRIQDLGKYCVEEYNRSLRKYEQILQEDNIEFLNFAEVVEAETQVVSGIKYYLKISASISSGTPKTFDAVLVIKPWEEKKELIQFSPFSANDKDSSISPQESQKRAVA

SEQ ID NO: 25 - protein, 230 aa, unidentified
MAKLVFSLCFLLFSGCCFASSFTSQNPIELPSASPKPNPVLDTNGNELNPNSSYRIISTFWGALGGDVYLGKSPRSSAPCLDGVFRYNSDVGTVGTPVRFIPLSGGIFEDQLMNLQFNIATVKLCVSYTIWKAGNLNAYYRAMLLETGGSIGQVDSSYFKIVKASTFGYNLLYCPITRPVLCPFCRGDDFCAKVGVINQDGRRRLALVNENPLGVYFKKVPQESQKRAVA

SEQ ID NO: 26 - protein, 140 aa, unidentified
MAKLVFSLCFLLFSGCCFAAHGGRREVLAGSWSPIKNITDPEVVEIGKIAVNEHNKEAKTKLEFEKIMKGESQVVAGTNYRLVIAAKDGGEPHQYMAEVWDKPWEKFRNLTSFKDLRCNEEKGGQNCLFAPQESQKRAVA

SEQ ID NO: 27 - protein, 139 aa, unidentified
MAKLVFSLCFLLFSGCCFAPKPCPRNCDGRIAYGICPLSEEKKNNRLCTNCCAGTKGCNYFSADGTFVCEGESDPRNPKACPRNCDPRIDYRVCPRSEEKKNDRICTNCCSGKKGCKYFSDDGTFVCEGPQESQKRAVA

SEQ ID NO: 28 - protein, 427 aa, unidentified
MAKLVFSLCFLLFSGCCFAMATTDIRLSIAHQTRFAVRLASAISSPSHAKGSSGNAAFSPLSLHVALSLVAAGAAATRDQLAATLGAAEKGDAEGLHALAEQVVQVVLADASGAGGPRSFANVFVDSSLKLKPSFKDLVVGKYKGETQSVDFQTKAPEVAGQVNSWVEKITTGLIKEILPAGSVDSTTRLVLGNALYFKGSWTEKFDASKTKDEKFHLLDGSSVQTPFMSSTKKQYISSYDSLKVLKLPYQQGGDKRQFSMYILLPEAQDGLWNLANKLSTEPEFMEKHMPMQKVPVGQFKLPKFKISFGFEASDMLKGLGLQLPFSSEADLSEMVDSPAARSLYVSSVFHKSFVEVNEEGTEAAARTARVVTLRSLPVEPVKVDFVADHPFLFLIREDLTGVVLFVGHVFNPLVSAPQESQKRAVA

SEQ ID NO: 29 - protein, 59 aa, unidentified
MAKLVFSLCFLLFSGCCFAYKTSISTITIEDNGRCTKSIPPICFPDGRPPQESQKRAVA

SEQ ID NO: 30 - protein, 123 aa, unidentified
MAKLVFSLCFLLFSGCCFANPGGITNVPFPNLPQFKDLARFAVQDYNKKENAHLEFVENLNVKEPVVAGIIYYITLVATDAGKKKIYETKILVKGWENFKEVQEFKLVGDATKPQESQKRAVA

SEQ ID NO: 31 - protein, 141 aa, unidentified
MAKLVFSLCFLLFSGCCFATYHLYNPQNINWDLRTASAYCATWDADKPLEWRQRYGWTAFCGPAGPTGQAACGRCLRVTNTGTGTQATVRIVDQCSNGGLDLDVNVFNQLDTDRRGYQQGHLIVNYEFVNCPQESQKRAVA

SEQ ID NO: 32 - protein, 92 aa, unidentified
MAKLVFSLCFLLFSGCCFAKHLWPELVGMPGKTAKEIIEKENPLVKVQFLFPGMPKPLDLVCGRVIVVVNWEFIVIITPTVGPQESQKRAVA

SEQ ID NO: 33 - protein, 123 aa, unidentified
MAKLVFSLCFLLFSGCCFANPGGITNVPFPNLPQFKDLARFAVQDYNKKENAHLEFVENLNVKEQVVAGIIYYITLVATDAGKKKIYETKILVKGWENFKEVQEFKLVGDATKPQESQKRAVA

SEQ ID NO: 34 - protein, 225 aa, unidentified
MAKLVFSLCFLLFSGCCFAACSCAPAHPQQHICHSALVIRAKISSEKVVPASADPADTEKMLRYEIKQIKMFKGFEKVKDVQYIYTPFDSSLCGVKLEANSQKQYLLTGQVLSDGKVFIHLCNYIEPWEDLSLVQRESLNHHYHLNCGCQITTCYTVPCTISAPNECLWTDWLLERKLYGYQAQHYVCMKHVDGTCSWYRGHLPLRKEFVDIVQPPQESQKRAVA

SEQ ID NO: 35 - protein, 224 aa, unidentified
MAKLVFSLCFLLFSGCCFAACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPPQESQKRAVA

SEQ ID NO: 36 - protein, 223 aa, unidentified
MAKLVFSLCFLLFSGCCFACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDPPQESQKRAVA

SEQ ID NO: 37 - protein, 109 aa, unidentified
MAKLVFSLCFLLFSGCCFAQSATNVRSTYHLYNPQNINWDLRAASAFCTTWDADKPLTWRQKYGWTAFCDAAGPQGQDSCGRCLRVRHLQFFVTISILSPQESQKRAVA

SEQ ID NO: 38 - protein, 116 aa, unidentified
MERVNNYYKLCMELFIMSMMVAMAAAQSATNVRSTYHLYNPQNINWDLRAASAFCTTWDADKPLTWRQKYGWTAFCDAAGPQGQDSCGRCLRVRHLQFFVTISILSPQESQKRAVA

SEQ ID NO: 39 - DNA, 80 bp, synthetic construct
  source 1..80: mol_type = other DNA
tctgagaaggagtctcatgctttgtggagagcaaactcattacggtgtttgcatatttctcaagtaaccgtcataaatca

SEQ ID NO: 40 - DNA, 135 bp, synthetic construct
  source 1..135: mol_type = other DNA
aaagaagaaagaacctaagtgagaaacaaagaaaggaaataattgatctttgaaatctgagaaggagtctcatgctttgtggagagcaaactcattacggtgtttgcatatttctcaagtaaccgtcataaatca

SEQ ID NO: 41 - DNA, 46 bp, synthetic construct
  source 1..46: mol_type = other DNA
gagttcgaactaaacaattcaattcaatctgcctcgatccaccaaa

SEQ ID NO: 42 - DNA, 61 bp, synthetic construct
  source 1..61: mol_type = other DNA
aaacaaagaagaaaagatatactggatagaacaacacgctggaattctagtatactaaacc

SEQ ID NO: 43 - DNA, 136 bp, synthetic construct
  source 1..136: mol_type = other DNA
gacaagggtaaatttccttttctcatttcttctactctgctatccggccatttatataaattccaagtcgcaaatccaaagccatattcgaaccacacaaaattgaactgtaattgtagtagtagacagtagcacc

SEQ ID NO: 44 - DNA, 60 bp, synthetic construct
  source 1..60: mol_type = other DNA
tctcctacagattcttcagcaactgcgattttggatcggaacctcttctgcggcgtagca

SEQ ID NO: 45 - DNA, 41 bp, synthetic construct
  source 1..41: mol_type = other DNA
gcagtggcagtgttcttcatagttcagtgtgtcgtttaaaa

SEQ ID NO: 46 - DNA, 44 bp, synthetic construct
  source 1..44: mol_type = other DNA
attcaaacctttctctatacacacacacacacacactaagaata

SEQ ID NO: 47 - DNA, 48 bp, synthetic construct
  source 1..48: mol_type = other DNA
actcaaacaaaaaacatacacaagtaattaactaacaacaacaccaaa

SEQ ID NO: 48 - DNA, 115 bp, synthetic construct
  source 1..115: mol_type = other DNA
aaagtaagaaagtaaatcaataaagaggaagaaaatctgagaaggagtctcatgctttgtggagagcaaactcattacggtgtttgcatatttctcaagtaaccgtcataaatca

SEQ ID NO: 49 - DNA, 52 bp, synthetic construct
  source 1..52: mol_type = other DNA
tcacgggttcttttctcttcacacttgacaggaaaaacagtaaagcaccaaa

SEQ ID NO: 50 - DNA, 17 bp, synthetic construct
  source 1..17: mol_type = other DNA
atccctcaaacacagtc

SEQ ID NO: 51 - DNA, 138 bp, synthetic construct
  source 1..138: mol_type = other DNA
accatatacaattgtagcaccgatcgaaaagtttccttgacaatctaattctgcaaaatctgagaaggagtctcatgctttgtggagagcaaactcattacggtgtttgcatatttctcaagtaaccgtcataaatca

SEQ ID NO: 52 - DNA, 138 bp, synthetic construct
  source 1..138: mol_type = other DNA
tgtgttgtccgttccagtaacagcattcccacttttattatcacttccggtactggcaggggcaataacaatatacacatctttttgatcaatctctcattcaaaatctcattctctctagtaaacaagaacaaaaaa

SEQ ID NO: 53 - DNA, 17 bp, synthetic construct
  source 1..17: mol_type = other DNA
ctcatcacaacaacaag

SEQ ID NO: 54 - DNA, 82 bp, synthetic construct
  source 1..82: mol_type = other DNA
gtataagagctcatttttacaacaattaccaacaacaacaaacaacaaacaacattacaattacatttacaattatcgatac

SEQ ID NO: 55 - DNA, 54 bp, synthetic construct
  source 1..54: mol_type = other DNA
ctttattcgagttaacttggtgcaggtagcatccctggtgttacatttgatcac

SEQ ID NO: 56 - DNA, 131 bp, synthetic construct
  source 1..131: mol_type = other DNA
acaactcaaacattctctccattggtccttaaacactcatcagtcatcacctctgagaaggagtctcatgctttgtggagagcaaactcattacggtgtttgcatatttctcaagtaaccgtcataaatca

SEQ ID NO: 57 - DNA, 51 bp, synthetic construct
  source 1..51: mol_type = other DNA
attcaaacctttctctatacacacacacacacacactaagaatacaccaaa

SEQ ID NO: 58 - DNA, 161 bp, synthetic construct
  source 1..161: mol_type = other DNA
ctctccatcattttcgtaggaaaaaacattataggagtaagaaaaaagcaaaagttttgtctgtttaaggttttgtatatacccagctctacttggcagcaattacccgtcttgcttgctacttacgagacacgtacattaacacttgtcctagctagtgc

SEQ ID NO: 59 - DNA, 127 bp, synthetic construct
  source 1..127: mol_type = other DNA
atatataagaggtggtgcaaagactttaattaacaaatatatcataaaaaagttccaccaagttaagtgtgaacaatcgtgaagttttttctttgagaagtaaaagatcttagtgcaagtaattaac

SEQ ID NO: 60 - DNA, 13 bp, synthetic construct
  source 1..13: mol_type = other DNA
acctcgagggccc

SEQ ID NO: 61 - DNA, 45 bp, synthetic construct
  source 1..45: mol_type = other DNA
acctcgagtttttatttttaattttctttcaaatacttccatccc

SEQ ID NO: 62 - DNA, 26 bp, synthetic construct
  source 1..26: mol_type = other DNA
agagtctcatattcactctcaatccc

SEQ ID NO: 63 - DNA, 28 bp, synthetic construct
  source 1..28: mol_type = other DNA
acacgctggaattctagtatactaaacc

SEQ ID NO: 64 - DNA, 37 bp, synthetic construct
  source 1..37: mol_type = other DNA
gggggactctagaggatccccgggtggtcagtccctt

SEQ ID NO: 65 - DNA, 139 bp, synthetic construct
  source 1..139: mol_type = other DNA
tgaagattgaccatctcacaaaagctgttacgtgcttgtaacacactacacactcgttttgtattcgagaagtagttgcaacaacggtccccttattgcctgacaagctgagggccacccttctatccccaccgccacc

SEQ ID NO: 66 - DNA, 37 bp, synthetic construct
  source 1..37: mol_type = other DNA
atacctaaccagtgtctcagtgattaagtaatcagct

SEQ ID NO: 67 - DNA, 92 bp, synthetic construct
  source 1..92: mol_type = other DNA
gggtatttatagagatcagtatgaactgtgtcgctaggatcaagcggtggttcacacctgacttcacccctggcgagggcgtgaagtctacc

SEQ ID NO: 68 - DNA, 70 bp, synthetic construct
  source 1..70: mol_type = other DNA
acacgtatttttacaacaataccaacaacaacaacaacaaacaacattacaattacgtatttctctctct

SEQ ID NO: 69 - DNA, 24 bp, synthetic construct
  source 1..24: mol_type = other DNA
atccctcaaacacagtccaccaaa

SEQ ID NO: 70 - DNA, 87 bp, synthetic construct
  source 1..87: mol_type = other DNA
agtaatattttatttgcactactactactactttatttaagcagaagcaggcgagtgaggaagagcgaagagtgaagagacaccaaa

SEQ ID NO: 71 - DNA, 150 bp, synthetic construct
  source 1..150: mol_type = other DNA
acaccaccaacacaaccaaacccaccacgcccaattgttacacacccgcttggaaaagcaagtctaacaatctgagaaggagtctcatgctttgtggagagcaaactcattacggtgtttgcatatttctcaagtaaccgtcataaatca

SEQ ID NO: 72 - DNA, 137 bp, synthetic construct
  source 1..137: mol_type = other DNA
aaatcttcactcactaccaccaaagactgagaagaggggttgaattaactgcttgcaagcacctcgagtatttttacaacaattaccaacaacaacaaacaacaaacaacattacaattactatttacaattacacc

SEQ ID NO: 73 - DNA, 32 bp, synthetic construct
  source 1..32: mol_type = other DNA
agtgcaatttagctgaagcaaagcacaccaaa

SEQ ID NO: 74 - DNA, 52 bp, synthetic construct
  source 1..52: mol_type = other DNA
agagtctcatattcactctcaatcccagagtctcatattcactctcaatccc

SEQ ID NO: 75 - DNA, 131 bp, synthetic construct
  source 1..131: mol_type = other DNA
accgcaaaccaaaaaaagcttcatcatcatcttcttcttctctgcatcatcacaacctcgagtatttttacaacaattaccaacaacaacaaacaacaaacaacattacaattactatttacaattacacc

SEQ ID NO: 76 - DNA, 105 bp, synthetic construct
  source 1..105: mol_type = other DNA
acacttgacaggaaaaacagtaaagtctgagaaggagtctcatgctttgtggagagcaaactcattacggtgtttgcatatttctcaagtaaccgtcataaatca

SEQ ID NO: 77 - DNA, 104 bp, synthetic construct
  source 1..104: mol_type = other DNA
aaaccaaaactcatcatcacaaacgagtaagaatacaaacacaaatagcaaaaaacttgagaccagacccctgctttgcattgaagggaagctcattacaagat

SEQ ID NO: 78 - DNA, 80 bp, synthetic construct
  source 1..80: mol_type = other DNA
ctcgcacgcaaggagatgcatttctggtactagacaagctctattggaataatctctttcttattttttcctttttaccc

SEQ ID NO: 79 - DNA, 98 bp, synthetic construct
  source 1..98: mol_type = other DNA
aattctaatcagtagttaaacttttccctacaccaaaatcatctcttcactaaagtactcagcactcagcagcagatactattcaagagttgaaggaa

SEQ ID NO: 80 - DNA, 87 bp, synthetic construct
  source 1..87: mol_type = other DNA
atcaaataaattccctataaatagcttcacataaagcaagtgagaaaccacaaaaagtaagaaatataagaaataacaaacaccaaa

SEQ ID NO: 81 - DNA, 102 bp, synthetic construct
  source 1..102: mol_type = other DNA
aattctaatcagtagttaaacttttccctacaccaaaatcatctcttcactatgtggaagcagttgcagaccgagctagctagctagcaagcaagcacagag

SEQ ID NO: 82 - DNA, 87 bp, synthetic construct
  source 1..87: mol_type = other DNA
ttcaagataacttactgaggctgtttcagagaggggacagtaaagggggacaggtgttacttattttgaacctcaatttccaccaaa

SEQ ID NO: 83 - DNA, 80 bp, synthetic construct
  source 1..80: mol_type = other DNA
taagtatcaattgaatcaaagactattcaagattccataattgaatcaattacatactggatccaagctaaaggaaagtt

SEQ ID NO: 84 - DNA, 120 bp, synthetic construct
  source 1..120: mol_type = other DNA
ccgttctcttcatccatctctcacaacaaacaaaaagaaatctgagaaggagtctcatgctttgtggagagcaaactcattacggtgtttgcatatttctcaagtaaccgtcataaatca

SEQ ID NO: 85 - DNA, 61 bp, synthetic construct
  source 1..61: mol_type = other DNA
ctttattcgagttaacttggtgcaggtagcatccctggtgttacatttgatcaccaccaaa

SEQ ID NO: 86 - DNA, 126 bp, synthetic construct
  source 1..126: mol_type = other DNA
aagtactcagcactcagcagcagatactattcaagagttgaaggaatctgagaaggagtctcatgctttgtggagagcaaactcattacggtgtttgcatatttctcaagtaaccgtcataaatca

SEQ ID NO: 87 - DNA, 74 bp, synthetic construct
  source 1..74: mol_type = other DNA
gagttcgaactaaacaattcaattcaatctgcctcgatcaaagtaagaaagtaaatcaataaagaggaagaaaa

SEQ ID NO: 88 - DNA, 970 bp, synthetic construct
  source 1..970: mol_type = other DNA
gcccggggatcctctagagtaccctgcaatgtgaccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattattattattattttgatgttcaatgaagttttttttaatttcatatgaagtatacaaaaattcttcatagatttttgtttctatgccgtagttatctttaatatatttgtggttgaagaaatttattgctagaaacgaatggattgtcaatttttttttaaagcaaatatatatgaaattatactgtatattattttagtcatgattaaaatgtggccttaattgaatcatctttctcattcattttttcaaaagcatatcaggatgattgatatttatctattttaaaaattaatttaagggttcaaattaaatttaacttaaaagtgtcctaaccgtagttaaaggtttactttaaaaaaatactatgaaaaatctaatcttctatgaatcgacctgcag

SEQ ID NO: 89 - DNA, 684 bp, synthetic construct
  source 1..684: mol_type = other DNA
cccaattcgccctatagtgagtcgtattacgcgcggagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaagctatc

SEQ ID NO: 90 - DNA, 731 bp, synthetic construct
  source 1..731: mol_type = other DNA
gaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgac

SEQ ID NO: 91 - DNA, 480 bp, synthetic construct
  source 1..480: mol_type = other DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgac

SEQ ID NO: 92 - DNA, 441 bp, synthetic construct
  source 1..441: mol_type = other DNA
actgaggaaatatatagacaaattaagtttggttctatgagttctaatttggacttaagagttgtttgaaattctattttatagtgatgcttataatgtatttggactgttttctgctgtgtgtaagaccttttggtctgtgaactggaaacatacatgaataaatttctttgaatttactggaatttttgcatcaacaaaagaaaaattgaagttactaacttgtaaatggaacaattgtaatgttaaaggatataaatatcttaatatagtgcgatacgaatcacacgaatgcaagactttctctctctgctcccgctcatgctctcggtgcatgttagctaaatatacatcggtgcatccatggcaggagcatgaggacggggatgaggaagggagtgaggagggccaaaagaagtacacatagtttcctttgggagc

SEQ ID NO: 93 - DNA, 1038 bp, synthetic construct
  source 1..1038: mol_type = other DNA
atacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctcatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcat

SEQ ID NO: 94 - DNA, 205 bp, synthetic construct
  source 1..205: mol_type = other DNA
cgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcctag

SEQ ID NO: 95 - DNA, 501 bp, synthetic construct
  source 1..501: mol_type = other DNA
tgacaacatcagcaagaacgccctcctagtatattactgttggatgtcagatactatgtcaaaggcatctacttttgtatcgtttgaccttgattatatcggttgattaatgataattgtaataaaaagctattattgaactttcaattcctcaacaaagaaattattgcaacgatttgggctgataagccttacagttactatttatacactcctggacagtgtttttcactagctcgtttaattgccccatcgacatagtaatgttggattccgctctctgggcccctacaattgaggcagactcccctgggtctaagacgcttgttccaagcctgctgagatgcctatatggatgcattgcgttttccacctctgagtcggcatcggagttgctgagcccaattgtactccgtgaagcccatgattcacccggcttgatctctattgggcctggtagtccaatccttgacatggatgcgcatcttatgggtttccttt

SEQ ID NO: 96 - DNA, 249 bp, synthetic construct
  source 1..249: mol_type = other DNA
atatgaagatgaagatgaaatatttggtgtgtcaaataaaaagcttgtgtgcttaagtttgtgtttttttcttggcttgttgtgttatgaatttgtggctttttctaatatcaaatgaatgtaagatctcattataatgaataaacaaatgtttctataatccattgtgaatgttttgttggatctcttctgcagcatataactactgtatgtgctatggtatggactatggaatatgattaaagataa

SEQ ID NO: 97 - DNA, 663 bp, synthetic construct
  source 1..663: mol_type = other DNA
taataggttgccagtctgatttcactgtcaaccctaaatatggaaaaaagaagaaaataaaaggtgggatcccttctataattctttggaatcctgacgaagactggatgttatcaatgacaagtcaacagaaggattactttgaagataattgcgtcacccactatatgtgtgacggggagactttttttgctcgggaatcgtcgagtcactgaacgtgcctctcctcatacgaggatatatatagtgttattttgtttggggtgtttgttttggattgtgtttttttgtttatttcgtgtgttatgtaacatatgtaatttctatctacttgcacaatgaaatatattcataaaataatcattttattgatttccaactgacttgaagtacactcggaaataaccatttacatatacatcggacttttgggaaggagcaccaacaatgtaaagggctccttcctttatatcaccaacatcacccgttgcactatttttccactctgtggacacacccaatctcttaaagaacttattacaggacttaacctgattacaaggcccaggcccataataagtagctccttgacctttggttgggtcaataccattgctctccaccacgcatgaccacgttttcttcaccacaaaacgatggcaa

SEQ ID NO: 98 - DNA, 451 bp, synthetic construct
  source 1..451: mol_type = other DNA
tgaacgtgcctctcctcatacgaggatatatatagtgttattttgtttggggtgtttgttttggattgtgtttttttgtttatttcgtgtgttatgtaacatatgtaatttctatctacttgcacaatgaaatatattcataaaataatcattttattgatttccaactgacttgaagtacactcggaaataaccatttacatatacatcggacttttgggaaggagcaccaacaatgtaaagggctccttcctttatatcaccaacatcacccgttgcactatttttccactctgtggacacacccaatctcttaaagaacttattacaggacttaacctgattacaaggcccaggcccataataagtagctccttgacctttggttgggtcaataccattgctctccaccacgcatgaccacgttttcttcaccacaaaacgatggcaa

SEQ ID NO: 99 - DNA, 1169 bp, synthetic construct
  source 1..1169: mol_type = other DNA
tcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 100 - DNA, 1199 bp, synthetic construct
  source 1..1199: mol_type = other DNA
aggtaatatttagaaatttaattaacataaccaaggatttttatatcggtaataactctaatatggtatccaaatcagtctagaactctcttacctctaataagtaaaagtacttctaataaattcatatactttttctctcttctccgatctctctttgctcttctttttatgtatcctttcctttctaatagccttttatgagaagtaaacttttagggttggccccccctccccccacaattatatagtttcttactcagttgttggaatataattcaaattcttaaataattgacggtgacattgagttttactttgtggaagagaattagattctcgtgttagtaaaatcggttagtaattgatgatgcattatttttactctataatagagatgcaattttatttttgcattttgggatcaaattgtaatgcagtcatatattgatttcataaatgtttgggatattgttggttatttaactagaaatagacttcttatttcatatttattgttaaaatcctttattggagatgaattatttgttcaacgattagaagttgatagtcgcttttgttttagaagaaattttaccgtagaccaagttaaggagttttagaagcactttgcatgggagcattagtgtatgttatggctttatcaaatataggttttgaagattcagagagccaagaaaagctagaacccaagaactaggaagttagagtaattcacaataccataacgtgatataaaactttttattgtaactcaaatcggtaatattttttgctttagtcttaatcgataaattatttttttatattgattagttataggaggctcaaaaagttgggaataattaaaatatcatattttgtatttgaacaatttatgaaatagtaattggtaaaaaatcactttaaatttttatcctatatccagaaggattatggtgtctggcatagttgtttggaagatttgaatcagggtaaaagtatgttgtaatttttattttgttataggcattttttgtgcttgattgttttgttgtcattatattttattatttggaagtgtatatatatgtttgattaaaatatagataatcaattttataagaaatttgcaacaattacaaaaggataaagtctacaatatgcgagtaaaatttgattgaacctaggatgtccct

SEQ ID NO: 101 - DNA, 869 bp, synthetic construct
  source 1..869: mol_type = other DNA
agtgaagacaacaccactagcacaaatcggatcctgggaaacaggcagaacttcggttcataagctcgggtaggctgtcaacctaccgccgtatcgtattgtgtttggccgatggaggatcttcacgttatcgccgtttgtattcttgccttgactgtgctctctggggtaggcgctgttttgagttgctgccgttggtgctgcagcaatccttttcctccctccctctcttctgtttaagcaaaagactctcgatctgtgcgagagacaatcaaaaatatcgagggagcttcggctcagtgaggggattaacgacccccagtaatggccggtcctggcggacataaataacccgctataggacgaagtggtagccaccactgatcaaatggcaaacatgcttctgtgttgtacactgccccggagcctaccgggtcaacaaggctatcccaccaacccgatgaaatgagggtggagtgagcggagtgggtgacttcgtgatgtacacccgatcgtcaggattgaagacgttaaaactcgacgacctggtacaagtcgttaaactgactcgggtggatacaccacacccggcccagcatgttggcatacccacgatacgaaacgtgggtctcttggagccactacctgtgatgcaaggtagggtatgagtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccctcggggtactcttaacgctgacactcgaaagagcagttcggcaaccc

SEQ ID NO: 102 - DNA, 234 bp, synthetic construct
  source 1..234: mol_type = other DNA
ttgctttcatagatccgtcttcccagagacgttaagaagaagctggagaaaaatattaggttagaagcttgggcgtgacaaacccaagttgcatctcttacgtggttaatcacactgtatgttgacgtacaagccggatcctgggaaacaggtttaacggctcactgtggtggtgggccgtcgatacacttgtatgtgccccaatattggttgtcgagatctctctaggaaccc

SEQ ID NO: 103 - DNA, 869 bp, synthetic construct; identical to SEQ ID NO: 101
  source 1..869: mol_type = other DNA

SEQ ID NO: 104 - DNA, 211 bp, synthetic construct
  source 1..211: mol_type = other DNA
gtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgac

SEQ ID NO: 105 - DNA, 1649 bp, synthetic construct
  source 1..1649: mol_type = other DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 106 - DNA, 1518 bp, synthetic construct
  source 1..1518: mol_type = other DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctcatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcat

SEQ ID NO: 107 - DNA, 921 bp, synthetic construct
  source 1..921: mol_type = other DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacactgaggaaatatatagacaaattaagtttggttctatgagttctaatttggacttaagagttgtttgaaattctattttatagtgatgcttataatgtatttggactgttttctgctgtgtgtaagaccttttggtctgtgaactggaaacatacatgaataaatttctttgaatttactggaatttttgcatcaacaaaagaaaaattgaagttactaacttgtaaatggaacaattgtaatgttaaaggatataaatatcttaatatagtgcgatacgaatcacacgaatgcaagactttctctctctgctcccgctcatgctctcggtgcatgttagctaaatatacatcggtgcatccatggcaggagcatgaggacggggatgaggaagggagtgaggagggccaaaagaagtacacatagtttcctttgggagc

SEQ ID NO: 108 - DNA, 685 bp, synthetic construct
  source 1..685: mol_type = other DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcctag

SEQ ID NO: 109 - DNA, 691 bp, synthetic construct
  source 1..691: mol_type = other DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgac

SEQ ID NO: 110 - DNA, 2418 bp, synthetic construct
  source 1..2418: mol_type = other DNA
gtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctcatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcattcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 111 - DNA, 1860 bp, synthetic construct
  source 1..1860: mol_type = other DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 112 - DNA, 2687 bp, synthetic construct
  source 1..2687: mol_type = other DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctcatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcattcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 113 - DNA, 2648 bp, synthetic construct
  source 1..2648: mol_type = other DNA
actgaggaaatatatagacaaattaagtttggttctatgagttctaatttggacttaagagttgtttgaaattctattttatagtgatgcttataatgtatttggactgttttctgctgtgtgtaagaccttttggtctgtgaactggaaacatacatgaataaatttctttgaatttactggaatttttgcatcaacaaaagaaaaattgaagttactaacttgtaaatggaacaattgtaatgttaaaggatataaatatcttaatatagtgcgatacgaatcacacgaatgcaagactttctctctctgctcccgctcatgctctcggtgcatgttagctaaatatacatcggtgcatccatggcaggagcatgaggacggggatgaggaagggagtgaggagggccaaaagaagtacacatagtttcctttgggagcatacagcattcccagaaagagaaacagaagaaatatacaaactttcattttgagagcagcacctcgtctattgattgcagataatatgcttctcatttgtatttccttttgattatttttgtttctatccctttgtttgagtcaatctcaaatattcggtcattgttggtatgaaaaatcaagcagttcatgttaagagtcaatttaaaattaatatttttatatagagttgtatgtgaaatgatgttgtgatttggtatatatggataaagagcttgtcagttcattttggtctcatttttttggtatccaaataagaaacacaaaagggatatgtccctctactatcaaatattagttataagtattcatgttatactattcgatattttctaccccaatcgttacctatttaaaagtatttacccctccatctatcaaacccctggacccagctttcctattacatgtggcttcatcttaagcccccaaacctttttcttatttttgatttttaaaggctcatcttaaaatttattactcaaattaatacctcttaataacccacctcaaggacccagtaattaaatatccaattagctccagtaattggggttcatattagctccagtcttaaattttaaaggcgatgatcgtattcctccacttggttcatttatactcaaagaatactcaatgtctttagtgtttagataactttttgtaaatcatatagattgttttaacaaaaaacaattcaatagtagattttcacatgaaagttacataaaaattctttaaaattactttctcaaaaaattgttccaaacatattatcccacaattaaactcaatctgtttttcgaaacctaaatcaaaaccaatccaactaccttatataatatataatcaatacattgtaaagaactgcatgttcttttaaattttgggggcaaagttattccgtacgttcacacatgtactaataggaggtaataaatgatatgtgaaacaatcgaggtgtaaacaagctagcattcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 114 - DNA, 1900 bp, synthetic construct
  source 1..1900: mol_type = other DNA
gaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 115 - DNA, 1648 bp, synthetic construct
  source 1..1648: mol_type = other DNA
aaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgactcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 116 - DNA, 2094 bp, synthetic construct
  source 1..2094: mol_type = other DNA
aaagcagaatgctgagctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacactgaggaaatatatagacaaattaagtttggttctatgagttctaatttggacttaagagttgtttgaaattctattttatagtgatgcttataatgtatttggactgttttctgctgtgtgtaagaccttttggtctgtgaactggaaacatacatgaataaatttctttgaatttactggaatttttgcatcaacaaaagaaaaattgaagttactaacttgtaaatggaacaattgtaatgttaaaggatataaatatcttaatatagtgcgatacgaatcacacgaatgcaagactttctctctctgctcccgctcatgctctcggtgcatgttagctaaatatacatcggtgcatccatggcaggagcatgaggacggggatgaggaagggagtgaggagggccaaaagaagtacacatagtttcctttgggagctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 117 - DNA, 1107 bp, synthetic construct
  source 1..1107: mol_type = other DNA
atcaaaacgtttttctttttcttaataaagtatggtcagtttgtaatcacgtccctttacctttaacgtacgtgtaaaatatgtgtctgcggcacctcacttgtaataacactttcttctcataaataaaagggaagtttcgagttacatactataatatagcgccagttttttcgtctattccacaaaacataagtttgtgtccatctactgtagcttgagcttcgtatatatacttggtctttattcttattttattcaaaatacggctttctgctttctgtgggcctaaacggaggcccaaacgaattaaagttccgtcacttggaaacgttatcctagattactcttgcggggaaaagtgtagattagttccaatttttgaagtgaaaaattgtgttggattctatgtcgtaacaagaaatagcatggcctccaaagattattctctctttctttaggctttagcttctaaagctaagctacggaaaaactgtatcattcttctcttttttttggcttatgatccatataagagagcttggaagagctgcttttcatctcctgatcactagctcatacagtttttgtaatttattaacgaaactatataaaaaaagggaagccgaaaaaacaaaacaaaaaacaaccttcatcggctgctcaaatgccaatcatcccaattgatctccattattgttttctcgttactactcctccatctttgatttaataattttttgaaaaaagttttactatatgaatgatattctctgttagggaaatataaaaattgtaagcaaatgattctatcgagctataaaagaaaatataatatgcaaaggaaagatgataatctaaaatattatttacaacaaagaaaactaattgtattcatagtaatgggttttcatataattttaaaatgagaataaatcctaaaaacacgctaaatataacaacatacatcgtcctaaaaacctagaatcacacaaaccctatacccaaatgccacacaacttacatgtttgataatgtgataaatctatagataaaatgtttgtataattcacgaatcaacaaattggcgaaccccttgaattatgt

SEQ ID NO: 118 - protein, 22 aa, synthetic construct
KQMEAESISSSEEIVPNSVQEK

SEQ ID NO: 119 - protein, 43 aa, synthetic construct
RQFYQLDAYPSGAWYYVPLGTQYTDAPSFSDIPNPIGSENSEK

SEQ ID NO: 120 - protein, 25 aa, synthetic construct
RELEELNVPGEIVESLSSSEESITR

SEQ ID NO: 121 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 122 - protein, 22 aa, synthetic construct
KYPVQPFTESQSLTLTDVENLH

SEQ ID NO: 123 - protein, 1060 aa, Homo sapiens
MELWAPQRLPQTRGKATAPSKDPDRGFRRDGHHRPVPHSWHNGERFHQWQDNRGSPQPQQEPRADHQQQPHYASRPGDWHQPVSGVDYYEGGYRNQLYSRPGYENSYQSYQSPTMREEYAYGSYYYHGHPQWLQEERVPRQRSPYIWHEDYREQKYLDEHHYENQHSPFGTNSETHFQSNSRNPCKDSPASNSGQEWPGELFPGSLLAEAQKNKPSLASESNLLQQRESGLSSSSYELSQYIRDAPERDDPPASAAWSPVQADVSSAGPKAPMKFYIPHVPVSFGPGGQLVHVGPSSPTDGQAALVELHSMEVILNDSEEQEEMRSFSGPLIREDVHKVDIMTFCQQKAAQSCKSETLGSRDSALLWQLLVLLCRQNGSMVGSDIAELLMQDCKKLEKYKRQPPVANLINLTDEDWPVLSSGTPNLLTGEIPPSVETPAQIVEKFTRLLYYGRKKEALEWAMKNHLWGHALFLSSKMDPQTYSWVMSGFTSTLALNDPLQTLFQLMSGRIPQAATCCGEKQWGDWRPHLAVILSNQAGDPELYQRAIVAIGDTLAGKGLVEAAHFCYLMAHVPFGHYTVKTDHLVLLGSSHSQEFLKFATTEAIQRTEIFEYCQMLGRPKSFIPSFQVYKLLYASRLADYGLVSQALHYCEAIGAAVLSQGESSHPVLLVELIKLAEKLKLSDPLVLERRSGDRDLEPDWLAQLRRQLEQKVAGDIGDPHPTRSDISGAGGTTTENTFYQDFSGCQGYSEAPGYRSALWLTPEQTCLLQPSPQQPFPLQPGSYPAGGGAGQTGTPRPFYSVPETHLPGTGSSVAVTEATGGTVWEEMLQTHLGPGENTVSQETSQPPDGQEVISKPQTPLAARPRSISESSASSAKEDEKESSDEADKNSPRNTAQRGKLGDGKEHTKSSGFGWFSWFRSKPTKNASPAGDEDSSDSPDSEETPRASSPHQAGLGLSLTPSPESPPLPDVSAFSRGRGGGEGRGSASSGGAAAGAGVGGLSGPESVSFELCSNPGVLLPPPALKGAVPLYNPSQVPQLPTATSLNRPNRLAQRRYPTQPC

SEQ ID NO: 124 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 125 - protein, 541 aa, Homo sapiens
MPGLRRDRLLTLLLLGALLSADLYFHLWPQVQRQLRPRERPRGCPCTGRASSLARDSAAAASDPGTIVHNFSRTEPRTEPAGGSHSGSSSKLQALFAHPLYNVPEEPPLLGAEDSLLASQEALRYYRRKVARWNRRHKMYREQMNLTSLDPPLQLRLEASWVQFHLGINRHGLYSRSSPVVSKLLQDMRHFPTISADYSQDEKALLGACDCTQIVKPSGVHLKLVLRFSDFGKAMFKPMRQQRDEETPVDFFYFIDFQRHNAEIAAFHLDRILDFRRVPPTVGRIVNVTKEILEVTKNEILQSVFFVSPASNVCFFAKCPYMCKTEYAVCGNPHLLEGSLSAFLPSLNLAPRLSVPNPWIRSYTLAGKEEWEVNPLYCDTVKQIYPYNNSQRLLNVIDMAIFDFLIGNMDRHHYEMFTKFGDDGFLIHLDNARGFGRHSHDEISILSPLSQCCMIKKKTLLHLQLLAQADYRLSDVMRESLLEDQLSPVLTEPHLLALDRRLQTILRTVEGCIVAHGQQSVIVDGPVEQLAPDSGQANLTS

SEQ ID NO: 126 - protein, 198 aa, Homo sapiens
MDAILNYRSEDTEDYYTLLGCDELSSVEQILAEFKVRALECHPDKHPENPKAVETFQKLQKAKEILTNEESRARYDHWRRSQMSMPFQQWEALNDSVKTSMHWVVRGKKDLMLEESDKTHTTKMENEECNEQRERKKEELASTAEKTEQKEPKPLEKSVSPQNSDSSGFADVNGWHLRFRWSKDAPSELLRKFRNYEI

SEQ ID NO: 127 - protein, 324 aa, Homo sapiens
MASYYEILDVPRSASADDIKKAYRRKALQWHPDKNPDNKEFAEKKFKEVAEAYEVLSDKHKREIYDRYGREGLTGTGTGPSRAEAGSGGPGFTFTFRSPEEVFREFFGSGDPFAELFDDLGPFSELQNRGSRHSGPFFTFSSSFPGHSDFSSSSFSFSPGAGAFRSVSTSTTFVQGRRITTRRIMENGQERVEVEEDGQLKSVTINGVPDDLALGLELSRREQQPSVTSRSGGTQVQQTPASCPLDSDLSEDEDLQLAMAYSLSEMEAAGKKPAGGREAQHRRQGRPKAQHQDPGLGGTQEGARGEATKRSPSPEEKASRCLIL

SEQ ID NO: 128 - protein, 100 aa, Homo sapiens
MEEASEGGGNDRVRNLQSEVEGVKNIMTQNVERILARGENLEHLRNKTEDLEATSEHFKTTSQKVARKFWWKNVKMIVLICVIVFIIILFIVLFATGAFS

SEQ ID NO: 129 - protein, 116 aa, Homo sapiens
MAGIELERCQQQANEVTEIMRNNFGKVLERGVKLAELQQRSDQLLDMSSTFNKTTQNLAQKKCWENIRYRICVGLVVVGVLLIILIVLLVVFLPQSSDSSSAPRTQDAGIASGPGN

SEQ ID NO: 130 - protein, 388 aa, Homo sapiens
MNTMYVMMAQILRSHLIKATVIPNRVKMLPYFGIIRNRMMSTHKSKKKIREYYRLLNVEEGCSADEVRESFHKLAKQYHPDSGSNTADSATFIRIEKAYRKVLSHVIEQTNASQSKGEEEEDVEKFKYKTPQHRHYLSFEGIGFGTPTQREKHYRQFRADRAAEQVMEYQKQKLQSQYFPDSVIVKNIRQSKQQKITQAIERLVEDLIQESMAKGDFDNLSGKGKPLKKFSDCSYIDPMTHNLNRILIDNGYQPEWILKQKEISDTIEQLREAILVSRKKLGNPMTPTEKKQWNHVCEQFQENIRKLNKRINDFNLIVPILTRQKVHFDAQKEIVRAQKIYETLIKTKEVTDRNPNNLDQGEGEKTPEIKKGFLNWMNLWKFIKIRSF

SEQ ID NO: 131 - protein, 141 aa, Homo sapiens
MPPKFKRHLNDDDVTGSVKSERRNLLEDDSDEEEDFFLRGPSGPRFGPRNDKIKHVQNQVDEVIDVMQENITKVIERGERLDELQDKSESLSDNATAFSNRSKQLRRQMWWRGCKIKAIMALVAAILLLVIIILIVMKYRT

SEQ ID NO: 132 - protein, 744 aa, Homo sapiens
MAGRSMQAARCPTDELSLTNCAVVNEKDFQSGQHVIVRTSPNHRYTFTLKTHPSVVPGSIAFSLPQRKWAGLSIGQEIEVSLYTFDKAKQCIGTMTIEIDFLQKKSIDSNPYDTDKMAAEFIQQFNNQAFSVGQQLVFSFNEKLFGLLVKDIEAMDPSILKGEPATGKRQKIEVGLVVGNSQVAFEKAENSSLNLIGKAKTKENRQSIINPDWNFEKMGIGGLDKEFSDIFRRAFASRVFPPEIVEQMGCKHVKGILLYGPPGCGKTLLARQIGKMLNAREPKVVNGPEILNKYVGESEANIRKLFADAEEEQRRLGANSGLHIIIFDEIDAICKQRGSMAGSTGVHDTVVNQLLSKIDGVEQLNNILVIGMTNRPDLIDEALLRPGRLEVKMEIGLPDEKGRLQILHIHTARMRGHQLLSADVDIKELAVETKNFSGAELEGLVRAAQSTAMNRHIKASTKVEVDMEKAESLQVTRGDFLASLENDIKPAFGTNQEDYASYIMNGIIKWGDPVTRVLDDGELLVQQTKNSDRTPLVSVLLEGPPHSGKTALAAKIAEESNFPFIKICSPDKMIGFSETAKCQAMKKIFDDAYKSQLSCVVVDDIERLLDYVPIGPRFSNLVLQALLVLLKKAPPQGRKLLIIGTTSRKDVLQEMEMLNAFSTTIHVPNIATGEQLLEALELLGNFKDKERTTIAQQVKGKKVWIGIKKLLMLIEMSLQMDPEYRVRKFLALLREEGASPLDFD

SEQ ID NO: 133 - protein, 198 aa, Homo sapiens
MSFIFDWIYSGFSSVLQFLGLYKKTGKLVFLGLDNAGKTTLLHMLKDDRLGQHVPTLHPTSEELTIAGMTFTTFDLGGHVQARRVWKNYLPAINGIVFLVDCADHERLLESKEELDSLMTDETIANVPILILGNKIDRPEAISEERLREMFGLYGQTTGKGSISLKELNARPLEVFMCSVLKRQGYGEGFRWMAQYID

SEQ ID NO: 134 - protein, 304 aa, Homo sapiens
MAVTKELLQMDLYALLGIEEKAADKEVKKAYRQKALSCHPDKNPDNPRAAELFHQLSQALEVLTDAAARAAYDKVRKAKKQAAERTQKLDEKRKKVKLDLEARERQAQAQESEEEEESRSTRTLEQEIERLREEGSRQLEEQQRLIREQIRQERDQRLRGKAENTEGQGTPKLKLKWKCKKEDESKGGYSKDVLLRLLQKYGEVLNLVLSSKKPGTAVVEFATVKAAELAVQNEVGLVDNPLKISWLEGQPQDAVGRSHSGLSKGSVLSERDYESLVMMRMRQAAERQQLIARMQQEDQEGPPT

SEQ ID NO: 135 - protein, 223 aa, Homo sapiens
MATPQSIFIFAICILMITELILASKSYYDILGVPKSASERQIKKAFHKLAMKYHPDKNKSPDAEAKFREIAEAYETLSDANRRKEYDTLGHSAFTSGKGQRGSGSSFEQSFNFNFDDLFKDFGFFGQNQNTGSKKRFENHFQTRQDGGSSRQRHHFQEFSFGGGLFDDMFEDMEKMFSFSGFDSTNQHTVQTENRFHGSSKHCRTVTQRRGNMVTTYTDCSGQ

SEQ ID NO: 136 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 137 - protein, 192 aa, Homo sapiens
MVRAGAVGAHLPASGLDIFGDLKKMNKRQLYYQVLNFAMIVSSALMIWKGLIVLTGSESPIVVVLSGSMEPAFHRGDLLFLTNFREDPIRAGEIVVFKVEGRDIPIVHRVIKVHEKDNGDIKFLTKGDNNEVDDRGLYKEGQNWLEKKDVVGRARGFLPYVGMVTIIMNDYPKFKYALLAVMGAYVLLKRES

SEQ ID NO: 138 - protein, 232 aa, Homo sapiens
MASSAASSEHFEKLHEIFRGLHEDLQGVPERLLGTAGTEEKKKLIRDFDEKQQEANETLAEMEEELRYAPLSFRNPMMSKLRNYRKDLAKLHREVRSTPLTATPGGRGDMKYGIYAVENEHMNRLQSQRAMLLQGTESLNRATQSIERSHRIATETDQIGSEIIEELGEQRDQLERTKSRLVNTSENLSKSRKILRSMSRKVTTNKLLLSIIILLELAILGGLVYYKFFRSH

SEQ ID NO: 139 - protein, 375 aa, Homo sapiens
MESNKDEAERCISIALKAIQSNQPDRALRFLEKAQRLYPTPRVRALIESLNQKPQTAGDQPPPTDTTHATHRKAGGTDAPSANGEAGGESTKGYTAEQVAAVKRVKQCKDYYEILGVSRGASDEDLKKAYRRLALKFHPDKNHAPGATEAFKAIGTAYAVLSNPEKRKQYDQFGDDKSQAARHGHGHGDFHRGFEADISPEDLFNMFFGGGFPSSNVHVYSNGRMRYTYQQRQDRRDNQGDGGLGVFVQLMPILILILVSALSQLMVSSPPYSLSPRPSVGHIHRRVTDHLGVVYYVGDTFSEEYTGSSLKTVERNVEDDYIANLRNNCWKEKQQKEGLLYRARYFGDTDMYHRAQKMGTPSCSRLSEVQASLHG

SEQ ID NO: 140 - protein, 504 aa, Homo sapiens
MVAPGSVTSRLGSVFPFLLVLVDLQYEGAECGVNADVEKHLELGKKLLAAGQLADALSQFHAAVDGDPDNYIAYYRRATVFLAMGKSKAALPDLTKVIQLKMDFTAARLQRGHLLLKQGKLDEAEDDFKKVLKSNPSENEEKEAQSQLIKSDEMQRLRSQALNAFGSGDYTAAIAFLDKILEVCVWDAELRELRAECFIKEGEPRKAISDLKAASKLKNDNTEAFYKISTLYYQLGDHELSLSEVRECLKLDQDHKRCFAHYKQVKKLNKLIESAEELIRDGRYTDATSKYESVMKTEPSIAEYTVRSKERICHCFSKDEKPVEAIRVCSEVLQMEPDNVNALKDRAEAYLIEEMYDEAIQDYETAQEHNENDQQIREGLEKAQRLLKQSQKRDYYKILGVKRNAKKQEIIKAYRKLALQWHPDNFQNEEEKKKAEKKFIDIAAAKEVLSDPEMRKKFDDGEDPLDAESQQGGGGNPFHRSWNSWQGFNPFSSGGPFRFKFHFN

SEQ ID NO: 141 - protein, 215 aa, Homo sapiens
MVLLTMIARVADGLPLAASMQEDEQSGRDLQQYQSQAKQLFRKLNEQSPTRCTLEAGAMTFHYIIEQGVCYLVLCEAAFPKKLAFAYLEDLHSEFDEQHGKKVPTVSRPYSFIEFDTFIQKTKKLYIDSRARRNLGSINTELQDVQRIMVANIEEVLQRGEALSALDSKANNLSSLSKKYRQDAKYLNMRSTYAKLAAVAVFFIMLIVYVRFWWL

SEQ ID NO: 142 - protein, 200 aa, synthetic construct
MRPKHPIKHQGLPQEVLNENLLRFFVAPFPEVFGKEKVNELDKDIGDEDTEDQAMEDIKQMEAEDIDDDEEIVPNDVEQKHIQKEDVPSERYLGYLEQLLRLKKYKVPQLEIVPNDAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 143 - protein, 210 aa, synthetic construct
MRELEELNVPGEIVEDLDDDEESITRINKKIEKFQDEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 144 - protein, 170 aa, synthetic construct
MQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIADGEPTSTPTTEAVESTVADLEDDPEVIESPPEINTVQVTDTAV

SEQ ID NO: 145 - DNA, 600 bp, synthetic construct
  source 1..600: mol_type = other DNA
atgcgtccgaagcacccaatcaagcatcaagggcttccccaagaggtactgaacgaaaatctactcagattcttcgtggctccatttcctgaggtatttgggaaggaaaaggtgaatgagctggataaagacataggcgatgaggacactgaggaccaagctatggaggatataaagcaaatggaggccgaagatatagacgatgacgaggaaatcgttccaaatgatgtcgaacagaagcatattcaaaaggaggatgtgccttctgagcgttaccttggatatcttgaacagttgctgagactcaagaaatacaaggtcccccaacttgaaattgttcctaacgacgccgaagagaggttacactctatgaaagaggggattcatgctcaacagaaggagcctatgatcggtgtgaatcaagaactggcatacttctatcccgaactgtttcgtcaattttatcaattagatgcctatccatccggggcttggtattatgtgcctcttgggacacagtatactgatgcaccaagtttttcagacatcccaaacccaataggcagtgagaattcagaaaagaccacaatgcctctttgg

SEQ ID NO: 146 - DNA, 630 bp, synthetic construct
  source 1..630: mol_type = other DNA
atgagagaacttgaagaattgaatgttccaggagagattgttgaagatctcgacgacgatgaggaaagcatcacaagaatcaacaagaaaattgagaagtttcaggatgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccaaaataccctgtggagcctttcacagaatctcaaagcttgactctcactgatgttgagaatcttcatttgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtt

SEQ ID NO: 147 - DNA, 510 bp, synthetic construct
  source 1..510: mol_type = other DNA
atgcaggagcaaaaccaagagcaacctattcgttgtgaaaaagacgagagattttttagtgataaaattgcaaagtacatcccaatccagtacgttttaagtagatatcccagctacggtcttaattactatcaacagaagccagttgccttgataaacaaccagtttctcccatatccctactatgctaagcctgccgccgtccgttctcctgctcagatattgcagtggcaggttttatccaacactgtcccagccaagtcttgccaggcacaacccaccaccatggctagacacccacatccacacctttcctttatggctattcctcccaaaaagaaccaagacaagacagagatccccaccataaatactatcgctgacggtgagccaacttcaacacctactactgaagctgtggagagcactgtcgccgatcttgaagatgatccagaggtgattgaatcaccccccgagataaacacagtacaggtcaccgacactgctgta

SEQ ID NO: 148 - protein, 200 aa, synthetic construct
MRPKHPIKHQGLPQEVLNENLLRFFVAPFPEVFGKEKVNELAKDIGAEATEDQAMEDIKQMEAEAIAAAEEIVPNAVEQKHIQKEDVPSERYLGYLEQLLRLKKYKVPQLEIVPNAAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 149 - protein, 210 aa, synthetic construct
MRELEELNVPGEIVEALAAAEESITRINKKIEKFQAEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 150 - protein, 170 aa, synthetic construct
MQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIAAGEPTSTPTTEAVESTVAALEDAPEVIESPPEINTVQVTATAV

SEQ ID NO: 151 - DNA, 600 bp, synthetic construct
  source 1..600: mol_type = other DNA
atgcgtccgaagcacccaatcaagcatcaagggcttccccaagaggtactgaacgaaaatctactcagattcttcgtggctccatttcctgaggtatttgggaaggaaaaggtgaatgagctggctaaagacataggcgctgaagctactgaggaccaagctatggaggatataaagcaaatggaggccgaagctatagctgctgctgaggaaatcgttccaaatgctgtcgaacagaagcatattcaaaaggaggatgtgccttctgagcgttaccttggatatcttgaacagttgctgagactcaagaaatacaaggtcccccaacttgaaattgttcctaacgctgccgaagagaggttacactctatgaaagaggggattcatgctcaacagaaggagcctatgatcggtgtgaatcaagaactggcatacttctatcccgaactgtttcgtcaattttatcaattagatgcctatccatccggggcttggtattatgtgcctcttgggacacagtatactgatgcaccaagtttttcagacatcccaaacccaataggcagtgagaattcagaaaagaccacaatgcctctttgg

SEQ ID NO: 152 - DNA, 630 bp, synthetic construct
  source 1..630: mol_type = other DNA
atgagagaacttgaagaattgaatgttccaggagagattgttgaagctctcgctgctgctgaggaaagcatcacaagaatcaacaagaaaattgagaagtttcaggctgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccaaaataccctgtggagcctttcacagaatctcaaagcttgactctcactgatgttgagaatcttcatttgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtt

SEQ ID NO: 153 - DNA, 510 bp, synthetic construct
  source 1..510: mol_type = other DNA
atgcaggagcaaaaccaagagcaacctattcgttgtgaaaaagacgagagattttttagtgataaaattgcaaagtacatcccaatccagtacgttttaagtagatatcccagctacggtcttaattactatcaacagaagccagttgccttgataaacaaccagtttctcccatatccctactatgctaagcctgccgccgtccgttctcctgctcagatattgcagtggcaggttttatccaacactgtcccagccaagtcttgccaggcacaacccaccaccatggctagacacccacatccacacctttcctttatggctattcctcccaaaaagaaccaagacaagacagagatccccaccataaatactatcgctgctggtgagccaacttcaacacctactactgaagctgtggagagcactgtcgccgctcttgaagatgctccagaggtgattgaatcaccccccgagataaacacagtacaggtcaccgctactgctgta

SEQ ID NO: 154 - DNA, 27 bp, synthetic construct
  source 1..27: mol_type = other DNA
ccaatgagacctcgttggtctcgggtt

SEQ ID NO: 155 - DNA, 211 bp, synthetic construct
  source 1..211: mol_type = other DNA
ccaattggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagttgggaaattgggtt

SEQ ID NO: 156 - DNA, 135 bp, synthetic construct; identical to SEQ ID NO: 40
  source 1..135: mol_type = other DNA

SEQ ID NO: 157 - DNA, 46 bp, synthetic construct; identical to SEQ ID NO: 41
  source 1..46: mol_type = other DNA

SEQ ID NO: 158 - DNA, 80 bp, synthetic construct
  source 1..80: mol_type = other DNA
tcattcattatcactacgattgttctaaaagcaattattactcttgagaagaattaaaagatcatcatcacaataacaag

SEQ ID NO: 159 - DNA, 61 bp, synthetic construct; identical to SEQ ID NO: 42
  source 1..61: mol_type = other DNA

SEQ ID NO: 160 - DNA, 100 bp, synthetic construct
  source 1..100: mol_type = other DNA
tgtggaagcagttgcagaccgagctagctagctagcaagcaagcacagagtgtggaagcagttgcagaccgagctagctagctagcaagcaagcacagag

SEQ ID NO: 161 - DNA, 48 bp, synthetic construct; identical to SEQ ID NO: 47
  source 1..48: mol_type = other DNA

SEQ ID NO: 162 - DNA, 115 bp, synthetic construct; identical to SEQ ID NO: 48
  source 1..115: mol_type = other DNA

SEQ ID NO: 163 - DNA, 52 bp, synthetic construct; identical to SEQ ID NO: 49
  source 1..52: mol_type = other DNA

SEQ ID NO: 164 - DNA, 89 bp, synthetic construct
  source 1..89: mol_type = other DNA
gccgtcgggggggggtctgtccccgcatagctgaacatctatataataccggttatccagcgacaaccgtcaataacttaacattgaac

SEQ ID NO: 165 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 166 - protein, 584 aa, Homo sapiens
MKMMLVRRFRVLILMVFLVACALHIALDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 167 - DNA, 3495 bp, synthetic construct
  source 1..3495: mol_type = other DNA
atggaccccattcggtcaaggacgccatctccggctcgggaacttctacctggaccacaacccgatggggttcagcctacggccgataggggggtatcaccccccgcgggcggccccttggatggacttcccgcacgtcgaacgatgagtcgtacacgactaccgagcccgcctgcaccttctccagcattttctgctggctcttttagcgacctcctacgccagttcgacccatcattattcaatacatcccttttcgattctctcccccctttcggtgcacatcatacagaggctgcaactggcgaatgggatgaggtccaatctggtctgagggccgctgacgcaccccctccaaccatgagagttgctgtgactgctgcacgtcctccccgtgcgaaaccagcacctagacgacgtgcggcacagccttcggacgctagccctgctgcccaggttgatctcaggacactcggctacagtcaacagcaacaggagaaaataaaaccgaaagtccgatcaacggttgctcaacaccatgaggctctcgtgggtcatggctttacacatgctcacatcgttgcgctatcgcaacatcccgctgcacttggaaccgtcgcagttaaatatcaagatatgattgcagcactacctgaggctacacatgaagcaatcgtgggcgttggtaagcagtggtccggagctcgggcgttggaagcccttctcactgtagccggcgagttacgcggtccccctcttcagttggacactggtcaattgctcaagatcgcgaagcgtggaggagtgaccgcggtagaggcagtacacgcttggagaaatgccctgacaggcgctccattaaatctcacgccagaacaagtggtcgctatcgcctcccatgatggtggaaaacaagcactagaaactgtccaaagattattgcctgttctttgtcaggcacacggacttaccccacaacaagtcgttgctatagcctcccatgatggagggaagcaagcgttagaaacagtgcagcggctactccctgtattatgccaggctcatggtctaactccacaacaagtggtggctatagcctcacacgatggtggtaaacaggcacttgaaaccgtccaaagactcctgccggtcctctgccaggcacacggcctcacccccgaacaagtggtggctattgcttcgcatgatggaggtaagcaggctttagagacagtccagagactactacccgttctatgccaggcccatggtttgaccccggaacaggttgttgctattgcgtcaaataatggcggcaagcaagcgttggaaaccgttcaagcattactccctgttctctgtcaagcacatgggctaacgcccgagcaggttgttgcaattgcatcacatgatggaggaaagcaggccttagaaacggtacaggcacttttaccagtcctttgccaagcacacgggcttacacccgaacaagtggtcgctattgcaagtaatataggtggaaaacaagcactggaaaccgtgcaggcgcttttgccggtattatgccaagctcacggcctaactcctgaacaggtggttgcgattgcctcaaatggtgggggtaaacaggcactggagactgtgcagcggcttttgcctgttttgtgtcaagctcatggattgacaccagagcaggtggtcgctatagctagtaacattggaggtaaacaagcgcttgaaaccgtgcaacgtctgctgccagttctatgtcaagctcatgggttgaccccacaacaggttgtagcgatcgcttccaataacggaggaaagcaagctctagaaacggtgcagaggctcctcccggttctttgtcaggcgcatggattgaccccggagcaggtggtcgcaatcgccagtcatgatggaggtaagcaggccttggaaaccgttcaggcgttactcccggttctatgccaggcgcatggcctgacccctgaacaggttgtggcgatagccagtaacggcgggggaaagcaggcacttgaaaccgtacaacgactcctcccagtcctttgtcaagcccacggattgactccagaacaagtagttgctatagcttcgaataatggaggaaagcaggcccttgaaacagttcagcgtcttttgccagtgttgtgtcaagcacacggattgactcctgaacaggttgtcgccattgcatctaatatcggtggtaagcaagctctcgaaaccgtacagcgactcttgcctgttctatgccaagcgcatggcttgacgccggaacaggtggtagccatagcaagcaacataggtggcaaacaagctcttgaaacagttcaaaggttgttacctgtgctttgccaagcccacggtttgacccctcaacaggtggttgctatagcatcacatgatgggggacggcctgctcttgagacagtgcagcgcctgttgcccgtgttgtgtcaagcgcatggcttaacaccggaacaggtcgtggcaattgcgtcaaatattggcggcaaacaagcgctggaaaccgttcagcgactcttgcctgttctgtgccaagctcacggtctgacgccccaacaggttgttgccattgcttcaaatggaggagggaggccagcccttgagtcgattgtcgcacagctatctcggcccgaccctgctttagccgctctgacaaatgatcatcttgtggctctcgcctgcttaggaggtcgcccagctttagacgcagtaaaaaagggtctacctcatgctccggccttaatcaagaggacgaatcgtagaatcccagaacgaacgagccatcgcgtagccgatcacgctcaagttgttagggttttaggtttttttcagtgtcattcacatccggcacaagctttcgatgatgccatgacccagtttggtatgtcaaggcatggattactgcaacttttcagaagagtaggagtgacagagctcgaagccagaagcggaactctgccacccgctagccaaagatgggataggatattgcaggcgagtggaatgaagcgcgcgaaaccatctccaacaagcactcaaaccccggatcaagcgagtttgcacgctttcgcagattctctcgaacgagatttggatgccccttctccaatgcacgaaggtgatcaaactagggcgagtagcaggaagaggtctaggagtgatcgtgcagttacgggcccctcagcacaacagtcttttgaggtcagggtgccagaacaaagggacgctttacatctcccattgtcttggcgtgtaaaaaggccgcgaactagtattggagggggattaccggacccagggacccccactgctgctgatctagctgcttctagtacggtaatgcgcgagcaagacgaggatccatttgctggggcagctgatgacttccccgcattcaacgaagaagaattagcatggttgatggagttactgccacagtaa

SEQ ID NO: 168 - protein, 1164 aa, synthetic construct
MDPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASHDGGKQALETVQRLLPVLCQAHGLTPQQVVAIASHDGGKQALETVQRLLPVLCQAHGLTPEQVVAIASHDGGKQALETVQRLLPVLCQAHGLTPEQVVAIASNNGGKQALETVQALLPVLCQAHGLTPEQVVAIASHDGGKQALETVQALLPVLCQAHGLTPEQVVAIASNIGGKQALETVQALLPVLCQAHGLTPEQVVAIASNGGGKQALETVQRLLPVLCQAHGLTPEQVVAIASNIGGKQALETVQRLLPVLCQAHGLTPQQVVAIASNNGGKQALETVQRLLPVLCQAHGLTPEQVVAIASHDGGKQALETVQALLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPQQVVAIASHDGGRPALETVQRLLPVLCQAHGLTPEQVVAIASNIGGKQALETVQRLLPVLCQAHGLTPQQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASSRKRSRSDRAVTGPSAQQSFEVRVPEQRDALHLPLSWRVKRPRTSIGGGLPDPGTPTAADLAASSTVMREQDEDPFAGAADDFPAFNEEELAWLMELLPQ

SEQ ID NO: 169 - protein, 15 aa, synthetic construct
EFELNNSIQSASIHQ

SEQ ID NO: 170 - protein, 17 aa, synthetic construct
SRVLFSSHLTGKTVKHQ

SEQ ID NO: 171 - protein, 20 aa, synthetic construct
KQRRKDILDRTTRWNSSILN

SEQ ID NO: 172 - DNA, 1168 bp, synthetic construct
  source 1..1168: mol_type = other DNA
tcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagt

SEQ ID NO: 173 - DNA, 33 bp, synthetic construct
  source 1..33: mol_type = other DNA
acccccgcctcccaaaaagaaccaagacaagac

SEQ ID NO: 174 - DNA, 26 bp, synthetic construct
  source 1..26: mol_type = other DNA
gggggcggaaggtgcggatgcgggtg

SEQ ID NO: 175 - DNA, 25 bp, synthetic construct
  source 1..25: mol_type = other DNA
acccccgcaagacaagacagagatc

SEQ ID NO: 176 - DNA, 27 bp, synthetic construct
  source 1..27: mol_type = other DNA
gggggcggaatagccataaaggaaagg

SEQ ID NO: 177 - DNA, 28 bp, synthetic construct
  source 1..28: mol_type = other DNA
acccccgatcccgaccataaatactatc

SEQ ID NO: 178 - DNA, 28 bp, synthetic construct
  source 1..28: mol_type = other DNA
gggggcgggttctttttgggaggaatag

SEQ ID NO: 179 - DNA, 25 bp, synthetic construct
  source 1..25: mol_type = other DNA
acccccgactatcgcttctggtgag

SEQ ID NO: 180 - DNA, 27 bp, synthetic construct
  source 1..27: mol_type = other DNA
gggggcggctctgtcttgtcttggttc

SEQ ID NO: 181 - DNA, 25 bp, synthetic construct
  source 1..25: mol_type = other DNA
acccccggagccaacttcaacaccg

SEQ ID NO: 182 - DNA, 28 bp, synthetic construct
  source 1..28: mol_type = other DNA
gggggcggatttatggtcgggatctctg

SEQ ID NO: 183 - DNA, 25 bp, synthetic construct
  source 1..25: mol_type = other DNA
acccccgccgactactgaagcggtg

SEQ ID NO: 184 - DNA, 31 bp, synthetic construct
  source 1..31: mol_type = other DNA
gggggcggaccagaagcgatagtatttatgg

SEQ ID NO: 185 - DNA, 25 bp, synthetic construct
  source 1..25: mol_type = other DNA
acccccggtggagagcacggtcgcc

SEQ ID NO: 186 - DNA, 31 bp, synthetic construct
  source 1..31: mol_type = other DNA
gggggcggtgttgaagttggctcaccagaag

SEQ ID NO: 187 - DNA, 25 bp, synthetic construct
  source 1..25: mol_type = other DNA
acccccggccacacttgaagattcc

SEQ ID NO: 188 - DNA, 26 bp, synthetic construct
  source 1..26: mol_type = other DNA
gggggcggcgcttcagtagtcggtgt

SEQ ID NO: 189 - DNA, 30 bp, synthetic construct
  source 1..30: mol_type = other DNA
acccccgtccccagaggtgattgaatcacc

SEQ ID NO: 190 - DNA, 26 bp, synthetic construct
  source 1..26: mol_type = other DNA
gggggcgggaccgtgctctccaccgc

SEQ ID NO: 191 - DNA, 25 bp, synthetic construct
  source 1..25: mol_type = other DNA
acccccggaatcacccccggagata

SEQ ID NO: 192 - DNA, 26 bp, synthetic construct
  source 1..26: mol_type = other DNA
gggggcggatcttcaagtgtggcgac

SEQ ID NO: 193 - DNA, 25 bp, synthetic construct
  source 1..25: mol_type = other DNA
acccccgataaacacggtacaggtc

SEQ ID NO: 194 - DNA, 26 bp, synthetic construct
  source 1..26: mol_type = other DNA
gggggcggaatcacctctggggaatc

SEQ ID NO: 195 - DNA, 25 bp, synthetic construct
  source 1..25: mol_type = other DNA
acccccggtcacctcaacggcttcg

SEQ ID NO: 196 - DNA, 27 bp, synthetic construct
  source 1..27: mol_type = other DNA
gggggcggctccgggggtgattcaatc

SEQ ID NO: 197 - DNA, 25 bp, synthetic construct
  source 1..25: mol_type = other DNA
acccccgcaggagcaaaaccaagag

SEQ ID NO: 198 - DNA, 26 bp, synthetic construct
  source 1..26: mol_type = other DNA
gggggcggctgtaccgtgtttatctc

SEQ ID NO: 199 - DNA, 513 bp, synthetic construct
  source 1..513: mol_type = other DNA
atgaaacttctcatccttacctgtcttgtggctgttgctcttgcccgtccgaagcacccaatcaagcatcaagggcttccccaagaggtactgaacgaaaatctactcagattcttcgtggctccatttcctgaggtatttgggaaggaaaaggtgaatgagctgtctaaagacataggctctgaatcaactgaggaccaagctatggaggatataaagcaaatggaggccgaatccatatcctcaagcgaggaaatcgttccaaattccgtcgaacagaagcatattcaaaaggaggatgtgccttctgagcgttaccttggatatcttgaacagttgctgagactcaagaaatacaaggtcccccaacttgaaattgttcctaactcagccgaagagaggttacactctatgaaagaggggattcatgctcaacagaaggagcctatgatcggtgtgaatcaagaactggcatacttctatcccgaactgtttaccacaatgcctctttgg

SEQ ID NO: 200 - protein, 171 aa, synthetic construct
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFTTMPLW

SEQ ID NO: 201 - DNA, 579 bp, synthetic construct
  source 1..579: mol_type = other DNA
atgaaacttctcatccttacctgtcttgtggctgttgctcttgcccgtccgaagcacccaatcaagcatcaagggcttccccaagaggtactgaacgaaaatctactcagattcttcgtggctccatttcctgaggtatttgggaaggaaaaggtgaatgagctgtctaaagacataggctctgaatcaactgaggaccaagctatggaggatatacatattcaaaaggaggatgtgccttctgagcgttaccttggatatcttgaacagttgctgagactcaagaaatacaaggtcccccaacttgaaattgttcctaactcagccgaagagaggttacactctatgaaagaggggattcatgctcaacagaaggagcctatgatcggtgtgaatcaagaactggcatacttctatcccgaactgtttcgtcaattttatcaattagatgcctatccatccggggcttggtattatgtgcctcttgggacacagtatactgatgcaccaagtttttcagacatcccaaacccaataggcagtgagaattcagaaaagaccacaatgcctctttggtaa

SEQ ID NO: 202 - protein, 192 aa, synthetic construct
MKLLILTCLVAVALARPKHPIKHQGLPQEVLNENLLRFFVAPFPEVFGKEKVNELSKDIGSESTEDQAMEDIHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 203 - DNA, 450 bp, synthetic construct
  source 1..450: mol_type = other DNA
atgaaacttctcatccttacctgtcttgtggctgttgctcttgcccgtccgaagcacccaatcaagcatcaagggcttccccaagaggtactgaacgaaaatctactcagattcttcgtggctccatttcctgaggtatttgggaaggaaaaggtgaatgagctgtctaaagacataggctctgaatcaactgaggaccaagctatggaggatatacatattcaaaaggaggatgtgccttctgagcgttaccttggatatcttgaacagttgctgagactcaagaaatacaaggtcccccaacttgaaattgttcctaactcagccgaagagaggttacactctatgaaagaggggattcatgctcaacagaaggagcctatgatcggtgtgaatcaagaactggcatacttctatcccgaactgtttaccacaatgcctctttggtaa

SEQ ID NO: 204 - protein, 149 aa, synthetic construct
MKLLILTCLVAVALARPKHPIKHQGLPQEVLNENLLRFFVAPFPEVFGKEKVNELSKDIGSESTEDQAMEDIHIQKEDVPSERYLGYLEQLLRLKKYKVPQLEIVPNSAEERLHSMKEGIHAQQKEPMIGVNQELAYFYPELFTTMPLW

SEQ ID NO: 205 - DNA, 645 bp, Bos taurus
  source 1..645: mol_type = other DNA
atgaaacttctcatccttacctgtcttgtggctgttgctcttgcccgtccgaagcacccaatcaagcatcaagggcttccccaagaggtactgaacgaaaatctactcagattcttcgtggctccatttcctgaggtatttgggaaggaaaaggtgaatgagctgtctaaagacataggctctgaatcaactgaggaccaagctatggaggatataaagcaaatggaggccgaatccatatcctcaagcgaggaaatcgttccaaattccgtcgaacagaagcatattcaaaaggaggatgtgccttctgagcgttaccttggatatcttgaacagttgctgagactcaagaaatacaaggtcccccaacttgaaattgttcctaactcagccgaagagaggttacactctatgaaagaggggattcatgctcaacagaaggagcctatgatcggtgtgaatcaagaactggcatacttctatcccgaactgtttcgtcaattttatcaattagatgcctatccatccggggcttggtattatgtgcctcttgggacacagtatactgatgcaccaagtttttcagacatcccaaacccaataggcagtgagaattcagaaaagaccacaatgcctctttggtaa

SEQ ID NO: 206 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 207 - DNA, 600 bp, synthetic construct
  source 1..600: mol_type = other DNA
atgaaggtcctcatccttgcctgcctggtggctctggcccttgcaatcaacaagaaaattgagaagtttcagagtgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccaaaataccctgtggagcctttcacagaatctcaaagcttgactctcactgatgttgagaatcttcatttgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtttaa

SEQ ID NO: 208 - protein, 199 aa, synthetic construct
MKVLILACLVALALAINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 209 - DNA, 609 bp, synthetic construct
  source 1..609: mol_type = other DNA
atgaaggtcctcatccttgcctgcctggtggctctggcccttgcaagagaacttgaagaattgaatgttccaggagagattgttgaaagtctctcctcttctgaggaaagcatcacaagaatcaacaagaaaattgagaagtttcagagtgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccattgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtttaa

SEQ ID NO: 210 - protein, 202 aa, synthetic construct
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPLPLPLLQSWMHQPHQPLPPTVMFPPQSVLSLSQSKVLPVPQKAVPYPQRDMPIQAFLLYQEPVLGPVRGPFPIIV

SEQ ID NO: 211 - DNA, 534 bp, synthetic construct
  source 1..534: mol_type = other DNA
atgaaggtcctcatccttgcctgcctggtggctctggcccttgcaatcaacaagaaaattgagaagtttcagagtgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccattgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtttaa

SEQ ID NO: 212 - protein, 177 aa, synthetic construct
MKVLILACLVALALAINKKIEKFQSEEQQQTEDELQDKIHPFAQTQSLVYPFPGPIPNSLPQNIPPLTQTPVVVPPFLQPEVMGVSKVKEAMAPKHKEMPFPLPLPLLQSWMHQPHQPLPPTVMFPPQSVLSLSQSKVLPVPQKAVPYPQRDMPIQAFLLYQEPVLGPVRGPFPIIV

SEQ ID NO: 213 - DNA, 675 bp, Bos taurus
  source 1..675: mol_type = other DNA
atgaaggtcctcatccttgcctgcctggtggctctggcccttgcaagagaacttgaagaattgaatgttccaggagagattgttgaaagtctctcctcttctgaggaaagcatcacaagaatcaacaagaaaattgagaagtttcagagtgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccaaaataccctgtggagcctttcacagaatctcaaagcttgactctcactgatgttgagaatcttcatttgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtttaa

SEQ ID NO: 214 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 215 - DNA, 573 bp, synthetic construct
  source 1..573: mol_type = other DNA
atgatgaagagttttttcctagttgtgactatcctagcattaaccctgccatttttgggtgcccaggagcaaaaccaagagcaacctattcgttgtgaaaaagacgagagattttttagtgataaaattgcaaagtacatcccaatccagtacgttttaagtagatatcccagctacggtcttaattactatcaacagaagccagttgccttgataaacaaccagtttctcccatatccctactatgctaagcctgccgccgtccgttctcctgctcagatattgcagtggcaggttttatccaacactgtcccagccaagtcttgccaggcacaacccaccaccatggctagacacccacatccacacctttcctttatggctattccgcccccacccccgcaagacaagacagagatccccaccataaatactatcgcttctggtgagccaacttcaacacctactactgaagctgtggagagcactgtcgccacacttgaagattccccagaggtgattgaatcaccccccgagataaacacagtacaggtcacctcaactgctgtataa

SEQ ID NO: 216 - protein, 190 aa, synthetic construct
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPPPPQDKTEIPTINTIASGEPTSTPTTEAVESTVATLEDSPEVIESPPEINTVQVTSTAV

SEQ ID NO: 217 - protein, 237 aa, Glycine max
MFLTRTEYDRGVNTFSPEGRLFQVEYAIEAIKLGSTAIGLKTKEGVVLAVEKRITSPLLEPSSVEKIMEIDEHIGCAMSGLIADARTLVEHARVETQNHRFSYGEPMTVESTTQALCDLALRFGEGDEESMSRPFGVSLLIAGHDENGPSLYYTDPSGTFWQCNAKAIGSGSEGADSSLQEQFNKDLTLQEAETIALSILKQVMEEKVTPNNVDIAKVAPTYHLYTPSEVEAVISRL

SEQ ID NO: 218 - protein, 405 aa, Glycine max
MVLEATMICIDNSEWMRNGDYSPSRFQAQTDAVNLICGAKTQSNPENTVGVLTMAGKGVRVLVTPTSDLGKILACMHGLEIGGEMNLAAGIQVAQLALKHRQNKKQQQRIIVFAGGPVKHEKKMLEMIGRKLKKNSVALDIVNFGEEDEGKTEKLEALLSAVNNNDTSHIVHVPSGPNALSDVLISTPIFTGDGEGGSGFAAAAAAAAAGGVSGFEFGVDPNLDPELALALRVSMEEERARQEAAAKKASEDASKQEKDGEQQASPQDTTMTEGASAAASEAETKRTDLTDDENALLQQALAMSMDDPTINHEVRDTDMSEAAAEDPELALALQLSVEDSSKDSASQSDVSKLLADQSFVSSILASLPGVDPNDPSVKDLLASMQNQSEPQQKNEDKPPNEEEKK

SEQ ID NO: 219 - protein, 881 aa, Glycine max
MENSDNDEPEKKRPHLTFVSSRTPRNSINTPATNKTADPGVLQFQNQQLVQQIDIQKHALHDLEEKIRELKGKQSSYDDLLIALNQLWTQLVDDMILLGIRAGRGKGKDTLQHLTDIDNPQGSLPLCPAEDIFLCRLIQKDSIKGISDDEIISCVKEALALRQSSTRELLKLLKDTIDYQMERAGSIAQVLHEDLSSEDAKIQMSKIDDMTKEEADNFREVVDTLHAKHKEYTVAIQNSINECSQDQSEIKRLAGELDEIVAELEESRRKLVNLEMQKDAAIGMNSPNADAVNGNLSPENIADRTMGLRELKDSIEEAKIVEADRLSELQDAQEDNQTLTKQFQDLQNELKDDKYVCTSRIYSLANDQLQHWMTELTRYKTSVESLQAGNVHVAKWENELNLKLESADSARQILGNSDHRIDDLELQLQKCIIEKNDLEIKMEEAKQDTGRKDIKSEFHVMASALSKEMGMMEGQLKRWKDAAHEAVSLREKTHSLREALSMKTSELKSLTNKCAEQVLEIKSLKTLTEKLQKENQELEFILDMHGQENYDKRHSEVRESERKAHSQAEMLKNALDEHSLELRVKAANEAEAACEQRLSAAEAEIEDLRSKLDASERDILELTEAVKVKDAEAEAYISEIETIGQAYEDMQTQNQNLLDQVIERDDYNIKLVSDSVKTKQAHNTLMSQKQALAKQLQQINTSIEHSKTRITHSEEQTKAILSDAIKCNQEEKHLAVTLEFAKWELGDTEKELKLLKSAVSSSEKEYDQIPKDTEAIQMELESERSLRKKLEEELGELNSKIDELTSETGETTIQKLEKEIRICKNMIKCTVCTDRPKEVVIVKCYHLFCNPCIQRNLELRHRKCPACGTAFGQSDVRFVKI

SEQ ID NO: 220 - protein, 735 aa, Glycine max
MAQAAGRSRVVGDYVVGKQIGAGSFSVVWHGRHKVHGTEVAIKEIATLRLNKKLQESLMSEIFILKRINHPNIISLHDIINQVHGKIHLVLEYCKGGDLSLYIQRHGKVPEATAKHFMLQLAAGLQVLRDNNLIHRDLKPQNLLLSRNDEKSVLKIADFGFARSLQPRGLAETLCGSPLYMAPEIMQLQKYDAKADLWSVGAILFQLVTGRTPFTGNNQIQLLQNIMKSTELQFPSDSQSLSFECKDLCQKLLRRNPVERLTFEEFFNHPFLSQKQTEQDEPLRNRSSPRLVGGFCSTGSDPLRRTEENYQEDCLPFMLDDDSSGPEGSPSFSRKKSSMKSTYGFDLNAKLDKAESSSPISNNINHTSGFGSVTQRSENTTKRLDNHKISRNLTNPLESPEQLFTSPYPKAAVTDSLENIDQEYVLVSGPPIDVSSSSVGASRPSHTPYRSGSLPQESSSTITRLSVPMPIVGVPSNSICQIGSSGSQDSAPGTSLGSMDTGDEQPSAHCMTRVKSLQQCASSITELVNEKMEAGKHLEAFSIQLVILAIWKQALHICHTQAASAMEGSPNQETSRYRRSTSRKHGSPDSEECLDGNTLGPKDILSQIESEFLREFEHAEELAKTIEPGNTEMPDAMETIFQSALAFGRHGGVEELMGEMESAAALYSKAVRLLVFLLVEGPSLILNPPFSLTNSDRYRLRNYIDILNNRQGYSRSQRMTLLKCDDSRGILKEKF

SEQ ID NO: 221 - protein, 508 aa, Glycine max
MGQKHLVTVLCLWALTCSLLPSFSFGILRIGLKKRPLDIDSINAARKAREGLRSGRSMMGAHDQYIGKSKGEDLVPLKNYMDAQYFGEIGIGTPPQPFTVVFDTGSSNLWVPSSKCYFTLACYTHNWYTAKKSKTHAKNGTSCKISYGTGSISGFFSQDNVKVGSAVVKHQDFIEATHEGSLTFLSAKFDGILGLGFQEISVENSVPVWYKMVEQKLISEKVFSFWLNGDPNAKKGGELVFGGVDPKHFKGNHTYVPITEKGYWQIEIGDFFIGGVSTGVCEGGCAAIVDSGTSLLAGPTPVVAEINHAIGAEGVLSVECKEVVSQYGELIWDLLVSGVKPDDICSQVGLCSSKRHESKSAGIEMVTEKEQGELTARDNPLCSSCQMLVLWIQNQLKQKATKDRVFNYVNQLCESLPSPSGESVISCNSLSKMPNITFTIGNKPFVLTPEQYILKTGEGITEVCLSGFIAFDVPPPKGPLWILGDVFMRAYHTVFDYGNLQVGFAEAV

SEQ ID NO: 222 - protein, 440 aa, Glycine max
MEAKLATTIILIFSVIWLMRVNGIDPCASQADNSDLNVIPIYSKCSPFKPPKSDSSWDNRIINMASKDPLRFKYLSTLVGQKTVSTAPIASGQTFNIGNYVVRVKLGTPGQLLFMVLDTSTDEAFVPCSGCTGCSDTTFSPKASTSYGPLDCSVPQCGQVRGLSCPATGTGACSFNQSYAGSSFSATLVQDSLRLATDVIPNYSFGCVNAITGASVPAQGLLGLGRGPLSLLSQSGSNYSGIFSYCLPSFKSYYFSGSLKLGPVGQPKSIRTTPLLRSPHRPSLYYVNFTGISVGRVLVPFPSEYLGFNPNTGSGTIIDSGTVITRFVEPVYNAVREEFRKQVGGTTFTSIGAFDTCFVKTYETLAPPITLHFEGLDLKLPLENSLIHSSAGSLACLAMAAAPDNVNSVLNVIANFQQQNLRILFDTVNNKVGIAREVCN

SEQ ID NO: 223 - protein, 200 aa, Glycine max
MGKKKLEIKRIENKSNRQITFSKRRKGLMKKARELSILCDAKLALLIFSSTGKLYELCNGDSLAEVVQRYWDNLGASGTDTKGLRFEIADIWSDEAFSQLVQSHFGVSELEHLSVTDLMELEKLVHSALSRIRSAKMRLMMESVENLKKKEHILRNENELLEKQVKMYTKSCTESYMSILSFYVVNILFFYSILELSINK

SEQ ID NO: 224 - protein, 341 aa, Glycine max
MSSFSQNPYVLVLFLVLTVWISRVMSRGLITSERHEKWMAQYGKVYKDAAEKEKRFQVFKNNVQFIESFNAAGDKPFNLSINQFADLHDEEFKALLNNVQKKASRVETATETSFRYENVTKIPSTMDWRKRGAVTPIKDQGYTCGSCWAFATVATVESLHQITTGELVSLSEQELVDCVRGDSEGCRGGYVENAFEFIANKGGITSEAYYPYKGKDRSCKVKKETHGVARIIGYESVPSNSEKALLKAVANQPVSVYIDAGAIAFKFYSSGIFEARNCGTHLDHAVAVVGYGKLRDGTKYWLVKNSWSTAWGEKGYMRIKRDIRAKKGLCGIASNASYPIA

SEQ ID NO: 225 - protein, 664 aa, Glycine max
MTMMMELGGPRVIGDYIVGPRIGSGSFAVVWRARNRSSGLEYAVKEIDKRHLSPKVRENLLKEISILSTIHHPNIIRLFEAIQTNDRIYLVLEYCAGGDLAAYIHRHGKVSEPVAHHFMRQLAAGLQVLQEKNLIHRDLKPQNLLLATTAATPVMKIGDFGFARSLTPQGLADTLCGSPYYMAPEIIENQKYDAKADLWSVGAILYQLVIGRPPFDGNSQLQLFQNILASTELHFPPDALKVLHSDCLDLCRNLLRRNPDERLTFKAFFNHNFLREPRPTMNVEQFQLHQSERLTNHQLGGSTSEKISESHSKYNPMVVSSAADETMLLQRKDGKITAGTTNAKVSHLMESIEKDYVFVNSHFASLEAFSDYFEASVQDSSSHRISLFPSKRTNMEVRDAKQTKDLPSSSTEGLENLKSNKLEACAASCEFAALRKEHQISPLHPSNRLQLLHQYVQIIAELSQEKYNTGLYLESLAVELVVLAIWKQTLEICSSWMASITKSELPGSSSANESISARDINLPQSTEQKINFSDPSSISLWAKHEFIDAVDRAEKLSCHVQNMDKATEMPDAMEIIFQKALLIGTSGAVDEYMEIRDRAAASYSKAMLLLSFIVAEAENLPLNPPFSLVATNKERISQYIHSLQSRKESSSESSSKKVLILLSK

SEQ ID NO: 226 - protein, 515 aa, Glycine max
MVSLTPLCFFFTVLIAGGSVIRCAAGADAAAEPETVRFDTGGLSRETFPKGFLFGTATSAYQVEGMAHKDGRGPSIWDLFIKKPGIVANNGTGEVSVDQYHRYKEDIDLMASLNFDAYRFSISWSRIFPNGTGQVNWKGVAYYNRLINYLLEKGITPYANLYHYDLPLALEERYNGLLSRQVVNDFADYAEFCFKTFGDRVKNWMTFNEPRVVAALGYDNGFFAPGRCSKEYGNCTAGNSGTEPYIVAHNLILSHAAAVQRYREKYQEKQKGRIGILLDFVWYEPLTRSKADNLAAQRARDFHVGWFIHPLVYGEYPTTIQNIVGNRLPKFTSEEVKIVKGSIDFVGINQYTTYYMYDPHQAKPKVPGYQMDWNAGFAYAKNGVPIGPRAYSYWLYNVPWGMYKSLMYIKERYGNPTVFLSENGMDDPGNVTLPKGLHDTTRINYYKGYLTQLKKAVDDGANVVGYFAWSLLDNFEWRLGYTSRFGIVYVDFKTLKRYPKMSAYWFKQLIAKKKY

SEQ ID NO: 227 - protein, 379 aa, Glycine max
MGFLVLLLFSLLGLSSSSSISTHRSILDLDLTKFTTQKQVSSLFQLWKSEHGRVYHNHEEEAKRLEIFKNNLNYIRDMNANRKSPHSHRLGLNKFADITPQEFSKKYLQAPKDVSQQIKMANKKMKKEQYSCDHPPASWDWRKKGVITQVKYQGGCGSGWAFSATGAIEAAHAIATGDLVSLSEQELVDCVEESEGCYNGWHYQSFEWVLEHGGIATDDDYPYRAKEGRCKANKIQDKVTIDGYETLIMSDESTESETEQAFLSAILEQPISVSIDAKDFHLYTGGIYDGENCTSPYGINHFVLLVGYGSADGVDYWIAKNSWGEDWGEDGYIWIQRNTGNLLGVCGMNYFASYPTKEESETLVSARVKGHRRVDHSPL

SEQ ID NO: 228 - protein, 506 aa, Glycine max
MVSLTPLCFFITLLIAGADAAAEPQTVRFDTGGLSRDTFPKGFLFGTATSAYQVEGMAHKDGRGPSIWDVFIKKPGIVANNGTGEVSVDQYHRYKEDIDLMASLNFDAYRFSISWSRIFPNGTGQVNWKGVAYYNRLINYLLEKGITPYANLYHYDLPLALEERYNGLLSRQVVKDFADYAEFCFKTFGDRVKNWMTFNEPRVVAALGYDNGFFAPGRCSKEYGNCTAGNSGTEPYIVAHNLILSHAAAVQRYRAKYQEKQKGRIGILLDFVWYEPLTRSKADNFAAQRARDFHIGWFIHPLVYGEYPKTIQNIVGNRLPKFTSEEVKIVKGSIDFVGINQYTTFFIYDPHQSKPKVPGYQMDWNAGFAYAKNGVPIGPRANSYWLYNVPWGMYKSLMYIKERYGNPTVILSENGMDDPGNVTLPKGLHDTTRINYYKGYLTQLKKAVDDGANVVGYFAWSLLDNFEWRLGYTSRFGIVYVDFKTLKRYPKMSAYWFKQLITKKKY

SEQ ID NO: 229 - protein, 594 aa, Glycine max
MPEPAPSSSNSHSVEDNVNVEATGTPQNRHNKDGAGASEINTVDGSDRVASSSKAEEGDGDDNDDYIQARVDKLLCKIEEPELSEEQIRINDQSQQDEILVVESIYGENVFSLERWKGLRCFQIHINVDVLGEIAITANLNSINQLETLSSNSDEFLYTFKVQYLPPIVLTCLLPKSYPSDQPPIFTLSVKWLEPVKILNLCYKLDSIWEEQQGQEVIYPWVEWLHSSSLSHLGFDEEIILGPYGMNYVQDERVISGAECIDVDIPFLRSYNDERHNENFLKELHDCNICFSEYAGSQFIRLPCEHFFCLKCLQTFAQIHVKEGTVSNLKCPEAKCAIMIPPGLLKQLLDDTDYERWESMMLEKTLASMSDVVYCPRCETPCIEDEDQHAQCPKCYFSFCTLCRERRHVGIACMSLDMKLQILQDRQNLSQLKEDQKRREREKINEMLNMKEIHRDSKLCPSCDMAISRTEGCNKMKCGNCEQYFCYRCNKAIDASDPYGHFRDGSCELFPREMVDSWEERINHRQAVGQLQAELFPQHGLACPSCRQYNPKIGNNNHLFCWACQRHYCYLCKAIVRRGTKHYGPKGCKQHSEG

SEQ ID NO: 230 - protein, 1373 aa, Glycine max
MALGDLMASRFSQSTVLVVPTSHNHLDDSTTASSSSSAAAAVAALNNNSSSNDDADFAHRRDSEAAIAIISSGNYAGNAATSMAYLPHTVVLCELRHDAFEAAVPAGPSDSGLVSKWRPKDRMKTGCVALVLCLNISVDPPDVIKISPCARMECWIDPFSMAPQKALESIGKTLSSQYERWQPKARYKCQLDPTVDEVKKLCTTCRKYAKSERVLFHYNGHGVPKPTANGEIWVFNKSYTQYIPLPINELDSWLKTPSIYVFDCSAAGMIVNSFIELHEWSASNSSVSQRDCILLAACEAHETLPQSAEFPADVFTSCLTTPIKMALRWFCTRSLLRESLDYSLIDKIPGRPNDRKTLLGELNWIFTAVTDTIAWNVLPHDLFQRLFRQDLLVASLFRNFLLAERIMRSANCSPVSHPMLPPTHQHHMWDAWDMAAELCLSQLPSLVEDPNAEFQPSTFFTEQLTAFEVWLDHGSEHKKPPEQLPIVLQVLLSQCHRFRALVLLGRFLDMGPWAVDLALSVGIFPYVLKLLQTTTPELRQILVFIWTKILALDKSCQVDLVKDGGHIYFIKFLDSMEAYPEQRAMAAFVLAVIVDGHRRGQEACIEAGLIHVCLKHLQSSCPNDSQTEPLFLQWLCLCLGKLWEDFSEAQTIGLQEDATTIFAPLLSEPQPEVRASAVFALGTLLDVGFDSCRSVGGDEECDDDDKFRAEVSIVKSMLDVASDGSPLVRAEVAVALARFAFGHNKHLKSIAAAYWKPQANSLINSLPSLTNIKGSVGGYAKQNQHMPHGSIVSPQIGPIRVGNDNSPVVRDGRVSSSSPLAGSGIMHGSPLSDDSSHHSDSGILNDGFSNGVANHTGPKPFDNALYSQCVLAMCTLAKDPSPRIANLGRRVLSIIGIEQVVAKPLKSSGVRTAESTASPLARSSSWFDMNGGHLPLTFRTPPVSPPRPSYITRMRRVCSLEFRPHLMDSPDSGLADPLLGSGGASGTSDRSFLPQSTIYSWSCGHFSKPLLTAADDSEEVSARREEREKFALEHIAKCQHSAVSRLTNPIAKWDIKGTQTALLQPFSPIVIAADENERIRIWNHEEATLLNSFDNHDFPDKGISKLCLVNELDESLLLAASSDGNIRIWKDYTLRGKQKLVTAFSSIHGHKPGVRNLNAVVDWQQQCGYLYASGEISSIMLWDVDKEQLVNSKSSSSDCSVSALAASQVHGGQFTAGFIDGSVRLYDVRTPDMLVCGLRPHTQRVEKVVGIGFQPGLDQGKIVSASQAGDIQFLDIRNHSSAYLTIEAHRGSLTALAVHRHAPIIASGSAKQLIKVFSLEGDQLGTIRYYPTLMAQKIGSVSCLNFHPYQVLLAAGAADACVCIYADDNTQAR

SEQ ID NO: 231 - protein, 627 aa, Glycine max
MGRYSIPFMGVLLLFCLVSSSEAEYLKYKDPKAPLNVRISDLLKRMSLEEKIGQMTQIERSVATPDVMNKYFIGSVLSGGGSVPATKASAASWQQMVNQMQKAALSTRLGIPMIYGIDAVHGHNNVYNATVFPHNVGLGVTRDPVLIKKIGEATALEVRATGIPYVFAPCIAVCRDPRWGRCYESYSEDPKIVKTMTEIIPGLQGDIPGNSIKGVPFVAGKNKVAACAKHYLGDGGTNKGINENNTLISYNGLLSIHMPAYYDSIIKGVSTVMISYSSWNGMKMHANKKLITGYLKNKLHFKGFVISDWQGIDRITSPPHANYSYSVQAGVSAGIDMIMVPFNYTEFIDELTRQVKNNIIPISRIDDAVARILRVKFVMGLFENPYADPSLANQLGSKEHREIAREAVRKSLVLLKNGKSYKKPLLPLPKKSTKILVAGSHANNLGYQCGGWTITWQGLGGNDLTSGTTILDAVKQTVDPATEVVFNENPDKNFVKSYKFDYAIVVVGEHTYAETFGDSLNLTMADPGPSTITNVCGAIRCVVVLVTGRPVVIKPYLPKIDALVAAWLPGTEGQGVADVLYGDYEFTGKLARTWFKTVDQLPMNVGDKHYDPLFPFGYGLTTNITKY

SEQ ID NO: 232 - protein, 380 aa, Glycine max
MEAKRGHALMCLARVSLFLCALTLSAAHGSTTVQDIARKLKLGDNELLRTEKKFKVFMENYGRSYSTEEEYLRRLGIFAQNMVRAAEHQALDPTAVHGVTQFSDLTEDEFEKLYTGVNGGFPSSNNAAGGIAPPLEVDGLPENFDWREKGAVTEVKLQGRCGSCWAFSTTGSIEGANFLATGKLVSLSEQQLLDCDNKCDITEKTSCDNGCNGGLMTNAYNYLLESGGLEEESSYPYTGERGECKFDPEKIAVKITNFTNIPADENQIAAYLVKNGPLAMGVNAIFMQTYIGGVSCPLICSKKRLNHGVLLVGYGAKGFSILRLGNKPYWIIKNSWGEKWGEDGYYKLCRGHGMCGINTMVSAAMVPQPQTTPTKNYASY

SEQ ID NO: 233 - protein, 773 aa, Glycine max
MGKQVLNSFRPPVVFLLFFIVFSVVSCDEASKTFIFRVDSQSKPTIFPTHYHWYTSEFAQETSILHVYDTVFHGFSAVLTHQQVASISQHPSVLAVFEDRRRQLHTTRSPQFLGLRNQRGLWSESDYGSDVIIGVFDTGVWPERRSFSDLNLGPIPRRWKGACETGVRFSPKNCNRKLIGARFFSKGHEAGAGSGPLNPINDTVEFRSPRDADGHGTHTASTAAGRYAFQASMSGYAAGIAKGVAPKARLAAYKVCWKNSGCFDSDILAAFDAAVNDGVDVISISIGGGDGIASPYYLDPIAIGSYGAVSRGVFVSSSAGNDGPSGMSVTNLAPWLTTVGAGTIDRDFPSQVILGDGRRLSGVSLYAGAALKGKMYQLVYPGKSGILGDSLCMENSLDPNMVKGKIVICDRGSSPRVAKGLVVKKAGGVGMILANGISNGEGLVGDAHLLPACAVGANEGDVIKKYISSSTNPTATLDFKGTILGIKPAPVIASFSARGPNGLNPQILKPDFIAPGVNILAAWTQAVGPTGLDSDTRRTEFNILSGTSMACPHVSGAAALLKSAHPDWSPAALRSAMMTTATVLDNRNQIMTDEATGNSSTPYDFGAGHLNLGRAMDPGLVYDITNNDYVNFLCGIGYGPKVIQVITRAPASCPVRRPAPENLNYPSFVAMFPASSKGVASKTFIRTVTNVGPANSVYRVSVEAPASGVSVTVKPSRLVFSEAVKKRSYVVTVAGDTRKLKMGPSGAVFGSLTWTDGKHVVRSPIVVTQIEPL

SEQ ID NO: 234 - protein, 476 aa, Glycine max
MEEKLKYPFLSSFLTLFFCISATSIAPHNSQTQTLLLRTLPNPPTLSWPESTISGSDPEPDPFSLSVHLNHIDALSPNKTPSQLFHLRLLRDGARVKTLNSLAAATNQTRPTNTGSGFSSSVVSGLSQGSGEYFTRLGVGTPPKYLYIVLDTGSDVVWLQCKPCTKCYSQTDQIFDPSKSKTFAGIPCSSPLCRRLDSPGCNTKNNLCQYQVSYGDGSFTVGDFSIETLTFRRAEVPRVALGCGHDNEGLFVGAAGLLGLGRGGLSFPTQTGTRFNNKFSYCLTDRTASAKPSSVVFGDSAVSRTARFTPLVKNPKLDTFYYVELLGFSVGGAPVRGISASLFRLDSTGNGGVIIDSGTSVTRLTRPGYVALRDAFRVGASHLKRASEFSLFDTCYDLSGLSEVKVPTVVLHFRGADVSLPASNYLIPVDNDGTFCFAFAGTMSGLSIVGNIQQQGFRVVFDLAGSRVGFAPRGCA

SEQ ID NO: 235 - protein, 760 aa, Glycine max
MGPFRKPFLAFLSVVLFLGLYEAAAEQTQTHKSTYIVHVAKSEMPESFEHHAVWYESSLKTVSDSAEMIYTYDNAIHGYATRLTAEEARLLQRQTGILAVLPETRYELFTTRTPLFLGLDKSADLFPESSSGSDVIVGVLDTGVWPESKSFDDTGLGPVPSTWKGACETGTNFTASNCNRKLIGARFFAKGVEAMLGPINETEESRSARDDDGHGTHTSSTAAGSVVSGASLLGYASGTARGMATRARVAAYKVCWKGGCFSSDILAAIERAILDNVNVLSLSLGGGISDYYRDSVAIGAFSAMEKGILVSCSAGNSGPGPYSLSNVAPWITTVGAGTLDRDFPAYVALGNGLNFSGVSLYRGNALPDSSLPLVYAGNVSNGAMNGNLCITGTLSPEKVAGKIVLCDRGLTARVQKGSVVKSAGALGMVLSNTAANGEELVADAHLLPATAVGQKAGDAIKKYLVSDAKPTVKIFFEGTKVGIQPSPVVAAFSSRGPNSITPQILKPDLIAPGVNILAGWSKAVGPTGLPVDNRRVDFNIISGTSMSCPHVSGLAALIKSAHPDWSPAAVRSALMTTAYTVYKTGEKLQDSATGKPSTPFDHGSGHVDPVAALNPGLVYDLTVDDYLGFLCALNYSAAEISTLAKRKFQCDAGKQYSVTDLNYPSFAVLFESSGSVVKHTRTLTNVGPAGTYKASVTSDTASVKISVEPQVLSFKENEKKTFTVTFSSSGSPQHTENAFGRVEWSDGKHLVGSPISVNWG

SEQ ID NO: 236 - protein, 426 aa, Glycine max
MATLLLPTLLLLIPFATLVSARPHLAGDFLRLPSETDNDDNVQGTRWAVLLAGSNGYWNYRHQADVCHAYQILRKGGLKEENIIVFMYDDIAFNGENPRPGVIINKPDGGDVYEGVPKDYTGEDVTVGNFFAALLGNKSALTGGSGKVVDSGPDDHIFVYYTDHGGPGVLGMPAGPYLYADDLIEVLKKKHASGTYKNLVFYLEACESGSIFEGLLPEDINIYATTASNAEESSWGTYCPGEYPSPPPEYSTCLGDLYSVAWMEDSDRHNLRTETLHQQYKLVKERTISGDSYYGSHVMQYGDVRLSSDVLFHYLGTDPANDNFTFVDENSLWSPSKPVNQRDADLIHFWDKFRKAPEGSLRKNAAQKQVLEAMSHRMHVDNSVKLIGKLLFGIEKGPEVLNAVRPAGSALVDDWHCLKTMVSFLF

SEQ ID NO: 237 - protein, 1112 aa, Glycine max
MLPTKRPCEGLVAEEETHNNNDNNNNSNNNNNNNNNNNNNNNSNNSSSSSSSLKKNRIAAARTADSTVKNYESTDQSFNNNNSNSGNASEGASDMALGESNQPDIDEDLHSRQLAVYGRETMRRLFGSNVLVSGMQGLGVEIAKNLILAGVKSVTLHDEGTVELWDLSSNFVFSENDVGKNRAAASVSKLQELNNAVIVQSLTTQLTKEHLSNFQAVVFTDISLEKAFEFNDYCHSHQPPIAFIKTEVRGLFGSVFCDFGPEFTVVDVDGEEPHTGIIASISNDNPALVSCVDDERLEFQDGDLVVFSEVHGMKELNDGKPRKIKDARAYSFTLEEDTTNYGTYEKGGIVTQVKQPKVLNFKPLKEAITDPGDFLLSDFSKFDRPPLLHLAFQALDKFISELGRFPVAGSEDDAQKLISVASHINDSLRDGKLEDINPKLLRYFAFGSRAVLNPMAAMFGGIVGQEVVKACSGKFYPLFQFFYFDSVESLPSEPVDPNDFRPVNGRYDAQISVFGQKLQKKLEDSKVFVVGSGALGCEFLKNLALMGVSCGSQGKLTITDDDVIEKSNLSRQFLFRDWNIGQAKSTVAASAAAAINPSFNIEALQNRVGTETENVFNDTFWENLSVVVNALDNVNARLYVDQRCLYFQKPLLESGTLGAKCNTQMVIPHLTENYGASRDPPEKQAPMCTVHSFPHNIDHCLTWARSEFEGLLEKTPAEVNAYLSNPSEYTNAMKNAGDAQARDNLERVLECLDQEKCETFEDCITWARLKFEDYFVNRVKQLIYTFPEDAATSTGAPFWSAPKRFPRPLQFSASDLGHLNFVSSASILRAETFGIPIPDWGKNPRKMAEAVDRVIVPDFQPKKDVKIVTDEKATSLSTASIDDAAVINDLVIKLERCRANLPPVFMMKPIQFEKDDDTNYHMDVIAGLANMRARNYSIPEVDKLKAKFIAGRIIPAIATSTAMATGLVCLELYKVLDGGHKVEDYRNTFANLALPLFSMAEPVPPKIIKHQDMSWTVWDRWILGDNPTLRELLEWLKAKGLNAYSISCGSCLLYNSMFPRHKDRMDKKVADLAREVAKFEILAYRRHLDVVVACEDDEDNDIDIPQISIYFR

SEQ ID NO: 238 - protein, 514 aa, Glycine max
MDGPAGRGGAGLDMFLPNYKLGKTLGIGSFGKVKIAEHVLTGHKVAIKILNRRKIKNMEMEEKVRREIKILRLFMHPHIIRLYEVIETPTDIYVVMEYVKSGELFDYIVEKGRLQEDEARNFFQQIISGVEYCHRNMVVHRDLKPENLLLDSKCNVKIADFGLSNIMRDGHFLKTSCGSPNYAAPEVISGKLYAGPEVDVWSCGVILYALLCGTLPFDDENIPNLFKKIKGGIYTLPSHLSPGARDLIPGMLVVDPMRRMTIPEIRQHPWFQARLPRYLAVPPPDTMQQAKKIDEEILQEVVKMGFDRNQLVESLGNRIQNEGTVAYYLLLDNRFRVSSGYLGAEFQETMDSGFNQMHSSELASSVVGNRFPGYMEYPGVGSRQQFPVERKWALGLQSRAHPREIMTEVLKALQELNVCWKKIGHYNMKCRWVAGIPGHHEGMVNNNVHSNHYFGDDSNIIENDAVSTSNVVKFEVQLYKTREEKYLLDLQRVQGPQFLFLDLCAAFLAQLRVL

SEQ ID NO: 239 - protein, 472 aa, Glycine max
MEGKKKTTNCLLFLFLSLTLSLSLSAALQLQTQTLPLHSLPHPPAISWPESESEPDPEEEALSLHLHHIDALSSNKTPEQLFQLRLQRDAKRVEGVVALAALNQSHARRSGSSFSSSIISGLAQGSGEYFTRIGVGTPARYVYMVLDTGSDVVWLQCAPCRKCYTQADPVFDPTKSRTYAGIPCGAPLCRRLDSPGCNNKNKVCQYQVSYGDGSFTFGDFSTETLTFRRTRVTRVALGCGHDNEGLFIGAAGLLGLGRGRLSFPVQTGRRFNQKFSYCLVDRSASAKPSSVVFGDSAVSRTARFTPLIKNPKLDTFYYLELLGISVGGSPVRGLSASLFRLDAAGNGGVIIDSGTSVTRLTRPAYIALRDAFRVGASHLKRAAEFSLFDTCFDLSGLTEVKVPTVVLHFRGADVSLPATNYLIPVDNSGSFCFAFAGTMSGLSIIGNIQQQGFRVSFDLAGSRVGFAPRGCV

SEQ ID NO: 240 - protein, 438 aa, Glycine max
MESATKNQVFFCFFLCLLLVSASSLSASKSTHTFPKKALPTKHGHLPISPTSTSSIFYAFYEAQNSTLPLSRTPLLIWLQGGPGCSSMIGNFYELGPWRVTESLTLQRNHGAWNRIFSLLFLDSPIGTGFSVASTRQEIPTDQNHVAKHLFAAITRFVQLDPLFKHRPIYITGESYGGKYVPAIGYHILKKNAQLHVSQRVNLAGVAIGDGLTDPETQVVTHALNAYYVGLINEKQKNELEKAQLEAVRLAQMGNWSEATDARNNVMNMLRNMTGLATLYDYTKKARYQDYLVEKFLNIAKVKKALGVNESFVYELCSDVVEAALHADVMKSVKYMVEYLVRRSKVLLYQGQNDLRAGVVQSEVWVKTMKWEGIVEFVNAERKIWKVNGELAGYVQNWKSLTNVVVLGAGHILPADQVVRSQAMIEDWVLERGLFKSV

SEQ ID NO: 241 - protein, 539 aa, Glycine max
MMYVMTEIKMRSESEVEFKEKMASITGPQILLHSGSPWLKQTMKPSSSPFLLFPKHTPRRTFCSINSNSKGSSSIVPDLLHYLNHSWTQFHATAEAKRQLLAAGFHMLDENHDWHLKPGGRYFFTRNMSCLVAFAIGDKYNVGDGFHVIAAHTDSPCLKLKPKTASCKCNYSMVNVQTYGAGLWYTWFDRDLSVAGRVILRSSHNSYVHKLVKINRPILRIPTLAIHLDRTVNQDGFKPNLETHLHPLLSMKPEDTSLESNSKEKNSALSSKAHHHRLLMQVLSDELNCDIDDIVNIELNVCDTQPSCLGGGNNEFIFSGRLDNLASSYCALRALIDSCQSPGDLASESAIRMVALFDNEEVGSGSVQGAGAPTMFQAMRRIVGDLANNYVSEGSFERTIRQSFLVSADMAHGVHPNFMDKHEELHRPELQKGLVIKHNANQRYATSGITSFLFKEVGKIHNLPTQEFAVRNDMGCGSTIGPILASGVGIRTVDCGIAQLSMHSIREICGKEDIDIAYKHFKAFYQNFSSVDKMLTVDT

SEQ ID NO: 242 - DNA, 354 bp, Glycine max
  source 1..354: mol_type = genomic DNA
atgaaggtcctcatccttgcctgcctggtggctctggcccttgcagccaacttgaggctgagtaagcttggcctcctcatgaaaagtgatcatcaacactcaaatgatgatgagtcatcaaaaccatgctgtgatcaatgcgcatgcacaaagtcaaaccctcctcaatgccgctgttcagatatgagactgaattcgtgccattcagcttgtaaatcgtgtatttgcgcattatcgtatcctgcacagtgtttttgtgttgacataaccgatttctgctatgaaccttgcaagcccagtgaggatgacaaggaaaacgcttcgcctcaggagtctcagaagagagctgtggct

SEQ ID NO: 243 - DNA, 366 bp, Glycine max
  source 1..366: mol_type = genomic DNA
atggccaagctagttttttccctttgttttctgcttttcagtggctgctgcttcgcagccaacttgaggctgagtaagcttggcctcctcatgaaaagtgatcatcaacactcaaatgatgatgagtcatcaaaaccatgctgtgatcaatgcgcatgcacaaagtcaaaccctcctcaatgccgctgttcagatatgagactgaattcgtgccattcagcttgtaaatcgtgtatttgcgcattatcgtatcctgcacagtgtttttgtgttgacataaccgatttctgctatgaaccttgcaagcccagtgaggatgacaaggaaaacgcttcgcctcaggagtctcagaagagagctgtggct

SEQ ID NO: 244 - DNA, 330 bp, Glycine max
  source 1..330: mol_type = genomic DNA
atggtggtgctaaaggtgtgtttggtgctacttttccttgtggggggtactactagtgccaacttgaggctgagtaagcttggcctcctcatgaaaagtgatcatcaacactcaaatgatgatgagtcatcaaaaccatgctgtgatcaatgcgcatgcacaaagtcaaaccctcctcaatgccgctgttcagatatgagactgaattcgtgccattcagcttgtaaatcgtgtatttgcgcattatcgtatcctgcacagtgtttttgtgttgacataaccgatttctgctatgaaccttgcaagcccagtgaggatgacaaggaaaac

SEQ ID NO: 245 - DNA, 330 bp, Glycine max; identical to SEQ ID NO: 244
  source 1..330: mol_type = genomic DNA

SEQ ID NO: 246 - DNA, 12977 bp, Glycine max
  source 1..12977: mol_type = genomic DNA
gtgccgaattcggatccggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggccaagctagttttttccctttgttttctgcttttcagtggctgctgcttcgcaatgcgtccgaagcacccaatcaagcatcaagggcttccccaagaggtactgaacgaaaatctactcagattcttcgtggctccatttcctgaggtatttgggaaggaaaaggtgaatgagctgtctaaagacataggctctgaatcaactgaggaccaagctatggaggatataaagcaaatggaggccgaatccatatcctcaagcgaggaaatcgttccgaattccgtcgaacagaagcatattcaaaaggaggatgtgccttctgagcgataccttggatatcttgaacagttgctgagactaaagaaatacaaggtcccccaactagaaattgttcctaactcagccgaagagcgattacactctatgaaagaggggattcatgctcaacagaaggagcctatgatcggtgtgaatcaagaactggcatacttctatcccgaactgtttcgacaattttatcaattagatgcgtatccatccggggcgtggtattatgtgcctcttgggacacagtatacggatgcgccgagtttttcagacatcccaaacccaataggcagtgagaattcagaaaagacgacaatgcctctttggagttcggggaagccgatcccgaatccgttgttgggtcttgacagtacgcatcatcaccatcaccaccctcaggagtctcagaagagagctgtggcttaggcttatatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttcccccattcaagttacttatgaaatagaaatcctcttctccgactactttatttgtccgaaagtcttgtggctgctatataacgctgcaagaattcaagcttagcggtcctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcttctcgcatatctcattaaagcaggacaagcttacttgtacagctcgtccatgccgtgagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtagctgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccactagaattcgagctcggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggccaagctagttttttccctttgttttctgcttttcagtggctgctgcttcgcaatgagagaacttgaagaattgaatgttccaggagagattgttgaaagtctctcctcttctgaggaaagcatcacaagaatcaacaagaaaattgagaagtttcagagtgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccaaaataccctgtggagcctttcacagaatctcaaagcttgactctcactgatgttgagaatcttcatttgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtttcgcctcaggagtctcagaagagagctgtggcttaagcttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatccgctttacgaattcccatggggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggccaagctagttttttccctttgttttctgcttttcagtggctgctgcttcgcaatgcaggagcaaaaccaagagcaacctattcgttgtgaaaaagacgagagattttttagtgataaaattgcaaagtacatcccaatccagtacgttttaagtcgatatccgagctacggtcttaattactatcaacagaagccagttgccttgataaacaaccagtttctcccatatccctactatgctaagccggccgccgtccgttctcctgctcagatattgcagtggcaggttttatccaacactgtcccggcgaagtcttgccaggcgcaacccaccaccatggctagacacccgcatccgcacctttcctttatggctattcctcccaaaaagaaccaagacaagacagagatcccgaccataaatactatcgcttctggtgagccaacttcaacaccgactactgaagcggtggagagcacggtcgccacacttgaagattccccagaggtgattgaatcacccccggagataaacacggtacaggtcacctcaacggctgtttcggattacaaagatgacgacgataagcctcaggagtctcagaagagagctgtggcttaggcttttatgttggtgatatggtggtaaatgtagggatttagtttacaattgcgtatgtctgtgttggatatctgtagtgctgttcttatggcttagatcttgtaatttctcattacagtatcaatgaatagatatcagtttctagtgatgacattggttcgtcttttagctgttgattaatttttcttaattgattcatcctattgcaattcttctgaatttaaattgtatactgtgaaattaagaaaattcttgaaattaatgagaatttgagtaatagcgctcagagaattcgcatgcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgataccatggcaaacaagctcttcctcgtctgcgcaactttcgccctctgcttcctcctcaccaacgcaatgttgttacctaaactcgaaaggtcagctgccagaccatccggggaaccaggttgttcatgtgcccaaccagctgccgaagccgcagcacctggctgggcccaagcaaggggtcaccccggtggagaacttgaggcagctgcatctgcagctggagatgcagggtggccaaacaagcacaccctgagaatactccaggatttctctagtgatccatctagtaatcttacctcccatagtcttgagaagctgccacctgctgccgaagcagccgagggcgcacccccaggacaagatcctggcgttaggcgtcctcctgacccagcccaccgcccacttccccgcgatcctggcccaaggggcccagtgctcccacccggtttgagtggtgatggatctctgctgaccaggttgtttcaacaccccttgtaccaggtacctattcctccactgacagagggtgatgtgttgtttaacgttaacagcgacataagatttaatccaaaggcagcaaccgctgaaaaccctgattggccacatgaaggaccagaagatgagttcttacctactggagaggcagcagtcgattcttatccaaattggttaaagttccatataggtattaaccgctatgagctttacagtaggcacaatcctgctgttggagctttgctccaagatttgggtacacaaaagatcactagcgtagctatgaagtcaggtggcacccaacttaagctgatcatgacatttcagaattacggccaagcattgttcaaacctatgaagcagactagggaacaagagacacctcccgattttttctatttcagtgactacgaaaggcacaatgccgagatcgctgccttccacctcgacagaatccttgacttccgtcgcgttccaccagttgcaggaaggcttgttaacatgacaaaggaaattagggacgttactcgcgataagaagctttggaggactttcttcatatcaccagcaaacaacgtgtgcttctacggagaatgttcatattactgttccactgaacatgcactgtgtgggaaaccagatcaaatcgaagggtcactcgccgcttttttacctgatctcgcactcgccaagagaaaaacatggcgcaatccttggagaaggagttaccataaacgcaaaaaagctgagtgggaggttgatccagattactgtgaggaagtgagacaaactccaccatatgactctagtcatcgtttgctcgatgtcatggatatgactatctttgacttcttgatggggaatatggatagacaccactatgaaacatttgaaaagttcggtaatgaaactttcattattcaccttgataacggaagaggttttggaaagcactcccatgatgagctttctatattagtaccattgcagcagtgctgtcgcataaggaggtcaacctatctcagattgcagttgctcgcacaggaagagcacagactcagcctgctcatggcagaggctttgcgtgccgatagagtcgctccagtccttttccaaccacacctggaggcccttgatagaagattgagaattgttctccgtgcagtaggggattgcgtcgaaaaggacggtttgcacagtgtggttgaggacgacctgggacccgagcatcgcgccgcagcaggcaggggaggttcaggtggttctggaggttcgtggagccacccccaatttgagaaacctcaggagtctcagaagagagctgtggcttaggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgcttgtggaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 247 - DNA, 13981 bp, Glycine max
  source 1..13981: mol_type = genomic DNA
cacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactctgtgccgaattcggatccggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggccaagctagttttttccctttgttttctgcttttcagtggctgctgcttcgcaatgcgtccgaagcacccaatcaagcatcaagggcttccccaagaggtactgaacgaaaatctactcagattcttcgtggctccatttcctgaggtatttgggaaggaaaaggtgaatgagctgtctaaagacataggctctgaatcaactgaggaccaagctatggaggatataaagcaaatggaggccgaatccatatcctcaagcgaggaaatcgttccgaattccgtcgaacagaagcatattcaaaaggaggatgtgccttctgagcgataccttggatatcttgaacagttgctgagactaaagaaatacaaggtcccccaactagaaattgttcctaactcagccgaagagcgattacactctatgaaagaggggattcatgctcaacagaaggagcctatgatcggtgtgaatcaagaactggcatacttctatcccgaactgtttcgacaattttatcaattagatgcgtatccatccggggcgtggtattatgtgcctcttgggacacagtatacggatgcgccgagtttttcagacatcccaaacccaataggcagtgagaattcagaaaagacgacaatgcctctttggagttcggggaagccgatcccgaatccgttgttgggtcttgacagtacgcatcatcaccatcaccaccctcaggagtctcagaagagagctgtggcttaggcttatatgtcaacagtgagaaactgttcgcattttccgttttgcttctttctttctattcaatgtatgttgttggattccagttgaatttattatgagaactaataataatagtaataatcatttgtttctttactaatttgcattttcacatatgatttctggtgcatatcataattttcattccaccaatattaatttcccccattcaagttacttatgaaatagaaatcctcttctccgactactttatttgtccgaaagtcttgtggctgctatataacgctgcaagaattcaagcttagcggtcctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcttctcgcatatctcattaaagcaggacaagcttacttgtacagctcgtccatgccgtgagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtagctgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccactagaattcgagctcggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggccaagctagttttttccctttgttttctgcttttcagtggctgctgcttcgcaatgagagaacttgaagaattgaatgttccaggagagattgttgaaagtctctcctcttctgaggaaagcatcacaagaatcaacaagaaaattgagaagtttcagagtgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccaaaataccctgtggagcctttcacagaatctcaaagcttgactctcactgatgttgagaatcttcatttgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtttcgcctcaggagtctcagaagagagctgtggcttaagcttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatccgctttacgaattcccatggggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggccaagctagttttttccctttgttttctgcttttcagtggctgctgcttcgcaatgcaggagcaaaaccaagagcaacctattcgttgtgaaaaagacgagagattttttagtgataaaattgcaaagtacatcccaatccagtacgttttaagtcgatatccgagctacggtcttaattactatcaacagaagccagttgccttgataaacaaccagtttctcccatatccctactatgctaagccggccgccgtccgttctcctgctcagatattgcagtggcaggttttatccaacactgtcccggcgaagtcttgccaggcgcaacccaccaccatggctagacacccgcatccgcacctttcctttatggctattcctcccaaaaagaaccaagacaagacagagatcccgaccataaatactatcgcttctggtgagccaacttcaacaccgactactgaagcggtggagagcacggtcgccacacttgaagattccccagaggtgattgaatcacccccggagataaacacggtacaggtcacctcaacggctgtttcggattacaaagatgacgacgataagcctcaggagtctcagaagagagctgtggcttaggcttttatgttggtgatatggtggtaaatgtagggatttagtttacaattgcgtatgtctgtgttggatatctgtagtgctgttcttatggcttagatcttgtaatttctcattacagtatcaatgaatagatatcagtttctagtgatgacattggttcgtcttttagctgttgattaatttttcttaattgattcatcctattgcaattcttctgaatttaaattgtatactgtgaaattaagaaaattcttgaaattaatgagaatttgagtaatagcgctcagagaattcgcatgcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgataccatggcaaacaagctcttcctcgtctgcgcaactttcgccctctgcttcctcctcaccaacgcaatgttgttacctaaactcgaaaggtcagctgccagaccatccggggaaccaggttgttcatgtgcccaaccagctgccgaagccgcagcacctggctgggcccaagcaaggggtcaccccggtggagaacttgaggcagctgcatctgcagctggagatgcagggtggccaaacaagcacaccctgagaatactccaggatttctctagtgatccatctagtaatcttacctcccatagtcttgagaagctgccacctgctgccgaagcagccgagggcgcacccccaggacaagatcctggcgttaggcgtcctcctgacccagcccaccgcccacttccccgcgatcctggcccaaggggcccagtgctcccacccggtttgagtggtgatggatctctgctgaccaggttgtttcaacaccccttgtaccaggtacctattcctccactgacagagggtgatgtgttgtttaacgttaacagcgacataagatttaatccaaaggcagcaaccgctgaaaaccctgattggccacatgaaggaccagaagatgagttcttacctactggagaggcagcagtcgattcttatccaaattggttaaagttccatataggtattaaccgctatgagctttacagtaggcacaatcctgctgttggagctttgctccaagatttgggtacacaaaagatcactagcgtagctatgaagtcaggtggcacccaacttaagctgatcatgacatttcagaattacggccaagcattgttcaaacctatgaagcagactagggaacaagagacacctcccgattttttctatttcagtgactacgaaaggcacaatgccgagatcgctgccttccacctcgacagaatccttgacttccgtcgcgttccaccagttgcaggaaggcttgttaacatgacaaaggaaattagggacgttactcgcgataagaagctttggaggactttcttcatatcaccagcaaacaacgtgtgcttctacggagaatgttcatattactgttccactgaacatgcactgtgtgggaaaccagatcaaatcgaagggtcactcgccgcttttttacctgatctcgcactcgccaagagaaaaacatggcgcaatccttggagaaggagttaccataaacgcaaaaaagctgagtgggaggttgatccagattactgtgaggaagtgagacaaactccaccatatgactctagtcatcgtttgctcgatgtcatggatatgactatctttgacttcttgatggggaatatggatagacaccactatgaaacatttgaaaagttcggtaatgaaactttcattattcaccttgataacggaagaggttttggaaagcactcccatgatgagctttctatattagtaccattgcagcagtgctgtcgcataaggaggtcaacctatctcagattgcagttgctcgcacaggaagagcacagactcagcctgctcatggcagaggctttgcgtgccgatagagtcgctccagtccttttccaaccacacctggaggcccttgatagaagattgagaattgttctccgtgcagtaggggattgcgtcgaaaaggacggtttgcacagtgtggttgaggacgacctgggacccgagcatcgcgccgcagcaggcaggggaggttcaggtggttctggaggttcgtggagccacccccaatttgagaaacctcaggagtctcagaagagagctgtggcttaggcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgagcgaattccatatgggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggtggtgctaaaggtgtgtttggtgctacttttccttgtggggggtactactagtgccaacttgaggctgagtaagcttggcctcctcatgaaaagtgatcatcaacactcaaatgatgatgagtcatcaaaaccatgctgtgatcaatgcgcatgcacaaagtcaaaccctcctcaatgccgctgttcagatatgagactgaattcgtgccattcagcttgtaaatcgtgtatttgcgcattatcgtatcctgcacagtgtttttgtgttgacataaccgatttctgctatgaaccttgcaagcccagtgaggatgacaaggaaaacgcttcgcctcaggagtctcagaagagagctgtggcttaagcttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatccgct

SEQ ID NO: 248 - DNA, 1008 bp, Glycine max
  source 1..1008: mol_type = genomic DNA
atttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagttaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgccatggtggtgctaaaggtgtgtttggtgctacttttccttgtggggggtactactagtgccaacttgaggctgagtaagcttggcctcctcatgaaaagtgatcatcaacactcaaatgatgatgagtcatcaaaaccatgctgtgatcaatgcgcatgcacaaagtcaaaccctcctcaatgccgctgttcagatatgagactgaattcgtgccattcagcttgtaaatcgtgtatttgcgcattatcgtatcctgcacagtgtttttgtgttgacataaccgatttctgctatgaaccttgcaagcccagtgaggatgacaaggaaaacgcttcgcctcaggagtctcagaagagagctgtggcttaagcttggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatc

SEQ ID NO: 249 - DNA, 204 bp, Glycine max
  source 1..204: mol_type = genomic DNA
ctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagat

SEQ ID NO: 250 - DNA, 631 bp, Glycine max
  source 1..631: mol_type = genomic DNA
atgacaagcatgatggtgtgaggatgaggccatcttatgaaataataacaaataaataaattttgtatgataataaaaagtatggcccatgtaccatcccagcgagcctatgtttatatctgagtggcgttgtacctttcaatcgccttaataaaatgtcagtgttcacgttttgtctttattctgtgtttattttcttttttgtgggcaagctagcttttatctacttttaaatgagtaatactgattatatgtttactggggaattatttgatctccatataaatcttgtttgaaataaacggaagtaattaatttaaaatattttcttaatattttgagcacctcttgtgttctatcgtctttttgggaagagctctcttgtgttctatcctcttatgaaataataagatcagcacaacttacctaaattaaattatctttcaatttgatatgcatatatttatttgattttttaatatatttttaacttattttaataaattaataaaatacgtgaaatagcctctaatttacaacaatgtgcctactgagaagatatattgagctgctttttacagaaaatttggaagtcttgataaattataaggcaaggtattctgtgcaaagt